(19) United States
(12) Patent Application Publication
(10) Pub. No.: US 2023/0049379 A1
(43) Pub. Date: Feb. 16, 2023

(54) CHROMOSOME CONFORMATION MARKERS OF PROSTATE CANCER AND LYMPHOMA

(71) Applicant: Oxford BioDynamics PLC, Oxford (GB)

(72) Inventors: Ewan Hunter, Oxford (GB); Aroul Ramadass, Oxford (GB); Alexandre Akoulitchev, Oxford (GB)

(73) Assignee: Oxford BioDynamics PLC, Oxford (GB)

(21) Appl. No.: 17/609,273

(22) PCT Filed: May 6, 2020
(86) PCT No.: PCT/GB2020/051105

§ 371 Date: Nov. 5, 2021

(30) Foreign Application Priority Data
May 8, 2019 (GB) 1906487.2
Oct. 11, 2019 (GB) 1914729.7
Apr. 29, 2020 (GB) 2006286.5

(51) Int. Cl. C12Q 1/6886; G01N 33/50; C12Q 1/686

(52) U.S. Cl. CPC C12Q 1/6886; G01N 33/5011; C12Q 1/686; C12Q 2600/118; C12Q 2600/112

(57) ABSTRACT

A process for analysing chromosome regions and interactions relating to prognosis of prostate cancer or DLBCL.

### CROSS-REFERENCE

This application is a 371 National Stage filing and claims the benefit under 35 U.S.C. § 120 of International Application No. PCT/GB2020/051105, filed 6 May 2020, which claims priority to Great Britain Application No. GB1906487.2, filed 8 May 2019, Great Britain Application No. GB1914729.7, filed 11 Oct. 2019, and Great Britain Application No. GB2006286.5, filed 29 Apr. 2020, each of which is incorporated herein by reference in its entirety.

### SEQUENCE LISTING INCORPORATION BY REFERENCE

The application herein incorporates by reference in its entirety the sequence listing material in the ASCII text file named “20.05.20 P104645W001 Sequence Listing”, created May 13, 2020, and having the size of 75,431 bytes, filed with this application.

### FIELD OF THE INVENTION

The invention relates to disease processes.

### BACKGROUND OF THE INVENTION

The regulatory and causative aspects of the disease process in cancer are complex and cannot be easily elucidated using available DNA and protein typing methods.

Diffuse large B-cell lymphoma (DLBCL) is a cancer of B cells, a type of white blood cell responsible for producing antibodies. It is the most common type of non-Hodgkin lymphoma among adults, with an annual incidence of 7-8 cases per 100,000 people per year in the USA and the UK. However, there is a poor understanding of the outcomes of the disease process.

Prostate cancer is caused by the abnormal and uncontrolled growth of cells in the prostate. Whilst prostate cancer survival rates have been improving from decade to decade, the disease is still considered largely incurable. According to the American Cancer Society, for all stages of prostate cancer combined, the one-year relative survival rate is 20%, and the five-year rate is 7%.

### SUMMARY OF THE INVENTION

The inventors have identified subtypes of patients in prostate cancer, diffuse large B-cell lymphoma (DLBCL) and lymphoma defined by chromosome conformation signatures.

According the invention provides a process for detecting a chromosome state which represents a subgroup in a population comprising determining whether a chromosome interaction relating to that chromosome state is present or absent within a defined region of the genome; and
wherein said chromosome interaction has optionally been identified by a method of determining which chromosomal interactions are relevant to a chromosome state corresponding to the subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to the subgroup; and
wherein the subgroup relates to prognosis for prostate cancer and the chromosome interaction either:
   (i) is present in any one of the regions or genes listed in Table 6; and/or
   (ii) corresponds to any one of the chromosome interactions represented by any probe shown in Table 6, and/or
   (iii) is present in a 4,000 base region which comprises or which flanks (i) or (ii);
   or
wherein the subgroup relates to prognosis for DLBCL and the chromosome interaction either:
   a) is present in any one of the regions or genes listed in Table 5; and/or
   b) corresponds to any one of the chromosome interactions represented by any probe shown in Table 5, and/or
   c) is present in a 4,000 base region which comprises or which flanks (a) or (b);
   or
wherein the subgroup relates to prognosis for lymphoma and the chromosome interaction either:
   (iv) is present in any one of the regions or genes listed in Table 8; and/or
   (v) corresponds to any one of the chromosome interactions shown in Table 8, and/or
   (vi) is present in a 4,000 base region which comprises or which flanks (iv) or (v).

### BRIEF DESCRIPTION OF THE DRAWINGS

FIG. 1 shows a Principle Component Analysis (PCA) for the prostate cancer work.

FIG. 2 shows a VENN comparison of the two PCA prognostic classifiers.

FIG. 3 shows a PCA analysis for DLBCL.

FIG. 4 shows a PCA for the 7 BTK markers (OBD RD051) in DLBCL.

FIG. 5 shows an example of how the chromosome interaction typing may be carried out.

FIG. 6 shows markers from the canine lymphoma work which can be used in the method of the invention. The Figure shows marker reduction. 70% of 38 samples were used as a training set (28) and used for marker selection. The remaining 10 were used as a test set. Multiple training and test sets were used. Univariant analysis, Fisher's Exact test (column D and E results) and Multivariant analysis Penalized logistic modelling (GLMNET, columns B and C results). The markers 2 to 18 are lymphoma markers and 19 to 23 are controls. The top 11, which are all loops present in lymphoma were selected for classification.

FIG. 7 shows canine markers to human genes. The table shows the top 11 canine markers mapped to the human genome (Hg38) with the closest mapping genomic region. The network adjacent is built using the 11 markers (dark) the nodes which are a lighter colour and linker proteins using the NCI database.

FIG. 8 shows canine markers to human genes. As before but with pathway enrichment for the network. Only the 11 canine mapping loci were used for enrichment, the linking modes were omitted from enrichment. Nodes in lighter colour belong to the KEGG CML pathway.

FIG. 9 shows Training Set 1 and Test Set 1×GBoost 11 Mark Model FIG. 10 shows Training Set 2 and Test Set 2×GBoost 11 Mark Model FIG. 11 shows Training Set 3 and Test Set 3×GBoost 11 Mark Model FIG. 12 shows Training Set 1 Logistic PCA

FIG. 13 shows Training Set 1 and Test Set 1 Logistic PCA. The logistic PCA model was used to predict the Test set 1 (triangles). Darker triangles are lymphoma (labelled) from the test set, the lighter triangles are the controls from the test set. The training Lymphoma samples are in darker colour and Controls are in lighter colour.

FIG. 14 shows Training Set 1 and Test Set 1 ROC & AUC

FIG. 15 shows Patient PFS EpiSwitch™ Call and Loop dynamic at NFKB1. 118 patients called either ABC or GCB using EpiSwitch™ 10 marker human model, PFS modelling using this call and dynamic of loop, GCB with loop don't die, shows also that human model works well for disease prognostics.

FIG. 16 shows 118 patient PFS EpiSwitch™ Call and loop dynamic at NFATC1. As before but for NFATC1, again this shows that human model for prognostics using the marker as one of the 10 human markers is a very good at classification.

FIG. 17 shows three-step approach to identify, evaluate, and validate diagnostic and prognostic biomarkers for prostate cancer (PCa).

FIG. 18 shows PCA for the five-markers applied to 78 samples containing two groups. First group, 49 known samples (24 PCa and 25 healthy controls (Cntrl)) combined with a second group of 29 samples including, 24 PCa samples and 5 healthy Cntrl samples.

FIG. 19 shows the workflow to develop a classifier.

FIG. 20 shows relevant gene groups for the classifier.

FIG. 21 shows overlap of the EpiSwitch DLBCL-CCS and Fluidigm subtype calls and ROC Curve when applied to the Discovery cohort. A. Subtype calls made by the EpiSwitch DLBCL-CCS and the Fluidigm assays on samples of known subtypes. 60 out of 60 samples were identically called by both assays. B. The receiver operating curve (ROC) for the DLBCL-CCS when applied to the Discovery cohort. C. Kaplan-Meier survival analysis (by progression free survival) of samples called as ABC or GCB by the DLBCL-CCS. Samples called as ABC showed a significantly poorer long-term survival than those called as GCB.

FIG. 22 shows assignment of DLBCL subtypes in Type III samples by EpiSwitch and Fluidigm assays.

FIG. 23 shows comparison of baseline DLBCL subtype calls in Type III samples using EpiSwitch and Fluidigm with long term survival. Kaplan-Meier survival curves for the 58 DLBCL patients classified as either ABC, GCB or Unclassified by the Fluidigm assay (A) or the EpiSwitch DLBCL-CCS (B). Fluidigm classified 15 samples as ABC, 22 as GCB and 21 were UNC. EpiSwitch classified 34 as ABC and 24 as GCB.

FIG. 24 shows mean survival time by EpiSwitch and Fluidigm classification in the Validation cohort.

FIG. 25 shows initial assessment of likely DLBCL subtype.

FIG. 26 shows PCA of DLBCL patients with baseline ABC/GCB subtype calls by EpiSwitch in the Discovery cohort.

### DETAILED DESCRIPTION OF THE INVENTION

### Aspects of the Invention

The invention concerns determining prognosis in prostate cancer, particularly in respect to whether the cancer is aggressive or indolent. This determining is by typing any of the relevant markers discloses herein, for example in Table 6, or preferred combinations of markers, or markers in defined specific regions disclosed herein. Thus the invention relating to a method of typing a patient with prostate cancer to identify whether the cancer is aggressive or indolent.

The invention also concerns determining prognosis in DLBCL, particularly in respect to whether the prognosis is good or poor in respect of survival. This determining is by typing any of the relevant markers discloses herein, for example in Table 5, or preferred combinations of markers, or markers in defined specific regions disclosed herein. Thus the invention relates to a method of typing a patient with DLBCL to identify whether the patient has good or poor prognosis in respect of survival, for example to determine expected rate of development of disease and/or time to death.

Essentially in the method of the invention subpopulations of prostate cancer or DLBCL identified by typing of the markers. Therefore the invention, for example, concerns a panel of epigenetic markers which relates to prognosis in these conditions. The invention therefore allows personalised therapy to be given to the patient which accurately reflects the patient's needs.

The invention also relates to determining prognosis for lymphoma based on typing chromosome interactions defined by Tables 8 or 9.

Tables 5 to 7 preferably relate to determining prognosis in humans. Tables 8 and 9 preferably relate to determining prognosis in canines.

Any therapy, for example drug, which is mentioned herein may be administered to an individual based on the result of the method.

Marker sets are disclosed in the Tables and Figures. In one embodiment at least 10 markers from any disclosed marker set are used in the invention. In another embodiment at least 20% of the markers from any disclosed marker set are used in the invention.

The Process of the Invention

The process of the invention comprises a typing system for detecting chromosome interactions relevant to prognosis. This typing may be performed using the EpiSwitch™ system mentioned herein which is based on cross-linking regions of chromosome which have come together in the chromosome interaction, subjecting the chromosomal DNA to cleavage and then ligating the nucleic acids present in the cross-linked entity to derive a ligated nucleic acid with sequence from both the regions which formed the chromosomal interaction. Detection of this ligated nucleic acid allows determination of the presence or absence of a particular chromosome interaction.

The chromosomal interactions may be identified using the above described method in which populations of first and second nucleic acids are used. These nucleic acids can also be generated using EpiSwitch™ technology.

### The Epigenetic Interactions Relevant to the Invention

As used herein, the term ‘epigenetic’ and ‘chromosome’ interactions typically refers to interactions between distal regions of a chromosome, said interactions being dynamic and altering, forming or breaking depending upon the status of the region of the chromosome.

In particular processes of the invention chromosome interactions are typically detected by first generating a ligated nucleic acid that comprises sequence from both regions of the chromosomes that are part of the interactions. In such processes the regions can be cross-linked by any suitable means. In a preferred aspect, the interactions are cross-linked using formaldehyde, but may also be cross-linked by any aldehyde, or D-Biotinoyl-e-aminocaproic acid-N-hydroxysuccinimide ester or Digoxigenin-3-O-methylcarbonyl-e-aminocaproic acid-N-hydroxysuccinimide ester. Para-formaldehyde can cross link DNA chains which are 4 Angstroms apart. Preferably the chromosome interactions are on the same chromosome and optionally 2 to 10 Angstroms apart.

The chromosome interaction may reflect the status of the region of the chromosome, for example, if it is being transcribed or repressed in response to change of the physiological conditions. Chromosome interactions which are specific to subgroups as defined herein have been found to be stable, thus providing a reliable means of measuring the differences between the two subgroups.

In addition, chromosome interactions specific to a characteristic (such as prognosis) will normally occur early in a biological process, for example compared to other epigenetic markers such as methylation or changes to binding of histone proteins. Thus the process of the invention is able to detect early stages of a biological process. This allows early intervention (for example treatment) which may as a consequence be more effective. Chromosome interactions also reflect the current state of the individual and therefore can be used to assess changes to prognosis. Furthermore there is little variation in the relevant chromosome interactions between individuals within the same subgroup. Detecting chromosome interactions is highly informative with up to 50 different possible interactions per gene, and so processes of the invention can interrogate 500,000 different interactions.

Preferred Marker Sets

Herein the term ‘marker’ or ‘biomarker’ refers to a specific chromosome interaction which can be detected (typed) in the invention. Specific markers are disclosed herein, any of which may be used in the invention. Further sets of markers may be used, for example in the combinations or numbers disclosed herein. The specific markers disclosed in the tables herein are preferred as well as markers presents in genes and regions mentioned in the tables herein are preferred. These may be typed by any suitable method, for example the PCR or probe based methods disclosed herein, including a qPCR method. The markers are defined herein by location or by probe and/or primer sequences.

### Location and Causes of Epigenetic Interactions

Epigenetic chromosomal interactions may overlap and include the regions of chromosomes shown to encode relevant or undescribed genes, but equally may be in intergenic regions. It should further be noted that the inventors have discovered that epigenetic interactions in all regions are equally important in determining the status of the chromosomal locus. These interactions are not necessarily in the coding region of a particular gene located at the locus and may be in intergenic regions.

The chromosome interactions which are detected in the invention could be caused by changes to the underlying DNA sequence, by environmental factors, DNA methylation, non-coding antisense RNA transcripts, non-mutagenic carcinogens, histone modifications, chromatin remodelling and specific local DNA interactions. The changes which lead to the chromosome interactions may be caused by changes to the underlying nucleic acid sequence, which themselves do not directly affect a gene product or the mode of gene expression. Such changes may be for example, SNPs within and/or outside of the genes, gene fusions and/or deletions of intergenic DNA, microRNA, and non-coding RNA. For example, it is known that roughly 20% of SNPs are in non-coding regions, and therefore the process as described is also informative in non-coding situation. In one aspect the regions of the chromosome which come together to form the interaction are less than 5 kb, 3 kb, 1 kb, 500 base pairs or 200 base pairs apart on the same chromosome.

The chromosome interaction which is detected is preferably within any of the genes mentioned in Table 5. However it may also be upstream or downstream of the gene, for example up to 50,000, up to 30,000, up to 20,000, up to 10,000 or up to 5000 bases upstream or downstream from the gene or from the coding sequence.

The chromosome interaction which is detected is preferably within any of the genes mentioned in Table 6. However it may also be upstream or downstream of the gene, for example up to 50,000, up to 30,000, up to 20,000, up to 10,000 or up to 5000 bases upstream or downstream from the gene or from the coding sequence.

The chromosome interaction which is detected is preferably within any of the genes mentioned in Table 9. However it may also be upstream or downstream of the gene, for example up to 50,000, up to 30,000, up to 20,000, up to 10,000 or up to 5000 bases upstream or downstream from the gene or from the coding sequence.

Subgroups, Time Points and Personalised Treatment

The aim of the present invention is to determine prognosis. This may be at one or more defined time points, for example at at least 1, 2, 5, 8 or 10 different time points. The durations between at least 1, 2, 5 or 8 of the time points may be at least 5, 10, 20, 50, 80 or 100 days.

As used herein, a “subgroup” preferably refers to a population subgroup (a subgroup in a population), more preferably a subgroup in the population of a particular animal such as a particular eukaryote, or mammal (e.g. human, non-human, non-human primate, or rodent e.g. mouse or rat). Most preferably, a “subgroup” refers to a subgroup in the human population. The subgroup may be a canine subgroup, such as a dog.

The invention includes detecting and treating particular subgroups in a population. The inventors have discovered that chromosome interactions differ between subsets (for example at least two subsets) in a given population. Identifying these differences will allow physicians to categorize their patients as a part of one subset of the population as described in the process. The invention therefore provides physicians with a process of personalizing medicine for the patient based on their epigenetic chromosome interactions.

In one aspect the invention relates to testing whether an individual:
is a fast or slow ‘progressor’, and/or
has an aggressive or indolent form of disease.

The invention may also determine the expected survival time of the individual.

Such testing may be used to select how to subsequently treat the patient, for example the type of drug and/or its dose and/or its frequency of administration.

### Generating Ligated Nucleic Acids

Certain aspects of the invention utilise ligated nucleic acids, in particular ligated DNA. These comprise sequences from both of the regions that come together in a chromosome interaction and therefore provide information about the interaction. The EpiSwitch™ method described herein uses generation of such ligated nucleic acids to detect chromosome interactions.

Thus a process of the invention may comprise a step of generating ligated nucleic acids (e.g. DNA) by the following steps (including a method comprising these steps):

(i) cross-linking of epigenetic chromosomal interactions present at the chromosomal locus, preferably in vitro;
(ii) optionally isolating the cross-linked DNA from said chromosomal locus;
(iii) subjecting said cross-linked DNA to cutting, for example by restriction digestion with an enzyme that cuts it at least once (in particular an enzyme that cuts at least once within said chromosomal locus);
(iv) ligating said cross-linked cleaved DNA ends (in particular to form DNA loops); and
(v) optionally identifying the presence of said ligated DNA and/or said DNA loops, in particular using techniques such as PCR (polymerase chain reaction), to identify the presence of a specific chromosomal interaction.

These steps may be carried out to detect the chromosome interactions for any aspect mentioned herein. The steps may also be carried out to generate the first and/or second set of nucleic acids mentioned herein.

PCR (polymerase chain reaction) may be used to detect or identify the ligated nucleic acid, for example the size of the PCR product produced may be indicative of the specific chromosome interaction which is present, and may therefore be used to identify the status of the locus. In preferred aspects at least 1, 2 or 3 primers or primer pairs as shown in Table 5 are used in the PCR reaction. In other aspects at least 1, 10, 20, 30, 50 or 80 or the primers or primer pairs as shown in Table 6 are used in the PCR reaction. The skilled person will be aware of numerous restriction enzymes which can be used to cut the DNA within the chromosomal locus of interest. It will be apparent that the particular enzyme used will depend upon the locus studied and the sequence of the DNA located therein. A non-limiting example of a restriction enzyme which can be used to cut the DNA as described in the present invention is Taql.

### EpiSwitch™ Technology

The EpiSwitch™ Technology also relates to the use of microarray EpiSwitch™ marker data in the detection of epigenetic chromosome conformation signatures specific for phenotypes. Aspects such as EpiSwitch™ which utilise ligated nucleic acids in the manner described herein have several advantages. They have a low level of stochastic noise, for example because the nucleic acid sequences from the first set of nucleic acids of the present invention either hybridise or fail to hybridise with the second set of nucleic acids. This provides a binary result permitting a relatively simple way to measure a complex mechanism at the epigenetic level. EpiSwitch™ technology also has fast processing time and low cost. In one aspect the processing time is 3 hours to 6 hours.

### Samples and Sample Treatment

The process of the invention will normally be carried out on a sample. The sample may be obtained at a defined time point, for example at any time point defined herein. The sample will normally contain DNA from the individual. It will normally contain cells. In one aspect a sample is obtained by minimally invasive means, and may for example be a blood sample. DNA may be extracted and cut up with a standard restriction enzyme. This can pre-determine which chromosome conformations are retained and will be detected with the EpiSwitch™ platforms. Due to the synchronisation of chromosome interactions between tissues and blood, including horizontal transfer, a blood sample can be used to detect the chromosome interactions in tissues, such as tissues relevant to disease. For certain conditions, such as cancer, genetic noise due to mutations can affect the chromosome interaction ‘signal’ in the relevant tissues and therefore using blood is advantageous.

### Properties of Nucleic Acids of the Invention

The invention relates to certain nucleic acids, such as the ligated nucleic acids which are described herein as being used or generated in the process of the invention. These may be the same as, or have any of the properties of, the first and second nucleic acids mentioned herein. The nucleic acids of the invention typically comprise two portions each comprising sequence from one of the two regions of the chromosome which come together in the chromosome interaction. Typically each portion is at least 8, 10, 15, 20, 30 or 40 nucleotides in length, for example 10 to 40 nucleotides in length. Preferred nucleic acids comprise sequence from any of the genes mentioned in any of the tables. Typically preferred nucleic acids comprise the specific probe sequences mentioned in Table 5; or fragments and/or homologues of such sequences. The preferred nucleic acids may comprise the specific probe sequences mentioned in Table 6; or fragments and/or homologues of such sequences.

Preferably the nucleic acids are DNA. It is understood that where a specific sequence is provided the invention may use the complementary sequence as required in the particular aspect. Preferably the nucleic acids are DNA. It is understood that where a specific sequence is provided the invention may use the complementary sequence as required in the particular aspect.

The primers shown in Table 5 may also be used in the invention as mentioned herein. In one aspect primers are used which comprise any of: the sequences shown in Table 5; or fragments and/or homologues of any sequence shown in Table 5. The primers shown in Table 6 may also be used in the invention as mentioned herein. In one aspect primers are used which comprise any of: the sequences shown in Table 6; or fragments and/or homologues of any sequence shown in Table 6. The primers shown in Table 8 may also be used in the invention as mentioned herein. In one aspect primers are used which comprise any of: the sequences shown in Table 8; or fragments and/or homologues of any sequence shown in Table 8.

### The Second Set of Nucleic Acids—the ‘Index’ Sequences

The second set of nucleic acid sequences has the function of being a set of index sequences, and is essentially a set of nucleic acid sequences which are suitable for identifying subgroup specific sequence. They can represents the ‘background’ chromosomal interactions and might be selected in some way or be unselected. They are in general a subset of all possible chromosomal interactions.

The second set of nucleic acids may be derived by any suitable process. They can be derived computationally or they may be based on chromosome interaction in individuals. They typically represent a larger population group than the first set of nucleic acids. In one particular aspect, the second set of nucleic acids represents all possible epigenetic chromosomal interactions in a specific set of genes. In another particular aspect, the second set of nucleic acids represents a large proportion of all possible epigenetic chromosomal interactions present in a population described herein. In one particular aspect, the second set of nucleic acids represents at least 50% or at least 80% of epigenetic chromosomal interactions in at least 20, 50, 100 or 500 genes, for example in 20 to 100 or 50 to 500 genes.

The second set of nucleic acids typically represents at least 100 possible epigenetic chromosome interactions which modify, regulate or in any way mediate a phenotype in population. The second set of nucleic acids may represent chromosome interactions that affect a disease state (typically relevant to diagnosis or prognosis) in a species. The second set of nucleic acids typically comprises sequences representing epigenetic interactions both relevant and not relevant to a prognosis subgroup.

In one particular aspect the second set of nucleic acids derive at least partially from naturally occurring sequences in a population, and are typically obtained by in silico processes. Said nucleic acids may further comprise single or multiple mutations in comparison to a corresponding portion of nucleic acids present in the naturally occurring nucleic acids. Mutations include deletions, substitutions and/or additions of one or more nucleotide base pairs. In one particular aspect, the second set of nucleic acids may comprise sequence representing a homologue and/or orthologue with at least 70% sequence identity to the corresponding portion of nucleic acids present in the naturally occurring species. In another particular aspect, at least 80% sequence identity or at least 90% sequence identity to the corresponding portion of nucleic acids present in the naturally occurring species is provided.

### Properties of the Second Set of Nucleic Acids

In one particular aspect, there are at least 100 different nucleic acid sequences in the second set of nucleic acids, preferably at least 1000, 2000 or 5000 different nucleic acids sequences, with up to 100,000, 1,000,000 or 10,000,000 different nucleic acid sequences. A typical number would be 100 to 1,000,000, such as 1,000 to 100,000 different nucleic acids sequences. All or at least 90% or at least 50% or these would correspond to different chromosomal interactions.

In one particular aspect, the second set of nucleic acids represent chromosome interactions in at least 20 different loci or genes, preferably at least 40 different loci or genes, and more preferably at least 100, at least 500, at least 1000 or at least 5000 different loci or genes, such as 100 to 10,000 different loci or genes. The lengths of the second set of nucleic acids are suitable for them to specifically hybridise according to Watson Crick base pairing to the first set of nucleic acids to allow identification of chromosome interactions specific to subgroups. Typically the second set of nucleic acids will comprise two portions corresponding in sequence to the two chromosome regions which come together in the chromosome interaction. The second set of nucleic acids typically comprise nucleic acid sequences which are at least 10, preferably 20, and preferably still 30 bases (nucleotides) in length. In another aspect, the nucleic acid sequences may be at the most 500, preferably at most 100, and preferably still at most 50 base pairs in length. In a preferred aspect, the second set of nucleic acids comprises nucleic acid sequences of between 17 and 25 base pairs. In one aspect at least 100, 80% or 50% of the second set of nucleic acid sequences have lengths as described above. Preferably the different nucleic acids do not have any overlapping sequences, for example at least 100%, 90%, 80% or 50% of the nucleic acids do not have the same sequence over at least 5 contiguous nucleotides.

Given that the second set of nucleic acids acts as an ‘index’ then the same set of second nucleic acids may be used with different sets of first nucleic acids which represent subgroups for different characteristics, i.e. the second set of nucleic acids may represent a ‘universal’ collection of nucleic acids which can be used to identify chromosome interactions relevant to different characteristics.

### The First Set of Nucleic Acids

The first set of nucleic acids are typically from subgroups relevant to prognosis. The first nucleic acids may have any of the characteristics and properties of the second set of nucleic acids mentioned herein. The first set of nucleic acids is normally derived from samples from the individuals which have undergone treatment and processing as described herein, particularly the EpiSwitch™ cross-linking and cleaving steps. Typically the first set of nucleic acids represents all or at least 80% or 50% of the chromosome interactions present in the samples taken from the individuals.

Typically, the first set of nucleic acids represents a smaller population of chromosome interactions across the loci or genes represented by the second set of nucleic acids in comparison to the chromosome interactions represented by second set of nucleic acids, i.e. the second set of nucleic acids is representing a background or index set of interactions in a defined set of loci or genes.

### Library of Nucleic Acids

Any of the types of nucleic acid populations mentioned herein may be present in the form of a library comprising at least 200, at least 500, at least 1000, at least 5000 or at least 10000 different nucleic acids of that type, such as ‘first’ or ‘second’ nucleic acids. Such a library may be in the form of being bound to an array. The library may comprise some or all of the probes or primer pairs shown in Table 5 or 6. The library may comprise all of the probe sequence from any of the tables disclosed herein.

### Hybridisation

The invention requires a means for allowing wholly or partially complementary nucleic acid sequences from the first set of nucleic acids and the second set of nucleic acids to hybridise. In one aspect all of the first set of nucleic acids is contacted with all of the second set of nucleic acids in a single assay, i.e. in a single hybridisation step. However any suitable assay can be used.

### Labelled Nucleic Acids and Pattern of Hybridisation

The nucleic acids mentioned herein may be labelled, preferably using an independent label such as a fluorophore (fluorescent molecule) or radioactive label which assists detection of successful hybridisation. Certain labels can be detected under UV light. The pattern of hybridisation, for example on an array described herein, represents differences in epigenetic chromosome interactions between the two subgroups, and thus provides a process of comparing epigenetic chromosome interactions and determination of which epigenetic chromosome interactions are specific to a subgroup in the population of the present invention.

The term ‘pattern of hybridisation’ broadly covers the presence and absence of hybridisation between the first and second set of nucleic acids, i.e. which specific nucleic acids from the first set hybridise to which specific nucleic acids from the second set, and so it not limited to any particular assay or technique, or the need to have a surface or array on which a ‘pattern’ can be detected.

Selecting a Subgroup with Particular Characteristics

The invention provides a process which comprises detecting the presence or absence of chromosome interactions, typically 5 to 20 or 5 to 500 such interactions, preferably 20 to 300 or 50 to 100 interactions, in order to determine the presence or absence of a characteristic relating to prognosis in an individual. Preferably the chromosome interactions are those in any of the genes mentioned herein. In one aspect the chromosome interactions which are typed are those represented by the nucleic acids in Table 5. In another aspect the chromosome interactions are those represented in Table 6. In a further aspect the chromosome interactions which are typed are those represented by the nucleic acids in Table 8. The column titled ‘Loop Detected’ in the tables shows which subgroup is detected by each probe. Detection can either of the presence or absence of the chromosome interaction in that subgroup, which is what ‘1’ and ‘-1’ indicate.

The Individual that is Tested

Examples of the species that the individual who is tested is from are mentioned herein. In addition the individual that is tested in the process of the invention may have been selected in some way. The individual may be susceptible to any condition mentioned herein and/or may be in need of any therapy mentioned in. The individual may be receiving any therapy mentioned herein. In particular, the individual may have, or be suspected of having, prostate cancer or DLBCL. The individual may have, or be suspected of having, a lymphoma.

### Preferred Gene Regions, Loci, Genes and Chromosome Interactions for Prostate Cancer

For all aspects of the invention preferred gene regions, loci, genes and chromosome interactions are mentioned in the tables, for example in Table 6. Typically in the processes of the invention chromosome interactions are detected from at least 1, 2, 3, 4 or 5 of the relevant genes listed in Table 6. Preferably the presence or absence of at least 1, 2, 3, 4 or 5 of the relevant specific chromosome interactions represented by the probe sequences in Table 6 are detected. The chromosome interaction may be upstream or downstream of any of the genes mentioned herein, for example 50 kb upstream or 20 kb downstream, for example from the coding sequence.

For all aspects of the invention preferred gene regions, loci, genes and chromosome interactions are mentioned in Table 25. Typically in the processes of the invention chromosome interactions are detected from at least 2, 4, 8, 10, 14 or all of the relevant genes listed in Table 25. Preferably the presence or absence of at least 2, 4, 8, 10, 14 or all of the relevant specific chromosome interactions shown in Table 25 are detected. The chromosome interaction may be upstream or downstream of any of the genes mentioned herein, for example 50 kb upstream or 20 kb downstream, for example from the coding sequence.

In one embodiment a combination of specific markers disclosed herein and represented by (identified by) the following combination of genes is typed: ETS1, MAP3K14, SLC22A3 and CASP2. This may be to determine diagnosis. Preferably at least 2 or 3 of these markers are typed.

In another embodiment a combination of specific markers disclosed herein represented by (identified by) the following combination of genes is typed: BMP6, ERG, MSR1, MUC1, ACAT1 and DAPK1. This may be to determine prognosis (High-risk Category 3 vs Low Risk Category 1, by Nested PCR Markers). Preferably at least 2 or 3 of these markers are typed.

In a further embodiment a combination of specific markers disclosed herein represented by (identified by) the following combination of genes is typed: HSD3B2, VEGFC, APAF1, MUC1, ACAT1 and DAPK1. This may be to determine prognosis (High Risk Cat 3 vs Medium Risk Cat 2). Preferably at least 2 or 3 of these markers are typed.

### Preferred Gene Regions, Loci, Genes and Chromosome Interactions for DLBCL

Typically at least 10, 20, 30, 50 or 80 chromosome interactions are typed from any of genes or regions disclosed the tables herein, or parts of tables disclosed herein. Preferably at least 10, 20, 30, 50 or 80 chromosome interactions are typed from any of the genes or regions disclosed in Table 5.

Preferably at least 2, 3, 5, 8 of the markers of Table 7 are typed.

Preferably the presence or absence of at least 10, 20, 30, 50 or 80 chromosome interactions represented by the probe sequences in Table 5 are detected. The chromosome interaction may be upstream or downstream of any of the genes mentioned herein, for example 50 kb upstream or 20 kb downstream, for example from the coding sequence.

Preferably at least 1, 2, 5, 8 or all of the first 10 markers shown in Table 5 is typed. In one embodiment at least 1, 2, 3 or 6 markers from Table 5 are typed each corresponding to a different gene selected from STAT3, TNFRSF13B, ANXA11, MAP3K7, MEF2B and IFNAR1.

### Preferred Gene Regions, Loci, Genes and Chromosome Interactions for Lymphoma

Typically at least 10, 20, 30 or 50 chromosome interactions are typed from any of the genes or regions disclosed the tables herein, or parts of tables disclosed herein. Preferably at least 10, 20, 30 or 50 chromosome interactions are typed from any of the genes or regions disclosed in Table 8.

Preferably at least 5, 10 or 15 of the markers of Table 9 are typed.

The chromosome interaction may be upstream or downstream of any of the genes mentioned herein, for example 50 kb upstream or 20 kb downstream, for example from the coding sequence.

In one embodiment at least one of the first 11 markers shown in FIG. 6 is typed. In another embodiment at least 1, 2, 3 or 6 markers from Table 8 are typed each corresponding to a different gene selected from: STAT3, TNFRSF13B, ANXA11, MAP3K7, MEF2B and IFNAR1.

### Types of Chromosome Interaction

In one aspect the locus (including the gene and/or place where the chromosome interaction is detected) may comprise a CTCF binding site. This is any sequence capable of binding transcription repressor CTCF. That sequence may consist of or comprise the sequence CCCTC which may be present in 1, 2 or 3 copies at the locus. The CTCF binding site sequence may comprise the sequence CCGCGNGGNGGCAG (SEQ ID NO:1) (in IUPAC notation). The CTCF binding site may be within at least 100, 500, 1000 or 4000 bases of the chromosome interaction or within any of the chromosome regions shown Table 5 or 6. The CTCF binding site may be within at least 100, 500, 1000 or 4000 bases of the chromosome interaction or within any of the chromosome regions shown Table 5 or 6.

In one aspect the chromosome interactions which are detected are present at any of the gene regions shown Table 5 or 6. In the case where a ligated nucleic acid is detected in the process then sequence shown in any of the probe sequences in Table 5 or 6 may be detected.

Thus typically sequence from both regions of the probe (i.e. from both sites of the chromosome interaction) could be detected. In preferred aspects probes are used in the process which comprise or consist of the same or complementary sequence to a probe shown in any table. In some aspects probes are used which comprise sequence which is homologous to any of the probe sequences shown in the tables.

### Tables Provided Herein

Tables 5 and 6 shows probe (Episwitch™ marker) data and gene data representing chromosome interactions relevant to prognosis. The probe sequences show sequence which can be used to detect a ligated product generated from both sites of gene regions that have come together in chromosome interactions, i.e. the probe will comprise sequence which is complementary to sequence in the ligated product. The first two sets of Start-End positions show probe positions, and the second two sets of Start-End positions show the relevant 4 kb region. The following information is provided in the probe data table:
HyperG_Stats: p-value for the probability of finding that number of significant EpiSwitch™ markers in the locus based on the parameters of hypergeometric enrichment
Probe Count Total: Total number of EpiSwitch™ Conformations tested at the locus
Probe Count Sig: Number of EpiSwitch™ Conformations found to be statistically significant at the locus
FDR HyperG: Multi-test (Fimmunoresposivenesse Discovery Rate) corrected hypergeometric p-value
Percent Sig: Percentage of significant EpiSwitch™ markers relative the number of markers tested at the locus
logFC: logarithm base 2 of Epigenetic Ratio (FC)
AveExpr: average log 2-expression for the probe over all arrays and channels
T: moderated t-statistic
p-value: raw p-value
adj. p-value: adjusted p-value or q-value
B—B-statistic (lods or B) is the log-odds that that gene is differentially expressed.
FC—non-log Fold Change
FC_1—non-log Fold Change centred around zero
LS— Binary value this relates to FC_1 values. FC_1 value below −1.1 it is set to −1 and if the FC_1 value is above 1.1 it is set to 1. Between those values the value is 0

Tables 5 and 6 shows genes where a relevant chromosome interaction has been found to occur. The p-value in the loci table is the same as the HyperG Stats (p-value for the probability of finding that number of significant EpiSwitch™ markers in the locus based on the parameters of hypergeometric enrichment). The LS column shows presence or absence of the relevant interaction with that particular subgroup (prognosis status).

For table 5, DLBCL refers to prognosis marker, indicated with 1, and healthy refers to healthy control, indicated with −1.

The probes are designed to be 30 bp away from the Taq1 site. In case of PCR, PCR primers are typically designed to detect ligated product but their locations from the Taq1 site vary.

Probe locations:
Start 1—30 bases upstream of Taql site on fragment 1
End 1—Taql restriction site on fragment 1
Start 2—Taql restriction site on fragment 2
End 2—30 bases downstream of Taql site on fragment 2

### 4 kb Sequence Location:

Start 1—4000 bases upstream of Taql site on fragment 1
End 1—Taql restriction site on fragment 1
Start 2—Taql restriction site on fragment 2
End 2—4000 bases downstream of Taql site on fragment 2
GLMNET values related to procedures for fitting the entire lasso or elastic-net regularization (Lambda set to 0.5 (elastic-net)).

In the tables herein the prostate cancer aggressive subgroup refers to class 3 patients with the following description:
PSA level is more than 20 ng/ml, and
the Gleason score is between 8 and 10, and
the T stage is T2c, T3 or T4

In the tables herein the prostate cancer indolent subgroup refers to class 1 patient with the following description:
the PSA level is less than 10 ng per ml, and
the Gleason score is no higher than 6, and
the T stage is between T1 and T2a.

Table 7 shows preferred markers for DLBCL. Tables 8 and 9 show preferred markers for lymphoma.

Tables 5 to 7 are preferably for typing humans. Tables 8 and 9 are preferably for typing canines, for examples dogs.

### The Approach Taken to Identify Markers and Panels of Markers

The invention described herein relates to chromosome conformation profile and 3D architecture as a regulatory modality in its own right, closely linked to the phenotype. The discovery of biomarkers was based on annotations through pattern recognition and screening on representative cohorts of clinical samples representing the differences in phenotypes. We annotated and screened significant parts of the genome, across coding and non-coding parts and over large sways of non-coding 5″ and 3″ of known genes for identification of statistically disseminating consistent conditional disseminating chromosome conformations, which for example anchor in the non-coding sites within (intronic) or outside of open reading frames

In selection of the best markers we are driven by statistical data and p values for the marker leads. The reference to the particular genes is used for the ease of the position reference—the closest genes are usually used for the reference. It is impossible to exclude the possibility, that a chromosome conformation in the cis-position and relevant vicinity from a gene might be contributing a specific component of regulation into expression of that particular gene. At the point of marker selection or validation expression parameters are not needed on the genes referenced as location coordinates in the names of chromosome conformations. Selected and validated chromosome conformations within the signature are disseminating stratifying entities in their own right, irrespective of the expression profiles of the genes used in the reference. Further work may be done on relevant regulatory modalities, such as SNPs at the anchoring sites, changes in gene transcription profiles, changes at the level of H3K27ac.

We are taking the question of clinical phenotype differences and their stratification from the basis of fundamental biology and epigenetics controls over phenotype—including for example from the framework of network of regulation. As such, to assist stratification, one can capture changes in the network and it is preferably done through signatures of several biomarkers, for example through following a machine learning algorithm for marker reduction which includes evaluating the optimal number of markers to stratify the testing cohort with minimal noise. This usually ends with 3-17 markers, depending on case by case basis. Selection of markers for panels may be done by cross-validation statistical performance (and not for example by the functional relevance of the neighbouring genes, used for the reference name).

A panel of markers (with names of adjacent genes) is a product of clustered selection from the screening across significant parts of the genome, in non-biased way analysing statistical disseminating powers over 14,000-60,000 annotated EpiSwitch sites across significant parts of the genome. It should not be perceived as a tailored capture of a chromosome conformation on the gene of know functional value for the question of stratification. The total number of sites for chromosome interaction are 1.2 million, and so the potential number of combinations is 1.2 million to the power 1.2 million. The approach that we have followed nevertheless allows the identifying of the relevant chromosome interactions.

The specific markers that are provided by this application have passed selection, being statistically (significantly) associated with the condition. This is what the p-value in the relevant table demonstrates. Each marker can be seen as representing an event of biological epigenetic as part of network deregulation that is manifested in the relevant condition. In practical terms it means that these markers are prevalent across groups of patients when compared to controls. On average, as an example, an individual marker may typically be present in 80% of patients tested and in 10% of controls tested.

Simple addition of all markers would not represent the network interrelationships between some of the deregulations. This is where the standard multivariate biomarker analysis GLMNET (R package) is brought in. GLMNET package helps to identify interdependence between some of the markers, that reflect their joint role in achieving deregulations leading to disease phenotype. Modelling and then testing markers with highest GLMNET scores offers not only identify the minimal number of markers that accurately identifies the patient cohort, but also the minimal number that offers the least false positive results in the control group of patients, due to background statistical noise of low prevalence in the control group. Typically a group (combination) of selected markers (such as 3 to 10) offers the best balance between both sensitivity and specificity of detection, emerging in the context of multivariate analysis from individual properties of all the selected statistical significant markers for the condition.

The tables herein show the reference names for the array probes (60-mer) for array analysis that overlaps the juncture between the long range interaction sites, the chromosome number and the start and end of two chromosomal fragments that come into juxtaposition. The tables also show standard array readouts in competitive hybridisation of disease versus control samples (labeled with two different fluorescent colours) for each of the markers. As a standard readout it shows for each marker probe:
an average expression signal
t test for significant difference between fluorescent colour detection for controls and for disease samples
p value of significance of the marker readout
adjusted p-value (using Bonferroni correction for the large data set, B—background signal, FC—fold change for the colour detection in control sample
FC_1—fold change for the second colour detection in the case (disease or disease type) sample, LS (Loop Status)—prevalent fluorescent signal between two colours threshold in competitive hybridisations, with—1 meaning signal is prevent in patient samples with corresponding fluorescent colour, when tested against the probe on the CGH array
immediate genetic loci
Prob Count Total—how many different location probes on the array were tested across that genetic locus
Prob Count Sig—how many of them turned out to be significant in discriminating between case and control samples
Hypergeometric Stat is statistics of enrichment of the locus with significant probes for disease detection
FDR HyperG is the same statistics adjusted for the large data set by FDR (standard procedure)
percentage of probes that turned to be significant in that locus
logFC is logarithm of the fold change in array readout for that probe. Attention to the loci with high enrichment of significant probes helps selection of the top probes representing regulatory hubs with multiple inputs associated with disease providing markers with best coverage of for example network deregulation.

### Preferred Aspects for Sample Preparation and Chromosome Interaction Detection

Methods of preparing samples and detecting chromosome conformations are described herein. Optimised (non-conventional) versions of these methods can be used, for example as described in this section.

Typically the sample will contain at least 2×10⁵ cells. The sample may contain up to 5×10⁵ cells. In one aspect, the sample will contain 2×10⁵ to 5.5×10⁵ cells

Crosslinking of epigenetic chromosomal interactions present at the chromosomal locus is described herein. This may be performed before cell lysis takes place. Cell lysis may be performed for 3 to 7 minutes, such as 4 to 6 or about 5 minutes. In some aspects, cell lysis is performed for at least 5 minutes and for less than 10 minutes.

Digesting DNA with a restriction enzyme is described herein. Typically, DNA restriction is performed at about 55° C. to about 70° C., such as for about 65° C., for a period of about 10 to 30 minutes, such as about 20 minutes.

Preferably a frequent cutter restriction enzyme is used which results in fragments of ligated DNA with an average fragment size up to 4000 base pair. Optionally the restriction enzyme results in fragments of ligated DNA have an average fragment size of about 200 to 300 base pairs, such as about 256 base pairs. In one aspect, the typical fragment size is from 200 base pairs to 4,000 base pairs, such as 400 to 2,000 or 500 to 1,000 base pairs.

In one aspect of the EpiSwitch method a DNA precipitation step is not performed between the DNA restriction digest step and the DNA ligation step.

DNA ligation is described herein. Typically the DNA ligation is performed for 5 to 30 minutes, such as about 10 minutes.

The protein in the sample may be digested enzymatically, for example using a proteinase, optionally Proteinase K. The protein may be enzymatically digested for a period of about 30 minutes to 1 hour, for example for about 45 minutes. In one aspect after digestion of the protein, for example Proteinase K digestion, there is no cross-link reversal or phenol DNA extraction step.

In one aspect PCR detection is capable of detecting a single copy of the ligated nucleic acid, preferably with a binary read-out for presence/absence of the ligated nucleic acid.

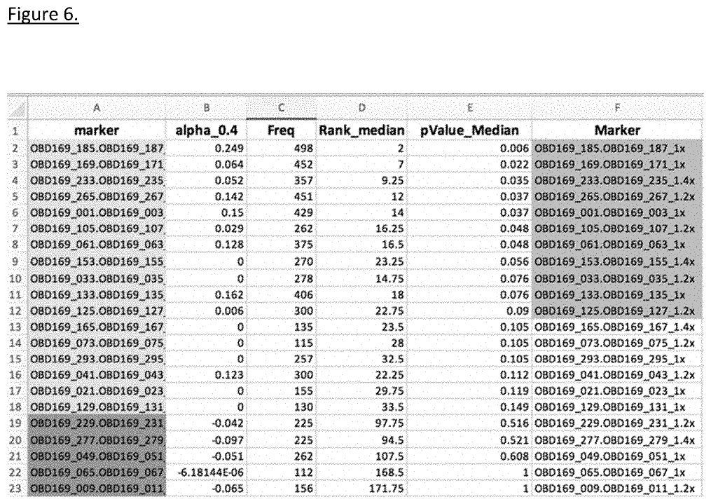

FIG. 5 shows a preferred method of detecting chromosome interactions.

### Processes and Uses of the Invention

The process of the invention can be described in different ways. It can be described as a method of making a ligated nucleic acid comprising (i) in vitro cross-linking of chromosome regions which have come together in a chromosome interaction; (ii) subjecting said cross-linked DNA to cutting or restriction digestion cleavage; and (iii) ligating said cross-linked cleaved DNA ends to form a ligated nucleic acid, wherein detection of the ligated nucleic acid may be used to determine the chromosome state at a locus, and wherein preferably:
the locus may be any of the loci, regions or genes mentioned in Table 5, and/or
wherein the chromosomal interaction may be any of the chromosome interactions mentioned herein or corresponding to any of the probes disclosed in Table 5, and/or
wherein the ligated product may have or comprise (i) sequence which is the same as or homologous to any of the probe sequences disclosed in Table 5; or (ii) sequence which is complementary to (ii).

The process of the invention can be described as a process for detecting chromosome states which represent different subgroups in a population comprising determining whether a chromosome interaction is present or absent within a defined epigenetically active region of the genome, wherein preferably:
the subgroup is defined by presence or absence of prognosis, and/or
the chromosome state may be at any locus, region or gene mentioned in Table 5; and/or
the chromosome interaction may be any of those mentioned in Table 5 or corresponding to any of the probes disclosed in that table.

The process of the invention can be described as a method of making a ligated nucleic acid comprising (i) in vitro cross-linking of chromosome regions which have come together in a chromosome interaction; (ii) subjecting said cross-linked DNA to cutting or restriction digestion cleavage; and (iii) ligating said cross-linked cleaved DNA ends to form a ligated nucleic acid, wherein detection of the ligated nucleic acid may be used to determine the chromosome state at a locus, and wherein preferably:
the locus may be any of the loci, regions or genes mentioned in Table 6, and/or
wherein the chromosomal interaction may be any of the chromosome interactions mentioned herein or corresponding to any of the probes disclosed in Table 6, and/or
wherein the ligated product may have or comprise (i) sequence which is the same as or homologous to any of the probe sequences disclosed in Table 6; or (ii) sequence which is complementary to (ii).

The process of the invention can be described as a process for detecting chromosome states which represent different subgroups in a population comprising determining whether a chromosome interaction is present or absent within a defined epigenetically active region of the genome, wherein preferably:
the subgroup is defined by presence or absence of prognosis, and/or
the chromosome state may be at any locus, region or gene mentioned in Table 6; and/or
the chromosome interaction may be any of those mentioned in Table 6 or corresponding to any of the probes disclosed in that table.

The invention includes detecting chromosome interactions at any locus, gene or regions mentioned Table 5. The invention includes use of the nucleic acids and probes mentioned herein to detect chromosome interactions, for example use of at least 1, 5, 10, 20 or 50 such nucleic acids or probes to detect chromosome interactions. The nucleic acids or probes preferably detect chromosome interactions in at least 1, 5, 10, 20 or 50 different loci or genes. The invention includes detection of chromosome interactions using any of the primers or primer pairs listed in Table 5 or using variants of these primers as described herein (sequences comprising the primer sequences or comprising fragments and/or homologues of the primer sequences).

The invention includes detecting chromosome interactions at any locus, gene or regions mentioned Table 6. The invention includes use of the nucleic acids and probes mentioned herein to detect chromosome interactions. The invention includes detection of chromosome interactions using any of the primers or primer pairs listed in Table 6 or using variants of these primers as described herein (sequences comprising the primer sequences or comprising fragments and/or homologues of the primer sequences).

When analysing whether a chromosome interaction occurs ‘within’ a defined gene, region or location, either both the parts of the chromosome which have together in the interaction are within the defined gene, region or location or in some aspects only one part of the chromosome is within the defined, gene, region or location.

Similarly the chromosome interactions of Tables 8 and 9 may be used in the processes and methods of the invention.

### Use of the Method of the Invention to Identify New Treatments

Knowledge of chromosome interactions can be used to identify new treatments for conditions. The invention provides methods and uses of chromosomes interactions defined herein to identify or design new therapeutic agents, for example relating to therapy of prostate cancer or DLBCL.

### Homologues

Homologues of polynucleotide/nucleic acid (e.g. DNA) sequences are referred to herein. Such homologues typically have at least 70% homology, preferably at least 80%, at least 85%, at least 90%, at least 95%, at least 97%, at least 98% or at least 99% homology, for example over a region of at least 10, 15, 20, 30, 100 or more contiguous nucleotides, or across the portion of the nucleic acid which is from the region of the chromosome involved in the chromosome interaction. The homology may be calculated on the basis of nucleotide identity (sometimes referred to as “hard homology”).

Therefore, in a particular aspect, homologues of polynucleotide/nucleic acid (e.g. DNA) sequences are referred to herein by reference to percentage sequence identity. Typically such homologues have at least 70% sequence identity, preferably at least 80%, at least 85%, at least 90%, at least 95%, at least 97%, at least 98% or at least 99% sequence identity, for example over a region of at least 10, 15, 20, 30, 100 or more contiguous nucleotides, or across the portion of the nucleic acid which is from the region of the chromosome involved in the chromosome interaction.

For example the UWGCG Package provides the BESTFIT program which can be used to calculate homology and/or % sequence identity (for example used on its default settings) (Devereux et al (1984) Nucleic Acids Research 12, p 387-395). The PILEUP and BLAST algorithms can be used to calculate homology and/or % sequence identity and/or line up sequences (such as identifying equivalent or corresponding sequences (typically on their default settings)), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W5 T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by 1, 2, 3, 4 or more bases, such as less than 10, 15 or 20 bases (which may be substitutions, deletions or insertions of nucleotides). These changes may be measured across any of the regions mentioned above in relation to calculating homology and/or % sequence identity.

Homology of a ‘pair of primers’ can be calculated, for example, by considering the two sequences as a single sequence (as if the two sequences are joined together) for the purpose of then comparing against the another primer pair which again is considered as a single sequence.

### Arrays

The second set of nucleic acids may be bound to an array, and in one aspect there are at least 15,000, 45,000, 100,000 or 250,000 different second nucleic acids bound to the array, which preferably represent at least 300, 900, 2000 or 5000 loci. In one aspect one, or more, or all of the different populations of second nucleic acids are bound to more than one distinct region of the array, in effect repeated on the array allowing for error detection. The array may be based on an Agilent SurePrint G3 Custom CGH microarray platform. Detection of binding of first nucleic acids to the array may be performed by a dual colour system.

Therapeutic Agents (for Example which are Selected Based on Typing Individuals or which are Selected Based on Testing According to the Invention)

Therapeutic agents are mentioned herein. The invention provides such agents for use in preventing or treating a disease condition in certain individuals, for example those identified by a process of the invention. This may comprise administering to an individual in need a therapeutically effective amount of the agent. The invention provides use of the agent in the manufacture of a medicament to prevent or treat a condition in certain individuals.

The formulation of the agent will depend upon the nature of the agent. The agent will be provided in the form of a pharmaceutical composition containing the agent and a pharmaceutically acceptable carrier or diluent. Suitable carriers and diluents include isotonic saline solutions, for example phosphate-buffered saline. Typical oral dosage compositions include tablets, capsules, liquid solutions and liquid suspensions. The agent may be formulated for parenteral, intravenous, intramuscular, subcutaneous, transdermal or oral administration.

The dose of an agent may be determined according to various parameters, especially according to the substance used; the age, weight and condition of the individual to be treated; the route of administration; and the required regimen. A physician will be able to determine the required route of administration and dosage for any particular agent. A suitable dose may however be from 0.1 to 100 mg/kg body weight such as 1 to 40 mg/kg body weight, for example, to be taken from 1 to 3 times daily.

The therapeutic agent may be any such agent disclosed herein, or may target any ‘target’ disclosed herein, including any protein or gene disclosed herein in any table (including Table 5 or 6). It is understood that any agent that is disclosed in a combination should be seen as also disclosed for administration individually.

### Prostate Cancer Therapy

Prostate cancer treatments are recommended depending on the stage of disease progression. Radiotherapy, Hormone treatment and Chemotherapy are the three options that are often used in prostate cancer treatment. A single treatment or a combination of treatments may be used.

### Chemotherapy

Chemotherapy is often used to treat prostate cancer that has invaded to other organs of the body (metastatic prostate cancer). Chemotherapy destroys cancer cells by interfering with the way they multiply. Chemotherapy does not cure prostate cancer, but it keeps it under control and reduce symptoms, therefore daily life is less effected.

### Radiotherapy

This treatment may be used to cure localized and locally-advanced prostate cancer. Radiotherapy can also be used to slow the progression of metastatic prostate cancer and relieve symptoms. Patients may receive hormone therapy before undergoing chemotherapy to increase the chance of successful treatment. Hormone therapy may also be recommended after radiotherapy to reduce the chances of relapsing.

Hormone therapy

Hormone therapy is often used in combination with radiotherapy. Hormone therapy alone should not normally be used to treat localised prostate cancer in men who are fit and willing to receive surgery or radiotherapy. Hormone therapy can be used to slow the progression of advanced prostate cancer and relieve symptoms. Hormones control the growth of cells in the prostate. In particular, prostate cancer needs the hormone testosterone to grow. The purpose of hormone therapy is to block the effects of testosterone, either by stopping its production or by stopping patient's body to use testosterone.

Other Treatments that May be Used in Prostate Cancer Therapy
Radical prostatectomy
High intensity focused ultrasound therapy
Cryotherapy
Brachytherapy
Watchful waiting
Trans-urethral resection of the prostate
Treating advanced prostate cancer
Steroid

### DLBCL Therapy

The following four treatments may be used to treat DLBCL:
Chemotherapy
Radiotherapy
Monocolonal antibody therapy
Steroid therapy

Any of the above therapies may also be used to treat lymphoma.

### Forms of the Substance Mentioned Herein

Any of the substances, such as nucleic acids or therapeutic agents, mentioned herein may be in purified or isolated form. They may be in a form which is different from that found in nature, for example they may be present in combination with other substance with which they do not occur in nature. The nucleic acids (including portions of sequences defined herein) may have sequences which are different to those found in nature, for example having at least 1, 2, 3, 4 or more nucleotide changes in the sequence as described in the section on homology. The nucleic acids may have heterologous sequence at the 5′ or 3′ end. The nucleic acids may be chemically different from those found in nature, for example they may be modified in some way, but preferably are still capable of Watson-Crick base pairing. Where appropriate the nucleic acids will be provided in double stranded or single stranded form. The invention provides all of the specific nucleic acid sequences mentioned herein in single or double stranded form, and thus includes the complementary strand to any sequence which is disclosed.

The invention provides a kit for carrying out any process of the invention, including detection of a chromosomal interaction relating to prognosis. Such a kit can include a specific binding agent capable of detecting the relevant chromosomal interaction, such as agents capable of detecting a ligated nucleic acid generated by processes of the invention. Preferred agents present in the kit include probes capable of hybridising to the ligated nucleic acid or primer pairs, for example as described herein, capable of amplifying the ligated nucleic acid in a PCR reaction.

The invention provides a device that is capable of detecting the relevant chromosome interactions. The device preferably comprises any specific binding agents, probe or primer pair capable of detecting the chromosome interaction, such as any such agent, probe or primer pair described herein.

### Detection Methods

In one aspect quantitative detection of the ligated sequence which is relevant to a chromosome interaction is carried out using a probe which is detectable upon activation during a PCR reaction, wherein said ligated sequence comprises sequences from two chromosome regions that come together in an epigenetic chromosome interaction, wherein said method comprises contacting the ligated sequence with the probe during a PCR reaction, and detecting the extent of activation of the probe, and wherein said probe binds the ligation site. The method typically allows particular interactions to be detected in a MIQE compliant manner using a dual labelled fluorescent hydrolysis probe.

The probe is generally labelled with a detectable label which has an inactive and active state, so that it is only detected when activated. The extent of activation will be related to the extent of template (ligation product) present in the PCR reaction. Detection may be carried out during all or some of the PCR, for example for at least 50% or 80% of the cycles of the PCR.

The probe can comprise a fluorophore covalently attached to one end of the oligonucleotide, and a quencher attached to the other end of the nucleotide, so that the fluorescence of the fluorophore is quenched by the quencher. In one aspect the fluorophore is attached to the 5′end of the oligonucleotide, and the quencher is covalently attached to the 3′ end of the oligonucleotide. Fluorophores that can be used in the methods of the invention include FAM, TET, JOE, Yakima Yellow, HEX, Cyanine3, ATTO 550, TAMRA, ROX, Texas Red, Cyanine 3.5, LC610, LC 640, ATTO 647N, Cyanine 5, Cyanine 5.5 and ATTO 680. Quenchers that can be used with the appropriate fluorophore include TAM, BHQ1, DAB, Eclip, BHQ2 and BBQ650, optionally wherein said fluorophore is selected from HEX, Texas Red and FAM. Preferred combinations of fluorophore and quencher include FAM with BHQ1 and Texas Red with BHQ2.

Use of the Probe in a qPCR Assay

Hydrolysis probes of the invention are typically temperature gradient optimised with concentration matched negative controls. Preferably single-step PCR reactions are optimized. More preferably a standard curve is calculated. An advantage of using a specific probe that binds across the junction of the ligated sequence is that specificity for the ligated sequence can be achieved without using a nested PCR approach. The methods described herein allow accurate and precise quantification of low copy number targets. The target ligated sequence can be purified, for example gel-purified, prior to temperature gradient optimization. The target ligated sequence can be sequenced. Preferably PCR reactions are performed using about 10 ng, or 5 to 15 ng, or 10 to 20 ng, or 10 to 50 ng, or 10 to 200 ng template DNA. Forward and reverse primers are designed such that one primer binds to the sequence of one of the chromosome regions represented in the ligated DNA sequence, and the other primer binds to other chromosome region represented in the ligated DNA sequence, for example, by being complementary to the sequence.

### Choice of Ligated DNA Target

The invention includes selecting primers and a probe for use in a PCR method as defined herein comprising selecting primers based on their ability to bind and amplify the ligated sequence and selecting the probe sequence based properties of the target sequence to which it will bind, in particular the curvature of the target sequence.

Probes are typically designed/chosen to bind to ligated sequences which are juxtaposed restriction fragments spanning the restriction site. In one aspect of the invention, the predicted curvature of possible ligated sequences relevant to a particular chromosome interaction is calculated, for example using a specific algorithm referenced herein. The curvature can be expressed as degrees per helical turn, e.g. 10.5° per helical turn. Ligated sequences are selected for targeting where the ligated sequence has a curvature propensity peak score of at least 5° per helical turn, typically at least 10°, 15° or 20° per helical turn, for example 5° to 20° per helical turn. Preferably the curvature propensity score per helical turn is calculated for at least 20, 50, 100, 200 or 400 bases, such as for 20 to 400 bases upstream and/or downstream of the ligation site. Thus in one aspect the target sequence in the ligated product has any of these levels of curvature. Target sequences can also be chosen based on lowest thermodynamic structure free energy.

### Particular Aspects

In one aspect only intrachromosomal interactions are typed/detected, and no extrachromosomal interactions (between different chromosomes) are typed/detected.

In particular aspects certain chromosome interactions are not typed, for example any specific interaction mentioned herein (for example as defined by any probe or primer pair mentioned herein). In some aspects chromosome interactions are not typed in any of the genes mentioned herein.

The data provided herein shows that the markers are ‘disseminating’ ones able to differentiate cases and non-cases for the relevant disease situation. Therefore when carrying out the invention the skilled person will be able to determine by detection of the interactions which subgroup the individual is in. In one embodiment a threshold value of detection of at least 70% of the tested markers in the form they are associated with the relevant disease situation (either by absence or presence) may be used to determine whether the individual is in the relevant subgroup.

### Screening Method

The invention provides a method of determining which chromosomal interactions are relevant to a chromosome state corresponding to an prognosis subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to an prognosis subgroup. The subgroup may be any of the specific subgroups defined herein, for example with reference to particular conditions or therapies.

### Publications

The contents of all publications mentioned herein are incorporated by reference into the present specification and may be used to further define the features relevant to the invention.

### Specific Aspects

The EpiSwitch™ platform technology detects epigenetic regulatory signatures of regulatory changes between normal and abnormal conditions at loci. The EpiSwitch™ platform identifies and monitors the fundamental epigenetic level of gene regulation associated with regulatory high order structures of human chromosomes also known as chromosome conformation signatures. Chromosome signatures are a distinct primary step in a cascade of gene deregulation. They are high order biomarkers with a unique set of advantages against biomarker platforms that utilize late epigenetic and gene expression biomarkers, such as DNA methylation and RNA profiling.

### EpiSwitch′ Array Assay

The custom EpiSwitch™ array-screening platforms come in 4 densities of, 15K, 45K, 100K, and 250K unique chromosome conformations, each chimeric fragment is repeated on the arrays 4 times, making the effective densities 60K, 180K, 400K and 1 Million respectively.

### Custom Designed EpiSwitch™ Arrays

The 15K EpiSwitch™ array can screen the whole genome including around 300 loci interrogated with the EpiSwitch™ Biomarker discovery technology. The EpiSwitch™ array is built on the Agilent SurePrint G3 Custom CGH microarray platform; this technology offers 4 densities, 60K, 180K, 400K and 1 Million probes. The density per array is reduced to 15K, 45K, 100K and 250K as each EpiSwitch™ probe is presented as a quadruplicate, thus allowing for statistical evaluation of the reproducibility. The average number of potential EpiSwitch™ markers interrogated per genetic loci is 50, as such the numbers of loci that can be investigated are 300, 900, 2000, and 5000.

### EpiSwitch™ Custom Array Pipeline

The EpiSwitch™ array is a dual colour system with one set of samples, after EpiSwitch™ library generation, labelled in Cy5 and the other of sample (controls) to be compared/analyzed labelled in Cy3. The arrays are scanned using the Agilent SureScan Scanner and the resultant features extracted using the Agilent Feature Extraction software. The data is then processed using the EpiSwitch™ array processing scripts in R. The arrays are processed using standard dual colour packages in Bioconductor in R: Limma *. The normalisation of the arrays is done using the normalisedWithinArrays function in Limma * and this is done to the on chip Agilent positive controls and EpiSwitch™ positive controls. The data is filtered based on the Agilent Flag calls, the Agilent control probes are removed and the technical replicate probes are averaged, in order for them to be analysed using Limma*. The probes are modelled based on their difference between the 2 scenarios being compared and then corrected by using False Discovery Rate. Probes with Coefficient of Variation (CV)<=30% that are <=−1.1 or =>1.1 and pass the p<=0.1 FDR p-value are used for further screening. To reduce the probe set further Multiple Factor Analysis is performed using the FactorMineR package in R.
Note: LIMMA is Linear Models and Empirical Bayes Processes for Assessing Differential Expression in Microarray Experiments. Limma is an R package for the analysis of gene expression data arising from microarray or RNA-Seq.

The pool of probes is initially selected based on adjusted p-value, FC and CV<30% (arbitrary cut off point) parameters for final picking. Further analyses and the final list are drawn based only on the first two parameters (adj. p-value; FC).

### Statistical Pipeline

EpiSwitch™ screening arrays are processed using the EpiSwitch™ Analytical Package in R in order to select high value EpiSwitch™ markers for translation on to the EpiSwitch™ PCR platform.

### Step 1

Probes are selected based on their corrected p-value (False Discovery Rate, FDR), which is the product of a modified linear regression model. Probes below p-value <=0.1 are selected and then further reduced by their Epigenetic ratio (ER), probes ER have to be <=−1.1 or =>1.1 in order to be selected for further analysis. The last filter is a coefficient of variation (CV), probes have to be below <=0.3.

### Step 2

The top 40 markers from the statistical lists are selected based on their ER for selection as markers for PCR translation. The top 20 markers with the highest negative ER load and the top 20 markers with the highest positive ER load form the list.

### Step 3

The resultant markers from step 1, the statistically significant probes form the bases of enrichment analysis using hypergeometric enrichment (HE). This analysis enables marker reduction from the significant probe list, and along with the markers from step 2 forms the list of probes translated on to the EpiSwitch™ PCR platform.

The statistical probes are processed by HE to determine which genetic locations have an enrichment of statistically significant probes, indicating which genetic locations are hubs of epigenetic difference.

The most significant enriched loci based on a corrected p-value are selected for probe list generation. Genetic locations below p-value of 0.3 or 0.2 are selected. The statistical probes mapping to these genetic locations, with the markers from step 2, form the high value markers for EpiSwitch™ PCR translation.

### Array Design and Processing

### Array Design

1. Genetic loci are processed using the SII software (currently v3.2) to:
   a. Pull out the sequence of the genome at these specific genetic loci (gene sequence with 50 kb upstream and 20 kb downstream)
   b. Define the probability that a sequence within this region is involved in CCs
   c. Cut the sequence using a specific RE
   d. Determine which restriction fragments are likely to interact in a certain orientation
   e. Rank the likelihood of different CCs interacting together.
2. Determine array size and therefore number of probe positions available (x)
3. Pull out x/4 interactions.
4. For each interaction define sequence of 30 bp to restriction site from part 1 and 30 bp to restriction site of part 2. Check those regions aren't repeats, if so exclude and take next interaction down on the list. Join both 30 bp to define probe.
5. Create list of x/4 probes plus defined control probes and replicate 4 times to create list to be created on array
6. Upload list of probes onto Agilent Sure design website for custom CGH array.
7. Use probe group to design Agilent custom CGH array.

### Array Processing

1. Process samples using EpiSwitch™ Standard Operating Procedure (SOP) for template production.
2. Clean up with ethanol precipitation by array processing laboratory.
3. Process samples as per Agilent SureTag complete DNA labelling kit—Agilent Oligonucleotide Array-based CGH for Genomic DNA Analysis Enzymatic labelling for Blood, Cells or Tissues
4. Scan using Agilent C Scanner using Agilent feature extraction software.

EpiSwitch™ biomarker signatures demonstrate high robustness, sensitivity and specificity in the stratification of complex disease phenotypes. This technology takes advantage of the latest breakthroughs in the science of epigenetics, monitoring and evaluation of chromosome conformation signatures as a highly informative class of epigenetic biomarkers. Current research methodologies deployed in academic environment require from 3 to 7 days for biochemical processing of cellular material in order to detect CCSs. Those procedures have limited sensitivity, and reproducibility; and furthermore, do not have the benefit of the targeted insight provided by the EpiSwitch™ Analytical Package at the design stage.

EpiSwitch™ Array in Silico Marker Identification CCS sites across the genome are directly evaluated by the EpiSwitch™ Array on clinical samples from testing cohorts for identification of all relevant stratifying lead biomarkers. The EpiSwitch™ Array platform is used for marker identification due to its high-throughput capacity, and its ability to screen large numbers of loci rapidly. The array used was the Agilent custom-CGH array, which allows markers identified through the in silico software to be interrogated.

### EpiSwitch™ PCR

Potential markers identified by EpiSwitch™ Array are then validated either by EpiSwitch™ PCR or DNA sequencers (i.e. Roche 454, Nanopore MinION, etc.). The top PCR markers which are statistically significant and display the best reproducibility are selected for further reduction into the final EpiSwitch™ Signature Set, and validated on an independent cohort of samples. EpiSwitch™ PCR can be performed by a trained technician following a standardised operating procedure protocol established. All protocols and manufacture of reagents are performed under ISO 13485 and 9001 accreditation to ensure the quality of the work and the ability to transfer the protocols. EpiSwitch™ PCR and EpiSwitch™ Array biomarker platforms are compatible with analysis of both whole blood and cell lines. The tests are sensitive enough to detect abnormalities in very low copy numbers using small volumes of blood.

### Paragraphs Showing Embodiments of the Invention

1. A process for detecting a chromosome state which represents a subgroup in a population comprising determining whether a chromosome interaction relating to that chromosome state is present or absent within a defined region of the genome; and
wherein said chromosome interaction has optionally been identified by a method of determining which chromosomal interactions are relevant to a chromosome state corresponding to the subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to the subgroup; and
wherein the subgroup relates to prognosis for prostate cancer and the chromosome interaction either:
   (i) is present in any one of the regions or genes listed in Table 6; and/or
   (ii) corresponds to any one of the chromosome interactions represented by any probe shown in Table 6, and/or
   (iii) is present in a 4,000 base region which comprises or which flanks (i) or (ii);
   or
wherein the subgroup relates to prognosis for DLBCL and the chromosome interaction either:
   a) is present in any one of the regions or genes listed in Table 5; and/or
   b) corresponds to any one of the chromosome interactions represented by any probe shown in Table 5, and/or
   c) is present in a 4,000 base region which comprises or which flanks (a) or (b).
   2. A process according to paragraph 1 wherein:
said prognosis for prostate cancer relates to whether or not the cancer is aggressive or indolent; and/or
said prognosis for DLBCL relates to survival.
   3. A process according to paragraph 1 or 2 wherein the subgroup relates to prostate cancer and a specific combination of chromosome interactions are typed:
   (i) comprising all of the chromosome interactions represented by the probes in Table 6; and/or
   (ii) comprising at least 1, 2, 3 or 4 of the chromosome interactions represented by the probes in Table 6; and/or
   (iii) which together are present in at least 1, 2, 3 or 4 of the regions or genes listed in Table 6; and/or
   (iv) wherein at least 1, 2, 3, or 4 of the chromosome interactions which are typed are present in a 4,000 base region which comprises or which flanks the chromosome interactions represented by the probes in Table 6.
   4. A process according to paragraph 1 or 2 wherein the subgroup relates to DLBCL and a specific combination of chromosome interactions are typed:
   (i) comprising all of the chromosome interactions represented by the probes in Table 5; and/or
   (ii) comprising at least 10, 20, 30, 50 or 80 of the chromosome interactions represented by the probes in Table 5; and/or
   (iii) which together are present in at least 10, 20, 30 or 50 of the regions or genes listed in Table 5; and/or
   (iv) wherein at least 10, 20, 30, 50 or 80 chromosome interactions are typed which are present in a 4,000 base region which comprises or which flanks the chromosome interactions represented by the probes in Table 5.
   5. A process according to paragraph 1 or 2 wherein the subgroup relates to DLBCL and a specific combination of chromosome interactions are typed:
   (i) comprising all of the chromosome interactions shown in Table 7; and/or
   (ii) comprising at least 1, 2, 5 or 8 of the chromosome interactions shown in Table 7.
   6. A process according to any one of the preceding paragraphs wherein at least 10, 20, 30, 40 or 50, chromosome interactions are typed, and preferably at least 10 chromosome interactions are typed.
   7. A process according to any one of the preceding paragraphs in which the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, wherein detection of the ligated nucleic acid is preferably by:
   (i) in the case of prognosis of prostate cancer by a probe that has at least 70% identity to any of the specific probe sequences mentioned in Table 6, and/or (ii) by a primer pair which has at least 70% identity to any primer pair in Table 6; or
   (ii) in the case of prognosis of DLBCL a probe that has at least 70% identity to any of the specific probe sequences mentioned in Table 5, and/or (b) by a primer pair which has at least 70% identity to any primer pair in Table 5.
   8. A process according to any one of the preceding paragraphs, wherein:
the second set of nucleic acids is from a larger group of individuals than the first set of nucleic acids; and/or
the first set of nucleic acids is from at least 8 individuals; and/or
the first set of nucleic acids is from at least 4 individuals from a first subgroup and at least 4 individuals from a second subgroup which is preferably non-overlapping with the first subgroup; and/or
the process is carried out to select an individual for a medical treatment.
   9. A process according to any one of the preceding paragraphs wherein:
the second set of nucleic acids represents an unselected group; and/or
wherein the second set of nucleic acids is bound to an array at defined locations; and/or
wherein the second set of nucleic acids represents chromosome interactions in least 100 different genes; and/or
wherein the second set of nucleic acids comprises at least 1,000 different nucleic acids representing at least 1,000 different chromosome interactions; and/or
wherein the first set of nucleic acids and the second set of nucleic acids comprise at least 100 nucleic acids with length 10 to 100 nucleotide bases.
   10. A process according to any one of the preceding paragraphs, wherein the first set of nucleic acids is obtainable in a process comprising the steps of: —
   (i) cross-linking of chromosome regions which have come together in a chromosome interaction;
   (ii) subjecting said cross-linked regions to cleavage, optionally by restriction digestion cleavage with an enzyme; and
   (iii) ligating said cross-linked cleaved DNA ends to form the first set of nucleic acids (in particular comprising ligated DNA).
   11. A process according to any one of the preceding paragraphs wherein said defined region of the genome:
   (i) comprises a single nucleotide polymorphism (SNP); and/or
   (ii) expresses a microRNA (miRNA); and/or
   (iii) expresses a non-coding RNA (ncRNA); and/or
   (iv) expresses a nucleic acid sequence encoding at least 10 contiguous amino acid residues; and/or
   (v) expresses a regulating element; and/or
   (vii) comprises a CTCF binding site.
   12. A process according to any one of the preceding paragraphs which is carried out to determine whether a prostate cancer is aggressive or indolent which comprises typing at least 5 chromosome interactions as defined in Table 6.
   13. A process according to any one of the preceding paragraphs which is carried out to determine prognosis of DLBLC which comprises typing at least 5 chromosome interactions as defined in Table 5.
   14. A process according to any one of the preceding paragraphs which is carried out to identify or design a therapeutic agent for prostate cancer;
wherein preferably said process is used to detect whether a candidate agent is able to cause a change to a chromosome state which is associated with a different level of prognosis;
wherein the chromosomal interaction is represented by any probe in Table 6; and/or
the chromosomal interaction is present in any region or gene listed in Table 6;
   and wherein optionally:
the chromosomal interaction has been identified by the method of determining which chromosomal interactions are relevant to a chromosome state as defined in paragraph 1, and/or
the change in chromosomal interaction is monitored using (i) a probe that has at least 70% identity to any of the probe sequences mentioned in Table 6, and/or (ii) by a primer pair which has at least 70% identity to any primer pair in Table 6.
   15. A process according to any one of preceding paragraphs 1 to 13 which is carried out to identify or design a therapeutic agent for DLBCL;
wherein preferably said process is used to detect whether a candidate agent is able to cause a change to a chromosome state which is associated with a different level of prognosis;
wherein the chromosomal interaction is represented by any probe in Table 5; and/or
the chromosomal interaction is present in any region or gene listed in Table 5;
   and wherein optionally:
the chromosomal interaction has been identified by the method of determining which chromosomal interactions are relevant to a chromosome state as defined in paragraph 1, and/or
the change in chromosomal interaction is monitored using (i) a probe that has at least 70% identity to any of the probe sequences mentioned in Table 5, and/or (ii) by a primer pair which has at least 70% identity to any primer pair in Table 5.
   16. A process according to paragraph 14 or 15 which comprises selecting a target based on detection of the chromosome interactions, and preferably screening for a modulator of the target to identify a therapeutic agent for immunotherapy, wherein said target is optionally a protein.
   17. A process according to any one of paragraphs 1 to 16, wherein the typing or detecting comprises specific detection of the ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:
   an oligonucleotide which specifically binds to said ligated product, and/or
   a fluorophore covalently attached to the 5′ end of the oligonucleotide, and/or
   a quencher covalently attached to the 3′ end of the oligonucleotide, and
   optionally
   said fluorophore is selected from HEX, Texas Red and FAM; and/or
   said probe comprises a nucleic acid sequence of length 10 to 40 nucleotide bases, preferably a length of 20 to 30 nucleotide bases.
   18. A process according to any one of paragraphs 1 to 17 wherein:
the result of the process is provided in a report, and/or
the result of the process is used to select a patient treatment schedule, and preferably to select a specific therapy for the individual.
   19. A therapeutic agent for use in a method of treating prostate cancer or DLBCL in an individual that has been identified as being in need of the therapeutic agent by a process according to any one of paragraphs 1 to 13 and 17.
   The invention is illustrated by the following Examples:

### Example 1

### Using EpiSwitch′ (Chromosome Conformation Signature) Markers

We have consistently observed highly disseminating EpiSwitch™ markers with high concordance to the primary and secondary affected tissues and strong validation results. EpiSwitch™ biomarker signatures demonstrated high robustness and high sensitivity and specificity in the stratification of complex disease phenotypes.

The EpiSwitch′ technology offers a highly effective means of screening; early detection; companion diagnostic; monitoring and prognostic analysis of major diseases associated with aberrant and responsive gene expression. The major advantages of the OBD approach are that it is non-invasive, rapid, and relies on highly stable DNA based targets as part of chromosomal signatures, rather than unstable protein/RNA molecules.

CCSs form a stable regulatory framework of epigenetic controls and access to genetic information across the whole genome of the cell. Changes in CCSs reflect early changes in the mode of regulation and gene expression well before the results manifest themselves as obvious abnormalities. A simple way of thinking of CCSs is that they are topological arrangements where different distant regulatory parts of the DNA are brought in close proximity to influence each other's function. These connections are not done randomly; they are highly regulated and are well recognised as high-level regulatory mechanisms with significant biomarker stratification power.

### Prognostic Stratification of Prostate Cancer

Markers were developed on the basis of retrospective annotations of Class I (low risk, indolent), Class II (intermediate), and Class III (aggressive high risk). The markers show robust classification of patients against healthy controls and also discriminate between Classes. The samples were from the United Kingdom.

To Identify EpiSwitch™ Biomarkers Able to Distinguish Between Blood from People with Prostate Cancer and Healthy Controls

A custom EpiSwitch™ Microarray investigation was initially used to identify and screen ^{˜}15,000 potential CCS over 425 genetic loci for discrimination between 8 Prostate Cancer (PCa) and 8 Control individuals. The top statistically significant markers were translated into Nested PCR assays and screened on a larger sample cohort of 24 PCa and 25 Healthy Control Samples. A classifier was developed using the top 5 CCS translated from the microarray which classified the PCa and Control samples with a Sensitivity and Specificity of 100% (95% CI— 86.2% to 100%) and 100% (95% CI— 86.7% to 100%) respectively.

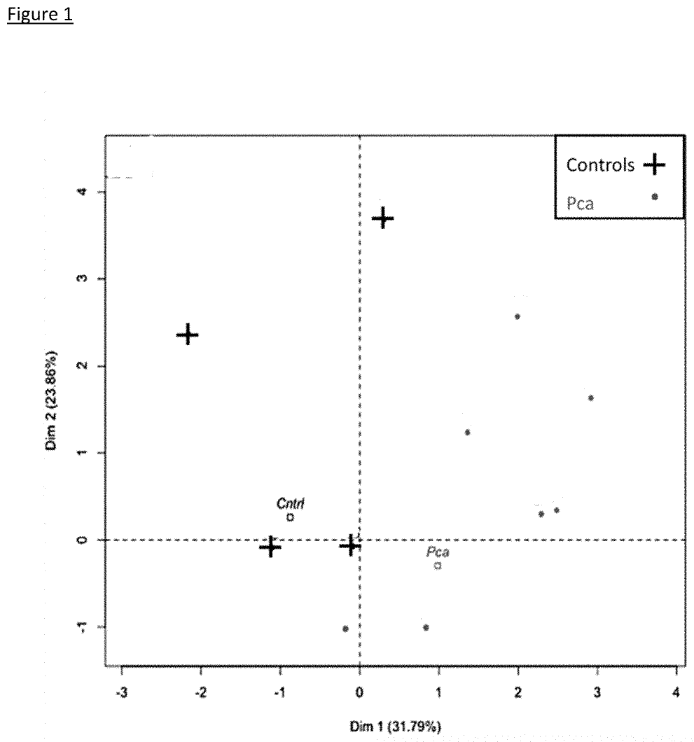

FIG. 1 shows a Principle Component Analysis of the top 5 markers on 49 samples of the development sample cohort.

The diagnostic classifier was used to classify an additional blinded independent cohort consisting of 24 PCa and 5 healthy control samples (n=29) with an accuracy of 83%. Further development of the EpiSwitch™ Prostate cancer assay was performed with an additional sample cohort of 95 PCa and 97 Controls (n=192). This in turn was validated with a blinded sample cohort of 20 samples (10 PCa, 10 Controls). The results of this validation are shown in Table 1.

TABLE 1
Results for the classification of the
blinded sample cohort (n = 20)

| | | 95% Confidence Interval (CI) |
|---|---|---|
| Sensitivity | 80.0% | 44.4%-97.5% |
| Specificity | 80.0% | 44.4%-97.5% |
| PPV | 80.0% | 44.4%-97.5% |
| NPV | 80.0% | 44.4%-97.5% |

The most recent project in the PCA programme developed an alternative PCR format for the PCa diagnosis utilising hydrolysis probe based Realtime quantitative PCR (qPCR). The performance of the 6-marker model is shown in Table 2.

TABLE 2
Performance of 6 marker qPCR model

| | | 95% Confidence Interval (CI) |
|---|---|---|
| Sensitivity | 90.0% | 73.47%-97.89% |
| Specificity | 85.0% | 62.11%-96.79% |
| PPV | 90.0% | 75.90%-96.26% |
| NPV | 85.0% | 65.60%-94.39% |

### SUMMARY

The three independent blinded validations of the EpiSwitch™ PCa Diagnostic Signatures developed during the PCa diagnostic program, using US and UK samples of varying disease stages, achieves sensitivity and specificity of >80% for the diagnosis of Prostate Cancer. The Prostate Specific Antigen (PSA) Blood test which is the Gold Standard clinical assay for detecting PCa, which in itself relies on various other variables, typically has a sensitivity and specificity range of 32-68%. In addition a parallel research track has resulted in the development of an EpiSwitch™ assay to assess Prostate cancer prognosis to aid in the clinical management and treatment selection for individual patients diagnosed with PCA.

An additional custom EpiSwitch™ Microarray investigation was performed to identify and screen ^{˜}15,000 potential CCS over 426 genetic loci for discrimination between 8 Aggressive Prostate Cancer (Class 3) and 8 Indolent PCa (Class 1) patients, PCa class descriptions can be found in the Appendix. The top statistically significant markers were translated into Nested PCR assays and screened on a larger sample cohort of 42 Class 1, 25 Class 2 and 19 Class 3 PCa samples.

The top 6 statistically significant markers were used to develop a prognostic classifier to classify Class 1 (low risk) and Class 3 (high Risk) PCa. The performance of the classifier on an independent sample cohort of 42 Class 1 and 25 Class 3 samples (n=27) is shown in Table 3.

TABLE 3
Performance of 6 marker prognostic
classifier (Class 1 vs Class 3)

| | | 95% Confidence Interval (CI) |
|---|---|---|
| Sensitivity | 80.0% | 59.3%-93.17% |
| Specificity | 92.86% | 80.52%-98.5% |
| PPV | 86.96% | 66.41%-97.22% |
| NPV | 88.64% | 75.44%-96.21% |

An alternative analysis found a further 6 markers that stratified between Class 2 and Class 3 PCa. The two classifiers share two markers, with each classifier also possessing 4 unique markers.

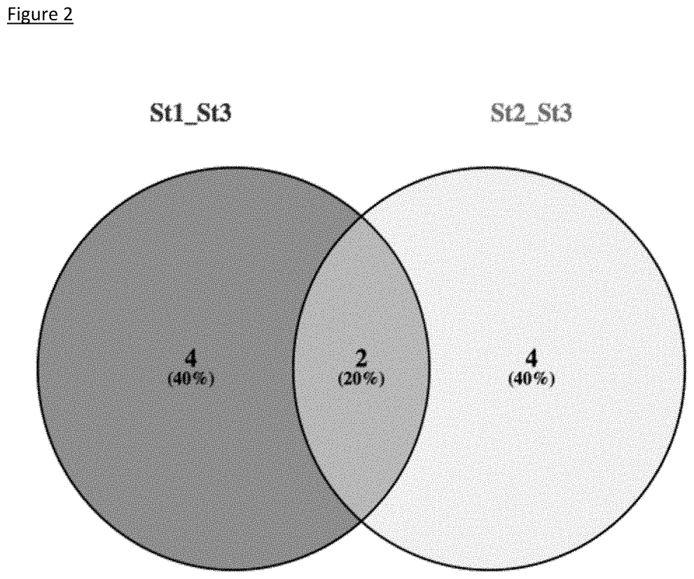

FIG. 2 shows a VENN comparison of the two PCA prognostic classifiers.

The performance of the Class 2 vs Class 3 PCa classifier is shown in Table 4.

TABLE 4
Performance of 6 marker prognostic classifier
(Class 2 vs Class 3) n = 44

| | | 95% Confidence Interval (CI) |
|---|---|---|
| Sensitivity | 84.0% | 63.92%-95.46% |
| Specificity | 88.89% | 65.29%-98.62% |
| PPV | 91.30% | 71.96%-98.93% |
| NPV | 80.00% | 56.34%-94.27% |

### Conclusions

The development of the diagnostic and prognostic biomarkers was achieved on multiple clinical sample cohorts. All conducted marker screening and selection was based on systemic, blood-based epigenetic changes as monitored through chromosome conformation signatures in patients with different stages of Prostate Cancer (stage 1 to 3) against healthy controls (diagnostic application), as well as patients with aggressive, high risk category 3 against indolent, low risk category 1 prostate cancers (prognostic application), or intermediate risk category 2.

The results of stratification development for PCa vs healthy controls showed sensitivity and specificity up to >80% in the testing cohort and a series of blind validations. Stratification of high-risk category 3 vs low risk category 1 PCa showed sensitivity up to 80% and specificity up to 92% on cohorts of up to 67 samples, while stratification of high-risk category 3 vs intermediate-risk category 2 showed sensitivity up to 84%, and specificity up to 88% on cohorts of up to 44 samples.

### Appendix

### Low Risk—Category 1

Localised prostate cancer is classified as low risk if
PSA level is less than 10 ng per ml, and
Gleason score is no higher than 6, and
The T stage is between T1 and T2a

### Intermediate Risk—Category 2

Localised prostate cancer is classed as intermediate risk if you have at least one of the following
PSA level is between 10 and 20 ng/ml
Gleason score is 7
The T stage is T2b

### High Risk—Category 3

Localised prostate cancer is classed as high risk if you have at least one of the following
PSA level is more than 20 ng/ml
Gleason score is between 8 and 10
The T stage is T2c, T3 or T4

If the cancer is T3 or T4 stage, this means it has broken through the outer fibrous covering (capsule) of the prostate gland, and so it is classed as locally advanced prostate cancer.

### Example 2. Identifying Markers for DLBCL

### Summary

This relates to identification of major groups of poor and good prognosis patients for subsequent selection of treatments (i.e. R-CHOP). The biomarkers have been developed on the basis of retrospective overall survival. Normally, patients are classified by biopsy based gene expression standards like Nanostring or Fluidigm, according to diseases subtypes such as ABC (poor prognosis) or GCB (better prognosis). However not all patients could be classified as ABC or GCB (the so called Type III, or Unclassified patients). We identified biomarkers to provide classification for prognosis of survival at the baseline, before treatments, irrespective of ABC or GCB standard classification.

### Identification of Markers

DLBCL shows distinct differences in patients survival (poor vs good prognosis) and is characterised by a number of molecular readouts into subtypes. Various subtypes are also treated differently in current clinical practice. This, for example includes combination of Rituximab and CHOP combination on chemotherapy. There are various approaches.

Currently practiced molecular readouts are based on gene expression profiling by arrays, performed on biological materials obtained by direct biopsies. Those include Nanostring and Fluidigm array-based tests for extreme types of ABC and GCB. ANC subtype normally is associated with poor prognosis. Not every patient could be classified as ABC or GCB, a number of patients remain unclassified (or Type III) in terms of the established gene expression profiles and any association with prognosis of poor survival. We built systemic biomarkers that will directly classify patients for poor vs good prognosis, irrespective of transcriptional gene expression profiling by other modalities.

Step one: We used the Episwitch screening array to compare the epigenetic profiles on groups of cell lines representing poor prognosis and good prognosis of survival for DLBCL. This allows identification of array based markers and designing of nested PCR primers to use for the same targets in PCR format.
Step two: We used top 10 nested PCR based markers read on baseline blood samples from 57-58 unclassified DLBCL patients with known retrospective survival annotations. Table 6 provides details for the markers, the final signature, and the stated performance by the classifier model.

Our work shows how base line calls on these patients for poor/good prognosis compared against the clinical survival data. This is a Cox estimate of hazard ratio, i.e. our baseline classification into poor prognosis shows higher probabilities for being in a poor prognosis survival group, rather than a good prognosis group by the clinical post factum annotation, with a particular value >1. The latter is of particular value and interest for clinical teams in trial designs.

### Detailed Writeup

Diffuse large B-cell lymphoma (DLBCL) is the most common type of non-Hodgkin's lymphoma in adults. It can occur anytime between adolescence and old age, affects 7-8 people per 100,000 in the US annually, although the incidence rate increases with age. Gene expression profiling has revealed two major types of DLBCL—germinal centre B-cell like (GCB) and activated B-cell like (ABC). GCB DLBCL arises from secondary lymphoid organs e.g. lymph nodes, where naïve B-cells do not stop dividing after infection is cleared. ABC DLBCL is thought to begin in a subset of B-cells which are ready to leave the germinal centre and become plasma cells i.e. plasmablastic B-cells, but the reality is more complicated with different forms of DLBCL occurring through the whole B-cell lifecycle.

The different subtypes have varying prognoses with a 5-year survival rate of 60% for GCB DLBCL, but only 35% for ABC DLBCL. Each of the subtypes is characterized by differential gene expression. In GCB DLBCL the transcriptional repressor BCL6 is often over-expressed whereas in ABC DLBCL the NF-κB pathway is often found to be constitutively activated. There is also a third type of DLBCL called type III which is currently less well understood but it is thought to have a gene expression profile situated between the two main types.

Current diagnostic methods involve excisional biopsy of the affected lymph node followed by immunohistochemistry (IHC). At present, treatment procedures for DLBCL are the same regardless of the subtype. Since the pathogenesis, treatment responses, and outcomes of the various subtypes differ enormously there remains a need to develop a robust, non-invasive assay to distinguish between the subtypes in order to assist in the development of differentiated treatment strategies. Although much research has been carried out to find predictive and prognostic biomarkers for DLBCL there is no consensus on a single test that can be used to distinguish between the subtypes.

To Identify EpiSwitch™ Biomarkers Able to Distinguish Between the Different Subtypes of DLBCL in Blood from Patients with DLBCL

We used the EpiSwitch™ array platform to look at DLBCL cell lines and blood samples and identify biomarkers that were absent in healthy control patients, before confirming these biomarkers in a 70 patient cohort consisting of 30 ABC, 30 GCB and 10 healthy control samples.

### EpiSwitch™ Array

The EpiSwitch™ custom array allows the screening of several thousand possible CCS's, with probes designed using pattern recognition software. Different long-range chromosomal interactions captured by EpiSwitch™ technology reflect the epigenetic regulatory framework imposed on the loci of interest and correspond to individual different inputs from signalling pathways contributing to the co-regulation of these loci. Altogether, the combination of the different inputs modulates gene expression. Identification of an aberrant or distinct chromosomal conformation signature under specific physiological condition offers important evidence for specific contribution to deregulation before all the input signals are integrated in the gene expression profile.

Using data from several sources 98 genetic loci were selected for analysis with the proprietary software and probes for 13,332 potential chromosomal conformations were tested. Looking at one locus does not equate to looking at one marker, as there may be one, multiple, or no high-order epigenetic chromosome conformation markers in a specific locus. After manufacture cell lines and blood samples from DLBCL patients and healthy controls were processed using the EpiSwitch protocol, labelled, and hybridized to the array.

### Samples for Diagnostic Development

We used 16 cell lines, which corresponded to different subtypes, and with different levels of confidence in subtyping. The most definite ABC and GCB subtyped cell lines were used for analysis. In addition, blood samples from four DLBCL patients and 11 healthy controls were used. After biomarker identification in part one 60 further samples were provided to OBD, consisting of 30 ABC and 30 GCB blood samples, well characterised by Fluidigm testing, and this was supplemented by ten healthy control samples provided by OBD.

Results

Array Analysis

72 chromosome signature sites from the microarray were chosen to be screened based on two criteria:
Their ability to stratify between ABC and GCB cells (highABC_highGCB)
   and/or
A low CV value (a median value of the 5 arrays analyzed, High ABC v High GCB, DLBCL1 v Healthy Control, DLBCL2 v Healthy Control, DLBCL3 v Healthy Control and DLBCL4 v Healthy Control)

### Translation of Array to EpiSwitch™ PCR Platform

After analysis of the sequence surrounding the probes of interest from the array 69 sets of primers were designed to interrogate the chromosome signature sites. These were then tested on pooled DLBCL blood samples, and of these 49 met the OBD criteria for PCR products for use in assays.

Each of these 49 potential markers were then tested on six DLBCL cell lines—three of which were ABC and three of which were GCB. The cell lines used were those which were most confident were ABC or GCB, due to the same categorisation being found using multiple different identification methods. This allowed for the markers to be selected that were most useful in differentiating ABC and GCB cell subtypes. 28 EpiSwitch™ markers were identified for use with the PCR platform that were consistent with the EpiSwitch™ microarray results. In addition, the potential markers were also tested against four DLBCL patients and pooled healthy controls to identify those that were present in DLBCL patients, but absent in healthy controls. 21 of the 28 EpiSwitch™ markers were absent in healthy control samples, but present in DLBCL samples such that it could be used as a marker of DLBCL, as well as for subtyping.

### Sample Testing

The 21 markers that translated well into the EpiSwitch™ PCR platform were then tested amongst the 70 patient blood sample cohort. Initially, each marker was tested in six new ABC samples, and six new GCB samples, and the 21-marker set narrowed down to ten markers that showed the greatest difference. These ten markers were then tested on the remaining 24 ABC, 24 GCB and ten healthy control samples.

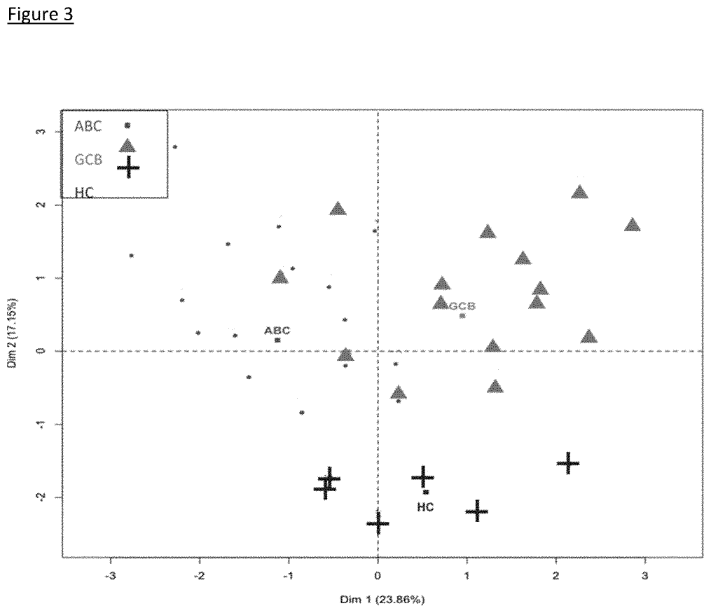

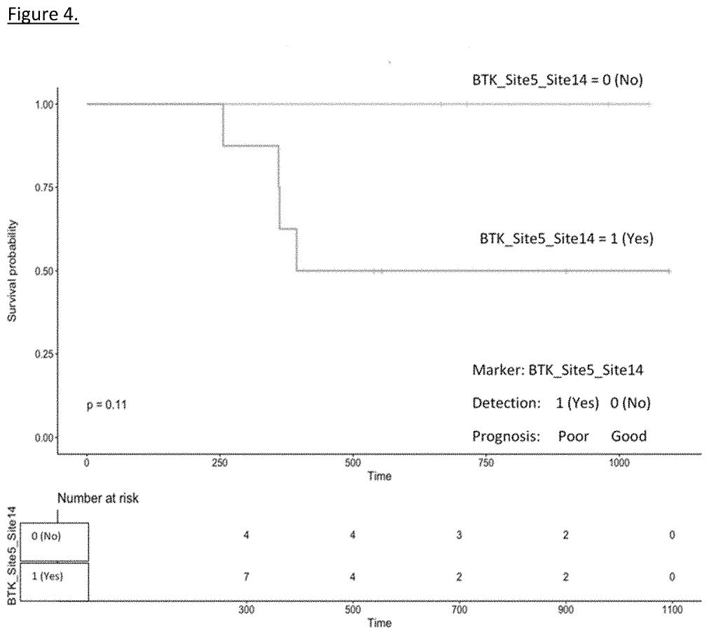

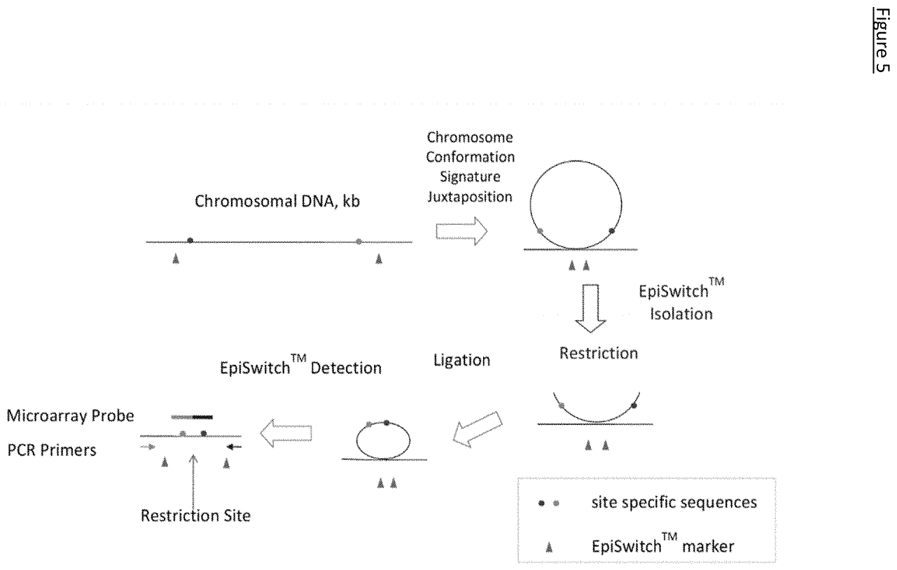

Each of the markers was then subjected to analysis of its power to differentiate subgroups, its collinearity with other markers, and also its ability to differentiate healthy from DLBCL. A subset of six of the markers was identified that provided the maximum possible information and these are markers in the ANXA11 IFNAR, MAP3K7, MEF2B, NFATc1, and TNFRS13C loci. FIG. 3 shows the ability of these markers to differentiate the different groups of samples on a PCA plot. This six-marker panel is able to clearly differentiate healthy control patients from DLBCL patients, a key characteristic of any blood-based assay for DLBCL.

FIG. 3 shows a PCA plot of 60 DLBCL and 10 healthy patients based on the six EpiSwitch™ marker binary data. Samples are characterized as ABC subtype or GCB subtype by Fluidigm data, and the healthy controls are also shown.

Classification: Identification of ABC and GCB Subtypes within DLBCL Patient Cohort (60 Samples)

Classification was performed using the logistic regression classifier with 5-fold cross-validation, and the following results were achieved. The following results were achieved in cross-validation:

| | |
|---|---|
| ABC subtype | 83.3% (95% CI-65.3% to 94.3%) |
| GCB subtype | 83.3% (95% CI-65.3% to 94.3%) |

In addition, the resultant six-marker logistic classifier model was tested on 50 permutations of the 60-patient data set. The data was randomized each time and the accuracy statistics were calculated with a ROC curve. An area under the curve (AUC) of 0.802 and p-value 0.0000037 (H0=“The AUC is equal to 0.5”), suggests that the model is accurate and performing efficiently.

### Conclusions

In this study we have demonstrated the power of their EpiSwitch™ technology to provide answers to difficult clinical questions, particularly the differentiation of the ABC and GCB subtypes of DLBCL. Using high-throughput array methods, and translation to the simple and cost-effect PCR platform more than 13,000 potential CCS's have been tested and refined to a six marker panel for DLBCL subtype differentiation. This panel was able to distinguish DLBCL patients from healthy controls, and was able to predict subtype accurately 83.3% of the time. This test also has greater than 80% concordance for class assignment between EpiSwitch™ (whole blood based), LPS (cell of origin, tissue) and Fluidigm (cell of origin, tissue)

EpiSwitch™ technology detects changes in long-range intergenic interactions—chromosomal conformation signatures, which result in changes in the epigenetic status and modulation of the expression mode of key genes involved in the pathogenesis of disease. The diagnostic procedure based on EpiSwitch™ technology is a simple and rapid technique that can be transferred to other laboratories. The test consists of several molecular biology reactions, followed by detection with nested PCR. The test does not require complicated procedures and can be performed in any laboratory that runs PCR-based assays.

### Example 3

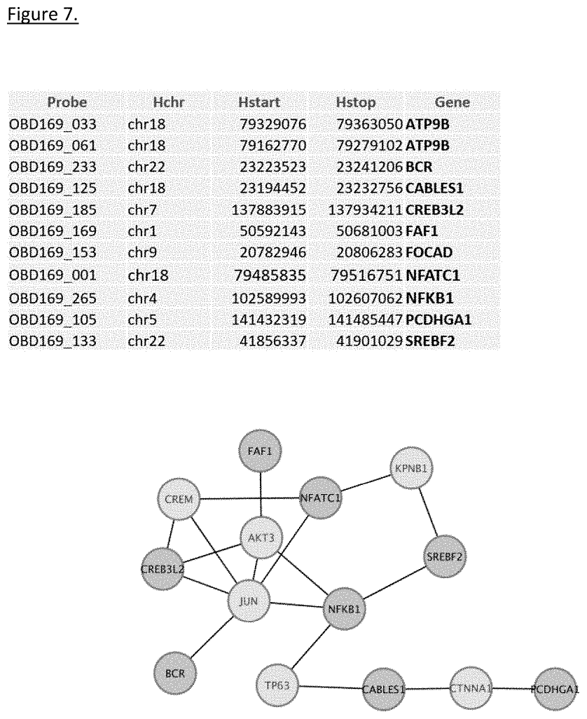

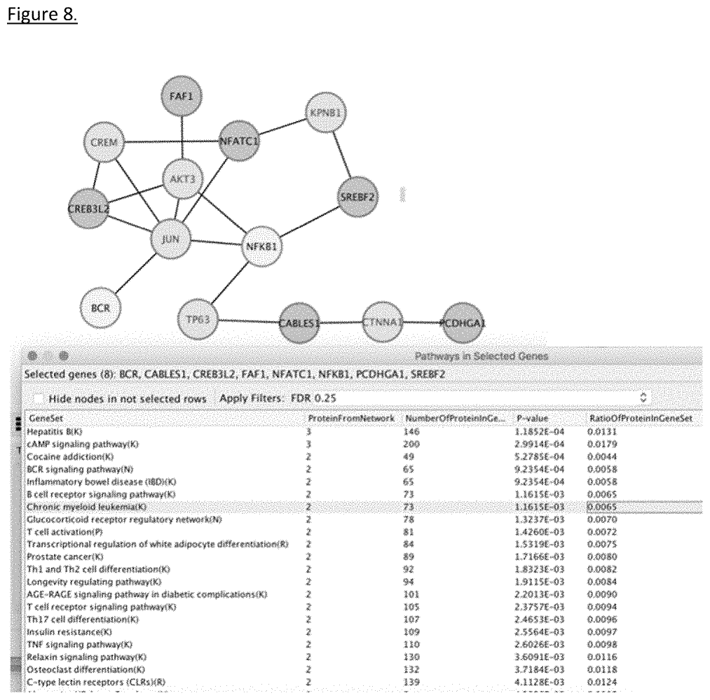

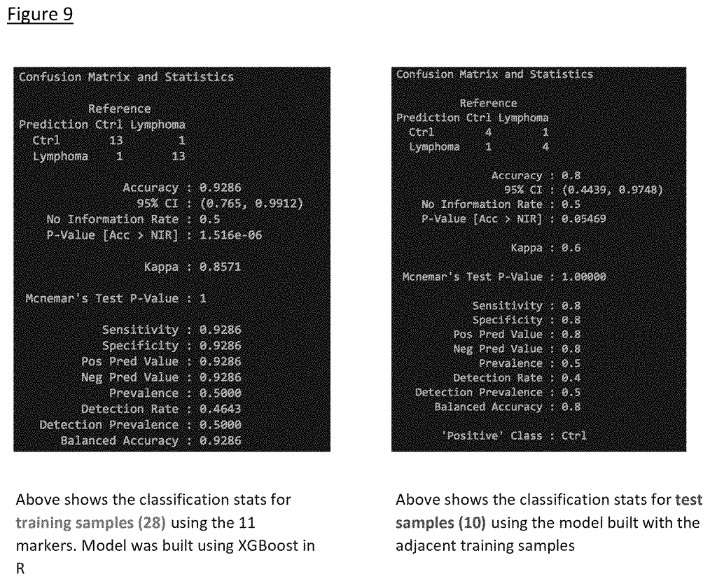

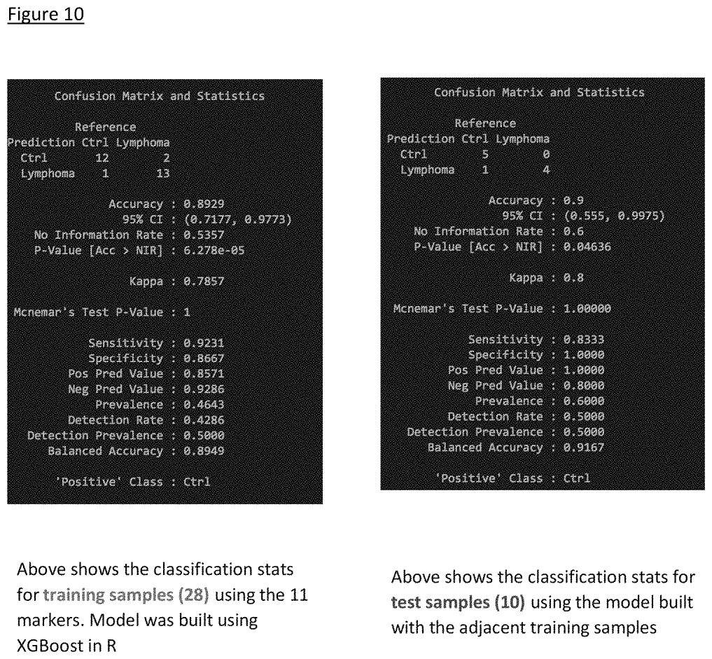

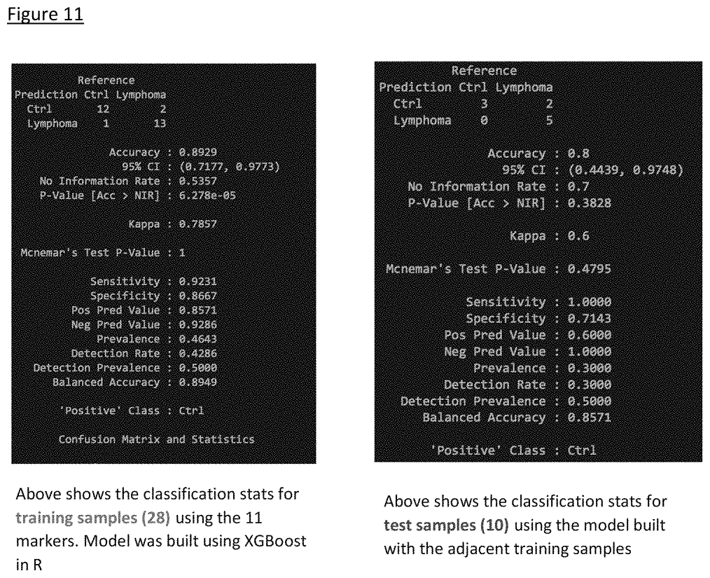

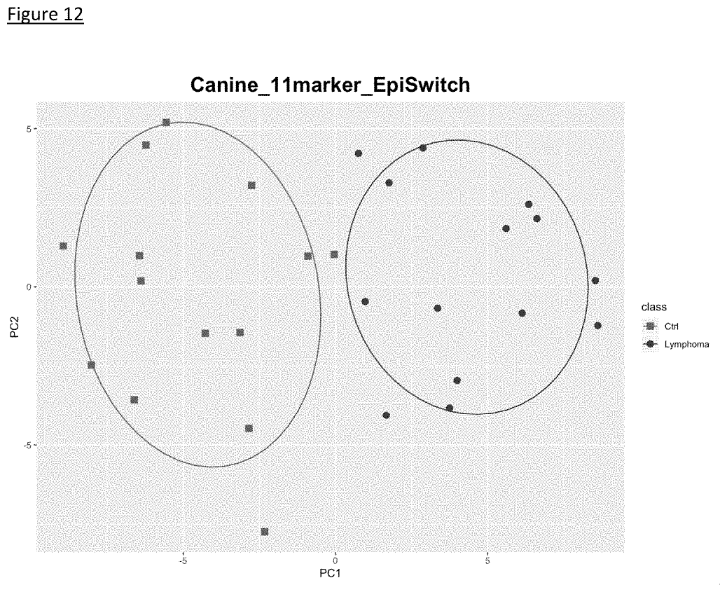

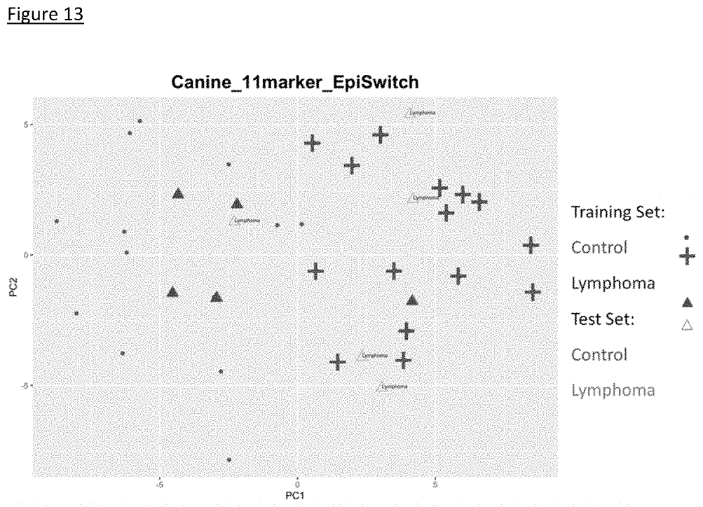

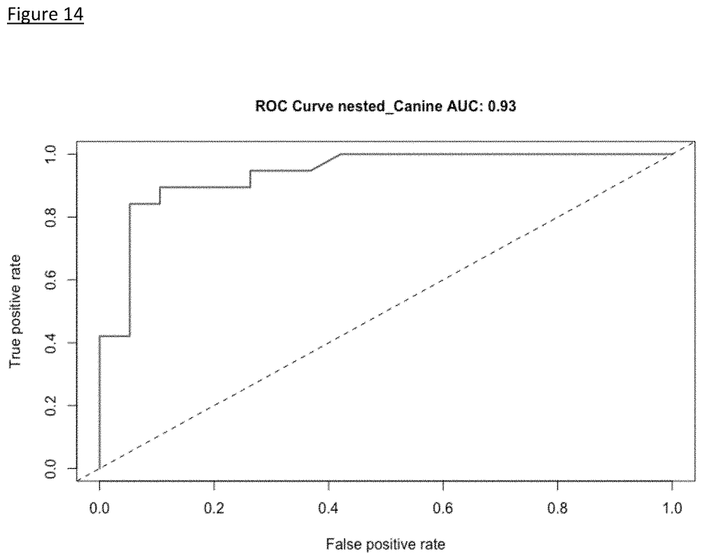

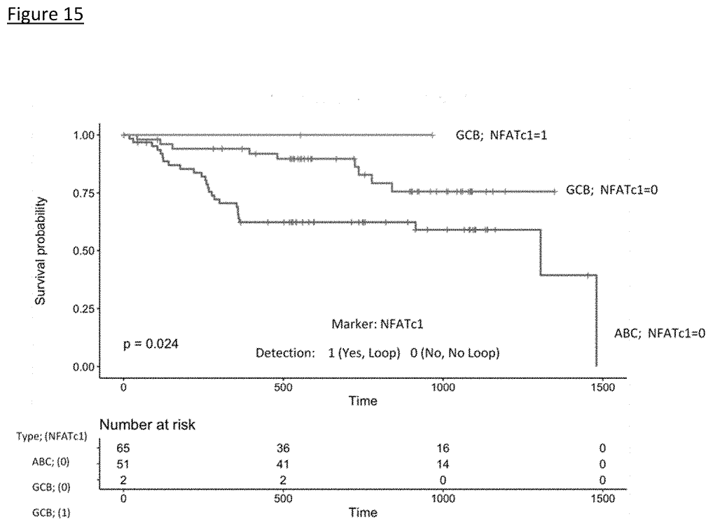

Further work was performed on canines. One aim was to investigate markers for aiding in the initial diagnosis of suspected lymphoma to inform veterinary clinicians on the requirements for performing follow up biopsies. In this study, the top 75 EpiSwitch Microarray DLBCL markers (previously identified) are translated from the Human Genome Build (Grch37) to the current canine genome. In total 38 Canine samples (consisting of the 19 patients with likely lymphoma and 19 matched control samples) were screened using all 75 DLBCL markers. To carry out this work the following were performed:
Based on 75 human DLBCL markers (associated with specific genes) orthologues in Dog genome (CanFam3.1) identified and genetic loci extracted from Biomart.
EpiSwitch™ software run to identify potential interactions in these loci
Primer design software and other filters added to reduce list to 75 markers for investigation.
   The work and results are shown in FIGS. 6 to 16 and in Tables 8 and 9.

### Example 4. Further Work on Prostate Cancer

Current diagnostic blood tests for prostate cancer (PCa) are unreliable for the early stage disease, resulting in numerous unnecessary prostate biopsies in men with benign disease and false reassurance of negative biopsies in men with PCa. Predicting the risk of PCa is pivotal for making an informed decision on treatment options as the five-year survival rate in the low-risk group is more than 95% and most men would benefit from less invasive therapy. Three-dimensional genome architecture and chromosome structures undergo early changes during tumorigenesis both in tumour and in circulating cells and can serve a disease biomarker.

In this prospective study we have performed chromosome conformation screening for 14,241 chromosomal loops in the loci of 425 cancer related genes in whole blood of newly diagnosed, treatment naïve PCa patients (n=140) and non-cancer controls (n=96).

Our data show that peripheral blood mononuclear cells (PBMCs) from PCa patients acquired specific chromosome conformation changes in the loci of ETS1, MAP3K14, SLC22A3 and CASP2 genes. Blind testing on an independent validation cohort yielded PCa detection with 80% sensitivity and 80% specificity. Further analysis between PCa risk groups yielded prognostic validation sets consisting of BMP6, ERG, MSR1, MUC1, ACAT1 and DAPK1 genes for high-risk category 3 vs low-risk category 1 and HSD3B2, VEGFC, APAF1, MUC1, ACAT1 and DAPK1 genes for high-risk category 3 vs intermediate-risk category 2, which had high similarity to conformations in primary prostate tumours. These sets achieved 80% sensitivity and 92% specificity stratifying high-risk category 3 vs low risk category 1 and 84% sensitivity and 88% specificity stratifying high risk category 3 vs intermediate risk category 2 disease.

Our results demonstrate specific chromosome conformations in the blood of PCa patients that allow PCa diagnosis and prognosis with high sensitivity and specificity. These conformations are shared between PBMCs and primary tumours. It is possible that these epigenetic signatures may potentially lead to development of a blood-based PCa diagnostic and prognostic tests.

### Introduction

In the Western world prostate cancer (PCa) is now the most commonly diagnosed non-cutaneous cancer in men and is the second leading cause of cancer-related death. Many men as young as 30 show evidence of histological PCa, most of which is microscopic and possibly will never show clinical manifestations. For the diagnosis and prognosis, prostate specific antigen (PSA), an invasive needle biopsy, Gleason score and disease stage are used. In a large multicentre study of 2,299 patients, a 12-site biopsy scheme outperformed all other schemes, with an overall PCa detection rate of only 44.4%.

The only available blood test for PCa in widespread clinical use involves measuring circulating levels of PSA (21% sensitivity and 91% specificity), however, the prostate size, benign prostatic hyperplasia and prostatitis may also increase PSA levels. At the current 4.0 ng/ml cut-off limit, only 20% of all PCa patients are being detected. In early PCa, PSA testing is not specific enough to differentiate between early-stage invasive cancers and latent, non-lethal tumours that might otherwise have remained asymptomatic during a man's lifetime. In advanced PCa, PSA kinetics are used as a clinical surrogate endpoint for outcome. However, while they do give a general prognosis they lack specificity for the individual. A number of more specific blood tests are emerging for PCa detection including 4K blood test (AUC 0.8) and PHI blood test (90% sensitivity, 17% specificity). PSA levels, disease stage and Gleason score are used to establish the severity of PCa and stratify patients to risk groups. To date, there is no prognostic blood test available that allows differentiation between low- and high-risk PCa.

There are multiple genetic changes associated with PCa, including mutations in p53 (up to 64% of tumours), p21 (up to 55%), p73 and MMAC1/PTEN tumour suppressor genes, but these mutations do not explain all the observed effects on gene regulation. Epigenetic mechanisms involving dynamic and multi-layered chromosomal loop interactions are powerful regulators of gene expression. Chromosome conformation capture (3C) technologies allow these signatures to be recorded. In this study, we used the EpiSwitch™ assay to screen for, define and evaluate specific chromosome conformations in the blood of PCa patients and to identify loci with potential to act as diagnostic and prognostic markers.

### Methods

A total of 140 PCa patients and 96 controls were recruited, in two cohorts. Cohort 1: men with (n=105) or without (n=77) PCa diagnosis attending a urology clinic were prospectively recruited from October 2010 through September 2013. Cohort 2: Patients' samples (19 controls and 35 PCa) obtained from the USA. Upon recruitment, a single blood sample (5 ml) was collected from PCa patients using the current practice for needle and blood collection methods into the BD Vacutainer® plastic EDTA tubes. Blood samples were passively frozen and stored at −80° C. until processed. Prostate tumour samples were obtained from previously recruited patients (n=5) that subsequently underwent radical prostatectomy. Patient clinical characteristics are shown in Table 17.

The primary endpoint of this study was to detect changes in chromosomal conformations in PBMCs from PCa patients in comparison to controls. Therefore, all treatment naïve PCa patients were eligible for this study irrespective of grade, stage and PSA levels. Patients that had previous chemotherapy or patients with other cancers were excluded from this study. PCa diagnosis was established as per clinical routine and patients were assigned to appropriate treatment. For prognostic study (secondary endpoint), patients were stratified according to the relevant NCCN risk groups (Table 10). No follow up study was conducted.

Based on the preliminary findings in melanoma, an a priori power analysis was performed using the pwr.t.test function in the R package pwd. Testing indicated 15 patients per group should be sufficient to detect correlation between variables (β=5% probability type II error, significance level; 95% power; 50% confidence interval and 40% standard deviation).

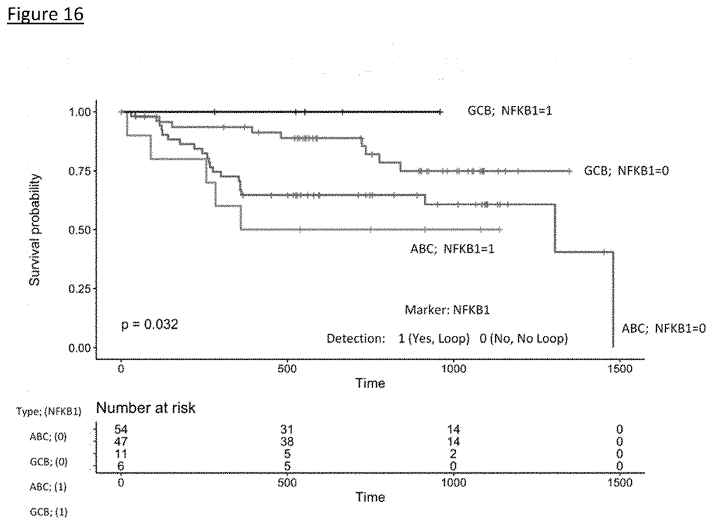

EpiSwitch™ technology platform pairs high resolution 3C results with regression analysis and a machine learning algorithm to develop disease classifications. To select epigenetic biomarkers that can diagnose cancers, samples from patients suffering from cancer, in comparison to healthy (control) samples were screened for statistically significant differences in conditional and stable profiles of genome architecture. The assay is performed on a whole blood sample by first fixing chromatin with formaldehyde to capture intrachromatin associations. The fixed chromatin is then digested into fragments with Taql restriction enzyme, and the DNA strands are joined favouring cross-linked fragments. The cross-links are reversed and polymerase chain reactions (PCR) performed using the primers previously established by the EpiSwitch™ software. EpiSwitch™ was used on blood samples in a three-step process to identify, evaluate, and validate statistically-significant differences in chromosomal conformations between PCa patients and healthy controls (FIG. 17). For the first step, sequences from 425 manually curated PCa-related genes (obtained from the public databases (www.ensembl.org)) were used as templates for this computational probabilistic identification of regulatory signals involved in chromatin interaction (Table 18). A customized CGH Agilent microarray (8×60k) platform was designed to test technical and biological repeats for 14,241 potential chromosome conformations across 425 genetic loci. Eight PCa and eight control samples were competitively hybridized to the array, and differential presence or absence of each locus was defined by LIMMA linear modelling, subsequent binary filtering and cluster analysis. This initially revealed 53 chromosomal interactions with the ability to best discriminate PCa patients from controls (FIG. 17).

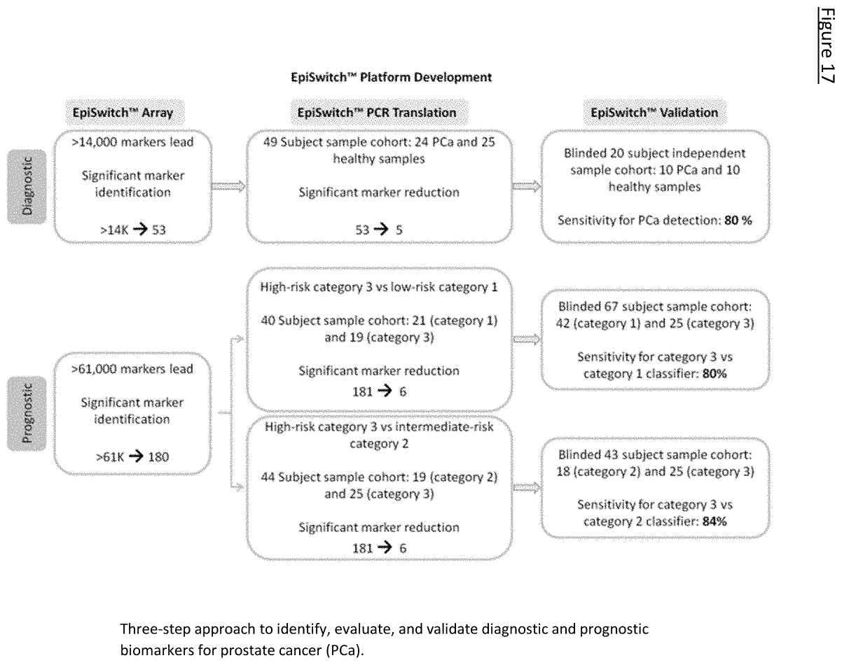

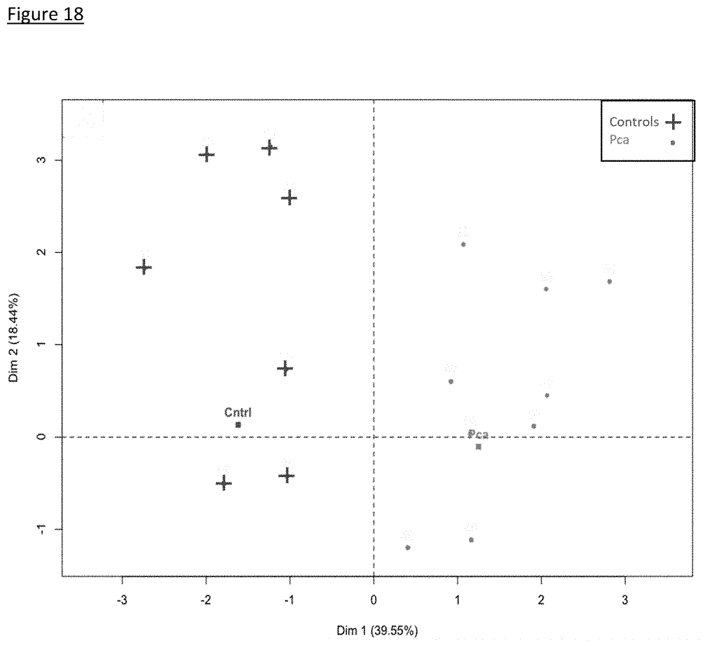

For the second evaluation stage, the 53 biomarkers selected from the array analysis were translated into EpiSwitch™ PCR based-detection probes and used in multiple rounds of biomarker evaluation. PCR primers were selected according to their ability to distinguish between PCa and healthy controls (n=6 in each group). The identity of PCR products generated using nested primers was confirmed by direct sequencing. Accordingly, the 53 biomarkers selected were reduced to 15 markers after the initial statistical analysis and finally a five-marker signature (Table 11). This selected chromosomal-conformation signature-biomarker set was then tested on a known cohort (n=49). Additionally, the five-marker signature developed from EpiSwitch™ PCR evaluation of array marker leads was tested on an independent blind validation cohort of 29 samples which were combined with the known 49 samples tested earlier (total 78 samples). Principal component analysis was also used to determine abundance levels and to identify potential outliers (FIG. 18).

For the last step, to further validate the chromosome conformation signature used to inform PCa diagnosis, the five-marker set was tested on a blinded, independent (n=20) cohort of blood samples. The results were analysed using Bayesian Logistic modelling, p-value null hypothesis (Pr(N|z|) analysis, Fisher-Exact P test and Glmnet (Table 12). The sample cohort sizes in the five-marker signature study were progressively increased to enable selection of the optimal markers for discriminating PCa samples from healthy controls. Cohort sizes were expanded to 95 PCa and 96 healthy control samples. Data analysis and presentation were performed in accordance with CONSORT recommendations. All measurements were performed in a blinded manner. STARD criteria have been used to validate the analytical procedures. A similar three-step approach was followed for the identification of prognostic markers (Table 13).

Sequence specific oligonucleotides were designed around the chosen sites for screening potential markers by nested PCR using Primer3. All PCR amplified samples were visualized by electrophoresis in the LabChip GX, using the LabChip DNA 1K Version2 kit (Perkin Elmer, Beaconsfield, UK) and internal DNA marker was loaded on the DNA chip according to the manufacturer's protocol using fluorescent dyes. Fluorescence was detected by laser and electropherogram read-outs translated into a simulated band on gel picture using the instrument software. The threshold we set for a band to be deemed positive was 30 fluorescence units and above.

Primary tumour samples were obtained from biopsies of selected patients (n=5). The pulverized tissue samples were incubated in 0.125% collagenase at 37° C. with gentle agitation for 30 minutes. The resuspended cells (250 ul) were then centrifuged at 800g for 5 minutes at room temperature in a fixed arm centrifuge, supernatant removed, and the pellets resuspended in phosphate-buffered saline (PBS). Primary tumours and matching blood samples were analysed for the presence of the six-markers set for categories 3 vs 1 and 3 vs 2 at a fixed range of assay sensitivity (dilution factor 1:2). When matching PCR bands of the correct size were detected, a score of 1 was assigned, detection of no band was assigned a score of 0 (Table 14).

We have applied a stepwise diagnostic biomarker discovery process using EpiSwitch™ technology as described in methods. A customized CGH Agilent microarray (8×60k) platform was designed to test technical and biological repeats for 14,241 potential chromosome conformations across 425 genetic loci (Table 18) in eight PCa and eight control samples (FIG. 17). The presence or absence of each locus was defined by LIMMA linear modelling, subsequent binary filtering and cluster analysis. In the second evaluation stage, nested PCR was used for the 53 selected biomarkers further reducing them to 15 markers and finally to a five-marker signature (FIG. 17). This distinct chromosome conformational disease classification signature for PCa comprised of chromosomal interactions in five genomic loci: ETS proto-oncogene 1, transcription factor (ETS1), mitogen-activated protein kinase kinase kinase 14 (MAP3K14), solute carrier family 22 member 3 (SLC22A3) and caspase 2 (CASP2) (Table 11). The genomic locations of specific chromosomal loops in ETS1, MAP3K14, SLC22A3 and CASP2 genes in the chromosome conformation signature (Table 11) were mapped on their relative chromosomes. The two genomic sites that corresponded to the junction of each chromosome conformation signature locus for ETS1, MAP3K14, SLC22A3 and CASP2 genes were mapped on chromosome 11 from 128,260,682 to 128,537,926; chromosome 17 from 43,303,603 to 43,432,282; chromosome 6 from 160,744,233 to 160,944,757 and chromosome 7 from 142,935,233 to 143,008,163. Circos plots of ETS1, MAP3K14, SLC22A3 and CASP2 chromosome conformation signature markers showing the chromosomal loops were produced.

Principal component analysis for the five-markers was used to determine abundance levels and to identify potential outliers. This analysis was applied to 78 samples containing two groups. First group, 49 known samples (24 PCa and 25 healthy controls) combined with a second group of 29 samples including, 24 PCa samples and 5 healthy control samples (FIG. 18). The final training set was built using 95 PCa and 96 control samples and then tested on an independent blinded validation cohort of 20 samples (10 controls and 10 PCa). The sensitivity and specificity for PCa detection using chromosomal interactions in five genomic loci were 80% (CI 44.39% to 97.48%) and 80% (CI 44.39% to 97.48%), respectively (Table 12).

To select epigenetic biomarkers that can stratify PCa, the samples from PCa patients categorised into risk group categories 1-3 (low, intermediate and high, respectively, Table 10) were screened for statistically significant differences in conditional and stable profiles of genome architecture. EpiSwitch™ was used on blood samples in a three-step process to identify, evaluate, and validate statistically-significant differences in chromosomal conformations between PCa patients at different stages of the disease (FIG. 17). For the first step, the array used covered 425 genetic loci, with testing probes for the total of 14,241 potential chromosomal conformations. Patients with high-risk PCa category 3 were compared to low-risk category 1 or intermediate-risk category 2. In total, 181 potential stratification marker leads for PCR evaluation were identified using enrichment statistics (Table 19). The top 70 top markers were then taken to the next stage of PCR detection for further evaluation of stratification of high-risk category 3, vs low-risk category 1 patient samples and finally a six-marker set for high category 3 vs low category 1 was established (Table 13). The best markers were identified using Chi-square and then built into a classifier on a testing set of category 1 (n=21) and category 3 (n=19). An independent cohort of category 1 (n=21) and category 3 (n=6) which were not used for any marker reduction were then used for first round of blind validation. Similarly, a six-marker set was evaluated for high-risk category 3 vs intermediate-risk category 2 on a testing set of category 3 and category 2 including, 25 and 19 samples, respectively. An independent cohort of category 2 and category 3 (n=6 in each group) which were not used for any marker reduction were then used for first round blind validation.

For the last step, to further validate the chromosome conformation signature used to inform PCa prognosis, the six-marker set for high-risk category 3 vs low-risk category 1 was tested on a larger, more representative cohort. The original blind cohort was expanded to 67 samples, including 40 samples used in marker reduction (Table 15). Similarly, the six-marker set for high-risk category 3 vs intermediate-risk category 2 was tested on a on a larger, more representative cohort. The original blind cohort was expanded to 43 samples (Table 16).

A six-marker set for category 3 vs category 1 was established. This set contained bone morphogenetic protein 6 (BMP6), ETS transcription factor ERG (ERG), macrophage scavenger receptor 1 (MSR1), mucin 1 (MUC1), acetyl-CoA acetyltransferase 1 (ACAT1) and death-associated protein kinase 1 (DAPK1) genes (Table 13). Six-biomarkers were identified for high-risk category 3 vs intermediate-risk category 2, including hydroxy-delta-5-steroid dehydrogenase, 3 beta- and steroid delta-isomerase 2 (HSD3B2), vascular endothelial growth factor C (VEGFC), apoptotic peptidase activating factor 1 (APAF1), MUC1, ACAT1 and DAPK1. Notably, the last three-biomarkers (MUC1, ACAT1 and DAPK1) were common between categories 1 vs 3 and 3 vs 2 (Table 13). Stratification of high-risk category 3 vs low-risk category 1 PCa using chromosomal interactions in six genomic loci showed sensitivity of 80% (CI 59.30% to 93.17%) and specificity of 92% (CI 80.52% to 98.50%) in the blind cohort of 67 samples (Table 15). Similarly, the six-marker set for high-risk category 3 vs intermediate-risk category 2 was tested on a on a larger, more representative cohort of 43 samples demonstrating sensitivity of 84% (CI 63.92% to 95.46%), and specificity of 88% (CI 65.29% to 98.62%) (Table 16).

Using five matching peripheral blood and primary tumour samples, we have compared the epigenetic markers identified in peripheral circulation (Table 13) to the tumour tissue. Our results showed that a number of deregulation markers detected in the blood as part of stratifying signatures for category 1 vs 3 and category 2 vs 3 could be detected in the tumour tissue (Table 14). This demonstrates that the chromosome interactions that can be detected systemically could be detected under same conditions in the primary site of tumorigenesis.

Timely diagnosis of prostate cancer is crucial to reducing mortality. The European randomised study of screening for PCa has shown significant reduction in PCa mortality in men who underwent routine PSA screening. Total screening, however, leads to overdiagnosis of clinically insignificant disease and new less invasive tests capable of discriminating low-from high-risk disease are urgently required.

Our epigenetic analysis approach provides a potentially powerful means to address this need. The binary nature of the test (the chromosomal loop is either present or not) and the enormous combinatorial power (>10¹⁰ combinations are possible with ^{˜}50,000 loops screened) may allow creating signatures that accurately fit clinically well-defined criteria. In PCa that would be discerning low-risk vs high-risk disease or identifying small but aggressive tumours and determining most appropriate therapeutic options. In addition, epigenetic changes are known to manifest early in tumourigenesis, making them useful for both diagnosis and prognosis.

In this study, we identified and validated chromosome conformations as distinctive biomarkers for a non-invasive blood-based epigenetic signature for PCa. Our data demonstrate the presence of stable chromatin loops in the loci of ETS1, MAP3K14, SLC22A3 and CASP2 genes present only in PCa patients (Table 11). Validation of these markers in an independent set of 20 blinded samples showed 80% sensitivity and 80% specificity (Table 12), which is remarkable for a PCa blood test. Interestingly, the expression of some of these genes has already been linked to cancer pathophysiology. ETS1 is a member of ETS transcription factor family. ETS1-overexpressing prostate tumours are associated with increased cell migration, invasion and induction of epithelial-to-mesenchymal transition. MAP3K14 (also known as nuclear factor-kappa-beta (NF-kβ)-inducing kinase (NIK)) is a member of MAP3K group (or MEKK). Physiologically, MAP3K14/NIK can activate noncanonical NF-113 signalling and induce canonical NF-kβ signalling, particularly when MAP3K14/NIK is overexpressed. A novel role for MAP3K14/NIK in regulating mitochondrial dynamics to promote tumour cell invasion has been described. SLC22A3 (also known as organic cation transporter 3 (OCT3)) is a member of SLC group of membrane transport proteins. SLC22A3 expression is associated with PCa progression. CASP2 is a member of caspase activation and recruitment domains group. Physiologically, CASP2 can act as an endogenous repressor of autophagy. Two of the identified genes (SLC22A3 and CASP2) were previously shown to be inversely correlated with cancer progression. Importantly, the presence of the chromatin loop can have indeterminate effect on gene expression.

To screen for PCa prognostic markers we performed the EpiSwitch™ custom array to analyse competitive hybridization of DNA from peripheral blood from patients with low-risk PCa (classification 1) and high risk PCa (classification 3). Six-marker set was identified for high-risk category 3 vs low-risk category 1, including BMP6, ERG, MSR1, MUC1, ACAT1 and DAPK1. Six-biomarkers were identified for high-risk category 3 vs intermediate-risk category 2, including HSD3B2, VEGFC, APAF1, MUC1, ACAT1 and DAPK1. Three of these biomarkers (MUC1, ACAT1 and DAPK1) were shared between these sets. Our data show high concordance between chromosomal conformations in the primary tumour and in the blood of matched PCa patients at stages 1 and 3 (Table 14). The prognostic significance and diagnostic value of some of these genes have previously been suggested. BMP6 plays an important role in PCa bone metastasis. In addition to ETS1, ERG is another member of the ETS family of transcription factors. Overwhelming evidence, suggesting that ERG is implicated in several processes relevant to PCa progression including metastasis, epithelial—mesenchymal transition, epigenetic reprogramming, and inflammation. MSR1 may confer a moderate risk to PCa. MUC1 is a membrane-bound glycoprotein that belongs to the mucin family. MUC1 high expression in advanced PCa is associated with adverse clinicopathological tumour features and poor outcomes. ACAT1 expression is elevated in high-grade and advanced PCa and acts as an indicator of reduced biochemical recurrence-free survival. DAPK1 could function either as a tumour suppressor or as an oncogenic molecule in different cellular context. HSD3B2 plays a crucial role in steroid hormone biosynthesis and it is up-regulated in a relevant fraction of PCa that are characterized by an adverse tumour phenotype, increased androgen receptor signalling and early biochemical recurrence. VEGFC is a member of VEGF family and its increased expression is associated with lymph node metastasis in PCa specimens. In a comprehensive biochemical approach, APAF1 has been described as the core of the apoptosome.

Despite the identification of these loci, the mechanism of cancer-related epigenetic changes in PBMCs remains unidentified. The interaction, however, can be detected systemically and could be detected under same conditions in the primary site of tumorigenesis (Table 14). Thus for us to be able to measure the changes, chromatin conformation in PBMCs must be directed by an external factor; presumably something generated by the cells of the PCa tumour. It is known that a significant proportion of chromosomal conformations are controlled by non-coding RNAs, which regulate the tumour-specific conformations. Tumour cells have been shown to secrete non-coding RNAs that are endocytosed by neighbouring or circulating cells and may change their chromosomal conformations, and are possible regulators in this case. While RNA detection as a biomarker remains highly challenging (low stability, background drift, continuous basis for statistical stratification analysis), chromosome conformation signatures offer well recognized stable binary advantages for the biomarker targeting use, specifically when tested in the nuclei, since the circulating DNA present in plasma does not retain 3D conformational topological structures present in the intact cellular nuclei. It is important to mention, that looking at one genetic locus does not equate to looking at one marker, as there may be multiple chromosome conformations present, representing parallel pathways of epigenetic regulation over the locus of interest.

One of the key challenges in the present clinical practice of PCa diagnosis is the time it takes to make a definitive diagnosis. So far, there is no single, definitive test for PCa. High levels of PSA will set the patient on a long journey of uncertainty where he will undergo magnetic resonance imaging scan followed by biopsy, if needed. Although a biopsy is more reliable than a PSA test, it is a major procedure where missing the cancer lesions can still be an issue. The five-set biomarker panel described here is based on a relatively inexpensive and well-established molecular biology technique (PCR). The samples are based on biofluid, which is simple to collect and provides clinicians with rapidly available clinical readouts within few hours. This in turn, offers a substantial time and cost savings and aids an informative diagnostic decision which fills the gap in the current protocols for assertive diagnosis of PCa.

Predicting the risk of PCa is pivotal for making an informed decision on treatment options. Five-year survival rate in the low risk group is more than 95% and most men would benefit from less invasive therapy. Currently, PCa risk stratification is based on combined assessment of circulating PSA, tumour grade (from biopsy) and tumour stage (from imaging findings). The ability to derive similar information using a simple blood test would allow significant reduction in costs and would speed up the diagnostic process. Of particular importance in PCa treatment is identifying the few tumours that initially present as low-risk, but then progress to high-risk. This subset would therefore benefit from a quicker and more-radical intervention.

In conclusion, here, we have identified subsets of chromosomal conformations in patients' PBMCs that are strongly indicative of PCa presence and prognosis. These signatures have a significant potential for the development of quick diagnostic and prognostic blood tests for PCa and significantly exceed the specificity of currently used PSA test. Preferred markers and combinations include
ETS1, MAP3K14, SLC22A3 and CASP2. This is Diagnostic, by nested PCR markers
BMP6, ERG, MSR1, MUC1, ACAT1 and DAPK1. This is Prognostic Signature (High-risk Category 3 vs Low Risk Category 1, by Nested PCR Markers)
HSD3B2, VEGFC, APAF1, MUC1, ACAT1 and DAPK1. This is Prognostic (High Risk Cat 3 vs Medium Risk Cat 2)

### Example 5. Further Work on DLBLC

Diffuse large B-cell lymphoma (DLBCL) is a heterogenous blood cancer, but can be broadly classified into two main subtypes, germinal center B-cell-like (GCB) and activated B-cell-like (ABC). GCB and ABC subtypes have very different clinical courses, with ABC having a much worse survival prognosis. It has been observed that patients with different subtypes also respond differently to therapeutic intervention, in fact, some have argued that ABC and GCB can be thought of as separate diseases altogether. Due to this variability in response to therapy, having an assay to determine DLBCL subtypes has important implications in guiding the clinical approach to the use of existing therapies, as well as in the development of new drugs. The current gold standard assay for subtyping DLBCL uses gene expression profiling on formalin fixed, paraffin embedded (FFPE) tissue to determine the “cell of origin” and thus disease subtype. However, this approach has some significant clinical limitations in that it 1) requires a biopsy 2) requires a complex, expensive and time-consuming analytical approach and 3) does not classify all DLBCL patients.

Here, we took an epigenomic approach and developed a blood-based chromosome conformation signature (CCS) for identifying DLBCL subtypes. An iterative approach using clinical samples from 118 DLBCL patients was taken to define a panel of six markers (DLBCL-CCS) to subtype the disease. The performance of the DLBCL-CCS was then compared to conventional gene expression profiling (GEX) from FFPE tissue.

The DLBCL-CCS was accurate in classifying ABC and GCB in samples of known status, providing an identical call in 100% (60/60) samples in the discovery cohort used to develop the classifier. Also, in the assessment cohort the DLBCL-CCS was able to make a DLBCL subtype call in 100% (58/58) of samples with intermediate subtypes (Type III) as defined by GEX analysis. Most importantly, when these patients were followed longitudinally throughout the course of their disease, the EpiSwitch′ associated calls tracked better with the known patterns of survival rates for ABC and GCB subtypes.

This study provides an initial indication that a simple, accurate, cost-effective and clinically adoptable blood-based diagnostic for identifying DLBCL subtypes is possible.

### Background

Diffuse large B-cell lymphoma (DLBCL) is the most common type of blood cancer and numerous studies using different methodologies have demonstrated it to be genetically and biologically heterogeneous. The two principal DLBCL molecular subtypes are germinal center B-cell-like (GCB) and activated B-cell-like (ABC), although more granular definitions of molecular subtypes have also been proposed. These two primary subtypes have a high degree of clinical relevance, as it has been observed that they have dramatically different disease courses, with the ABC subtype having a far worse survival prognosis. Perhaps more importantly, as novel investigational agents to treat GCB and ABC (or non-GCB) subtypes are evaluated in clinical settings and the historical observation that overall response rates in unselected patients is low, there is a pressing need to identify patient subtypes prior to the initiation of therapy. Historically, DLBCL subtypes are determined by identifying the “cell of origin” (COO). The original COO classification was based on the observed similarity of DLBCL gene expression to activated peripheral blood B cells or normal germinal center B-cells by hierarchical clustering analysis (3). This COO-classification by whole-genome expression profiling (GEP) classifies DLBCL into activated B-cell like (ABC), germinal center B-cell like (GCB), and Type-III (unclassified) subtypes, with the ABC-DLBCL characterized by a poor prognosis and constitutive NF-kB activation. In their seminal work, Wright et al. identified 27 genes that were most discriminative in their expression between ABC and GCB-DLBCL, and developed a linear predictor score (LPS) algorithm for COO-classification. These original studies are entirely based on retrospective investigations of fresh-frozen (FF) lymphoma tissues. A major challenge for the application of this COO-classification in clinical practice has been an establishment of a robust clinical assay amenable to routine formalin-fixed paraffin-embedded (FFPE) diagnostic biopsies. Several studies have also investigated the possibility of COO classification of DLBCL using FFPE tissues by quantitative measurement of mRNA expression, including quantitative nuclease protection assay, GEP with the Affymetrix HG U133 Plus 2.0 platform or the Illumina whole-genome DASLassay, and NanoString Lymphoma Subtyping Test (LST) technology. Several immunohistochemistry (IHC)-based algorithms have also been investigated to recapitulate the COO-classification by GEP. In general, these studies demonstrated high confidence of COO-classification of DLBCL using FFPE tissues and a robust separation in overall survival between ABC and GCB subtypes, but suffer from reproducibility issues, particularly lack of concordance between assays. In addition, any IHC-based measure requires baseline tissue, which is not always available and current turnaround times from sample collection to assay readout are long, making implementation in clinical practice a challenge.

Among the approaches that have been used historically to subtype DLBCL, one method for COO assessment uses an assay that measures the expression of 27 genes from FFPE tissue by quantitative reverse transcription PCR (qRT-PCR) using the Fluidigm BioMark HD system. While there are some advantages to this methodology over existing techniques, the approach still faces some major obstacles that limit its clinical application in that it 1) requires a tissue biopsy 2) relies on expensive, non-standard and time-consuming laboratory procedures. As such, having a blood-based assay would advance the field by providing a simple, reliable and cost-effective method for DCBCL subtyping with enhanced clinical applicability.

In this study, we used a novel blood-based assay to determine COO classification in DLBCL patients by focusing on detecting changes in genomic architecture. As part of the epigenetic regulatory framework, genomic regions can alter their 3-dimensional structure as a way of functionally regulating gene expression. A result of this regulatory mechanism is the formation of chromatin loops at distinct genomic loci. The absence or presence of these loops can be empirically measured using chromosome conformation capture (3C). Multiple genomic regions contribute to epistatic modulation through the formation of stable, conditional long-range chromosome interactions. The collective measurement of chromosome conformations at multiple genomic loci results in a chromosome conformation signature (CCS), or a molecular barcode that reflects the genomes response to its external environment. For detection, screening and monitoring of CCS we utilized the EpiSwitch platform, an established, high resolution and high throughput methodology for detecting CCSs. Based on 3C, the EpiSwitch platform has been developed to assess changes in chromatin structure at defined genetic loci as well as long-range non-coding cis- and trans-regulatory interactions. Among the advantages of using EpiSwitch for patient stratification are its binary nature, reproducibility, relatively low cost, rapid turnaround time (samples can be processed in under 24 hours), the requirement of only a small amount of blood (^{˜}50 mL) and compliance with FDA standards of PCR-based detection methodologies. Thus, chromosome conformations offer a stable, binary, readout of cellular states and represent an emerging class of biomarkers.

Here, we used an approach based on the assessment of changes in chromosomal architecture to develop a blood-based diagnostic test for DLBCL COO subtyping. We hypothesized that interrogation of genomic architecture changes in blood samples from DLBCL patients could offer an alternative method to tissue-based COO classification approaches and provide a novel, non-invasive, and more clinically applicable methodology to guide clinical decision making and trial design.

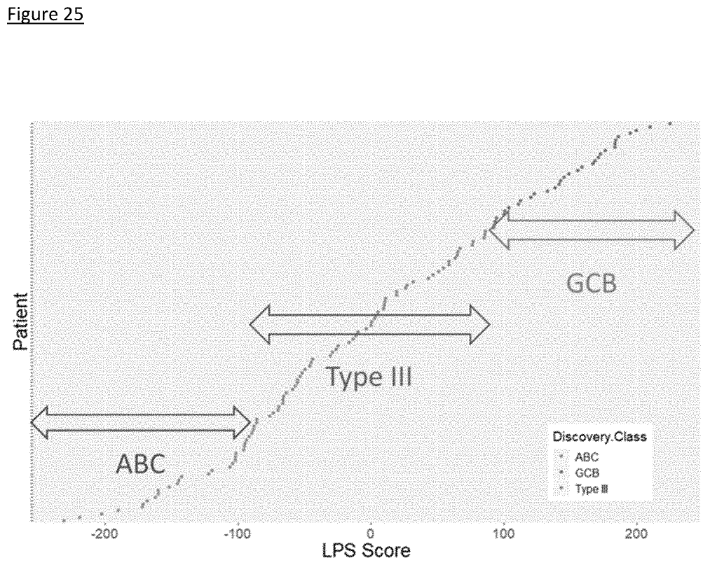

A total of 118 DLBCL patients with a known COO subtype and 10 healthy controls (HC) were used in this study. The samples were a subset of those collected in a phase III, randomized, placebo-controlled, trial of rituximab plus bevacizumab in aggressive Non-Hodgkin lymphoma. Briefly, adult patients aged 1.8 years with newly-diagnosed CD20-positive DLBCL were randomized to R-CHOP or R-CHOP plus bevacizumab (RA-CHOP). Blood samples collected from 60 DLBCL patients were used as a development cohort to identify, evaluate, and refine the CCS biomarker leads. The patients from this cohort were all typed as high/strong GCB (30) or ABC (30) with a high subtype specific LPS (linear predictor scores). The remaining 58 DLBCL samples had intermediate LPS and were determined as ABC, GCB or Unclassified by Fluidigm testing (FIG. 25). These patient samples were not used for CCSs biomarker discovery and development; but were used at a later stage to assess the resultant classifier. The Fluidigm testing was done using tissue obtained from lymph nodes (either as punch biopsies or removed during surgery), and the EpiSwitch analysis was done using matched peripheral whole blood collected from the patients prior to receiving any therapy.

In addition to patient samples, 12 cell lines (six ABC and six GCB) were also used in the initial stage of the biomarker screening to identify the set of chromosome conformations that could best discriminate between ABC and GCB disease subtypes (Table 20). Cell lines were obtained from the American Type Culture Collection (ATCC), the German Collection of Microorganisms and Cell Cultures (DSMZ), and the Japan Health Sciences Foundation Resource Bank (JHSF).

RNA was isolated and purified from pre-treatment FFPE biopsies. DLBCL subtypes were determined by adaption of the Wright et al. algorithm to expression data from a custom Fluidigm gene expression panel containing the 27 genes of the DLBCL subtype predictor. Validation of the COO assay by comparing Fludigm qRT-PCR to Affymetrix data in a cohort of 15 non-trial subjects revealed a high correlation between qRT-PCR measurements from matched fresh frozen (FF) and FFPE samples across 19 classifier genes used. We also found a high correlation between Affymetrix microarray and Fluidigm qRT-PCR measurements from the same FF samples. Classifier gene weights calculated from qRT-PCR data from the Fluidigm COO assay were highly concordant with weights obtained from previous microarray data in an independent patient cohort. We observed high correlation (76% concordance) between LPS derived from the Fluidigm assay, data in FFPE tumor, and LPS derived from Affymetrix microarray data in matched FF tissue in the technical registry cohort.

A pattern recognition algorithm was used to annotate the human genome for sites with the potential to form long-range chromosome conformations. The pattern recognition software operates based on Bayesian-modelling and provides a probabilistic score that a region is involved in long-range chromatin interactions. Sequences from 97 gene loci (Table 21) were processed through the pattern recognition software to generate a list of the 13,322 chromosomal interactions most likely to be able to discriminate between DLBCL subtypes. For the initial screening, array-based comparisons were performed. 60-mer oligonucleotide probes were designed to interrogate these potential interactions and uploaded as a custom array to the Agilent SureDesign website. Each probe was present in quadruplicate on the EpiSwitch microarray. To subsequently evaluate a potential CCS, nested PCR (EpiSwitch PCR) was performed using sequence-specific oligonucleotides designed using Primer3. Oligonucleotides were tested for specificity using oligonucleotide specific BLAST.

The top ten genomic loci that were identified as being dysregulated in DLBCL were uploaded as a protein list to the Reactome Functional Interaction Network plugin in Cytoscape to generate a network of epigenetic dysregulation in DLBCL. The ten loci were also uploaded to STRING (Search Tool for the Retrieval of Interacting Genes/Proteins DB) (https.//string-db.org/), a database containing over 9 million known and predicted protein-protein interactions. Restricting to only human interactions, the main network (i.e. non-connected nodes were excluded) was generated. The top false discovery rate (FDR)-corrected functional enrichments were identified by Gene Ontology (GO) and the Kyoto Encyclopedia of Genes and Genomes (KEGG) databases. The top ten genomic loci were also uploaded to the KEGG Pathway Database (https://www.genome.jp/kegg/pathway.html) to identify specific biological pathways that exhibit dysregulation in DLBCL.

Exact and Fisher's exact test (for categorical variables) were used to identify discerning markers. The level of statistical significance was set at p 0.05, and all tests were 2-sided. The Random Forest classifier was used to assess the ability of the EpiSwitch markers to identify DLBCL subtypes. Long term survival analysis was done by Kaplan-Meier analysis using the survival and survminer packages in R (38). Mean survival time was calculated using a two-tailed t-test.

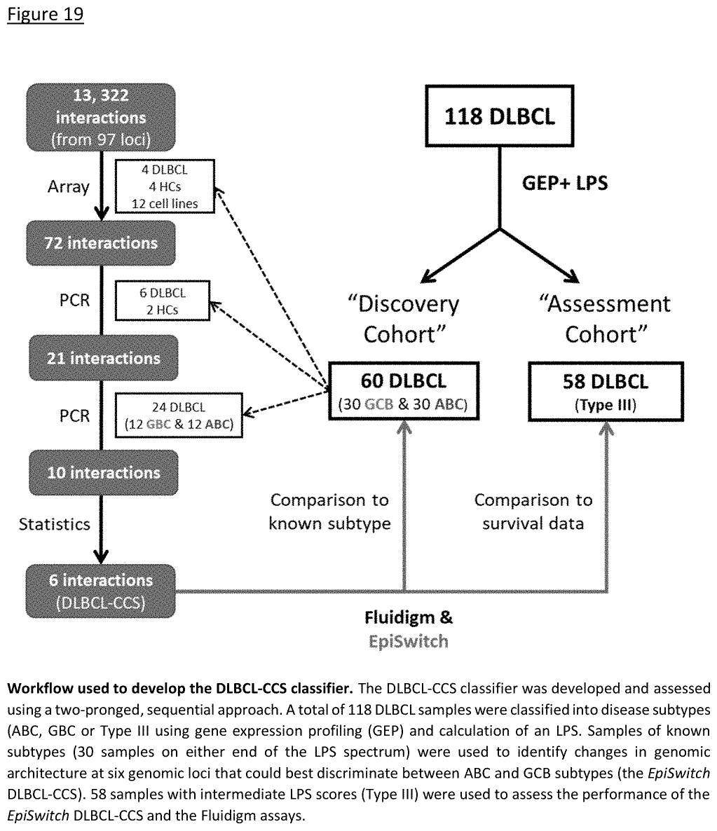

We employed a step-wise approach to discover and validate a CCS biomarker panel that could differentiate between DLBCL subtypes (FIG. 19). As a first step in the discovery of the EpiSwitch classifier, 97 genetic loci (Table 21) were selected and annotated for the predicted presence of chromosome conformation interaction sites and screened for their empirical presence using the EpiSwitch CGH Agilent array. The annotated array design represented 13,322 chromosome interaction candidates, with an average of 99 distinct cis-interactions tested at each locus (99±64; mean±SD). This discovery array was used to screen and identify a smaller pool of chromosome conformations that could differentiate between the two main DLBCL subtypes. The samples used for this step were from GCB and ABC cell lines (Table 20) as well as whole blood from four typed DLBCL patients (two GCB and two ABC) and four HCs. The cell lines were grouped into high ABC and GCB and low ABC and GCB based on gene expression analysis. The comparisons used on the array were: 1) individual comparisons of DLBCL patients to pooled HCs 2) pooled DLBCL samples to pooled HC samples 3) pooled high ABC compared to pooled high GCB cell lines, and 4) pooled low ABC versus pooled low GCB cell lines.

From the array analysis, we identified 1,095 statistically significant chromosomal interactions that differentiated between high ABC and GCB cell lines and were present in blood samples from DLBCL patients, but absent in HCs. These were further reduced to the top 293 interactions using a set of statistical filters, 151 of which were associated with the ABC subtype and 143 of which were associated with the GCB subtype. The top 72 interactions from either subtype (36 interactions for ABC and 36 interactions for GCB) were selected for further refinement using the EpiSwitch PCR platform on 60 typed DLBCL patient samples. For all 118 DLBCL samples, initial subtype classification was assigned based on the Wright algorithm, which calculates a linear predictor score (LPS) from the expression of a panel of 27 genes. 60 samples were classified as either ABC or GBC and used to develop the EpiSwitch classifier (the “Discovery Cohort”) and 58 samples were of intermediate LPS scores and used to evaluate the performance of the EpiSwitch classifier (the “Assessment Cohort”) (FIG. 19).

The 72 interactions identified in the initial screen were narrowed to a smaller pool using both the DLBCL patient samples during the discovery step and a second cohort of 60 DLBCL typed (30 ABC and 30 GCB) patient samples along with 12 HC (FIG. 19). The DLBCL subtype calls made by the EpiSwitch assay were confirmed using the Fluidigm platform. The Fluidigm gene expression analysis was performed on tissue biopsy samples, whereas whole blood from the same patients was used for the EpiSwitch PCR assay. The initial steps in refinement were to confirm by PCR that the 72 chromosomal interactions identified in the initial screen were specific to DLBCL and were absent in the HC samples. This was first tested on six untyped DLBCL samples and two HCs and resulted in identification of 21 interactions that were specific for DLBCL. Next, we used EpiSwitch PCR to test 24 blood samples from typed DLBCL patient samples (12 ABC and 12 GCB) to identify DLBCL-specific chromosome interactions using Fisher's test. This resulted in a set of 10 discriminating chromosome conformation interactions that could accurately discriminate between ABC and GCB subtypes and were further evaluated on blood samples from an additional set of 36 DLBCL samples (18 ABC and 18 GCB) (FIG. 19).

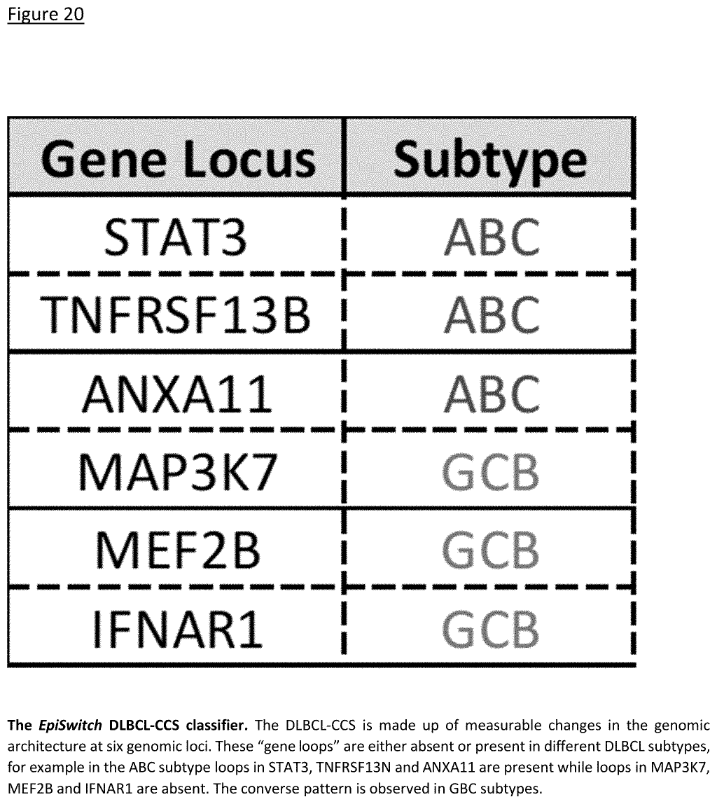

To test the accuracy, performance and robustness of the 10-marker panel, we used Exact test for feature selection on 80% of the complete sample cohort (Total 48 samples: 24 ABC and 24 GCB), with the remaining 20% (12 samples, 6 ABC and 6 GCB) used for later testing of the final selected CCSs markers. The data was split 10 times and the Exact test run on each of the splits using the 80% training set of each split. The composite p-value for the 10 markers over the 10 splits was then used to rank the markers. This analysis identified six chromosome conformations in the IFNAR1, MAP3K7, STAT3, TNFRSF13B, MEF2B, and ANXA11 genetic loci. Collectively, these six interactions formed the DLBCL chromosome conformation signature (DLBCL-CCS) (FIG. 20).

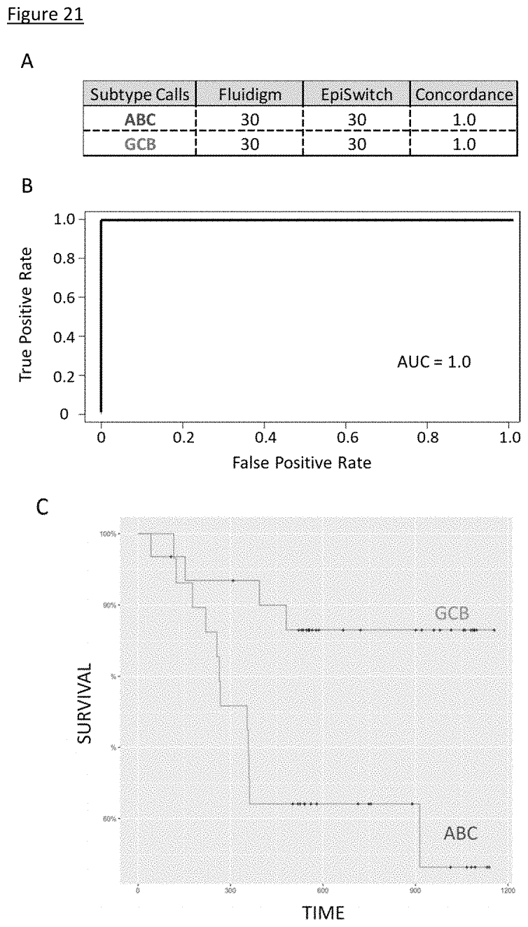

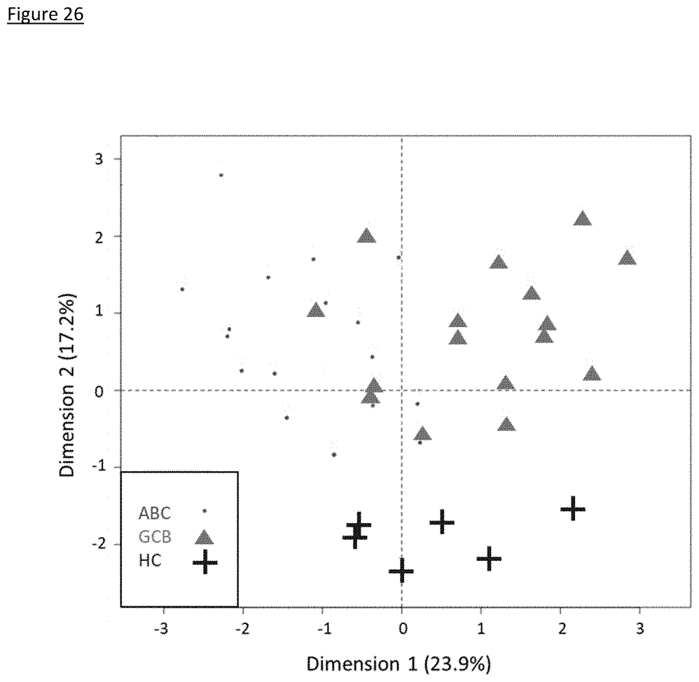

The six markers in the DLBCL-CCS were used to generate a Random forest classifier model and applied to classify the test sets for each of the data splits (12 samples, 6 ABC and 6 GCB) in the Discovery Cohort of known disease subtypes. By principal component analysis (PCA), the DLBCL-CCS classifier was able to separate ABC and GCB patients from healthy controls (FIG. 26). The composite prediction probabilities for the DLBCL-CCS is shown in Table 22 along with the odds ratio for each marker and the odd ratio for the model generated using logistic regression. The model provided a prediction probability score for ABC and GCB, ranging from 0.186 to 0.81 (0=ABC, 1=GCB). The probability cut-off values for correct classification were set at ≤0.30 for ABC and ≥0.70 for GCB. The score of ≤0.30 had a true positive rate (sensitivity) of ≤100% (95% confidence interval [95% CI] 88.4-100%), while a score of ≥0.70 had a true negative response rate (specificity) of 96.7% (95% CI 82.8-99.9%). With the DLBCL-CCS classifier, 60 out of 60 patients (100%) were correctly classified as either ABC or GCB, when compared to the Fluidigm calls for subtyping (FIG. 21A, Table 22). The AUC under the receiver operating characteristic (ROC) curve for the DLBCL-CCS classifier on this sample cohort was 1 (FIG. 21B). Last, we compared the DLBCL subtype calls made by the DLBCL-CCS to the long-term survival curves of the patients with known disease subtype. The patients called as ABC showed significantly worse survival than those patients called as GBC (FIG. 21C).

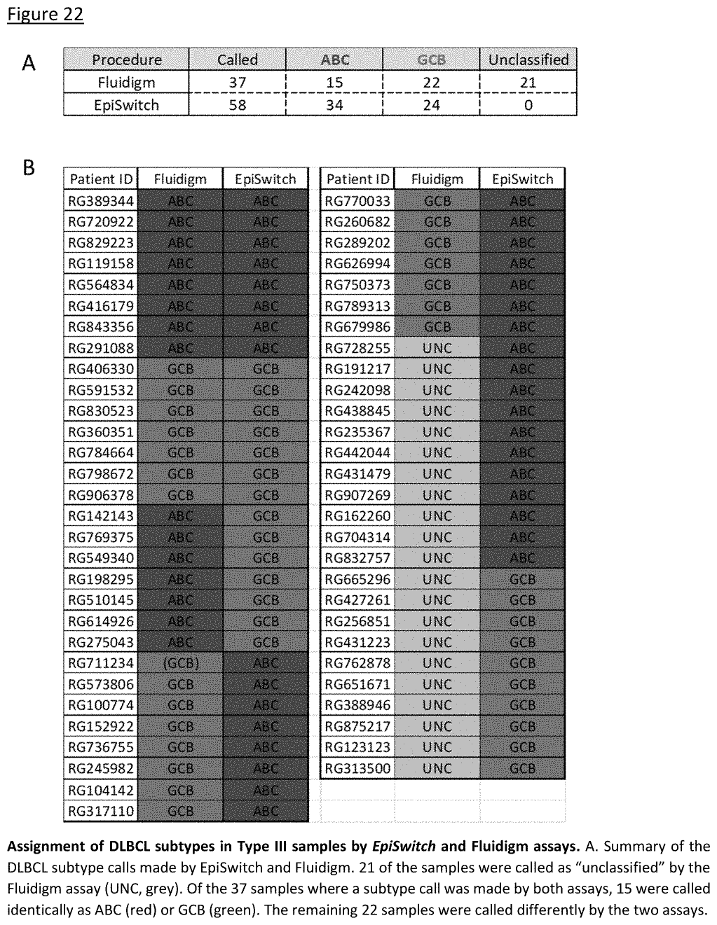

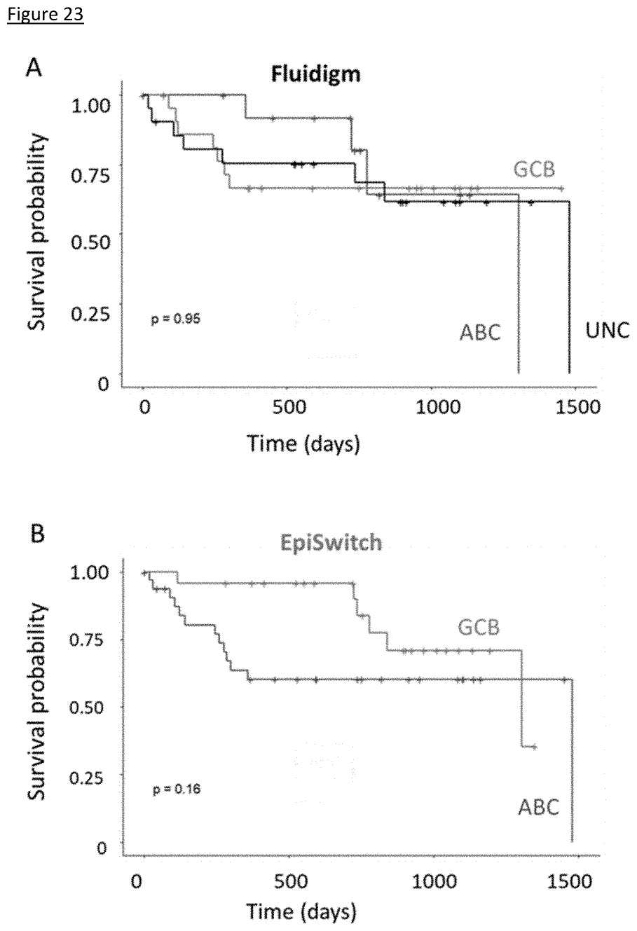

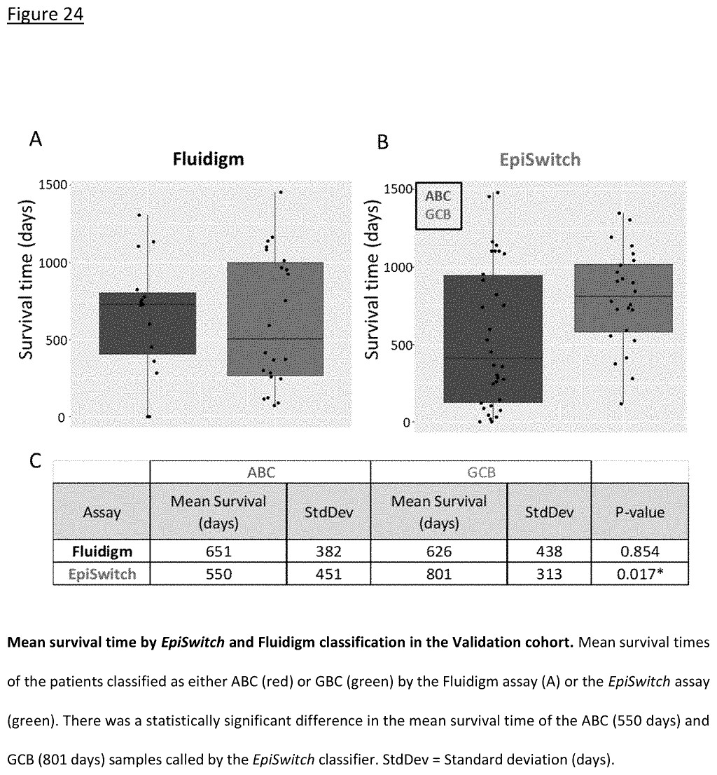

Next, we evaluated the performance of the DLBCL-CCS the Assessment Cohort of 58 DLBCL patients with a more intermediate LPS value. We applied the DLBCL-CCS to assign these patients into DLBCL subtypes and compared the readouts to those made by Fluidigm. The DLBCL-CCS made subtyping calls for all 58 samples, whereas the Fluidigm assay made subtyping calls for 37 of the samples, leaving 21 as “unclassified” (FIG. 22). Of the 37 samples where subtype calls for both assays was available, 15 samples (40%) were called similarly by both assays (8 ABC and 7 GCB) (FIG. 22). Next, we evaluated the performance of the DLBCL subtype calls made by the DLBCL-CCS and Fluidigm by comparing the subtype calls made at diagnosis with the long-term survival curves of the Type III patients. As shown in the Kaplan-Meier survival curves in FIG. 23, the ABC/GBC calls made by the DLBCL-CCS was able to separate the two populations based on the known survival trends in DLBCL, with the ABC subtype having a worse prognosis. In contrast, the ABC and GCB populations as defined by Fluidigm showed the opposite of what has been observed clinically, with samples classified as ABC having longer survival times than those classified as GCB. Though not statistically significant, the subtype calls made by the DLBCL-CCS matched historical clinical observations of survival differences between the subtypes by Hazard ratio analysis. We did find a significant difference in mean survival time between the two methods. The mean survival of patients classified as ABC and GCB by Fluidigm was 651 and 626 days, respectively (p=0.854), while the mean survival of patients classified as ABC and GCB by the DLBCL-CCS assay was 550 and 801 days (p=0.017) (FIG. 24).

In order to explore the relationship between the loci that were observed to be epigenetically dysregulated in this study and biological mechanisms that have previously been reported to be linked to DLBCL, we performed a series of network and pathway analyses using the top 10 dysregulated loci as inputs. First, we explored how these loci were biologically related by building a Reactome Functional Interaction Network in Cytoscape which revealed a network centred on NFKB1, STAT3 and NFATC1. A similar picture emerged when the 10 loci were used to build a network using STRING DB, with the most connected hubs centring on NFKB1, STAT3 and MAP3K7 and CD40. The top enriched GO term for biological process was “positive regulation of transcription, DNA-templated”, the top enriched GO term for molecular function was “transcriptional activator activity, RNA polymerase II transcription regulatory region sequence-specific binding” and the “Toll-like receptor signalling pathway” was the most enriched KEGG pathway (Table 22). When we mapped the top ten loci to the KEGG Toll-like receptor signalling pathway, we found that specific cascades related to the production of proinflammatory cytokines and costimulatory molecules through the NF-kB and the interferon mediated JAK-STAT signalling cascades.

Due to the observed differences in disease progression for the different DLBCL subtypes, there is a pressing clinical need for a simple and reliable test that can differentiate between ABC and GBC disease subtypes. Given the aggressive nature of the disease, DLBCL requires immediate treatment. The two main subtypes have different clinical management paradigms and with several therapeutic modalities in development that target specific subtypes, having a rapid and accurate disease diagnostic is critical when clinical management depends on knowing disease subtype. The field of COO-classification in DLBCL has expanded from IHC based methodologies to DNA microarrays, parallel quantitative reverse transcription PCR (qRT-PCR) and digital gene expression. A current favoured method is based on identification of the COO by GEP on FFPE tissue and suffers from some technical and logistical limitations that limit its broad adoption in the clinical setting. In addition, there are many factors that affect the performance and reliability of COO-classification by GEP on FFPE tissue; including the nature/quality of lymphoma specimen, the experimental methods for data collection; data normalization and transformation, the type of classifier used, and the probability cut offs used for subtype assignment. Last, going from sample collection to an end readout using the Fluidigm approach is a complex and time-consuming process with many steps in between having the potential to introduce performance variability. All of these factors have an impact on the overall turnaround time of the assay and limits how it can be used clinically to diagnose and inform treatment of the disease using existing medications as well as select patients for late stage trials for novel DLBCL therapeutics. Thus, the need for a simple, minimally invasive and reliable assay to differentiate DLBCL subtypes is needed.

Using a stepwise discovery approach, we identified a 6-marker epigenetic biomarker panel, the DLBCL-CCS, that could accurately discriminate between DLBCL subtypes. When compared to the subtype results derived from the gene expression signature there was perfect concordance; which was expected as these were samples that were used to develop the classifier. The concordance between the two assays when applied to samples with an intermediate LPS was lower (just over 40%). This is perhaps expected, as it has been noted that there is a lack of overall concordance in DLBCL subtype calls with different methods of classification, and the Type III samples are perhaps a more heterogenous population reflecting a more intermediate biology to begin with. However, when we evaluated the predictive classification ability of the EpiSwitch assay in the Type III DLBCL patients followed longitudinally as their disease progressed, baseline predictions of disease subtype using the EpiSwitch assay was better at predicting actual disease subtype based on observed survival curves in patients with unclassified disease. The observation that the epigenetic readout based on regulatory 3D genomics used here is more consistent with actual clinical outcomes than the transcription-based gold-standard molecular approaches represents an actionable advance in the management of DLBCL. It is also consistent with a system biology evaluation of regulatory 3D genomics as a molecular modality closely linked to phenotypical differences in oncological conditions.

We do note that DLBCL operates on a biological continuum, with significant heterogeneity in disease biology between subtypes. By design, the DLBCL-CCS was set up to classify Type III samples into either ABC or GCB subtypes. By GEX analysis, the Type III samples were identified as having intermediate subtype biology so may represent a more heterogenous population of patients. However, the overall observation that the DLBCL-CCS was a better predictor of disease subtype as measured by clinical progression than using a GEX-based approach and the fact that the EpiSwitch assay was able to make subtype calls in all samples, provides an initial indication that this approach can be applied in a clinical setting to inform on prognostic outlook, potentially guide treatment decisions, and provide predictions for response to novel therapeutic agents currently in development.

In the network analysis, the NF-kB and STAT3 signalling cascades emerged as putative mediators that differentiate between DLBCL subtypes. The role of NF-kB signalling in DLBCL has been studied before, in fact, one of the discriminating features of the ABC subtype is constitutive expression of NF-kB target genes, a mechanism which has been hypothesized for the poor prognosis in these patients. In addition, mutations causing constitutive signalling activation have been observed predominantly in the ABC subtype for several NF-kB pathway genes, including TNFAIP3 and MYD88.

In addition to validating known mechanisms of DLBCL, the network analysis here identified a novel potential target for therapeutic intervention in DLBCL. For example, ANXA11, a calcium-regulated phospholipid-binding protein, has been implicated in other oncological conditions such as colorectal cancer, gastric cancer and ovarian cancer and could be a novel therapeutic intervention point in DLBCL.

One of the major clinical advantages of the approach to DLBCL subtyping described here lies in the simplified laboratory methodology and workflow. Conventional, gold-standard subtyping by GEP can be done using a variety of commercial platforms but all generally follow (and require) a four-step approach: 1) acquisition of a tissue biopsy, 2) preparation of FFPE tissue sections 3) gene expression analysis and 4) algorithmic classification of subtype. Obtaining a fine needle tissue biopsy of an enlarged, peripheral lymph node requires an inpatient visit to a clinical site and an invasive medical procedure requiring anaesthetic. Once obtained, the fresh biopsy needs to be prepared for paraffin embedding. This is a multi-step process, but generally involves immersion in liquid fixing agent (such as formalin) long enough for it to penetrate through the entire specimen, sequential dehydration through an ethanol gradient, followed by clearing in xylene, a toxic chemical. Last, the biospecimen needs to be infiltrated with paraffin wax and left to cool so that it solidifies and can be cut into micrometer sections using a microtome and mounted onto laboratory slides. The entire process of going from fresh tissue to FFPE sections on a slide can take several days. Next, in order to perform gene expression analysis, inherently unstable RNA is extracted from slide-mounted tissue sections and prepared for hybridization to microarrays according to the array manufacturer's specifications, a process that can take over a day. Following microarray hybridization, digital readouts of relative gene expression levels for the are obtained and fed into a classification algorithm to determine DLBCL subtype. All told, the process of going from a patient with suspected DLBCL to a subtype readout can take up to a week or longer, involves many different experimental steps using expensive technologies, each of which has the potential to introduce experimental variability along the way. In the approach described here, the time and the number of steps from biofluid collection to subtype readout are dramatically decreased. A patient with suspected DLBCL can present to an outpatient clinic for a routine, small volume (— 1 mL) blood draw. Fresh frozen blood can then be shipped to a central, accredited reference lab for analysis of the absence/presence of the chromosome conformations identified in this study; a process that uses an even smaller volume (^{˜}50 mL) of whole blood as input along with specific PCR primer sets and reaction conditions to detect the chromosome conformations using simple and routine PCR instrumentation in less than 24 hours from sample receipt. The approach to DLBCL subtyping described here offers an additional advantage in that the potential for further refinement using the proposed methodology exists. In this study, final readout of the DLBCL-CCS was done using a set of nested PCR reactions to detect chromosome conformations making up the classifier. This PCR-based output can be further refined to utilize quantitative PCR as a readout and operate under the minimum information for publication of quantitative real-time PCR experiments (MIQE) guidelines, designed to enhance experimental reproducibility and reliability across reference labs and testing sites. Last, the approach described here is adaptable to the evolving understanding of the disease itself, such as the different physiologically heterogeneous forms of it.

In conclusion, here we developed a robust complementary method for non-invasive COO assignment from whole blood samples using EpiSwitch CCSs readouts. We demonstrated the clinical validity of this classification approach on a large cohort of DLBCL patients. The EpiSwitch platform has several attractive features as a biomarker modality with clinical utility. CCSs have very high biochemical stability, can be detected using very small amounts of blood (typically around 50 μl) and detection utilizes established laboratory methodologies and standard PCR readouts (including MIQE-compliant qPCR). Finally, the rapid turnaround time (^{˜}8-16 hours) of the EpiSwitch assay compares favourably to the over 48 hours for the Fluidigm platform.

### Example 6. Further Work on Canine DLBCL

Here, we used the EpiSwitch™ platform technology to evaluate chromosome conformation signatures (CCS) as biomarkers for detection of canine diffuse large B-cell lymphoma (DLBCL). We examined whether established, systemic liquid biopsy biomarkers previously characterized in human DLBCL patients by EpiSwitch™ would translate to dogs with the homologous disease. Orthologous sequence conversion of CCS from humans to dogs was first verified and validated in control and lymphoma canine cohorts.

Blood samples from dogs with DLBCL and from apparently healthy dogs were obtained. All of the dogs diagnosed with DLBCL, were part of the LICKing Lymphoma trial. Blood samples were obtained from each dog prior to initiating treatment and at day +5 after the experimental intervention, but prior to initiating doxorubicin chemotherapy. EpiSwitch™ technology was used to monitor systemic epigenetic biomarkers for CCS.

A 11-marker classifier was generated with whole blood from 28 dogs, 14 diagnosed with DLBCL and 14 controls with no apparent disease, from a pool of 75 EpiSwitch CCSs identified in human DLBCL. Validation of the developed diagnostic markers was performed on a second cohort of 10 dogs: 5 with DLBCL and 5 controls. The classifier delivered stratifications for DLBCL vs. non-DLBCL with 80% accuracy, 80% sensitivity, 80% specificity, 80% positive predictive value (PPV) and 80% negative predictive value (NPV) on the second cohort.

The established EpiSwitch™ classifier contains strong systemic binary markers of epigenetic deregulation with features normally attributed to genetic markers: the binary status of these classifying markers is statistically significant for diagnosis.

TABLE 5a

| | | | Probe_ |
|---|---|---|---|
| | | | Count_ |
| | Probe | GeneLocus | Total |
| 1 | STAT3_17_40446029_40448202_ | STAT3 | 1108 |
| | 40557923_40558616_RR | | |
| 2 | ANXA11_10_81889664_81892389_ | ANXA11 | 136 |
| | 81927417_81929312_FR | | |
| 3 | CD40_20_44739847_44744687_ | CD40 | 148 |
| | 44767157_44770555_FR | | |
| 4 | IFNAR1_21_34696683_34697716_ | IFNAR1 | 80 |
| | 34777569_34779811_RF | | |
| 5 | MAP3K7_6_91275515_91285706_ | MAP3K7 | 308 |
| | 91312237_91314731_FF | | |
| 6 | MEF2B_19_19271977_19273500_ | MEF2B | 448 |
| | 19302232_19303741_RF | | |
| 7 | MLLT3_9_20556478_20560948_ | MLLT3 | 120 |
| | 20658310_20666368_FF | | |
| 8 | NFATc1_18_77133931_77135912_ | NFATc1 | 608 |
| | 77218993_77220063_RF | | |
| 9 | NFKB1_4_103425293_103430397_ | NFKB1 | 96 |
| | 103512508_103516923_FR | | |
| 10 | TNFRSF13C_22_42302849_ | TNFRSF13C | 488 |
| | 42305750_42342568_42346797_FR | | |
| 11 | BAX_19_49421750_49425644_ | BAX | 92 |
| | 49457303_49458439_RF | | |
| 12 | BCL6_3_187438677_187439687_ | BCL6 | 240 |
| | 187454088_187455426_FF | | |
| 13 | IL22RA1_1_24467543_24471444_ | IL22RA1 | 48 |
| | 24512238_24513959_RF | | |
| 14 | TNFRSF13C_22_42313974_ | TNFRSF13C | 488 |
| | 42315085_42342568_42346797_RR | | |
| 15 | FOXO1_13_41184194_41191166_ | FOXO1 | 308 |
| | 41219134_41220693_FR | | |
| 16 | HLF_17_53402207_53403714_ | HLF | 104 |
| | 53420274_53422428_FF | | |
| 17 | PAK1_11_77028527_77036211_ | PAK1 | 180 |
| | 77090325_77094591_RF | | |
| 18 | FOS_14_75744954_75746643_ | FOS | 80 |
| | 75795718_75799884_FF | | |
| 19 | MTHFR_1_11807586_11814341_ | MTHFR | 52 |
| | 11843522_11845650_RF | | |
| 20 | WNT9A_1_228068849_228075473_ | WNT9A | 40 |
| | 228135088_228140421_RR | | |
| 21 | NFATc1_18_77229964_77232215_ | NFATc1 | 608 |
| | 77280170_77283702_FR | | |
| 22 | BRCA1_17_41162341_41168331_ | BRCA1 | 297 |
| | 41242678_41245761_RR | | |
| 23 | TET2_4_106047220_106052671_ | TET2 | 104 |
| | 106063962_106067377_FF | | |
| 24 | TNF_6_31525914_31529267_ | TNF | 68 |
| | 31542458_31544282_RF | | |
| 25 | NFATc1_18_77158863_77160420_ | NFATc1 | 608 |
| | 77229964_77232215_FF | | |
| 26 | BCL6_3_187454088_187455426_ | BCL6 | 240 |
| | 187484009_187486420_FF | | |
| 27 | MAPK13_6_36066232_36072387_ | MAPK13 | 44 |
| | 36102587_36105090_FR | | |
| 28 | MLLT3_9_20319606_20322797_ | MLLT3 | 120 |
| | 20621547_20622617_FR | | |
| 29 | TOP1_20_39656117_39657610_ | TOP1 | 164 |
| | 39725920_39729106_FR | | |
| 30 | IFNAR1_21_34696683_34697716_ | IFNAR1 | 80 |
| | 34717312_34717993_RF | | |
| 31 | SKP1_5_133465952_133470062_ | SKP1 | 136 |
| | 133512403_133513591_RR | | |
| 32 | FZD10_12_130601147_130601992_ | FZD10 | 124 |
| | 130676699_130678204_FR | | |
| 33 | ITGA5_12_54787051_54795949_ | ITGA5 | 80 |
| | 54806686_54808428_FR | | |
| 34 | TNFRSF13B_17_16842268_ | TNFRSF13B | 128 |
| | 16844133_16924802_16926550_RR | | |
| 35 | BCL6_3_187438677_187439687_ | BCL6 | 240 |
| | 187454088_187455426_RR | | |
| 36 | ITPR3_6_33600698_33604388_ | ITPR3 | 100 |
| | 33678436_33680494_RR | | |
| 37 | MAP3K7_6_91275515_91285706_ | MAP3K7 | 308 |
| | 91312237_91314731_FF | | |
| 38 | IFNAR1_21_34696683_34697716_ | IFNAR1 | 80 |
| | 34777569_34779811_RF | | |
| 39 | NFATc1_18_77156086_77157023_ | NFATc1 | 608 |
| | 77218993_77220063_RF | | |
| 40 | PRDM1_6_106483435_106485826_ | PRDM1 | 120 |
| | 106500642_106506822_RF | | |
| 41 | IL-2RB_22_37532051_37533547_ | IL-2RB | 72 |
| | 37544442_37546723_FR | | |
| 42 | STAT3_17_40446029_40448202_ | STAT3 | 1108 |
| | 40557923_40558616_RR | | |
| 43 | NFKB1_4_103405171_103418579_ | NFKB1 | 96 |
| | 103512508_103516923_FR | | |
| 44 | CABLES1_18_20774415_20775705_ | CABLES1 | 136 |
| | 20863570_20868210_RF | | |
| 45 | JDP2_14_75883183_75893682_ | JDP2 | 80 |
| | 75936165_75936958_FF | | |
| 46 | NFATc1_18_77133931_77135912_ | NFATc1 | 608 |
| | 77218993_77220063_RF | | |
| 47 | CASP3_4_185504966_185506889_ | CASP3 | 88 |
| | 185543536_185552493_FR | | |
| 48 | REL_2_61074693_61075565_ | REL | 92 |
| | 61108479_61109187_FR | | |
| 49 | BTK_X_100610457_100612966_ | BTK | 404 |
| | 100667570_100670929_RF | | |
| 50 | BCL2A1_15_80256742_80257692_ | BCL2A1 | 302 |
| | 80285499_80286865_RR | | |
| 51 | TNFRSF13C_22_42302849_42305750_ | TNFRSF13C | 488 |
| | 42318166_42319783_FF | | |
| 52 | CDKN2C_1_51402271_51403526_ | CDKN2C | 72 |
| | 51439728_51440611_RR | | |

TABLE 5.b

| | Probe_Count_Sig | HyperG_Stats | FDR_HyperG | Percent_Sig |
|---|---|---|---|---|
| 1 | 615 | 0.000000000125197189743782 | 0.0000000113929442666842 | 55.51 |
| 2 | 83 | 0.000391435 | 0.005936759 | 61.03 |
| 3 | 64 | 0.802231212 | 0.999999997 | 43.24 |
| 4 | 34 | 0.79036009 | 0.999999997 | 42.5 |
| 5 | 113 | 0.999793469 | 0.999999997 | 36.69 |
| 6 | 216 | 0.227265083 | 0.590889215 | 48.21 |
| 7 | 39 | 0.999297311 | 0.999999997 | 32.5 |
| 8 | 213 | 0.999999997 | 0.999999997 | 35.03 |
| 9 | 27 | 0.999920864 | 0.999999997 | 28.12 |
| 10 | 280 | 0.000000444123116904245 | 0.0000202076018191431 | 57.38 |
| 11 | 61 | 0.0000870841188293703 | 0.001584931 | 66.3 |
| 12 | 86 | 0.999659646 | 0.999999997 | 35.83 |
| 13 | 14 | 0.995140179 | 0.999999997 | 29.17 |
| 14 | 280 | 0.000000444123116904245 | 0.0000202076018191431 | 57.38 |
| 15 | 148 | 0.294116072 | 0.669114065 | 48.05 |
| 16 | 44 | 0.824486728 | 0.999999997 | 42.31 |
| 17 | 89 | 0.224299285 | 0.590889215 | 49.44 |
| 18 | 31 | 0.931715515 | 0.999999997 | 38.75 |
| 19 | 30 | 0.066847306 | 0.221674157 | 57.69 |
| 20 | 21 | 0.267230575 | 0.639946904 | 52.5 |
| 21 | 213 | 0.999999997 | 0.999999997 | 35.03 |
| 22 | 173 | 0.0000217239097176038 | 0.000658959 | 58.25 |
| 23 | 58 | 0.033733587 | 0.145711753 | 55.77 |
| 24 | 18 | 0.999770934 | 0.999999997 | 26.47 |
| 25 | 213 | 0.999999997 | 0.999999997 | 35.03 |
| 26 | 86 | 0.999659646 | 0.999999997 | 35.83 |
| 27 | 18 | 0.81000526 | 0.999999997 | 40.91 |
| 28 | 39 | 0.999297311 | 0.999999997 | 32.5 |
| 29 | 71 | 0.808882973 | 0.999999997 | 43.29 |
| 30 | 34 | 0.79036009 | 0.999999997 | 42.5 |
| 31 | 72 | 0.072624208 | 0.221674157 | 52.94 |
| 32 | 43 | 0.996927827 | 0.999999997 | 34.68 |
| 33 | 40 | 0.293962898 | 0.669114065 | 50 |
| 34 | 76 | 0.002030583 | 0.01918923 | 59.38 |
| 35 | 86 | 0.999659646 | 0.999999997 | 35.83 |
| 36 | 48 | 0.409333853 | 0.903779884 | 48 |
| 37 | 113 | 0.999793469 | 0.999999997 | 36.69 |
| 38 | 34 | 0.79036009 | 0.999999997 | 42.5 |
| 39 | 213 | 0.999999997 | 0.999999997 | 35.03 |
| 40 | 47 | 0.9542933 | 0.999999997 | 39.17 |
| 41 | 24 | 0.991079692 | 0.999999997 | 33.33 |
| 42 | 615 | 0.000000000125197189743782 | 0.0000000113929442666842 | 55.51 |
| 43 | 27 | 0.999920864 | 0.999999997 | 28.12 |
| 44 | 59 | 0.784673088 | 0.999999997 | 43.38 |
| 45 | 36 | 0.639258785 | 0.999999997 | 45 |
| 46 | 213 | 0.999999997 | 0.999999997 | 35.03 |
| 47 | 39 | 0.688804514 | 0.999999997 | 44.32 |
| 48 | 30 | 0.997411174 | 0.999999997 | 32.61 |
| 49 | 182 | 0.722716922 | 0.999999997 | 45.05 |
| 50 | 166 | 0.00150308 | 0.01918923 | 54.97 |
| 51 | 280 | 0.000000444123116904245 | 0.0000202076018191431 | 57.38 |
| 52 | 30 | 0.821366544 | 0.999999997 | 41.67 |

TABLE 5.c

| | logFC | AveExpr | t | P. Value | adj. P. Val |
|---|---|---|---|---|---|
| 1 | 0.102545415 | 0.102545415 | 2.181691533 | 0.06115714 | 0.124690581 |
| 2 | 0.146814815 | 0.146814815 | 3.078806942 | 0.015395162 | 0.044697142 |
| 3 | 0.247739738 | 0.247739738 | 4.372950932 | 0.002449301 | 0.012749359 |
| 4 | 0.098641538 | 0.098641538 | 1.475225491 | 0.178893946 | 0.27926686 |
| 5 | 0.098390923 | 0.098390923 | 2.270415909 | 0.053292308 | 0.112564482 |
| 6 | 0.246810388 | 0.246810388 | 5.953590771 | 0.000359019 | 0.0048119 |
| 7 | 0.194400918 | 0.194400918 | 2.492510608 | 0.037760627 | 0.086786653 |
| 8 | 0.117865744 | 0.117865744 | 1.424258285 | 0.19268713 | 0.295560888 |
| 9 | 0.253919456 | 0.253919456 | 1.95465634 | 0.086862876 | 0.161472968 |
| 10 | 0.210247736 | 0.210247736 | 2.234440593 | 0.056352274 | 0.11719604 |
| 11 | −0.050897988 | −0.050897988 | −0.745725763 | 0.477453286 | 0.587468355 |
| 12 | −0.030722825 | −0.030722825 | −1.143761644 | 0.28622615 | 0.400334573 |
| 13 | −0.019224434 | −0.019224434 | −0.418322829 | 0.686861761 | 0.767615441 |
| 14 | −0.014186527 | −0.014186527 | −0.069260125 | 0.946505227 | 0.961540839 |
| 15 | −0.010289959 | −0.010289959 | −0.21611469 | 0.834379395 | 0.881581711 |
| 16 | 0.007162022 | 0.007162022 | 0.173838071 | 0.866368809 | 0.905364254 |
| 17 | 0.008581354 | 0.008581354 | 0.16838944 | 0.870512196 | 0.90817036 |
| 18 | 0.009594682 | 0.009594682 | 0.192008457 | 0.852583784 | 0.895087864 |
| 19 | 0.013062105 | 0.013062105 | 0.2898133 | 0.779427179 | 0.840001078 |
| 20 | 0.027459614 | 0.027459614 | 0.78838239 | 0.453500365 | 0.565397223 |
| 21 | 0.0309953 | 0.0309953 | 0.401906143 | 0.698417087 | 0.776651512 |
| 22 | 0.03119071 | 0.03119071 | 0.46421066 | 0.655032665 | 0.743235083 |
| 23 | 0.031952076 | 0.031952076 | 0.423263408 | 0.683401141 | 0.76482198 |
| 24 | 0.036397064 | 0.036397064 | 1.012029864 | 0.341541187 | 0.45879384 |
| 25 | 0.036449121 | 0.036449121 | 0.881245015 | 0.404223195 | 0.519301224 |
| 26 | 0.039792262 | 0.039792262 | 1.09873788 | 0.304267251 | 0.419987316 |
| 27 | 0.044981037 | 0.044981037 | 0.742965178 | 0.479031667 | 0.588704315 |
| 28 | 0.048157816 | 0.048157816 | 1.006274544 | 0.344135022 | 0.461283259 |
| 29 | 0.05692752 | 0.05692752 | 0.774540503 | 0.461182853 | 0.572336767 |
| 30 | 0.068999319 | 0.068999319 | 1.087503347 | 0.308907593 | 0.424307342 |
| 31 | 0.073674257 | 0.073674257 | 1.37062465 | 0.208203798 | 0.314180912 |
| 32 | 0.07496163 | 0.07496163 | 1.975529647 | 0.084116625 | 0.157842633 |
| 33 | 0.077618589 | 0.077618589 | 1.073493376 | 0.314772637 | 0.430505827 |
| 34 | 0.080234671 | 0.080234671 | 1.659676531 | 0.136077489 | 0.226348123 |
| 35 | 0.090602356 | 0.090602356 | 2.111185324 | 0.068216955 | 0.135104089 |
| 36 | 0.098319301 | 0.098319301 | 1.04377977 | 0.327501414 | 0.444140656 |
| 37 | 0.098390923 | 0.098390923 | 2.270415909 | 0.053292308 | 0.112564482 |
| 38 | 0.098641538 | 0.098641538 | 1.475225491 | 0.178893946 | 0.27926686 |
| 39 | 0.099162732 | 0.099162732 | 1.292936726 | 0.232594904 | 0.342880489 |
| 40 | 0.101277922 | 0.101277922 | 1.410506969 | 0.196565747 | 0.3002884 |
| 41 | 0.101676827 | 0.101676827 | 1.927111422 | 0.090619506 | 0.166768298 |
| 42 | 0.102545415 | 0.102545415 | 2.181691533 | 0.06115714 | 0.124690581 |
| 43 | 0.103364871 | 0.103364871 | 1.06297419 | 0.319233722 | 0.435153018 |
| 44 | 0.106978686 | 0.106978686 | 1.092750486 | 0.30673336 | 0.422367497 |
| 45 | 0.116604657 | 0.116604657 | 2.102936835 | 0.06909355 | 0.136425239 |
| 46 | 0.117865744 | 0.117865744 | 1.424258285 | 0.19268713 | 0.295560888 |
| 47 | 0.12582798 | 0.12582798 | 4.245528735 | 0.002904342 | 0.014151256 |
| 48 | 0.125971304 | 0.125971304 | 1.693676294 | 0.129294906 | 0.217785184 |
| 49 | 0.127634089 | 0.127634089 | 5.070996787 | 0.001004634 | 0.007593027 |
| 50 | 0.132678146 | 0.132678146 | 2.405792667 | 0.043193622 | 0.095959776 |
| 51 | 0.141794844 | 0.141794844 | 2.06833857 | 0.072892271 | 0.142181615 |
| 52 | 0.143309126 | 0.143309126 | 2.511399626 | 0.036672127 | 0.085032085 |

TABLE 5.d

| | B | FC | FC_1 | LS | Loop Detected |
|---|---|---|---|---|---|
| 1 | −4.804209212 | 1.073666112 | 1.073666112 | 1 | DBLCL |
| 2 | −3.422371897 | 1.107122465 | 1.107122465 | 1 | DBLCL |
| 3 | −1.514951748 | 1.18734545 | 1.18734545 | 1 | DBLCL |
| 4 | −5.804534479 | 1.070764741 | 1.070764741 | 1 | DBLCL |
| 5 | −4.669835237 | 1.070578751 | 1.070578751 | 1 | DBLCL |
| 6 | 0.493437892 | 1.186580836 | 1.186580836 | 1 | DBLCL |
| 7 | −4.329420018 | 1.14424891 | 1.14424891 | 1 | DBLCL |
| 8 | −5.869405345 | 1.085128386 | 1.085128386 | 1 | DBLCL |
| 9 | −5.141601134 | 1.192442298 | 1.192442298 | 1 | DBLCL |
| 10 | −4.724458483 | 1.156886824 | 1.156886824 | 1 | DBLCL |
| 11 | −6.575035413 | 0.965335281 | −1.035909513 | −1 | Ctrl |
| 12 | −6.200305189 | 0.978929707 | −1.021523806 | −1 | Ctrl |
| 13 | −6.778021016 | 0.986763027 | −1.01341454 | −1 | Ctrl |
| 14 | −6.87182168 | 0.990214838 | −1.009881857 | −1 | Ctrl |
| 15 | −6.848536344 | 0.99289292 | −1.007157952 | −1 | Ctrl |
| 16 | −6.85768141 | 1.004976678 | 1.004976678 | 1 | DBLCL |
| 17 | −6.858716992 | 1.005965867 | 1.005965867 | 1 | DBLCL |
| 18 | −6.85399155 | 1.00667269 | 1.00667269 | 1 | DBLCL |
| 19 | −6.827923501 | 1.009095073 | 1.009095073 | 1 | DBLCL |
| 20 | −6.54111486 | 1.019215847 | 1.019215847 | 1 | DBLCL |
| 21 | −6.785369028 | 1.021716754 | 1.021716754 | 1 | DBLCL |
| 22 | −6.755996218 | 1.021855153 | 1.021855153 | 1 | DBLCL |
| 23 | −6.775754566 | 1.022394568 | 1.022394568 | 1 | DBLCL |
| 24 | −6.338031258 | 1.025549454 | 1.025549454 | 1 | DBLCL |
| 25 | −6.461788363 | 1.02558646 | 1.02558646 | 1 | DBLCL |
| 26 | −6.248771887 | 1.027965796 | 1.027965796 | 1 | DBLCL |
| 27 | −6.5771745 | 1.031669619 | 1.031669619 | 1 | DBLCL |
| 28 | −6.343758805 | 1.033943833 | 1.033943833 | 1 | DBLCL |
| 29 | −6.552299411 | 1.040248004 | 1.040248004 | 1 | DBLCL |
| 30 | −6.260644426 | 1.048988833 | 1.048988833 | 1 | DBLCL |
| 31 | −5.936215307 | 1.05239351 | 1.05239351 | 1 | DBLCL |
| 32 | −5.111051252 | 1.053333021 | 1.053333021 | 1 | DBLCL |
| 33 | −6.275323888 | 1.055274694 | 1.055274694 | 1 | DBLCL |
| 34 | −5.559658058 | 1.05718999 | 1.05718999 | 1 | DBLCL |
| 35 | −4.910091268 | 1.064814672 | 1.064814672 | 1 | DBLCL |
| 36 | −6.305987164 | 1.070525603 | 1.070525603 | 1 | DBLCL |
| 37 | −4.669835237 | 1.070578751 | 1.070578751 | 1 | DBLCL |
| 38 | −5.804534479 | 1.070764741 | 1.070764741 | 1 | DBLCL |
| 39 | −6.030168045 | 1.071151639 | 1.071151639 | 1 | DBLCL |
| 40 | −5.886680521 | 1.072723247 | 1.072723247 | 1 | DBLCL |
| 41 | −5.181746622 | 1.073019896 | 1.073019896 | 1 | DBLCL |
| 42 | −4.804209212 | 1.073666112 | 1.073666112 | 1 | DBLCL |
| 43 | −6.286252837 | 1.074276132 | 1.074276132 | 1 | DBLCL |
| 44 | −6.255110449 | 1.076970465 | 1.076970465 | 1 | DBLCL |
| 45 | −4.922419619 | 1.08418027 | 1.08418027 | 1 | DBLCL |
| 46 | −5.869405345 | 1.085128386 | 1.085128386 | 1 | DBLCL |
| 47 | −1.693141948 | 1.091133768 | 1.091133768 | 1 | DBLCL |
| 48 | −5.512969318 | 1.091242172 | 1.091242172 | 1 | DBLCL |
| 49 | −0.581917188 | 1.092500613 | 1.092500613 | 1 | DBLCL |
| 50 | −4.462886317 | 1.096326979 | 1.096326979 | 1 | DBLCL |
| 51 | −4.97398611 | 1.103276839 | 1.103276839 | 1 | DBLCL |
| 52 | −4.300278466 | 1.104435469 | 1.104435469 | 1 | DBLCL |

TABLE 5.e

| | SEQ | | Probe |
|---|---|---|---|
| | ID | Probe sequence | Location |
| | NO: | 60 mer | Chr |
| 1 | 2 | GGGTTTCACCATGTTGGCCAGGCTGGTCTC | 17 |
| | | GAACTCCCGACCTCAGGTGATCCGCCCGCC | |
| 2 | 3 | GAGGGGCCTCTGGAGGGGGCGGGTTCTCTC | 10 |
| | | GATGCCTGGCCTCCACAGCACATGTGAGCA | |
| 3 | 4 | GAGGCTTTTATGCAGGAAAGTGTCCCAGTC | 20 |
| | | GAGGGACTGGCAGCAGGGGGACAGCAAGG | |
| | | G | |
| 4 | 5 | ACCTCTCTTAATTTTCTCAGCCATTCTTTC | 21 |
| | | GACCGCCTCTGCCCCGCTCTCGCTCTGCAC | |
| 5 | 6 | TGTGAAGGGAGGGGAGGAGAAAAGAAAATC | 6 |
| | | GAAACAAGCTTAGAAGCAGACACTTGCCCA | |
| 6 | 7 | TGGGGGAGCTCTGGGGTGGGGGTAGCGGTC | 19 |
| | | GATGGGTCCTGATGCCTCTCAGAAGGCCTT | |
| 7 | 8 | ACATTTCAAATCCTCTCTTCTAGCTACCTC | 9 |
| | | GAACTTCTGAGCTCAAGCAATCTTCCACCT | |
| 8 | 9 | CTAGAGGAGAGAGGGATGCCAGGCTCTATC | 18 |
| | | GAGTCTGAGTTCGTCCACGTGGTGGCCATC | |
| 9 | 10 | TCTTTATGGTGTCTCTTTATATATTTACTC | 4 |
| | | GAGGCTGCAGTGAGCTATAATTGCACCACT | |
| 10 | 11 | ACGGGCAGACAGGACCCCAGCCCATGCCTC | 22 |
| | | GACCCACTCCCGGGGGGATCGGGACACCGC | |
| 11 | 12 | TCCCTGCCTCTCTGGCGCTCTCGGACCCTC | 19 |
| | | GAACCCTCCCTTTGATCTATTCCATTCTCA | |
| 12 | 13 | AGATCCGTGTCTGCCTGCAGATACAAAATC | 3 |
| | | GAGGTGGATCGCCCAGGGGCGGGCAGTCCC | |
| 13 | 14 | GGGGGGGGGAGCGCGCCGGTCCCCGCGCTC | 1 |
| | | GAAGGCTGCCCTCCTCTCTGAATTTGGGTT | |
| 14 | 15 | ACCCAAACACGCGCAGACACCCGCACACTC | 22 |
| | | GACCCACTCCCGGGGGGATCGGGACACCGC | |
| 15 | 16 | CACACCCGCCCTACTGGATCCAAGTCACTC | 13 |
| | | GAGACAACACTGAAAACACAAAGGCATTTA | |
| 16 | 17 | TAGACTAGCGCCAGCTTTGTGCACAAGGTC | 17 |
| | | GACACCCCTCTCCCCAACCCTCTGTCAGAA | |
| 17 | 18 | AGGGTTTCACCATGTTGCCAGGCTGGTCTC | 11 |
| | | GAGACCATCCTGGCTAATACGGTGAAACCC | |
| 18 | 19 | CAACTTCATTCCCACGGTCACTGCCATCTC | 14 |
| | | GACCCACCAATAGAGCAACTCCCTGAGAGG | |
| 19 | 20 | CCATCAGCAAAGATGAACCTGGCACCTCTC | 1 |
| | | GACGCCATAAGCATGGTGAGCCAGGGTGGG | |
| 20 | 21 | TTCCAGTTCATAAAGATTTAAACAACATTC | 1 |
| | | GAGAAGAGAAAGGGGGGGAAGCTGCTAGGT | |
| 21 | 22 | CCCCGTGCACAGATCCCACCACCCAGGGTC | 18 |
| | | GAAGCCCCTCCGGGCCCCTCACGGGAGGGG | |
| 22 | 23 | AATTGCTCCATTATGGCTCACTGCAGCCTC | 17 |
| | | GAAGGTTTAGCTTATTCATTAAAATCAGTA | |
| 23 | 24 | GCTGAAAGTTATTACTTTGTTTTTCCCATC | 4 |
| | | GAGGTCCCGCGCACACGCCCCCGCGCGCAC | |
| 24 | 25 | AGCTGTTCCTCCTTTAAGGGTGACTCCCTC | 6 |
| | | GACCCCCACGTGCTGAGGGCTCCAGCCAGA | |
| 25 | 26 | GCCATGACGGGGCTGGAGGACCAGGAGTTC | |
| | | GACCCTGGGTGGTGGGATCTGTGCACGGGG | |
| | | | 18 |
| 26 | 27 | GGGACTGCCCGCCCCTGGGCGATCCACCTC | 3 |
| | | GATGTCCAAATGGTTCTTGCCTTCACCTCT | |
| 27 | 28 | GGGTTTCACCGTGTTAGCCAGGATGGTCTC | 6 |
| | | GAGACCAGCCTGGCCAACATGGCAAAACCC | |
| 28 | 29 | CTGTATTAGATTTTCACATGCATGAGACTC | 9 |
| | | GAACCGAGCCCCCGCAACACACTTTCAAGA | |
| 29 | 30 | ACAGTCACCGCCGCTTACCTGCGCCTCCTC | 20 |
| | | GACCATGAATATACTACCAAGGAAATATTT | |
| 30 | 31 | CATGTGTTATTTCCCCAATCTGGAAGACTC | 21 |
| | | GACCGCCTCTGCCCCGCTCTCGCTCTGCAC | |
| 31 | 32 | TGCCCCTCAAGCCCTCAGACTACAACAATC | 5 |
| | | GACAACGCGATCCACCGGGCCCGAAAGAAG | |
| 32 | 33 | ACCAGGGGCCCCAAAGAGGGGGTCAGGCTC | 12 |
| | | GAATCAAAGGGTTTCTGGATCCCTAGGTGT | |
| 33 | 34 | TCTAGAGGGGTATCCTCCCAAATCCCACTC | 12 |
| | | GACCCAGCCTCTGGACCAGTGCTCCTGCCA | |
| 34 | 35 | CCTGTGGTGCCCCCATCTCACCAGGCTCTC | 17 |
| | | GATGATGCCACAAGTGCCGTGCCACAGCAG | |
| 35 | 36 | CCTGTGGTGCCCCCATCTCACCAGGCTCTC | 3 |
| | | GATGATGCCACAAGTGCCGTGCCACAGCAG | |
| 36 | 37 | CACAGACACAACCCAGGCCTCCATCTACTC | 6 |
| | | GATCACAGTACTTATCTGTCTTACGTACAC | |
| 37 | 38 | TGTGAAGGGAGGGGAGGAGAAAAGAAAATC | 6 |
| | | GAAACAAGCTTAGAAGCAGACACTTGCCCA | |
| 38 | 39 | TGTGAAGGGAGGGGAGGAGAAAAGAAAATC | 21 |
| | | GAAACAAGCTTAGAAGCAGACACTTGCCCA | |
| 39 | 40 | CTAGAGGAGAGAGGGATGCCAGGCTCTATC | 18 |
| | | GATGACTTTCCTCCGGGGCGCGCGGCGCTG | |
| 40 | 41 | TCAAGAACTCATGGTTCTTAAAGATCACTC | 6 |
| | | GAGGCTGCAGTGAGCTATGATAATGCCACA | |
| 41 | 42 | CCACCATCCACCTGGGGCTGAGGGGACCTC | 22 |
| | | GAGTTTGAGCACCCCCTCCTGGGTCCTCAG | |
| 42 | 43 | GGGTTTCACCATGTTGGCCAGGCTGGTCTC | 17 |
| | | GAACTCCCGACCTCAGGTGATCCGCCCGCC | |
| 43 | 44 | ATACCAACCCCAGAAATAAAGTCATTCCTC | 4 |
| | | GAGGCTGCAGTGAGCTATAATTGCACCACT | |
| 44 | 45 | GTTCCTCACCCTGATCACACCTGGTTTATC | 18 |
| | | GAACTCTCTCAGGTTCACCCAGACCAAAGA | |
| 45 | 46 | TATCTGGCTTAGGCAGAAGGTAGGGGGCTC | 14 |
| | | GAGTGATTATAGAAATCCATATATATATTG | |
| 46 | 47 | CTAGAGGAGAGAGGGATGCCAGGCTCTATC | 18 |
| | | GAGTCTGAGTTCGTCCACGTGGTGGCCATC | |
| 47 | 48 | AACAGCAGCATTAGATTCTCATAGGAACTC | 4 |
| | | GAGTGTCATGAACAATCTTTTTCTTTAACA | |
| 48 | 49 | CATTCCTAGTGCCAGGACCCATCTCAGGTC | 2 |
| | | GACCCCCTCCCAAGCCAGCCGCCGCAGCAG | |
| 49 | 50 | CACTACTACCCAGGAAAGTGATGGGAGGTC | X |
| | | GAGATTGCAGGAAATGGAGAGTACATGCCT | |
| 50 | 51 | AGTGGCGCAATCTTGGCTAACTGCAGCCTC | 15 |
| | | GAGACCATCCTACATGGTGAAACCCCGTCT | |
| 51 | 52 | ACGGGCAGACAGGACCCCAGCCCATGCCTC | 22 |
| | | GAGCTGAAGGAACATGCTGGCAGGTAGCTC | |
| 52 | 53 | ATATTAAATTGCTTACATAGAATGAAGGTC | 1 |
| | | GAGGATAATGAAGGGAACCTGCCCTTGCAC | |

TABLE 5.f

| | Probe Location | | | | 4 kb Sequence Location | | |
|---|---|---|---|---|---|---|---|
| | Start1 | End1 | Start2 | End2 | Chr | Start1 | End1 |
| 1 | 40446029 | 40446060 | 40557923 | 40557954 | 17 | 40446029 | 40450030 |
| 2 | 81892358 | 81892389 | 81927417 | 81927448 | 10 | 81888388 | 81892389 |
| 3 | 44744656 | 44744687 | 44767157 | 44767188 | 20 | 44740686 | 44744687 |
| 4 | 34696683 | 34696714 | 34779780 | 34779811 | 21 | 34696683 | 34700684 |
| 5 | 91285675 | 91285706 | 91314700 | 91314731 | 6 | 91281705 | 91285706 |
| 6 | 19271977 | 19272008 | 19303710 | 19303741 | 19 | 19271977 | 19275978 |
| 7 | 20560917 | 20560948 | 20666337 | 20666368 | 9 | 20556947 | 20560948 |
| 8 | 77133931 | 77133962 | 77220032 | 77220063 | 18 | 77133931 | 77137932 |
| 9 | 103430366 | 103430397 | 103512508 | 103512539 | 4 | 103426396 | 103430397 |
| 10 | 42305719 | 42305750 | 42342568 | 42342599 | 22 | 42301749 | 42305750 |
| 11 | 49421750 | 49421781 | 49458408 | 49458439 | 19 | 49421750 | 49425751 |
| 12 | 187439656 | 187439687 | 187455395 | 187455426 | 3 | 187435686 | 187439687 |
| 13 | 24467543 | 24467574 | 24513928 | 24513959 | 1 | 24467543 | 24471544 |
| 14 | 42313974 | 42314005 | 42342568 | 42342599 | 22 | 42313974 | 42317975 |
| 15 | 41191135 | 41191166 | 41219134 | 41219165 | 13 | 41187165 | 41191166 |
| 16 | 53403683 | 53403714 | 53422397 | 53422428 | 17 | 53399713 | 53403714 |
| 17 | 77028527 | 77028558 | 77094560 | 77094591 | 11 | 77028527 | 77032528 |
| 18 | 75746612 | 75746643 | 75799853 | 75799884 | 14 | 75742642 | 75746643 |
| 19 | 11807586 | 11807617 | 11845619 | 11845650 | 1 | 11807586 | 11811587 |
| 20 | 228068849 | 228068880 | 228135088 | 228135119 | 1 | 228068849 | 228072850 |
| 21 | 77232184 | 77232215 | 77280170 | 77280201 | 18 | 77228214 | 77232215 |
| 22 | 41162341 | 41162372 | 41242678 | 41242709 | 17 | 41162341 | 41166342 |
| 23 | 106052640 | 106052671 | 106067346 | 106067377 | 4 | 106048670 | 106052671 |
| 24 | 31525914 | 31525945 | 31544251 | 31544282 | 6 | 31525914 | 31529915 |
| 25 | 77160389 | 77160420 | 77232184 | 77232215 | 18 | 77156419 | 77160420 |
| 26 | 187455395 | 187455426 | 187486389 | 187486420 | 3 | 187451425 | 187455426 |
| 27 | 36072356 | 36072387 | 36102587 | 36102618 | 6 | 36068386 | 36072387 |
| 28 | 20322766 | 20322797 | 20621547 | 20621578 | 9 | 20318796 | 20322797 |
| 29 | 39657579 | 39657610 | 39725920 | 39725951 | 20 | 39653609 | 39657610 |
| 30 | 34696683 | 34696714 | 34717962 | 34717993 | 21 | 34696683 | 34700684 |
| 31 | 133465952 | 133465983 | 133512403 | 133512434 | 5 | 133465952 | 133469953 |
| 32 | 130601961 | 130601992 | 130676699 | 130676730 | 12 | 130597991 | 130601992 |
| 33 | 54795918 | 54795949 | 54806686 | 54806717 | 12 | 54791948 | 54795949 |
| 34 | 16842268 | 16842299 | 16924802 | 16924833 | 17 | 16842268 | 16846269 |
| 35 | 187438677 | 187438708 | 187454088 | 187454119 | 3 | 187438677 | 187442678 |
| 36 | 33600698 | 33600729 | 33678436 | 33678467 | 6 | 33600698 | 33604699 |
| 37 | 91285675 | 91285706 | 91314700 | 91314731 | 6 | 91281705 | 91285706 |
| 38 | 34696683 | 34696714 | 34779780 | 34779811 | 21 | 34696683 | 34700684 |
| 39 | 77156086 | 77156117 | 77220032 | 77220063 | 18 | 77156086 | 77160087 |
| 40 | 106483435 | 106483466 | 106506791 | 106506822 | 6 | 106483435 | 106487436 |
| 41 | 37533516 | 37533547 | 37544442 | 37544473 | 22 | 37529546 | 37533547 |
| 42 | 40446029 | 40446060 | 40557923 | 40557954 | 17 | 40446029 | 40450030 |
| 43 | 103418548 | 103418579 | 103512508 | 103512539 | 4 | 103414578 | 103418579 |
| 44 | 20774415 | 20774446 | 20868179 | 20868210 | 18 | 20774415 | 20778416 |
| 45 | 75893651 | 75893682 | 75936927 | 75936958 | 14 | 75889681 | 75893682 |
| 46 | 77133931 | 77133962 | 77220032 | 77220063 | 18 | 77133931 | 77137932 |
| 47 | 185506858 | 185506889 | 185543536 | 185543567 | 4 | 185502888 | 185506889 |
| 48 | 61075534 | 61075565 | 61108479 | 61108510 | 2 | 61071564 | 61075565 |
| 49 | 100610457 | 100610488 | 100670898 | 100670929 | X | 100610457 | 100614458 |
| 50 | 80256742 | 80256773 | 80285499 | 80285530 | 15 | 80256742 | 80260743 |
| 51 | 42305719 | 42305750 | 42319752 | 42319783 | 22 | 42301749 | 42305750 |
| 52 | 51402271 | 51402302 | 51439728 | 51439759 | 1 | 51402271 | 51406272 |

TABLE 5.g

| | 4 kb Sequence Location | | |
|---|---|---|---|
| | Start2 | End2 | Probe |
| 1 | 40557923 | 40561924 | STAT3_17_40446029_40448202_40557923_40558616_RR |
| 2 | 81927417 | 81931418 | ANXA11_10_81889664_81892389_81927417_81929312_FR |
| 3 | 44767157 | 44771158 | CD40_20_44739847_44744687_44767157_44770555_FR |
| 4 | 34775810 | 34779811 | IFNAR1_21_34696683_34697716_34777569_34779811_RF |
| 5 | 91310730 | 91314731 | MAP3K7_6_91275515_91285706_91312237_91314731_FF |
| 6 | 19299740 | 19303741 | MEF2B_19_19271977_19273500_19302232_19303741_RF |
| 7 | 20662367 | 20666368 | MLLT3_9_20556478_20560948_20658310_20666368_FF |
| 8 | 77216062 | 77220063 | NFATc1_18_77133931_77135912_77218993_77220063_RF |
| 9 | 103512508 | 103516509 | NFKB1_4_103425293_103430397_103512508_103516923_FR |
| 10 | 42342568 | 42346569 | TNFRSF13C_22_42302849_42305750_42342568_42346797_FR |
| 11 | 49454438 | 49458439 | BAX_19_49421750_49425644_49457303_49458439_RF |
| 12 | 187451425 | 187455426 | BCL6_3_187438677_187439687_187454088_187455426_FF |
| 13 | 24509958 | 24513959 | IL22RA1_1_24467543_24471444_24512238_24513959_RF |
| 14 | 42342568 | 42346569 | TNFRSF13C_22_42313974_42315085_42342568_42346797_RR |
| 15 | 41219134 | 41223135 | FOXO1_13_41184194_41191166_41219134_41220693_FR |
| 16 | 53418427 | 53422428 | HLF_17_53402207_53403714_53420274_53422428_FF |
| 17 | 77090590 | 77094591 | PAK1_11_77028527_77036211_77090325_77094591_RF |
| 18 | 75795883 | 75799884 | FOS_14_75744954_75746643_75795718_75799884_FF |
| 19 | 11841649 | 11845650 | MTHFR_1_11807586_11814341_11843522_11845650_RF |
| 20 | 228135088 | 228139089 | WNT9A_1_228068849_228075473_228135088_228140421_RR |
| 21 | 77280170 | 77284171 | NFATc1_18_77229964_77232215_77280170_77283702_FR |
| 22 | 41242678 | 41246679 | BRCA1_17_41162341_41168331_41242678_41245761_RR |
| 23 | 106063376 | 106067377 | TET2_4_106047220_106052671_106063962_106067377_FF |
| 24 | 31540281 | 31544282 | TNF_6_31525914_31529267_31542458_31544282_RF |
| 25 | 77228214 | 77232215 | NFATc1_18_77158863_77160420_77229964_77232215_FF |
| 26 | 187482419 | 187486420 | BCL6_3_187454088_187455426_187484009_187486420_FF |
| 27 | 36102587 | 36106588 | MAPK13_6_36066232_36072387_36102587_36105090_FR |
| 28 | 20621547 | 20625548 | MLLT3_9_20319606_20322797_20621547_20622617_FR |
| 29 | 39725920 | 39729921 | TOP1_20_39656117_39657610_39725920_39729106_FR |
| 30 | 34713992 | 34717993 | IFNAR1_21_34696683_34697716_34717312_34717993_RF |
| 31 | 133512403 | 133516404 | SKP1_5_133465952_133470062_133512403_133513591_RR |
| 32 | 130676699 | 130680700 | FZD10_12_130601147_130601992_130676699_130678204_FR |
| 33 | 54806686 | 54810687 | ITGA5_12_54787051_54795949_54806686_54808428_FR |
| 34 | 16924802 | 16928803 | TNFRSF13B_17_16842268_16844133_16924802_16926550_RR |
| 35 | 187454088 | 187458089 | BCL6_3_187438677_187439687_187454088_187455426_RR |
| 36 | 33678436 | 33682437 | ITPR3_6_33600698_33604388_33678436_33680494_RR |
| 37 | 91310730 | 91314731 | MAP3K7_6_91275515_91285706_91312237_91314731_FF |
| 38 | 34775810 | 34779811 | IFNAR1_21_34696683_34697716_34777569_34779811_RF |
| 39 | 77216062 | 77220063 | NFATc1_18_77156086_77157023_77218993_77220063_RF |
| 40 | 106502821 | 106506822 | PRDM1_6_106483435_106485826_106500642_106506822_RF |
| 41 | 37544442 | 37548443 | IL-2RB_22_37532051_37533547_37544442_37546723_FR |
| 42 | 40557923 | 40561924 | STAT3_17_40446029_40448202_40557923_40558616_RR |
| 43 | 103512508 | 103516509 | NFKB1_4_103405171_103418579_103512508_103516923_FR |
| 44 | 20864209 | 20868210 | CABLES1_18_20774415_20775705_20863570_20868210_RF |
| 45 | 75932957 | 75936958 | JDP2_14_75883183_75893682_75936165_75936958_FF |
| 46 | 77216062 | 77220063 | NFATc1_18_77133931_77135912_77218993_77220063_RF |
| 47 | 185543536 | 185547537 | CASP3_4_185504966_185506889_185543536_185552493_FR |
| 48 | 61108479 | 61112480 | REL_2_61074693_61075565_61108479_61109187_FR |
| 49 | 100666928 | 100670929 | BTK_X_100610457_100612966_100667570_100670929_RF |
| 50 | 80285499 | 80289500 | BCL2A1_15_80256742_80257692_80285499_80286865_RR |
| 51 | 42315782 | 42319783 | TNFRSF13C_22_42302849_42305750_42318166_42319783_FF |
| 52 | 51439728 | 51443729 | CDKN2C_1_51402271_51403526_51439728_51440611_RR |

TABLE 5.h

| | | Inner_primers | | |
|---|---|---|---|---|
| | | | SEQ ID | |
| | PCR-Primer1_ID | PCR_Primer1 | NO: | PCR-Primer2_ID |
| 1 | OBD RD048.001 | GGAAGACCCTTTGTGACCTGG | 54 | OBD RD048.003 |
| 2 | OBD RD048.005 | CAAGACCTCACCCAATGC | 55 | OBD RD048.007 |
| 3 | OBD RD048.009 | GAGGAAGGGTGTGCTTTG | 56 | OBD RD048.011 |
| 4 | OBD RD048.013 | TGGTCAGACGAGATGCCAAG | 57 | OBD RD048.015 |
| 5 | OBD RD048.017 | GTTTGGGACATCAGAAATACAG | 58 | OBD RD048.019 |
| 6 | OBD RD048.021 | CTAAGTCTTAAAGGGCCAGAG | 59 | OBD RD048.023 |
| 7 | OBD RD048.025 | CAGAGAGGATAGCCTTACAC | 60 | OBD RD048.027 |
| 8 | OBD RD048.029 | TGCTTCATGAAACTCAGATGG | 61 | OBD RD048.031 |
| 9 | OBD RD048.033 | ACAGCAGTCCAACAATAGTC | 62 | OBD RD048.035 |
| 10 | OBD RD048.037 | GTTGAGGCAGACAGAAGAG | 63 | OBD RD048.039 |
| 11 | OBD RD048.041 | TCGGAGGTTCCTGGCTCTCTGAT | 64 | OBD RD048.043 |
| 12 | OBD RD048.045 | TTTCTCAATAAAGATTCTCAGAT | 65 | OBD RD048.047 |
| 13 | OBD RD048.049 | TAGGATTCACTGAGAAGGTCCCT | 66 | OBD RD048.051 |
| 14 | OBD RD048.053 | CCTCTCTCTGAGTCTTGAGTTTC | 67 | OBD RD048.055 |
| 15 | OBD RD048.057 | GATGGAGAAAGGAGCAAGGAACCAGG | 68 | OBD RD048.059 |
| 16 | OBD RD048.061 | GGCTGATGGTATGGGAATGGGTGG | 69 | OBD RD048.063 |
| 17 | OBD RD048.065 | ACCCAGTTACTTGTTGTATTTGC | 70 | OBD RD048.067 |
| 18 | OBD RD048.069 | GGCTTTCCCCTTCTGTTTTGTTC | 71 | OBD RD048.071 |
| 19 | OBD RD048.073 | CTCTGACAAGCAACTCTGAATCC | 72 | OBD RD048.075 |
| 20 | OBD RD048.077 | GCTTCAAAGAGTGTGATTATGTAAAA | 73 | OBD RD048.079 |
| 21 | OBD RD048.081 | AATAACTGTGGCATCGGAGAGGT | 74 | OBD RD048.083 |
| 22 | OBD RD048.085 | AAGTCTCAATGCCACCCAGGCTG | 75 | OBD RD048.087 |
| 23 | OBD RD048.089 | TGTATCCCTCCTGTTATCATCCC | 76 | OBD RD048.091 |
| 24 | OBD RD048.093 | CAGACACCTCAGGGCTAAGAGCG | 77 | OBD RD048.095 |
| 25 | OBD RD048.097 | GGGAGAACCGAACCCCTGGCGGC | 78 | OBD RD048.099 |
| 26 | OBD RD048.101 | TACCCCACCCCGACCACTCCGTA | 79 | OBD RD048.103 |
| 27 | OBD RD048.105 | GGAATACAAGTGTGTGCCACCAC | 80 | OBD RD048.107 |
| 28 | OBD RD048.109 | CTTTGGGCTTGAAGGCTTTGTTC | 81 | OBD RD048.111 |
| 29 | OBD RD048.113 | AGCCTCAGCCGTTTCTGGAGTCTCGG | 82 | OBD RD048.115 |
| 30 | OBD RD048.117 | TCTAACCCCAGTTCTGCCAGTAA | 83 | OBD RD048.119 |
| 31 | OBD RD048.121 | CGGTTCTCACTTTCGTTCTTTGC | 84 | OBD RD048.123 |
| 32 | OBD RD048.125 | CAAATGAGAGCCTCCAAGACAGC | 85 | OBD RD048.127 |
| 33 | OBD RD048.129 | TGGTTCACGGCAAAGTAGTCACA | 86 | OBD RD048.131 |
| 34 | OBD RD048.133 | TCTATCACTTTCCTGGGCATCAG | 87 | OBD RD048.135 |
| 35 | OBD RD048.137 | CCTGCCTCAGCCTCCCAAGTAGC | 88 | OBD RD048.139 |
| 36 | OBD RD048.141 | TGGATGGAACCCCTGAGCCACACAGC | 89 | OBD RD048.143 |
| 37 | OBD RD048.145 | GGTTAGGTCTTCTGCCTTCAAAG | 90 | OBD RD048.147 |
| 38 | OBD RD048.149 | CAGACGAGATGCCAAGTGCTTTA | 91 | OBD RD048.151 |
| 39 | OBD RD048.153 | TGCTGGAGTGAAAACGCCTCTTT | 92 | OBD RD048.155 |
| 40 | OBD RD048.157 | TCATAATGTCAGTGTCCTGTTCA | 93 | OBD RD048.159 |
| 41 | OBD RD048.161 | GCTTTCTGAATCTTTCCCTGGTG | 94 | OBD RD048.163 |
| 42 | OBD RD048.165 | CCTGCCTCAGCCGCCCGAGTAGC | 95 | OBD RD048.167 |
| 43 | OBD RD048.169 | CCTCCCACTTTTGATGGCACTGC | 96 | OBD RD048.171 |
| 44 | OBD RD048.173 | CCCACATTTCGTTCTTTCCTGTT | 97 | OBD RD048.175 |
| 45 | OBD RD048.177 | CTTCTATGGGTGATGACCTGACA | 98 | OBD RD048.179 |
| 46 | OBD RD048.181 | TGCTGGAGTGAAAACGCCTCTTT | 99 | OBD RD048.183 |
| 47 | OBD RD048.185 | CCATCGCTCACATCATTACCTGA | 100 | OBD RD048.187 |
| 48 | OBD RD048.189 | ACATACAGTCAGTAGGAGCCTTG | 101 | OBD RD048.191 |
| 49 | OBD RD048.193 | GCTCCAACACTCACATCTAACAC | 102 | OBD RD048.195 |
| 50 | OBD RD048.197 | GTATTTTGTTTGTTTGTTTGTTTT | 103 | OBD RD048.199 |
| 51 | OBD RD048.201 | CTCCAAGACACCACTGCCGTTGAGGC | 104 | OBD RD048.203 |
| 52 | OBD RD048.205 | GCCTCATTTCTGTCCTCCTTTGA | 105 | OBD RD048.207 |

TABLE 5.i

| | Inner_primers | SEQ ID | | | |
|---|---|---|---|---|---|
| | PCR_Primer2 | NO: | Gene | Marker | GLMNET |
| 1 | TCACCATTCGTTCAACACAC | 106 | STAT3 | OBD RD048.001.003 | 2.08E−08 |
| 2 | CAGTTGTGGAGGCTCAATAC | 107 | ANXA11 | OBD RD048.005.007 | 0.00000056 |
| 3 | GGAAGGAAAGCCAGTGAAG | 108 | CD40 | OBD RD048.009.011 | 0.00000449 |
| 4 | ACCCTAGAGTCTTGGACAG | 109 | IFNAR1 | OBD RD048.013.015 | 0.000000838 |
| 5 | ATCCCTAGGGCACTGAAC | 110 | MAP3K7 | OBD RD048.017.019 | 0.00000156 |
| 6 | CATACAAGGATGGAGTGACC | 111 | MEF2B | OBD RD048.021.023 | 0.00000137 |
| 7 | AGTGTCTTGCCCTGTAATC | 112 | MLLT3 | OBD RD048.025.027 | 0.0000046 |
| 8 | AGCCTAAGCTGAGGAACTC | 113 | NFATc1 | OBD RD048.029.031 | 0.00000181 |
| 9 | AACTCCTAATGAGAAAGTCTGC | 114 | NFKB1 | OBD RD048.033.035 | 0.00000178 |
| 10 | GGTCGGGTAGTAGAGAGTG | 115 | TNFRSF13C | OBD RD048.037.039 | 0.000000402 |
| 11 | GGACAGGTAACTACGGGTCTCCC | 116 | BAX | OBD RD048.041.043 | −0.000000273 |
| 12 | TACCCCACCCCGACCACTCCGTA | 117 | BCL6 | OBD RD048.045.047 | 0.000000154 |
| 13 | CACCTTGCGTAGAGGCAGTAGACCCC | 118 | IL22RA1 | OBD RD048.049.051 | −0.000000967 |
| 14 | AATGTCCTCCGAGCCGCCTGCTGG | 119 | TNFRSF13C | OBD RD048.053.055 | 2.13E−08 |
| 15 | GGTGTGAGGTAAGAAGTCATAGCCAT | 120 | FOXO1 | OBD RD048.057.059 | 0.00000117 |
| 16 | CACAGAGCCTGCCATCCTCACAT | 121 | HLF | OBD RD048.061.063 | 0.000000324 |
| 17 | ACTACAGGTGCCCGCCACAAGGC | 122 | PAK1 | OBD RD048.065.067 | −4.86E−08 |
| 18 | GGGATGGAGCAGGAAGGAGAGAGAGG | 123 | FOS | OBD RD048.069.071 | 0.00000028 |
| 19 | TATGTCTTGCCCTGTGCTGCGGCTCC | 124 | MTHFR | OBD RD048.073.075 | 0.000000306 |
| 20 | ATCAGGTCCCGACTTCCTTGGGC | 125 | WNT9A | OBD RD048.077.079 | 0.00000596 |
| 21 | AACACCGAGACACACCGAGTCCCTCC | 126 | NFATc1 | OBD RD048.081.083 | 0.000000396 |
| 22 | GACTGCTCAGGGCTATCCTCTCAG | 127 | BRCA1 | OBD RD048.085.087 | −0.000000314 |
| 23 | AGAGGTGCCAGTGGGTGGAGGCG | 128 | TET2 | OBD RD048.089.091 | 0.000000105 |
| 24 | GCTCCTCCTCCTGCTGTCGCCAG | 129 | TNF | OBD RD048.093.095 | 0.00000119 |
| 25 | GGGCGGCTGTGAAACTGAGGTCC | 130 | NFATc1 | OBD RD048.097.099 | 0.00000232 |
| 26 | AGGAAAGGCTTCACTGAGCATCA | 131 | BCL6 | OBD RD048.101.103 | 0.00000193 |
| 27 | TTTGTATTCTTAGTAGAGACGGG | 132 | MAPK13 | OBD RD048.105.107 | −5.18E−08 |
| 28 | GCCCGCCGCCCTGCCTTTCTGAAT | 133 | MLLT3 | OBD RD048.109.111 | 0.00000156 |
| 29 | CTCTTGTTGGACAGAAACCCTAC | 134 | TOP1 | OBD RD048.113.115 | 4.09E−08 |
| 30 | TGAGCGACCAGACCGTTGCTGTGTGC | 135 | IFNAR1 | OBD RD048.117.119 | 0.000000517 |
| 31 | CGCCCACTGAACTGGAAAGGGTCGTG | 136 | SKP1 | OBD RD048.121.123 | 0.000000786 |
| 32 | AGAAGTGCCAGTCTACATACACC | 137 | FZD10 | OBD RD048.125.127 | 0.00000223 |
| 33 | AGGCAGACACAGAGCAGAGCAGAGGC | 138 | ITGA5 | OBD RD048.129.131 | 0.00000124 |
| 34 | GGTCTCCCCTCCTACCACACTGGCAT | 139 | TNFRSF13B | OBD RD048.133.135 | 0.000000638 |
| 35 | TGAAGTTTGGTAAAGACCGAGTT | 140 | BCL6 | OBD RD048.137.139 | 0.000000206 |
| 36 | TGTTCTTGCTTTCCTCCAGGTTG | 141 | ITPR3 | OBD RD048.141.143 | 0.000000731 |
| 37 | CTGTGGGTGGAAGAGGCTCAGGCATC | 142 | MAP3K7 | OBD RD048.145.147 | 0.00000156 |
| 38 | TGAGCGACCAGACCGTTGCTGTGTGC | 143 | IFNAR1 | OBD RD048.149.151 | 0.000000838 |
| 39 | TTTCTCCTCTCCCGAAGACCGCAGCC | 144 | NFATc1 | OBD RD048.153.155 | 0.00000132 |
| 40 | CTCTCTCTCTGTCACCCAGGCTG | 145 | PRDM1 | OBD RD048.157.159 | 0.00000243 |
| 41 | CGTAGGCATCCGTGGGTGTGACCAGT | 146 | IL-2RB | OBD RD048.161.163 | 0.000000378 |
| 42 | CGCCTGTAATCCCAGAACTTTGG | 147 | STAT3 | OBD RD048.165.167 | 2.08E−08 |
| 43 | GTCTCACTCTGTTGCCCAGGCTG | 148 | NFKB1 | OBD RD048.169.171 | 0.00000135 |
| 44 | TTCTTGATAAAATGAATCTTCTTA | 149 | CABLES1 | OBD RD048.173.175 | 0.000000717 |
| 45 | TGGAGTTTGCTGTGGGCACTGAGGCG | 150 | JDP2 | OBD RD048.177.179 | 0.00000334 |
| 46 | CCACCACCATCAGCCAGTGCCACG | 151 | NFATc1 | OBD RD048.181.183 | 0.00000181 |
| 47 | CAATGCCAGGTCTTCATACTCTA | 152 | CASP3 | OBD RD048.185.187 | 0.00000305 |
| 48 | ACCCAGCGTCGCCGTCCACCGTA | 153 | REL | OBD RD048.189.191 | 0.00000123 |
| 49 | GGTCCACATTCTCACGAACCGCCTCC | 154 | BTK | OBD RD048.193.195 | 0.00000409 |
| 50 | CATTCTCCTGCCTCAGCCTCCTG | 155 | BCL2A1 | OBD RD048.197.199 | 0.000000299 |
| 51 | CTAAATGTGCTGTGTCTTGGAGC | 156 | TNFRSF13C | OBD RD048.201.203 | 0.000000179 |
| 52 | TGCTTCACCAGGAACTCCACCACCCG | 157 | CDKN2C | OBD RD048.205.207 | 0.0000123 |

TABLE 5.j

| | | | Probe_ |
|---|---|---|---|
| | Probe | GeneLocus | Count_Total |
| 53 | ANXA11_10_81889664_81892389_81927417_81929312_FR | ANXA11 | 136 |
| 54 | CD40_20_44737133_44739370_44777294_44780862_RR | CD40 | 148 |
| 55 | CREB3L2_7_137532509_137535848_137608464_137613205_FR | CREB3L2 | 168 |
| 56 | MyD88_3_38159544_38161117_38182050_38188284_FR | MyD88 | 80 |
| 57 | MEF2B_19_19255724_19257122_19271977_19273500_FF | MEF2B | 448 |
| 58 | IL-2RB_22_37569072_37572860_37583052_37586677_RR | IL-2RB | 72 |
| 59 | FRAP1_1_11321482_11322337_11347781_11348658_FF | FRAP1 | 704 |
| 60 | BCL6_3_187438677_187439687_187452395_187454091_FF | BCL6 | 240 |
| 61 | MMP9_20_44635898_44638559_44669235_44671514_FF | MMP9 | 68 |
| 62 | MAP3K7_6_91275515_91285706_91296544_91297579_FR | MAP3K7 | 308 |
| 63 | MLLT3_9_20556478_20560948_20658310_20666368_FF | MLLT3 | 120 |
| 64 | HLF_17_53404056_53408147_53420274_53422428_RF | HLF | 104 |
| 65 | SIRT1_10_69650583_69655218_69676432_69678199_FR | SIRT1 | 96 |
| 66 | NFATc1_18_77124213_77127824_77280170_77283702_RF | NFATc1 | 608 |
| 67 | TNFRSF13C_22_42302849_42305750_42342568_42346797_FR | TNFRSF13C | 488 |
| 68 | STAT3_17_40456120_40457219_40580136_40581714_RF | STAT3 | 1108 |
| 69 | NFKB1_4_103512508_103516923_103561903_103565015_RF | NFKB1 | 96 |
| 70 | MEF2B_19_19271977_19273500_19302232_19303741_RF | MEF2B | 448 |
| 71 | CD40_20_44739847_44744687_44767157_44770555_FR | CD40 | 148 |
| 72 | MAPK10_4_87408248_87409426_87514697_87515355_RF | MAPK10 | 668 |
| 73 | FRAP1_1_1119O9O5_11194522_11269915_1127245O_RR | FRAP1 | 704 |
| 74 | NFKB1_4_103425293_103430397_103512508_103516923_FR | NFKB1 | 96 |
| 75 | MAPK10_4_87373087_87377906_87514697_87515355_RF | MAPK10 | 668 |
| 76 | JAK3_19_17889333_17890586_17934729_17936992_FR | JAK3 | 60 |
| 77 | TNFRSF13C_22_42329800_42332095_42352233_42353781_FR | TNFRSF13C | 488 |
| 78 | TET2_4_106058602_106063965_106118157_106119978_RR | TET2 | 104 |
| 79 | NAE1_16_66835284_66840537_66902726_66909724_RF | NAE1 | 64 |
| 80 | TNFRSF13C_22_42335475_42336871_42362266_42363517_RR | TNFRSF13C | 488 |
| 81 | NFATc1_18_77151077_77154182_77274975_77276499_RR | NFATc1 | 608 |
| 82 | BRCA1_17_41214832_41217070_41227254_41229572_RR | BRCA1 | 297 |
| 83 | MLLT3_9_20377197_20385409_20556478_20560948_RF | MLLT3 | 120 |
| 84 | PCDHGA6/B2/B4_5_140751685_140753982_140892508_l40893138_FF | PCDHGA6/B2/B4 | 108 |
| 85 | MAPK10_4_87166373_87167382_87408248_87409426_RR | MAPK10 | 668 |
| 86 | BTK_X_100646274_100647902_100689454_100691928_RR | BTK | 404 |
| 87 | BTK_X_100625073_100626595_100689454_100691928_RR | BTK | 404 |
| 88 | BTK_X_100587279_100590348_100627655_100629872_FR | BTK | 404 |
| 89 | BTK_X_100627655_100629872_100647899_100654354_RR | BTK | 404 |
| 90 | BTK_X_100647899_100654354_100673203_100675145_RF | BTK | 404 |
| 91 | BTK_X_100602468_100603585_100647899_100654354_RR | BTK | 404 |
| 92 | BTK_X_100610457_100612966_100667570_100670929_RF | BTK | 404 |

TABLE 5.k

| | Probe_Count_Sig | HyperG_Stats | FDR_HyperG | Percent_Sig |
|---|---|---|---|---|
| 53 | 83 | 0.000391435 | 0.005936759 | 61.03 |
| 54 | 64 | 0.802231212 | 0.999999997 | 43.24 |
| 55 | 47 | 0.999999703 | 0.999999997 | 27.98 |
| 56 | 27 | 0.991982598 | 0.999999997 | 33.75 |
| 57 | 216 | 0.227265083 | 0.590889215 | 48.21 |
| 58 | 24 | 0.991079692 | 0.999999997 | 33.33 |
| 59 | 359 | 0.006468458 | 0.042044978 | 50.99 |
| 60 | 86 | 0.999659646 | 0.999999997 | 35.83 |
| 61 | 25 | 0.957692148 | 0.999999997 | 36.76 |
| 62 | 113 | 0.999793469 | 0.999999997 | 36.69 |
| 63 | 39 | 0.999297311 | 0.999999997 | 32.5 |
| 64 | 44 | 0.824486728 | 0.999999997 | 42.31 |
| 65 | 64 | 0.0000456596437717468 | 0.001038757 | 66.67 |
| 66 | 213 | 0.999999997 | 0.999999997 | 35.03 |
| 67 | 280 | 0.000000444123116904245 | 0.0000202076018191431 | 57.38 |
| 68 | 615 | 0.000000000125197189743782 | 0.0000000113929442666842 | 55.51 |
| 69 | 27 | 0.999920864 | 0.999999997 | 28.12 |
| 70 | 216 | 0.227265083 | 0.590889215 | 48.21 |
| 71 | 64 | 0.802231212 | 0.999999997 | 43.24 |
| 72 | 244 | 0.999999947 | 0.999999997 | 36.53 |
| 73 | 359 | 0.006468458 | 0.042044978 | 50.99 |
| 74 | 27 | 0.999920864 | 0.999999997 | 28.12 |
| 75 | 244 | 0.999999947 | 0.999999997 | 36.53 |
| 76 | 31 | 0.243296934 | 0.615000583 | 51.67 |
| 77 | 280 | 0.000000444123116904245 | 0.0000202076018191431 | 57.38 |
| 78 | 58 | 0.033733587 | 0.145711753 | 55.77 |
| 79 | 36 | 0.071920785 | 0.221674157 | 56.25 |
| 80 | 280 | 0.000000444123116904245 | 0.0000202076018191431 | 57.38 |
| 81 | 213 | 0.999999997 | 0.999999997 | 35.03 |
| 82 | 173 | 0.0000217239097176038 | 0.000658959 | 58.25 |
| 83 | 39 | 0.999297311 | 0.999999997 | 32.5 |
| 84 | 36 | 0.997865052 | 0.999999997 | 33.33 |
| 85 | 244 | 0.999999947 | 0.999999997 | 36.53 |
| 86 | 182 | 0.722716922 | 0.999999997 | 45.05 |
| 87 | 182 | 0.722716922 | 0.999999997 | 45.05 |
| 88 | 182 | 0.722716922 | 0.999999997 | 45.05 |
| 89 | 182 | 0.722716922 | 0.999999997 | 45.05 |
| 90 | 182 | 0.722716922 | 0.999999997 | 45.05 |
| 91 | 182 | 0.722716922 | 0.999999997 | 45.05 |
| 92 | 182 | 0.722716922 | 0.999999997 | 45.05 |

TABLE 5.l

| | logFC | AveExpr | t | P. Value | adj. P. Val |
|---|---|---|---|---|---|
| 53 | 0.146814815 | 0.146814815 | 3.078806942 | 0.015395162 | 0.044697142 |
| 54 | 0.147791337 | 0.147791337 | 1.633707333 | 0.141477876 | 0.232950641 |
| 55 | 0.148349454 | 0.148349454 | 3.08222473 | 0.015316201 | 0.044558199 |
| 56 | 0.153758518 | 0.153758518 | 1.99378109 | 0.081784292 | 0.154640968 |
| 57 | 0.156103192 | 0.156103192 | 4.16566548 | 0.003235665 | 0.015172146 |
| 58 | 0.161073376 | 0.161073376 | 2.527153349 | 0.035788708 | 0.08344291 |
| 59 | 0.171050829 | 0.171050829 | 3.890660293 | 0.004727539 | 0.019533312 |
| 60 | 0.174144322 | 0.174144322 | 2.584598623 | 0.0327468 | 0.078039912 |
| 61 | 0.18112944 | 0.18112944 | 2.685388848 | 0.028032588 | 0.069592429 |
| 62 | 0.19092131 | 0.19092131 | 3.73604023 | 0.005879148 | 0.022572713 |
| 63 | 0.194400918 | 0.194400918 | 2.492510608 | 0.037760627 | 0.086786653 |
| 64 | 0.195707712 | 0.195707712 | 2.71102351 | 0.026948639 | 0.067475149 |
| 65 | 0.204252124 | 0.204252124 | 3.975216299 | 0.004202345 | 0.018041687 |
| 66 | 0.210054656 | 0.210054656 | 2.338335953 | 0.047960033 | 0.103796265 |
| 67 | 0.210247736 | 0.210247736 | 2.234440593 | 0.056352274 | 0.11719604 |
| 68 | 0.213090816 | 0.213090816 | 2.087748617 | 0.07073665 | 0.138732545 |
| 69 | 0.226250319 | 0.226250319 | 2.25409429 | 0.054659526 | 0.114573853 |
| 70 | 0.246810388 | 0.246810388 | 5.953590771 | 0.000359019 | 0.0048119 |
| 71 | 0.247739738 | 0.247739738 | 4.372950932 | 0.002449301 | 0.012749359 |
| 72 | 0.248261394 | 0.248261394 | 6.173828851 | 0.000282141 | 0.004322646 |
| 73 | 0.251556432 | 0.251556432 | 4.318526719 | 0.00263344 | 0.013280362 |
| 74 | 0.253919456 | 0.253919456 | 1.95465634 | 0.086862876 | 0.161472968 |
| 75 | 0.256754187 | 0.256754187 | 5.535121315 | 0.000577231 | 0.005715942 |
| 76 | 0.257160612 | 0.257160612 | 3.449233265 | 0.008887517 | 0.030040982 |
| 77 | 0.259132781 | 0.259132781 | 4.366813249 | 0.002469352 | 0.012816471 |
| 78 | 0.287279843 | 0.287279843 | 2.539709619 | 0.035100109 | 0.082104681 |
| 79 | 0.31600033 | 0.31600033 | 3.153558526 | 0.013761553 | 0.041279885 |
| 80 | 0.358221647 | 0.358221647 | 3.100524122 | 0.014900586 | 0.043739937 |
| 81 | 0.364193755 | 0.364193755 | 3.369619436 | 0.009987239 | 0.03271603 |
| 82 | 0.453457772 | 0.453457772 | 3.247156175 | 0.011968978 | 0.037200176 |
| 83 | 0.180533568 | 0.180533568 | 5.147835975 | 0.000914678 | 0.007187473 |
| 84 | 0.182697701 | 0.182697701 | 5.877748203 | 0.00039063 | 0.004938906 |
| 85 | −0.148364769 | −0.148364769 | −4.986366569 | 0.001115061 | 0.008026755 |
| 86 | −0.538084185 | −0.538084185 | −6.494881534 | 0.000200669 | 0.003807401 |
| 87 | −0.545447375 | −0.545447375 | −6.02027801 | 0.000333544 | 0.004684915 |
| 88 | −0.554745602 | −0.554745602 | −8.383072026 | 0.0000337 | 0.002483007 |
| 89 | 0.503059535 | 0.503059535 | 6.535294395 | 0.000192409 | 0.003731412 |
| 90 | 0.36623319 | 0.36623319 | 5.026075307 | 0.001061678 | 0.007815282 |
| 91 | 0.338959712 | 0.338959712 | 4.957835746 | 0.001155226 | 0.008192382 |
| 92 | 0.127634089 | 0.127634089 | 5.070996787 | 0.001004634 | 0.007593027 |

TABLE 5.m

| | B | FC | FC_1 | LS | Loop Detected |
|---|---|---|---|---|---|
| 53 | −3.422371897 | 1.107122465 | 1.107122465 | 1 | DBLCL |
| 54 | −5.595015473 | 1.1078721 | 1.1078721 | 1 | DBLCL |
| 55 | −3.417108405 | 1.108300771 | 1.108300771 | 1 | DBLCL |
| 56 | −5.084251662 | 1.112463898 | 1.112463898 | 1 | DBLCL |
| 57 | −1.806028568 | 1.114273349 | 1.114273349 | 1 | DBLCL |
| 58 | −4.275957963 | 1.118118718 | 1.118118718 | 1 | DBLCL |
| 59 | −2.201619835 | 1.125878252 | 1.125878252 | 1 | DBLCL |
| 60 | −4.187168091 | 1.128295002 | 1.128295002 | 1 | DBLCL |
| 61 | −4.031097147 | 1.133771131 | 1.133771131 | 1 | DBLCL |
| 62 | −2.428494364 | 1.141492444 | 1.141492444 | 1 | DBLCL |
| 63 | −4.329420018 | 1.14424891 | 1.14424891 | 1 | DBLCL |
| 64 | −3.991368624 | 1.145285841 | 1.145285841 | 1 | DBLCL |
| 65 | −2.078878648 | 1.152088963 | 1.152088963 | 1 | DBLCL |
| 66 | −4.56625722 | 1.156732005 | 1.156732005 | 1 | DBLCL |
| 67 | −4.724458483 | 1.156886824 | 1.156886824 | 1 | DBLCL |
| 68 | −4.945085913 | 1.159168918 | 1.159168918 | 1 | DBLCL |
| 69 | −4.694639254 | 1.169790614 | 1.169790614 | 1 | DBLCL |
| 70 | 0.493437892 | 1.186580836 | 1.186580836 | 1 | DBLCL |
| 71 | −1.514951748 | 1.18734545 | 1.18734545 | 1 | DBLCL |
| 72 | 0.744313598 | 1.187774853 | 1.187774853 | 1 | DBLCL |
| 73 | −1.590768631 | 1.190490767 | 1.190490767 | 1 | DBLCL |
| 74 | −5.141601134 | 1.192442298 | 1.192442298 | 1 | DBLCL |
| 75 | −0.002180366 | 1.194787614 | 1.194787614 | 1 | DBLCL |
| 76 | −2.856981615 | 1.195124248 | 1.195124248 | 1 | DBLCL |
| 77 | −1.523480143 | 1.196759104 | 1.196759104 | 1 | DBLCL |
| 78 | −4.25656399 | 1.220337199 | 1.220337199 | 1 | DBLCL |
| 79 | −3.307417374 | 1.24487452 | 1.24487452 | 1 | DBLCL |
| 80 | −3.388938618 | 1.281844844 | 1.281844844 | 1 | DBLCL |
| 81 | −2.977498786 | 1.287162103 | 1.287162103 | 1 | DBLCL |
| 82 | −3.164028291 | 1.369318233 | 1.369318233 | 1 | DBLCL |
| 83 | −0.483711076 | 1.13330295 | 1.13330295 | 1 | DBLCL |
| 84 | 0.405473595 | 1.135004251 | 1.135004251 | 1 | DBLCL |
| 85 | −0.691112407 | 0.902272569 | −1.108312537 | −1 | Ctrl |
| 86 | 1.098164859 | 0.688684835 | −1.452043008 | −1 | Ctrl |
| 87 | 0.570114643 | 0.685178898 | −1.459472852 | −1 | Ctrl |
| 88 | 2.923843236 | 0.680777092 | −1.46890959 | −1 | Ctrl |
| 89 | 1.14173325 | 1.417215879 | 1.417215879 | 1 | DBLCL |
| 90 | −0.639742862 | 1.288982958 | 1.288982958 | 1 | DBLCL |
| 91 | −0.728168867 | 1.26484422 | 1.26484422 | 1 | DBLCL |
| 92 | −0.581917188 | 1.092500613 | 1.092500613 | 1 | DBLCL |

TABLE 5.n

| | | SEQ | Probe |
|---|---|---|---|
| | Probe sequence | ID | Location |
| | 60 mer | NO: | Chr |
| 53 | GAGGGGCCTCTGGAGGGGGCGGGTTCTCTCGATGCCTGGCCTCCACAGCACATGTGAGCA | 158 | 10 |
| 54 | AATGAGGAACTAGCAGCAGGAGGCAGCATCGAAACCTGGGATGCTAGTAACCCTACCCTG | 159 | 20 |
| 55 | TCCAATCACCTCCCACCAGGTCCCTCCCTCGATCCTGTGCTTTTCCTGCTGCAGGTTTCA | 160 | 7 |
| 56 | AGTGGCGTGATCATGGTTCACTGAAGCCTCGAAAAGAGGTTGGCTAGAAGGCCACGGGGT | 161 | 3 |
| 57 | GAGGACACGGCGGGGGGCCCATCACCCCTCGAACAGGAGCTGTCCCTCCCAGGAGCAGGC | 162 | 19 |
| 58 | GAGCCAGGTTTTGCAGGACCTGGGATATTCGAGACCAGTCTGGGCAACATAGTGAGACCC | 163 | 22 |
| 59 | TGGGGGTCCCGGGGAGGTGGGCGTTGCCTCGAATCTGGTCAAACCCTACCCAAACTCATC | 164 | 1 |
| 60 | AGATCCGTGTCTGCCTGCAGATACAAAATCGAGTTGGGCTGGGGAGAGGAGGAGATAGGT | 165 | 3 |
| 61 | CACTCGGGTGGCAGAGATGCGTGGAGAGTCGATGTGTCCCAAATTGATCTCACCCTCCAC | 166 | 20 |
| 62 | TGTGAAGGGAGGGGAGGAGAAAAGAAAATCGATCATCTCACCGGCCGAAGACGAGGAGGA | 167 | 6 |
| 63 | ACATTTCAAATCCTCTCTTCTAGCTACCTCGAACTTCTGAGCTCAAGCAATCTTCCACCT | 168 | 9 |
| 64 | TTCTGACAGAGGGTTGGGGAGAGGGGTGTCGACCTCCTAAAGTGCTGGGATTACAGGCGT | 169 | 17 |
| 65 | GGAGGATGGGGAGGGTATGTAAATATTGTCGATAGAGCAAGGAAACCAGAAAGGTGTAAT | 170 | 10 |
| 66 | GTATGAGTGTGGGTGTGTGGATGTGGCCTCGAGATCGCGCCACTGCACTCCAGCCTGGGC | 171 | 18 |
| 67 | ACGGGCAGACAGGACCCCAGCCCATGCCTCGACCCACTCCCGGGGGGATCGGGACACCGC | 172 | 22 |
| 68 | AGTGGTGCGATCTCAGCTTGTTGCAGCCTCGAGGAATTTCTAATGATAGATCCAGACCTC | 173 | 17 |
| 69 | TCATTCTGGGGATTATCTTTTGATTTTCTCGAGGCTGCAGTGAGCTATAATTGCACCACT | 174 | 4 |
| 70 | TGGGGGAGCTCTGGGGTGGGGGTAGCGGTCGATGGGTCCTGATGCCTCTCAGAAGGCCTT | 175 | 19 |
| 71 | GAGGUTTTTATGCAGGAAAGTGTCCCAGTCGAGGGACTGGCAGCAGGGGGACAGCAAGGG | 176 | 20 |
| 72 | GAGCTGGATGCCAGGCGGGCCAATGAGGTCGATTGCAATGCAGGATCCTATGCTGGATTC | 177 | 4 |
| 73 | AACAGGCAGGAGCAGCTGTTCCTCAGCATCGAACCTATTTATTTACTTATTTTTTTGAGA | 178 | 1 |
| 74 | TCTTTATGGTGTCTCTTTATATATTTACTCGAGGCTGCAGTGAGCTATAATTGCACCACT | 179 | 4 |
| 75 | GAGCTGGATGCCAGGCGGGCCAATGAGGTCGAACACGATATGAACAGGACATCTGTTACA | 180 | 4 |
| 76 | GGGTGGAGTCAGGGAGGGGTGGGGGACGTCGAGTCTTGCTTGACCCCAGAGCAGCTCCCT | 181 | 19 |
| 77 | GGGTTTCACCGTGTTACTCAGGCTGGTCTCGAAGTCCTGGGCTCAAGCAATCCACCCGCT | 182 | 22 |
| 78 | CAAATACTCATGTGTATGGGCAAAAAACTCGAGTAGTTGGAACTTCAAGTGTCAAAACAT | 183 | 4 |
| 79 | GACGGGCCGATTGCCTGAGCTCAGGAGTTCGACCCTTCTCACGTGGGCTAAGGGCCTGAC | 184 | 16 |
| 80 | ACTAGCTGGGTGACCCTAGACAGTTTGTTCGAGGCTACAGTGAGCTGTGATAGTGCCACT | 185 | 22 |
| 81 | TGTTGTATCCATTATTGAAAGTGGAGTATCGAGGCTGCAGTGAGCTGAGATCATTCCACT | 186 | 18 |
| 82 | GACAGGCAGATTGCCTGAGCTCAGGAGTTCGACATCTCTACACTCATTCTTTCTACTCAG | 187 | 17 |
| 83 | ACATTTCAAATCCTCTCTTCTAGCTACCTCGAAACACCACTACTTGTCAGTTTACAATGA | 188 | 9 |
| 84 | CGGTGTCTGGTGAGTTTTAACATCCTTGTCGAGCTGCAGACTTGGCTTTGGAAGAATCAC | 189 | 5 |
| 85 | GCTCAGCAAATGAATGTTTTCAAAGCACTCGATTGCAATGCAGGATCCTATGCTGGATTC | 190 | 4 |
| 86 | GTGCTTCAAGCAGAGCTTCCTCCCTCCGTCGAACTCCTGACCTCGTGATCCGCCTGCCTC | 191 | X |
| 87 | ATCCTAACTGCTGAAGTCTGTGTTTTCATCGAACTCCTGACCTCGTGATCCGCCTGCCTC | 192 | X |
| 88 | GGCTTGCCTAAAAAAGTAAACAAAACAGTCGAACTCCTGCTCATGATCCGCCTGCCTTGG | 193 | X |
| 89 | CCAAGGCAGGCGGATCATGAGCAGGAGTTCGAGACCAGCCTGGCCAAGATAGTGAAACCC | 194 | X |
| 90 | GCCGAGGCGGGTGGATCAGGTCAGGAGTTCGAGACCAGCCTGGCCAAGATAGTGAAACCC | 195 | X |
| 91 | GGCGGGTGGATCACTTGAGGTCAGGAGTTCGAGACCAGCCTGGCCAAGATAGTGAAACCC | 196 | X |
| 92 | CACTACTACCCAGGAAAGTGATGGGAGGTCGAGATTGCAGGAAATGGAGAGTACATGCCT | 197 | X |

TABLE 5.o

| | Probe Location | | | | 4 kb Sequence Location | | |
|---|---|---|---|---|---|---|---|
| | Start1 | End1 | Start2 | End2 | Chr | Start1 | End1 |
| 53 | 81892358 | 81892389 | 81927417 | 81927448 | 10 | 81888388 | 81892389 |
| 54 | 44737133 | 44737164 | 44777294 | 44777325 | 20 | 44737133 | 44741134 |
| 55 | 137535817 | 137535848 | 137608464 | 137608495 | 7 | 137531847 | 137535848 |
| 56 | 38161086 | 38161117 | 38182050 | 38182081 | 3 | 38157116 | 38161117 |
| 57 | 19257091 | 19257122 | 19273469 | 19273500 | 19 | 19253121 | 19257122 |
| 58 | 37569072 | 37569103 | 37583052 | 37583083 | 22 | 37569072 | 37573073 |
| 59 | 11322306 | 11322337 | 11348627 | 11348658 | 1 | 11318336 | 11322337 |
| 60 | 187439656 | 187439687 | 187454060 | 187454091 | 3 | 187435686 | 187439687 |
| 61 | 44638528 | 44638559 | 44671483 | 44671514 | 20 | 44634558 | 44638559 |
| 62 | 91285675 | 91285706 | 91296544 | 91296575 | 6 | 91281705 | 91285706 |
| 63 | 20560917 | 20560948 | 20666337 | 20666368 | 9 | 20556947 | 20560948 |
| 64 | 53404056 | 53404087 | 53422397 | 53422428 | 17 | 53404056 | 53408057 |
| 65 | 69655187 | 69655218 | 69676432 | 69676463 | 10 | 69651217 | 69655218 |
| 66 | 77124213 | 77124244 | 77283671 | 77283702 | 18 | 77124213 | 77128214 |
| 67 | 42305719 | 42305750 | 42342568 | 42342599 | 22 | 42301749 | 42305750 |
| 68 | 40456120 | 40456151 | 40581683 | 40581714 | 17 | 40456120 | 40460121 |
| 69 | 103512508 | 103512539 | 103564984 | 103565015 | 4 | 103512508 | 103516509 |
| 70 | 19271977 | 19272008 | 19303710 | 19303741 | 19 | 19271977 | 19275978 |
| 71 | 44744656 | 44744687 | 44767157 | 44767188 | 20 | 44740686 | 44744687 |
| 72 | 87408248 | 87408279 | 87515324 | 87515355 | 4 | 87408248 | 87412249 |
| 73 | 11190905 | 11190936 | 11269915 | 11269946 | 1 | 11190905 | 11194906 |
| 74 | 103430366 | 103430397 | 103512508 | 103512539 | 4 | 103426396 | 103430397 |
| 75 | 87373087 | 87373118 | 87515324 | 87515355 | 4 | 87373087 | 87377088 |
| 76 | 17890555 | 17890586 | 17934729 | 17934760 | 19 | 17886585 | 17890586 |
| 77 | 42332064 | 42332095 | 42352233 | 42352264 | 22 | 42328094 | 42332095 |
| 78 | 106058602 | 106058633 | 106118157 | 106118188 | 4 | 106058602 | 106062603 |
| 79 | 66835284 | 66835315 | 66909693 | 66909724 | 16 | 66835284 | 66839285 |
| 80 | 42335475 | 42335506 | 42362266 | 42362297 | 22 | 42335475 | 42339476 |
| 81 | 77151077 | 77151108 | 77274975 | 77275006 | 18 | 77151077 | 77155078 |
| 82 | 41214832 | 41214863 | 41227254 | 41227285 | 17 | 41214832 | 41218833 |
| 83 | 20377197 | 20377228 | 20560917 | 20560948 | 9 | 20377197 | 20381198 |
| 84 | 140753951 | 140753982 | 140893107 | 140893138 | 5 | 140749981 | 140753982 |
| 85 | 87166373 | 87166404 | 87408248 | 87408279 | 4 | 87166373 | 87170374 |
| 86 | 100646274 | 100646305 | 100689454 | 100689485 | X | 100646274 | 100650275 |
| 87 | 100625073 | 100625104 | 100689454 | 100689485 | X | 100625073 | 100629074 |
| 88 | 100590317 | 100590348 | 100627655 | 100627686 | X | 100586347 | 100590348 |
| 89 | 100627655 | 100627686 | 100647899 | 100647930 | X | 100627655 | 100631656 |
| 90 | 100647899 | 100647930 | 100675114 | 100675145 | X | 100647899 | 100651900 |
| 91 | 100602468 | 100602499 | 100647899 | 100647930 | X | 100602468 | 100606469 |
| 92 | 100610457 | 100610488 | 100670898 | 100670929 | X | 100610457 | 100614458 |

TABLE 5.p

| | 4 kb Sequence Location | | |
|---|---|---|---|
| | Start2 | End2 | Probe |
| 53 | 81927417 | 81931418 | ANXA11_10_81889664_81892389_81927417_81929312_FR |
| 54 | 44777294 | 44781295 | CD40_20_44737133_44739370_44777294_44780862_RR |
| 55 | 137608464 | 137612465 | CREB3L2_7_137532509_137535848_137608464_137613205_FR |
| 56 | 38182050 | 38186051 | MyD88_3_38159544_38161117_38182050_38188284_FR |
| 57 | 19269499 | 19273500 | MEF2B_19_19255724_19257122_19271977_19273500_FF |
| 58 | 37583052 | 37587053 | IL-2RB_22_37569072_37572860_37583052_37586677_RR |
| 59 | 11344657 | 11348658 | FRAP1_1_11321482_11322337_11347781_11348658_FF |
| 60 | 187450090 | 187454091 | BCL6_3_187438677_187439687_187452395_187454091_FF |
| 61 | 44667513 | 44671514 | MMP9_20_44635898_44638559_44669235_44671514_FF |
| 62 | 91296544 | 91300545 | MAP3K7_6_91275515_91285706_91296544_91297579_FR |
| 63 | 20662367 | 20666368 | MLLT3_9_20556478_20560948_20658310_20666368_FF |
| 64 | 53418427 | 53422428 | HLF_17_53404056_53408147_53420274_53422428_RF |
| 65 | 69676432 | 69680433 | SIRT1_10_69650583_69655218_69676432_69678199_FR |
| 66 | 77279701 | 77283702 | NFATc1_18_77124213_77127824_77280170_77283702_RF |
| 67 | 42342568 | 42346569 | TNFRSF13C_22_42302849_42305750_42342568_42346797_FR |
| 68 | 40577713 | 40581714 | STAT3_17_40456120_40457219_40580136_40581714_RF |
| 69 | 103561014 | 103565015 | NFKB1_4_103512508_103516923_103561903_103565015_RF |
| 70 | 19299740 | 19303741 | MEF2B_19_19271977_19273500_19302232_19303741_RF |
| 71 | 44767157 | 44771158 | CD40_20_44739847_44744687_44767157_44770555_FR |
| 72 | 87511354 | 87515355 | MAPK10_4_87408248_87409426_87514697_87515355_RF |
| 73 | 11269915 | 11273916 | FRAP1_1_11190905_11194522_11269915_11272450_RR |
| 74 | 103512508 | 103516509 | NFKBl_4_103425293_103430397_103512508_103516923_FR |
| 75 | 87511354 | 87515355 | MAPK10_4_87373087_87377906_87514697_87515355_RF |
| 76 | 17934729 | 17938730 | JAK3_19_17889333_17890586_17934729_17936992_FR |
| 77 | 42352233 | 42356234 | TNFRSF13C_22_42329800_42332095_42352233_42353781_FR |
| 78 | 106118157 | 106122158 | TET2_4_106058602_106063965_106118157_106119978_RR |
| 79 | 66905723 | 66909724 | NAE1_16_66835284_66840537_66902726_66909724_RF |
| 80 | 42362266 | 42366267 | TNFRSF13C_22_42335475_42336871_42362266_42363517_RR |
| 81 | 77274975 | 77278976 | NFATc1_18_77151077_77154182_77274975_77276499_RR |
| 82 | 41227254 | 41231255 | BRCA1_17_41214832_41217070_41227254_41229572_RR |
| 83 | 20556947 | 20560948 | MLLT3_9_20377197_20385409_20556478_20560948_RF |
| 84 | 140889137 | 140893138 | PCDHGA6/B2/B4_5_140751685_140753982_140892508_140893138_FF |
| 85 | 87408248 | 87412249 | MAPK10_4_87166373_87167382_87408248_87409426_RR |
| 86 | 100689454 | 100693455 | BTK_X_100646274_100647902_100689454_100691928_RR |
| 87 | 100689454 | 100693455 | BTK_X_100625073_100626595_100689454_100691928_RR |
| 88 | 100627655 | 100631656 | BTK_X_100587279_100590348_100627655_100629872_FR |
| 89 | 100647899 | 100651900 | BTK_X_100627655_100629872_100647899_100654354_RR |
| 90 | 100671144 | 100675145 | BTK_X_100647899_100654354_100673203_100675145_RF |
| 91 | 100647899 | 100651900 | BTK_X_100602468_100603585_100647899_100654354_RR |
| 92 | 100666928 | 100670929 | BTK_X_100610457_100612966_100667570_100670929_RF |

TABLE 5.q

| | | Inner_primers | | |
|---|---|---|---|---|
| | | | SEQ | |
| | | | ID | |
| | PCR-Primer1_ID | PCR_Primer1 | NO: | PCR-Primer2_ID |
| 53 | OBD RD048.209 | GGCTCGTAACAAACCCCTGACCCCAG | 198 | OBD RD048.211 |
| 54 | OBD RD048.213 | TCCCCATTACCCCATCAGTGCTCCCC | 199 | OBD RD048.215 |
| 55 | OBD RD048.217 | GGAGAGGCAGAGCAGAGAGTGAAGGG | 200 | OBD RD048.219 |
| 56 | OBD RD048.221 | GACAGCAGTTTCTAAGCCTGGCA | 201 | OBD RD048.223 |
| 57 | OBD RD048.225 | TTTGGAGGACTGGGACTTGCCGT | 202 | OBD RD048.227 |
| 58 | OBD RD048.229 | AACTGAAAGAAAGACCCAGAGGC | 203 | OBD RD048.231 |
| 59 | OBD RD048.233 | GACCCAAAGGGCAATACCAGAGC | 204 | OBD RD048.235 |
| 60 | OBD RD048.237 | CACGCTCGCCCATCATTGAAAAC | 205 | OBD RD048.239 |
| 61 | OBD RD048.241 | TCCCTTCATCCACAGGAATACCT | 206 | OBD RD048.243 |
| 62 | OBD RD048.245 | GGTTAGGTCTTCTGCCTTCAAAG | 207 | OBD RD048.247 |
| 63 | OBD RD048.249 | GTGTAACAATCAAGTCAGGGAAT | 208 | OBD RD048.251 |
| 64 | OBD RD048.253 | CACAGAGCCTGCCATCCTCACAT | 209 | OBD RD048.255 |
| 65 | OBD RD048.257 | AAATAAGTAAGGACAAAGAGTGC | 210 | OBD RD048.259 |
| 66 | OBD RD048.261 | TCGCCTACGGCTTGTTTACGCACAGC | 211 | OBD RD048.263 |
| 67 | OBD RD048.265 | GCTTATTTACAAGACGAACCCGC | 212 | OBD RD048.267 |
| 68 | OBD RD048.269 | TTCTGTTGTCCAGGCTTGAGTGC | 213 | OBD RD048.271 |
| 69 | OBD RD048.273 | CACTATTGAGTTCTAAGAGTTCT | 214 | OBD RD048.275 |
| 70 | OBD RD048.277 | GGAACCCACGCCCTCCCCTAAGTCTT | 215 | OBD RD048.279 |
| 71 | OBD RD048.281 | GGTGTGCTTTGCCAGGATAAGAA | 216 | OBD RD048.283 |
| 72 | OBD RD048.285 | TCTCCCTGGCGACCTCGTCCCTA | 217 | OBD RD048.287 |
| 73 | OBD RD048.289 | TGTTTGCTTTATGGACACACAGA | 218 | OBD RD048.291 |
| 74 | OBD RD048.293 | CATTTACTCACTCTCATACCATA | 219 | OBD RD048.295 |
| 75 | OBD RD048.297 | ACTCTGCCGCTCGGTCACCAACCTGA | 220 | OBD RD048.299 |
| 76 | OBD RD048.301 | GACAAGGGAGGGAGGAGGATGGG | 221 | OBD RD048.303 |
| 77 | OBD RD048.305 | CCTGCCTCAGCCTCCCAAGTAGC | 222 | OBD RD048.307 |
| 78 | OBD RD048.309 | GTGAACTCAGCCAAGCACAGTGGTGG | 223 | OBD RD048.311 |
| 79 | OBD RD048.313 | TTCTTTACCCCTGTCACTCACCT | 224 | OBD RD048.315 |
| 80 | OBD RD048.317 | TGGTTGGAAGTAGCCCTGATTCA | 225 | OBD RD048.319 |
| 81 | OBD RD048.321 | GTTGCCTTGTTATCTGCCTGGTT | 226 | OBD RD048.323 |
| 82 | OBD RD048.325 | GTAATCCTAACACTGTGGGAGGC | 227 | OBD RD048.327 |
| 83 | OBD RD048.329 | GGGAGCATTGTGGGCTAACAGGAGAC | 228 | OBD RD048.331 |
| 84 | OBD RD048.333 | TCGTAGGCAACATCGTCAAGGAT | 229 | OBD RD048.335 |
| 85 | OBD RD048.337 | CTGGGCAACAGAGTGAGAGCCTG | 230 | OBD RD048.339 |
| 86 | OBD RD051.001 | TGCTACCTCTGACTACAGGGTGG | 231 | OBD RD051.003 |
| 87 | OBD RD051.005 | GCTGACTGAAGATTCTGCCTTTC | 232 | OBD RD051.007 |
| 88 | OBD RD051.009 | TAGGATGGCAAGCAGCATTGGCT | 233 | OBD RD051.011 |
| 89 | OBD RD051.013 | CACGCCTGTAATCCCAGCACTTTGG | 234 | OBD RD051.015 |
| 90 | OBD RD051.017 | CACGCCTGTAATCCCAGCACTCTG | 235 | OBD RD051.019 |
| 91 | OBD RD051.021 | ATGCCTGTAATCCCAGCACTTTGG | 236 | OBD RD051.023 |
| 92 | OBD RD051.025 | CCACCATTCGTGCTCCAACACTC | 237 | OBD RD051.027 |

TABLE 5.r

| | SEQ | | | | |
|---|---|---|---|---|---|
| | ID | Inner_primers | | | |
| | NO: | PCR_Primer2 | Gene | Marker | GLMNET |
| 53 | 238 | ACAGTTGTGGAGGCTCAATACCT | ANXA11 | OBD | 0.00000056 |
| | | | | RD048.209.211 | |
| 54 | 239 | CGGTAACAGACACGGAGTGAAAT | CD40 | OBD | 0.00000222 |
| | | | | RD048.213.215 | |
| 55 | 240 | GCAGGGACTGAGAAACATAGGAT | CREB3L2 | OBD | 3.82E−08 |
| | | | | RD048.217.219 | |
| 56 | 241 | TGGACCCCAGGGCAGGGCTTCAT | MyD88 | OBD | 0.000000196 |
| | | | | RD048.221.223 | |
| 57 | 242 | TCAGACCCTCCTTCCCACCTCTC | MEF2B | OBD | 0.00000288 |
| | | | | RD048.225.227 | |
| 58 | 243 | CCCCTTCTCCTGCTGCTACCATCCAG | IL-2RB | OBD | 0.000000645 |
| | | | | RD048.229.231 | |
| 59 | 244 | CTCAGGGAGACCAAGGCAGTGAC | FRAP1 | OBD | 0.00000196 |
| | | | | RD048.233.235 | |
| 60 | 245 | GGGACTGGAGGGAAGGAAGTGGG | BCL6 | OBD | 0.00000325 |
| | | | | RD048.237.239 | |
| 61 | 246 | GGAGCAGTGTAGGGCAGGGTGTCAGA | MMP9 | OBD | 0.00000227 |
| | | | | RD048.241.243 | |
| 62 | 247 | ATGTCTACAGCCTCTGCCGCCTCCTC | MAP3K7 | OBD | 0.000000566 |
| | | | | RD048.245.247 | |
| 63 | 248 | GCCCTGTAATCCCAGCACTTTGG | MLLT3 | OBD | 0.0000046 |
| | | | | RD048.249.251 | |
| 64 | 249 | CCCCAGGGACTGAGGACTTGTGT | HLF | OBD | 0.000000743 |
| | | | | RD048.253.255 | |
| 65 | 250 | AACAATCTATTTTACCAACCTAT | SIRT1 | OBD | 0.00000188 |
| | | | | RD048.257.259 | |
| 66 | 251 | CAGGTAGTGTGTTTTCCAACTCTGTT | NFATc1 | OBD | 0.000000147 |
| | | | | RD048.261.263 | |
| 67 | 252 | TAGTAGAGAGTGCGGTGCCCACAGGC | TNFRSF13C | OBD | 0.000000402 |
| | | | | RD048.265.267 | |
| 68 | 253 | GGCAAGGTCTCCAGTGGTGAGGT | STAT3 | OBD | 0.00000103 |
| | | | | RD048.269.271 | |
| 69 | 254 | GTCTCACTCTGTTGCCCAGGCTG | NFKB1 | OBD | 0.00000177 |
| | | | | RD048.273.275 | |
| 70 | 255 | TGGATTTTCTGCGGCTCTGTTTG | MEF2B | OBD | 0.00000137 |
| | | | | RD048.277.279 | |
| 71 | 256 | AGTCCCCTCTCTGGGTCTCAGCCAAG | CD40 | OBD | 0.00000449 |
| | | | | RD048.281.283 | |
| 72 | 257 | TATGGCATTTTCCCCTTCCAGTA | MAPK10 | OBD | 0.00000213 |
| | | | | RD048.285.287 | |
| 73 | 258 | CACTCCAGCCTGAGAGACAGAGC | FRAP1 | OBD | 0.00000287 |
| | | | | RD048.289.289 | |
| 74 | 259 | GTCTCACTCTGTTGCCCAGGCTG | NFKB1 | OBD | 0.00000178 |
| | | | | RD048.291.293 | |
| 75 | 260 | CAGGGTTGTTGTGAGGGTTATGT | MAPK10 | OBD | 0.00000339 |
| | | | | RD048.295.297 | |
| 76 | 261 | GTCCCTGCTCTCTTAGCCCCAGA | JAK3 | OBD | 0.000000206 |
| | | | | RD048.299.301 | |
| 77 | 262 | AGACCTTTGGTTTCTACATCTAT | TNFRSF13C | OBD | 0.000000144 |
| | | | | RD048.303.305 | |
| 78 | 263 | GGTATCAAATGTTCCACAAGTGTTGC | TET2 | OBD | 0.000000972 |
| | | | | RD048.307.309 | |
| 79 | 264 | CCAGGATGTCTTACCGCCCCGTCAG | NAE1 | OBD | 0.00000172 |
| | | | | RD048.311.313 | |
| 80 | 265 | GGGTCTCACTCTGTTGCCCAAGC | TNFRSF13C | OBD | 0.00000164 |
| | | | | RD048.315.317 | |
| 81 | 266 | CGTCTTGCTCTGTCTGTTGCCCAGGC | NFATc1 | OBD | 0.00000111 |
| | | | | RD048.319.321 | |
| 82 | 267 | GGCAATAGGGATGATTCTGTGAA | BRCA1 | OBD | 0.00000046 |
| | | | | RD048.323.325 | |
| 83 | 268 | GCACAGGAGGGTTACTTCACAAG | MLLT3 | OBD | 0.0000292 |
| | | | | RD048.327.329 | |
| 84 | 269 | GCTTCACGGGAGGAGGGTAGACTCTC | PCDHGA6/ | OBD | 0.0000208 |
| | | | B2/B4 | RD048.331.333 | |
| 85 | 270 | TATGGCATTTTCCCCTTCCAGTA | MAPK10 | OBD | −0.0000511 |
| | | | | RD048.335.337 | |
| 86 | 271 | ATGTTAGTCCCTTCCCACCCTAT | BTK | OBD | 0.000000091 |
| | | | | RD051.001.003 | |
| 87 | 272 | ATGTTAGTCCCTTCCCACCCTAT | BTK | OBD | −8.44E−08 |
| | | | | RD051.005.007 | |
| 88 | 273 | ACGCCTGTAATCCCAGCACTTTG | BTK | OBD | −0.0000019 |
| | | | | RD051.009.011 | |
| 89 | 274 | GATTCTCCTGCCTCAGCCTCCCG | BTK | OBD | 9.55E−08 |
| | | | | RD051.013.015 | |
| 90 | 275 | CGATTCTCCTGCCTCAGCCTCCCG | BTK | OBD | 5.07E−08 |
| | | | | RD051.017.019 | |
| 91 | 276 | CGATTCTCCTGCCTCAGCCTCCCG | BTK | OBD | 2.87E−08 |
| | | | | RD051.021.023 | |
| 92 | 277 | CTCACGAACCGCCTCCTTTCCTC | BTK | OBD | 0.00000409 |
| | | | | RD051.025.027 | |

TABLE 6.a

| | | | Probe_ | Probe_ |
|---|---|---|---|---|
| | Probe | GeneLocus | Count_Total | Count_Sig |
| 1 | MIR98_X_53608013_53611637_53628991_53630033_RR | MIR98 | 16 | 4 |
| 2 | DAPK1_9_90064560_90073617_90140806_90142738_FR | DAPK1 | 46 | 9 |
| 3 | HSD3B2_1_119912462_119915175_119959754_119963670_RR | HSD3B2 | 20 | 5 |
| 4 | ERG_21_39895678_39899145_39984806_39991905_RF | ERG | 52 | 4 |
| 5 | SRD5A3_4_56188038_56191526_56242301_56245314_RF | SRD5A3 | 12 | 4 |
| 6 | MMP1_11_102658858_102661735_102664717_102667643_FF | MMP1 | n/a | n/a |

TABLE 6.b

| | HyperG_Stats | FDR_HyperG | Percent_Sig | logFC | AveExpr | t |
|---|---|---|---|---|---|---|
| 1 | 0.064790053 | 0.737205743 | 25 | 0.67511652 | 0.67511652 | 13.76185645 |
| 2 | 0.032709022 | 0.548212211 | 19.57 | 0.299375751 | 0.299375751 | 7.197207444 |
| 3 | 0.040338404 | 0.548212211 | 25 | −0.168081632 | −0.168081632 | −3.274998031 |
| 4 | 0.765503518 | 1 | 7.69 | −0.425291613 | −0.425291613 | −11.67074071 |
| 5 | 0.024128503 | 0.483719041 | 33.33 | 0.266992266 | 0.266992266 | 4.835274287 |
| 6 | n/a | n/a | n/a | n/a | 4.72222828 | n/a |

TABLE 6.c

| | P.Value | adj.P.Val | B | FC | FC_1 | LS |
|---|---|---|---|---|---|---|
| 1 | 0.000000031 | 0.0000143 | 9.558686586 | 1.596725728 | 1.596725728 | 1 |
| 2 | 0.0000184 | 0.000805368 | 3.154114326 | 1.230611817 | 1.230611817 | 1 |
| 3 | 0.007481356 | 0.033194645 | −3.020586815 | 0.890025372 | −1.123563476 | −1 |
| 4 | 0.000000168 | 0.0000357 | 7.913111034 | 0.744688192 | −1.342843905 | −1 |
| 5 | 0.000536131 | 0.005815136 | −0.328887879 | 1.203296575 | 1.203296575 | 1 |
| 6 | 0.04505295 | 0.4547981 | n/a | n/a | n/a | n/a |

TABLE 6.d

| | | | SEQ |
|---|---|---|---|
| | Loop | Probe sequence | ID |
| | Detected | 60 mer | NO: |
| 1 | Agressive | AGTTGTATTTTTAGAAAG | 278 |
| | | TAGTGTTTAATCGATAGA | |
| | | AATATAACATGAAACACA | |
| | | TATATA | |
| 2 | Aggressive | ACTAATCCCCTGAAGAAG | 279 |
| | | CAAATTAACTTCGAGTAT | |
| | | CCCTTTAAGTTTGTTTTT | |
| | | AAAATA | |
| 3 | Indolent | TCAGTTTCTGCTCTCAAG | 280 |
| | | AAGCTTACAGTCGAAGGT | |
| | | CCCAAGTTAGATTACGGC | |
| | | AAAGCT | |
| 4 | Indolent | TCTTGAATGTGCTTAGTA | 281 |
| | | TTATTCAGACTCGAAAAC | |
| | | ATAATTTGAAAGGAATTC | |
| | | ATTCTG | |
| 5 | Aggressive | AGGAGGTAACGATTGGTC | 282 |
| | | AGCTGCTTAATCGAGGCA | |
| | | GAAGTCTATTTGAAACGT | |
| | | AAGATA | |
| 6 | n/a | GGCCTTTAAGGCCCCTCT | 283 |
| | | GAAATCCAGCATCGAAGA | |
| | | GGGAAACTGCATCACA | |
| | | GTTGATGG | |

TABLE 6.e

| | Probe Location | | | | | 4 kb Sequence Location | | |
|---|---|---|---|---|---|---|---|---|
| | Chr | Start 1 | End1 | Start2 | End2 | Chr | Start1 | End1 |
| 1 | X | 53608013 | 53608044 | 53628991 | 53629022 | X | 53608013 | 53612014 |
| 2 | 9 | 90073586 | 90073617 | 90140806 | 90140837 | 9 | 90069616 | 90073617 |
| 3 | 1 | 119912462 | 119912493 | 119959754 | 119959785 | 1 | 119912462 | 119916463 |
| 4 | 21 | 39895678 | 39895709 | 39991874 | 39991905 | 21 | 39895678 | 39899679 |
| 5 | 4 | 56188038 | 56188069 | 56245283 | 56245314 | 4 | 56188038 | 56192039 |
| 6 | 11 | 102661704 | 102661735 | 102667612 | 102667643 | 11 | 102657734 | 102661735 |

TABLE 6.f

| | 4 kb Sequence Location | | |
|---|---|---|---|
| | Start2 | End2 | Marker |
| 1 | 53628991 | 53632992 | MIR98_X_53608013_53611637_53628991_53630033_RR |
| 2 | 90140806 | 90144807 | DAPK1_9_90064560_90073617_90140806_90142738_FR |
| 3 | 119959754 | 119963755 | HSD3B2_1_119912462_119915175_119959754_119963670_RR |
| 4 | 39987904 | 39991905 | ERG_21_39895678_39899145_39984806_39991905_RF |
| 5 | 56241313 | 56245314 | SRD5A3_4_56188038_56191526_56242301_56245314_RF |
| 6 | 102663642 | 102667643 | MMP1 |

TABLE 6.g

| | | | SEQ | | | SEQ |
|---|---|---|---|---|---|---|
| | Primers_ | Primer_ | ID | | | ID |
| | names | sequences | NO: | | | NO: |
| 1 | PCa119- | AAGAAGGGATG | 284 | PCa119- | GGTACACGAATT | 290 |
| | 245 | GGACGGGACT | | 247 | AACTATTCCCTGT | |
| 2 | PCa119- | ACTGGTCACAG | 285 | PCa119- | AGGTGTGAATGT | 291 |
| | 165 | GGAACGATGG | | 167 | TACTGAACACAAA | |
| 3 | PCa119- | ACTTGGATTCC | 286 | PCa119- | CTCTTCCCCGGT | 292 |
| | 130 | CAAAACGCCA | | 132 | GAGTTTCCA | |
| 4 | PCa119- | CAGCCTACCTT | 287 | PCa119- | AAAGCCCAGTGA | 293 |
| | 065 | GCCTGACACT | | 067 | TGGCCCAT | |
| 5 | PCa119- | TCCATTTTCCT | 288 | PCa119- | CCACACAGGGC | 294 |
| | 154 | TTCCCTTTGCT | | 155 | CCTAATGACC | |
| | | CTG | | | | |
| 6 | MMP 1-4 | GGGGAGTGGAT | 289 | MMP | TGGGCCTGGT | 295 |
| | 2F | GGGATAAGGTG | | IF | TGAAAAGCAT | |

TABLE 6.h

| | | | SEQ | |
|---|---|---|---|---|
| | | | ID | |
| | Probe | Probe_sequence | NO: | Gene |
| 1 | OBD119F015 | AGTGTTTAATCGATA | 296 | MIR98 |
| | | GAAATATAACATGAA | | |
| | | ACACA | | |
| 2 | OBD119F06 | AGGGATACTCGAAGT | 297 | DAPK1 |
| | | TAATTTGCTTCTT | | |
| 3 | OBD119F09 | AAGAAGCTTACAGTC | 298 | HSD3B2 |
| | | GAAGGTCCCAA | | |
| 4 | OBD119F08 | ATTCCTTTCAAATTA | 299 | ERG |
| | | TGTTTTCGAGTCTGA | | |
| | | ATAATA | | |
| 5 | SRD5A3FAM7415RC | AAATAGACTTCTGCC | 300 | SRD5A3 |
| | | TCGATTAAGCA | | |
| 6 | MMP1F1b2 | ATCCAGCATCGAAGA | 301 | MMP1 |
| | | GGGAAACTGCATCA | | |

TABLE 6.i

| | Marker | GLMNET |
|---|---|---|
| 1 | PCa119-245.247 | −5.91743E−06 |
| 2 | PCa119-165.167 | −1.57185E−05 |
| 3 | PCa119-130.132 | 4.47291E−07 |
| 4 | PCa119-065.067 | 6.32136E−06 |
| 5 | PCa119-154.155 | −8.00857E−08 |
| 6 | MMP1-4 1F. MMP 1F | 0 |

TABLE 7
Preferred DLBCL markers

| Marker | GLMNET |
|---|---|
| OBD RD048.001.003 | 2.08E−08 |
| OBD RD048.005.007 | 5.6E−07 |
| OBD RD048.009.011 | 4.49E−06 |
| OBD RD048.013.015 | 8.38E−07 |
| OBD RD048.017.019 | 1.56E−06 |
| OBD RD048.021.023 | 1.37E−06 |
| OBD RD048.025.027 | 0.0000046 |
| OBD RD048.029.031 | 1.81E−06 |
| OBD RD048.033.035 | 1.78E−06 |
| OBD RD048.037.039 | 4.02E−07 |

TABLE 8.a

| | | Inner_ | | SEQ |
|---|---|---|---|---|
| | | Forward | Inner_Forward_ | ID |
| N | EpiSwitch_ID | Primer ID | Primer_Seq | NO: |
| 1 | ORF1_1_1034282_1037357_1049484_1054771_FF | OBD169_001 | GCCAGAGAACAGATGTGTGTGTCT | 302 |
| 2 | ORF5_1_1140030_1142517_1196191_1197234_RR | OBD169_005 | GCCTCTCTGGTGCCACATCTTATCTT | 303 |
| 3 | ORF5_1_1182474_1185271_1270569_1273244_RF | OBD169_009 | CTGCCTGTGTGTAGTCACGAGAAGC | 304 |
| 4 | ORF5_1_1182474_1185271_1196191_1197234_RR | OBD169_013 | CTGACAGCAGAAGCACGAAAAGGTC | 305 |
| 5 | ORF5_1_1283682_1285577_1335341_1338794_RF | OBD169_017 | CCATCCACCCCACAGTTCCTATGAAA | 306 |
| 6 | ORF5_1_1147651_1150121_1196191_1197234_RF | OBD169_021 | CCCAACGAGGTCAGGAAGGGAGA | 307 |
| 7 | ORF5_1_1140030_1142517_1289361_1294150_FF | OBD169_025 | TGTCTCAGTATCTATTTCCCAAGTGC | 308 |
| 8 | ORF1_1_1038521_1042933_1098468_1101242_RF | OBD169_029 | CAGGACCCAGACTTGCCCAAACC | 309 |
| 9 | ORF5_1_1146367_1147651_1165983_1167502_FF | OBD169_033 | AGACCCAATGCCTGCCACACGGA | 310 |
| 10 | ORF5_1_1140030_1142517_1270569_1273244_RF | OBD169_037 | CTGCCTGTGTGTAGTCACGAGAAGC | 311 |
| 11 | ORF5_1_1196191_1197234_1230936_1232838_RR | OBD169_041 | GCATAACTCAGAGAAAGCCACTGTGA | 312 |
| 12 | ORF5_1_1182474_1185271_1209527_1216771_RR | OBD169_045 | CTGACAGCAGAAGCACGAAAAGGTC | 313 |
| 13 | ORF5_1_1270569_1273244_1300933_1312034_FF | OBD169_049 | CTGCCTGTGTGTAGTCACGAGAAGC | 314 |
| 14 | ORF5_1_1157878_1159517_1196191_1197234_RF | OBD169_053 | CCCAACGAGGTCAGGAAGGGAGA | 315 |
| 15 | ORF5_1_1273244_1276010_1335341_1338794_RF | OBD169_057 | CACCCATCCACCCCACAGTTCCT | 316 |
| 16 | ORF5_1_1196191_1197234_1289361_1294150_FF | OBD169_061 | CCCAACGAGGTCAGGAAGGGAGA | 317 |
| 17 | ORF5_1_1140030_1142517_1230936_1232838_RR | OBD169_065 | CCTCTCTGGTGCCACATCTTATCTTA | 318 |
| 18 | ORF5_1_1142517_1146335_1270569_1273244_RR | OBD169_069 | TTGACCTGGGCTCACATCGCTGA | 319 |
| 19 | ORF5_1_1230936_1232838_1273244_1276010_RR | OBD169_073 | GTCTTCAAGCCACAGAGCAGGATTCC | 320 |
| 20 | ORF5_1_1157878_1159517_1300933_1312034_FF | OBD169_077 | GGTCTGAAAATGTGAATGTCTTGTGT | 321 |
| 21 | ORF5_1_1147651_1150121_1273244_1276010_RR | OBD169_081 | GTGCCCTTGAGTCCAGCCGTCAT | 322 |
| 22 | ORF1_1_1049484_1054771_1098468_1101242_FF | OBD169_085 | TGTCTCTCTCCTAAGGTGTCCCC | 323 |
| 23 | ORF5_1_1209527_1216771_1270569_1273244_RF | OBD169_089 | CTGCCTGTGTGTAGTCACGAGAAGC | 324 |
| 24 | ORF48_2_84841864_84843477_84864219_84866005_FF | OBD169_093 | GCACTTTCTCTCCAGGTCACCCT | 325 |
| 25 | ORF48_2_84864219_84866005_84885415_84887815_RR | OBD169_097 | CTGCTTGGGCTGGTCTTTGGTTG | 326 |
| 26 | ORF48_2_84841864_84843477_84925461_84928171_FF | OBD169_101 | GGCACTTTCTCTCCAGGTCACCC | 327 |
| 27 | ORF41_2_36413514_36415342_36452868_36458269_RR | OBD169_105 | TGAGCGGTCACTGCTGTTGTAGG | 328 |
| 28 | ORF48_2_84864219_84866005_84876440_84877895_RF | OBD169_109 | TTCCATCCTGCTGTCCGTCCTGC | 329 |
| 29 | ORF48_2_84864219_84866005_84925461_84928171_FF | OBD169_113 | CGGAGAGAAGGCGGAGAAACCGT | 330 |
| 30 | ORF41_2_36413514_36415342_36468165_36471683_RR | OBD169_117 | GAGCGGTCACTGCTGTTGTAGGC | 331 |
| 31 | ORF91_7_65033242_65035577_65065127_65067650_RF | OBD169_121 | CATTCCTGGTATCGTGTTGCCGC | 332 |
| 32 | ORF91_7_65032142_65033242_65065127_65067650_FF | OBD169_125 | GGACTTCCTCCTCGCCTAATGCG | 333 |
| 33 | ORF91_7_65037215_65039217_65065127_65067650_RF | OBD169_129 | TCCTCCCATCCTCACTGGACCAC | 334 |
| 34 | ORF9_10_23456592_23460302_23494817_23496168_RR | OBD169_133 | AGGGCTCTGCGTTTACTCCAGGC | 335 |
| 35 | ORF15_11_39960254_39968870_39992990_40001746_RR | OBD169_137 | CTGGAGCCTGAGTAATGAATAGGAGC | 336 |
| 36 | ORF16_11_40371218_40374048_40393587_40395559_RR | OBD169_141 | GCCCCAATCCCATCCAGAATCCA | 337 |
| 37 | ORF15_11_39932865_39938937_40079832_40084530_FF | OBD169_145 | CTTTCTCTCTTCCCTCGTCCCTGG | 338 |
| 38 | ORF15_11_39992990_40001746_40079832_40084530_RF | OBD169_149 | TTTGATAATGAGGGCTGGCTGGGCAT | 339 |
| 39 | ORF16_11_40371218_40374048_40393587_40395559_RF | OBD169_153 | GGATGCCTTAGTTCCTATTGACACT | 340 |
| 40 | ORF27_12_63568927_63574607_63596388_63598936_RR | OBD169_157 | CTGCTGGAGGAGTGACACAAAGTTTC | 341 |
| 41 | ORF27_12_63568927_63574607_63586940_63589534_RR | OBD169_161 | GCCTGCTGGAGGAGTGACACAAAGTT | 342 |
| 42 | ORF31_15_29619588_29621525_29646237_29648560_RR | OBD169_165 | CCTTTCCTCTTCCATCTACTCATTCC | 343 |
| 43 | ORF30_15_10476260_10484217_10545581_10548270_RR | OBD169_169 | TTCTATCCCTCCACAAGATGCTCATA | 344 |
| 44 | ORF32_16_10690178_10695010_10747182_10750815_RR | OBD169_173 | GGGAGACGGAGGAAAAGCCTATC | 345 |
| 45 | ORF32_16_10747182_10750815_10765838_10768877_RR | OBD169_177 | AACCTCCTCAAAGAGAGAGCCTTCCC | 346 |
| 46 | ORF32_16_10726068_10729293_10772875_10776021_FF | OBD169_181 | AGGTCTTCAACCAAACACCACCAGTG | 347 |
| 47 | ORF32_16_10747182_10750815_10792291_10794979_RF | OBD169_185 | CCTCCTGTATTTCTACTTCCACTCAG | 348 |
| 48 | ORF32_16_10726068_10729293_10792291_10794979_FF | OBD169_189 | GCAGGTCTTCAACCAAACACCACCAG | 349 |
| 49 | ORF32_16_10747182_10750815_10772875_10776021_RR | OBD169_193 | AACCTCCTCAAAGAGAGAGCCTTCCC | 350 |
| 50 | ORF32_16_10778964_10780903_10792291_10794979_FF | OBD169_197 | CAGTGTGAAAGCACCTTCGCTCTTGC | 351 |
| 51 | ORF68_25_630610_633794_676143_680436_FF | OBD169_201 | GGGCAATGTGAGGCTGTTATGCTTGT | 352 |
| 52 | ORF68_25_630610_633794_687567_692655_FF | OBD169_205 | CCAGGGCAATGTGAGGCTGTTATGCT | 353 |
| 53 | ORF70_26_27906620_27909025_27963114_27965001_RR | OBD169_209 | TTTGAGGGCAGAGCAGGAAGGGT | 354 |
| 54 | ORF70_26_27876428_27879774_27894296_27895372_RR | OBD169_213 | GTCCCTGCTCCACTGCCAATGAG | 355 |
| 55 | ORF70_26_27890569_27893929_27933912_27935209_RR | OBD169_217 | GTGCCCTGGATGGAGAACTTGCT | 356 |
| 56 | ORF70_26_27933912_27935209_27963114_27965001_RR | OBD169_221 | TACAGAAAGCCCTCGCTGGGAGC | 357 |
| 57 | ORF70_26_27876428_27879774_27890569_27893929_RR | OBD169_225 | AAGTGTAGCACGGACCAGAGAGC | 358 |
| 58 | ORF70_26_27894296_27895372_27963114_27965001_RR | OBD169_229 | CTGCCTCCAGAAGGTGTCTCAGA | 359 |
| 59 | ORF70_26_27890569_27893929_27906620_27909025_RR | OBD169_233 | GTGCCCTGGATGGAGAACTTGCT | 360 |
| 60 | ORF75_31_28027888_28030129_28041732_28043951_FF | OBD169_237 | GGACAAGCATCCTGGTTGAGCCA | 361 |
| 61 | ORF75_31_28027888_28030129_28043951_28045576_FF | OBD169_241 | GGACAAGCATCCTGGTTGAGCCA | 362 |
| 62 | ORF79_32_24013860_24017127_24039530_24040887_RF | OBD169_245 | GACCCAGAAATGAACCCAAAAGATGA | 363 |
| 63 | ORF79_32_23988046_23989457_24013860_24017127_RR | OBD169_249 | GCACTCCCTACACACAAATCCTTAGA | 364 |
| 64 | ORF79_32_23965697_23967743_24013860_24017127_RR | OBD169_253 | GCAACAGTTCATAACCGAGTGCCAAC | 365 |
| 65 | ORF79_32_23965697_23967743_24028587_24030780_RR | OBD169_257 | GCAACAGTTCATAACCGAGTGCCAAC | 366 |
| 66 | ORF79_32_23965697_23967743_24000345_24005192_RR | OBD169_261 | CAGTTCATAACCGAGTGCCAACAGAA | 367 |
| 67 | ORF79_32_24013860_24017127_24028587_24030780_RR | OBD169_265 | GGTGACTGATGAGACTCCAGGAAAGT | 368 |
| 68 | ORF79_32_23965697_23967743_24039530_24040887_RF | OBD169_269 | GACCCAGAAATGAACCCAAAAGATGA | 369 |
| 69 | ORF79_32_23988046_23989457_24039530_24040887_RF | OBD169_273 | GACCCAGAAATGAACCCAAAAGATGA | 370 |
| 70 | ORF82_32_9652472_9664654_9692674_9698030_RR | OBD169_277 | CCCACCTCCCTGCTCCAACAAGATTT | 371 |
| 71 | ORF79_32_24000345_24005192_24039530_24040887_RF | OBD169_281 | GACCCAGAAATGAACCCAAAAGATGA | 372 |
| 72 | ORF79_32_23988046_23989457_24000345_24005192_RR | OBD169_285 | GCAGCCTTTGGCAGCACTCTCTG | 373 |
| 73 | ORF104_X_109512943_109516164_109526507_109531763 | OBD169_289 | CCCTTCTGGAACTGGATGAGCCCTTA | 374 |
| | _RF | | | |
| 74 | ORF104_X_109508063_109510622_109526507_109531763 | OBD169_293 | TGAGCCCTTAGTCAATGGGACCG | 375 |
| | _FF | | | |
| 75 | ORF106_X_75279499_75281082_75297768_75302185_RF | OBD169_297 | CCAGTTCACCAAGGTTGAGTGCC | 376 |

TABLE 8.b

| | | | | | | SEQ |
|---|---|---|---|---|---|---|
| | Inner_Reverse | | | | | ID |
| N | Primer ID | Inner Reverse Primer Seq | Gene | Marker | GLMNET | NO: |
| 1 | OBD169_003 | AAAACTCCCACCTGTCTGTGTCAC | NFATC1 | OBD169_001.OBD169_003 | 0.150341207 | 377 |
| 2 | OBD169_007 | GCATAACTCAGAGAAAGCCACTGTGA | ATP9B | OBD169_005.OBD169_007 | 0 | 378 |
| 3 | OBD169_011 | GACAGCAGAAGCACGAAAAGGTCATT | ATP9B | OBD169_009.OBD169_011 | −0.065057056 | 379 |
| 4 | OBD169_015 | TGTCCCTCCAGCCTCTGTTACCC | ATP9B | OBD169_013.OBD169_015 | 0.011765488 | 380 |
| 5 | OBD169_019 | GGTCTGAAAGCACCTGTAACTCTGGA | ATP9B | OBD169_017.OBD169_019 | 0 | 381 |
| 6 | OBD169_023 | CCCTTGAGTCCAGCCGTCATTAC | ATP9B | OBD169_021.OBD169_023 | 0 | 382 |
| 7 | OBD169_027 | ACACGATGAGACAGAGCACCAGAGTC | ATP9B | OBD169_025.OBD169_027 | 0 | 383 |
| 8 | OBD169_031 | GGTGAGTTCTGACCTGGGCTTTC | NFATC1 | OBD169_029.OBD169_031 | 0 | 384 |
| 9 | OBD169_035 | TCTGAGGTCCTGATGGAGCACAG | ATP9B | OBD169_033.OBD169_035 | 0 | 385 |
| 10 | OBD169_039 | CCTCTCTGGTGCCACATCTTATCTTA | ATP9B | OBD169_037.OBD169_039 | 0 | 386 |
| 11 | OBD169_043 | GTCTTCAAGCCACAGAGCAGGATTCC | ATP9B | OBD169_041.OBD169_043 | 0.122625202 | 387 |
| 12 | OBD169_047 | CCATCTTCTGTAACCCTGAACGGAGT | ATP9B | OBD169_045.OBD169_047 | 0 | 388 |
| 13 | OBD169_051 | CGTTATCTATGGTCCCACTACTGTGT | ATP9B | OBD169_049.OBD169_051 | −0.050953035 | 389 |
| 14 | OBD169_055 | GCAGGTTATTAGAGGACCGAGGC | ATP9B | OBD169_053.OBD169_055 | 0 | 390 |
| 15 | OBD169_059 | CGCCACCAAGAATGTCATCTCCG | ATP9B | OBD169_057.OBD169_059 | 0 | 391 |
| 16 | OBD169_063 | CGATGAGACAGAGCACCAGAGTC | ATP9B | OBD169_061.OBD169_063 | 0.127785257 | 392 |
| 17 | OBD169_067 | GTCTTCAAGCCACAGAGCAGGATTCC | ATP9B | OBD169_065.OBD169_067 | −6.18E−06 | 393 |
| 18 | OBD169_071 | GTGGCTACCTGTGGTCCTCTCCT | ATP9B | OBD169_069.OBD169_071 | 0 | 394 |
| 19 | OBD169_075 | GCCACCAAGAATGTCATCTCCGATTT | ATP9B | OBD169_073.OBD169_075 | 0 | 395 |
| 20 | OBD169_079 | GGCTTCGTTATCTATGGTCCCACTAC | ATP9B | OBD169_077.OBD169_079 | 0 | 396 |
| 21 | OBD169_083 | CGCCACCAAGAATGTCATCTCCG | ATP9B | OBD169_081.OBD169_083 | 0 | 397 |
| 22 | OBD169_087 | CAGGACCCAGACTTGCCCAAACC | NFATC1 | OBD169_085.OBD169_087 | 0 | 398 |
| 23 | OBD169_091 | CTGTAACCCTGAACGGAGTAGAATAG | ATP9B | OBD169_089.OBD169_091 | 0 | 399 |
| 24 | OBD169_095 | GGCGGAGAAACCGTTCGTGTGTG | MTOR | OBD169_093.OBD169_095 | 0 | 400 |
| 25 | OBD169_099 | GGCAAGGGACCACTCTTAGTCTGC | MTOR | OBD169_097.OBD169_099 | 0 | 401 |
| 26 | OBD169_103 | TCCCCTTATCAACCAACTCGGGC | MTOR | OBD169_101.OBD169_103 | 0.003937173 | 402 |
| 27 | OBD169_107 | TTGGTGGTCAGGACTGGAGTGCC | PCDHGC5 | OBD169_105.OBD169_107 | 0.029250039 | 403 |
| 28 | OBD169_111 | CTGCTTGGGCTGGTCTTTGGTTG | MTOR | OBD169_109.OBD169_111 | 0 | 404 |
| 29 | OBD169_115 | TCCCCTTATCAACCAACTCGGGC | MTOR | OBD169_113.OBD169_115 | 0 | 405 |
| 30 | OBD169_119 | GAGGTCAAGGGAAGAGACAGGGA | PCDHGC5 | OBD169_117.OBD169_119 | 0 | 406 |
| 31 | OBD169_123 | TGTGGAATGAGCCTCCGTCCCTG | CABLES1 | OBD169_121.OBD169_123 | 0 | 407 |
| 32 | OBD169_127 | CATTCCTGGTATCGTGTTGCCGC | CABLES1 | OBD169_125.OBD169_127 | 0.005994639 | 408 |
| 33 | OBD169_131 | CCAGAACATCTCTTCGTGGTGGG | CABLES1 | OBD169_129.OBD169_131 | 0 | 409 |
| 34 | OBD169_135 | GATGCTGTCCCTGTGCTATGAGC | SREBF2 | OBD169_133.OBD169_135 | 0.161924686 | 410 |
| 35 | OBD169_139 | GTCATCAACACTCTTTCCCTGCTCCT | MLLT3 | OBD169_137.OBD169_139 | 0 | 411 |
| 36 | OBD169_143 | CCATTGCCTGAATCCTCCCTGGC | FOCAD | OBD169_141.OBD169_143 | 0 | 412 |
| 37 | OBD169_147 | TGAGGGCTGGCTGGGCATTCATA | MLLT3 | OBD169_145.OBD169_147 | 0 | 413 |
| 38 | OBD169_151 | GTCATCAACACTCTTTCCCTGCTCCT | MLLT3 | OBD169_149.OBD169_151 | 0 | 414 |
| 39 | OBD169_155 | CAGCCCCAATCCCATCCAGAATCCA | FOCAD | OBD169_153.OBD169_155 | 0 | 415 |
| 40 | OBD169_159 | CTGTGATTCCCTTGTTATGGTTTTGA | ATG5 | OBD169_157.OBD169_159 | 0 | 416 |
| 41 | OBD169_163 | GCCTCTGTCCTGTGTGTTATGAAACT | ATG5 | OBD169_161.OBD169_163 | 0 | 417 |
| 42 | OBD169_167 | CTACAAGGGAACTGCCTGCTTCGCTA | FAF1 | OBD169_165.OBD169_167 | 0 | 418 |
| 43 | OBD169_171 | AACAGGCTTACCTCTTCGGACTGCTC | KITLG | OBD169_169.OBD169_171 | 0.063674679 | 419 |
| 44 | OBD169_175 | CTCCTCAAAGAGAGAGCCTTCCCG | CREB3L2 | OBD169_173.OBD169_175 | 0 | 420 |
| 45 | OBD169_179 | GCGTGTGAGAGAGGAGATAAATGGAT | CREB3L2 | OBD169_177.OBD169_179 | 0.013500095 | 421 |
| 46 | OBD169_183 | CTGGCTGGCTCTTGACTTTGCTATTG | CREB3L2 | OBD169_181.OBD169_183 | 0 | 422 |
| 47 | OBD169_187 | AACCTCCTCAAAGAGAGAGCCTTCCC | CREB3L2 | OBD169_185.OBD169_187 | 0.248790766 | 423 |
| 48 | OBD169_191 | CCTCCTGTATTTCTACTTCCACTCAG | CREB3L2 | OBD169_189.OBD169_191 | 0 | 424 |
| 49 | OBD169_195 | GACTGATTGTAGGAGGACTCACAGAT | CREB3L2 | OBD169_193.OBD169_195 | 0 | 425 |
| 50 | OBD169_199 | CCTCCTGTATTTCTACTTCCACTCAG | CREB3L2 | OBD169_197.OBD169_199 | 0 | 426 |
| 51 | OBD169_203 | ATCATTGGTTTGGAGTGACAACTACT | FOXO1 | OBD169_201.OBD169_203 | 0 | 427 |
| 52 | OBD169_207 | GGTAGTGTCTGTTTTCTGGACTTTAC | FOXO1 | OBD169_205.OBD169_207 | 0 | 428 |
| 53 | OBD169_211 | GGTGTGGGTGTGTAAGAGGGACC | SPECC1L | OBD169_209.OBD169_211 | 0 | 429 |
| 54 | OBD169_215 | CTGCCTCCAGAAGGTGTCTCAGA | SPECC1L | OBD169_213.OBD169_215 | 0 | 430 |
| 55 | OBD169_219 | TACAGAAAGCCCTCGCTGGGAGC | SPECC1L | OBD169_217.OBD169_219 | 0 | 431 |
| 56 | OBD169_223 | AGGGTGTGGGTGTGTAAGAGGGA | SPECC1L | OBD169_221.OBD169_223 | 0 | 432 |
| 57 | OBD169_227 | CCACTGTGCCCTGGATGGAGAAC | SPECC1L | OBD169_225.OBD169_227 | 0 | 433 |
| 58 | OBD169_231 | GGTGTGGGTGTGTAAGAGGGACC | SPECC1L | OBD169_229.OBD169_231 | −0.042293888 | 434 |
| 59 | OBD169_235 | TTGAGGGCAGAGCAGGAAGGGTG | SPECC1L | OBD169_233.OBD169_235 | 0.052029568 | 435 |
| 60 | OBD169_239 | GGGATACCCAGAGAGAAGGGCAAG | IFNGR2 | OBD169_237.OBD169_239 | 0 | 436 |
| 61 | OBD169_243 | AGACCTGAGGAAGGAGGGTGGAC | IFNGR2 | OBD169_241.OBD169_243 | 0.043975004 | 437 |
| 62 | OBD169_247 | GTGAGAGGCAGAGACAGCACAGACTA | NFKB1 | OBD169_245.OBD169_247 | 0 | 438 |
| 63 | OBD169_251 | GTGAGAGGCAGAGACAGCACAGACTA | NFKB1 | OBD169_249.OBD169_251 | 0 | 439 |
| 64 | OBD169_255 | GGTGACTGATGAGACTCCAGGAAAGT | NFKB1 | OBD169_253.OBD169_255 | 0 | 440 |
| 65 | OBD169_259 | GCCTAAACTTTCTCTCTCAGTCAGCG | NFKB1 | OBD169_257.OBD169_259 | 0.01527689 | 441 |
| 66 | OBD169_263 | GCCTCTGTCATTCGTGCTTCCAGTGT | NFKB1 | OBD169_261.OBD169_263 | 0 | 442 |
| 67 | OBD169_267 | GCCTAAACTTTCTCTCTCAGTCAGCG | NFKB1 | OBD169_265.OBD169_267 | 0.141700302 | 443 |
| 68 | OBD169_271 | TGTTCACGCACAACCTCGGCTCTG | NFKB1 | OBD169_269.OBD169_271 | 0 | 444 |
| 69 | OBD169_275 | GCAGCCTTTGGCAGCACTCTCTG | NFKB1 | OBD169_273.OBD169_275 | 0 | 445 |
| 70 | OBD169_279 | CCCAGAAACTTTGCTAACTCCTATTG | MAPK10 | OBD169_277.OBD169_279 | −0.097352472 | 446 |
| 71 | OBD169_283 | GCCTCTGTCATTCGTGCTTCCAGTGT | NFKB1 | OBD169_281.OBD169_283 | 0 | 447 |
| 72 | OBD169_287 | GCCTCTGTCATTCGTGCTTCCAG | NFKB1 | OBD169_285.OBD169_287 | 0 | 448 |
| 73 | OBD169_291 | AAGTGCCTGTTTTATGGAGAACTGGC | F9 | OBD169_289.OBD169_291 | 0 | 449 |
| 74 | OBD169_295 | CCCTTCTGGAACTGGATGAGCCC | F9 | OBD169_293.OBD169_295 | 0 | 450 |
| 75 | OBD169_299 | CACAGCCGAAGAGCCACTGAAGC | BTK | OBD169_297.OBD169_299 | 0 | 451 |

TABLE 9.a

| N | Probe | marker | GLMNET |
|---|---|---|---|
| 1 | ORF1_1_1034282_1037357_1049484_1054771_FF | OBD169_001.OBD169_003 | 0.150341207 |
| 2 | ORF5_1_1182474_1185271_1270569_1273244_RF | OBD169_009.OBD169_011 | −0.065057056 |
| 3 | ORF5_1_1147651_1150121_1196191_1197234_RF | OBD169_021.OBD169_023 | 0 |
| 4 | ORF5_1_1146367_1147651_1165983_1167502_FF | OBD169_033.OBD169_035 | 0 |
| 5 | ORF5_1_1196191_1197234_1230936_1232838_RR | OBD169_041.OBD169_043 | 0.122625202 |
| 6 | ORF5_1_1270569_1273244_1300933_1312034_FF | OBD169_049.OBD169_051 | −0.050953035 |
| 7 | ORF5_1_1196191_1197234_1289361_1294150_FF | OBD169_061.OBD169_063 | 0.127785257 |
| 8 | ORF5_1_1140030_1142517_1230936_1232838_RR | OBD169_065.OBD169_067 | −6.18144E−06 |
| 9 | ORF5_1_1230936_1232838_1273244_1276010_RR | OBD169_073.OBD169_075 | 0 |
| 10 | ORF41_2_36413514_36415342_36452868_36458269_RR | OBD169_105.OBD169_107 | 0.029250039 |
| 11 | ORF91_7_65032142_65033242_65065127_65067650_FF | OBD169_125.OBD169_127 | 0.005994639 |
| 12 | ORF91_7_65037215_65039217_65065127_65067650_RF | OBD169_129.OBD169_131 | 0 |
| 13 | ORF9_10_23456592_23460302_23494817_23496168_RR | OBD169_133.OBD169_135 | 0.161924686 |
| 14 | ORF16_11_40371218_40374048_40393587_40395559_RF | OBD169_153.OBD169_155 | 0 |
| 15 | ORF31_15_29619588_29621525_29646237_29648560_RR | OBD169_165.OBD169_167 | 0 |
| 16 | ORF30_15_10476260_10484217_10545581_10548270_RR | OBD169_169.OBD169_171 | 0.063674679 |
| 17 | ORF32_16_10747182_10750815_10792291_10794979_RF | OBD169_185.OBD169_187 | 0.248790766 |
| 18 | ORF70_26_27894296_27895372_27963114_27965001_RR | OBD169_229.OBD169_231 | −0.042293888 |
| 19 | ORF70_26_27890569_27893929_27906620_27909025_RR | OBD169_233.OBD169_235 | 0.052029568 |
| 20 | ORF79_32_24013860_24017127_24028587_24030780_RR | OBD169_265.OBD169_267 | 0.141700302 |
| 21 | ORF82_32_9652472_9664654_9692674_9698030_RR | OBD169_277.OBD169_279 | −0.097352472 |
| 22 | ORF104_X_109508063_109510622_109526507_109531763_FF | OBD169_293.OBD169_295 | 0 |

TABLE 9.b

| N | Freq | Rank_median | pValue_Mean | pValue_Median | Classification |
|---|---|---|---|---|---|
| 1 | 429 | 14 | 0.061922326 | 0.036945939 | Presence in Lymphoma |
| 2 | 156 | 171.75 | 0.722387112 | 1 | Presence in Healthy Control |
| 3 | 155 | 29.75 | 0.137404255 | 0.119176434 | Presence in Lymphoma |
| 4 | 278 | 14.75 | 0.076727087 | 0.075561315 | Presence in Lymphoma |
| 5 | 300 | 22.25 | 0.11481488 | 0.111802994 | Presence in Lymphoma |
| 6 | 262 | 107.5 | 0.614169025 | 0.608053733 | Presence in Healthy Control |
| 7 | 375 | 16.5 | 0.07087785 | 0.048199002 | Presence in Lymphoma |
| 8 | 112 | 168.5 | 0.749906059 | 1 | Presence in Healthy Control |
| 9 | 115 | 28 | 0.185541633 | 0.104567082 | Presence in Lymphoma |
| 10 | 262 | 16.25 | 0.099987147 | 0.048199002 | Presence in Lymphoma |
| 11 | 300 | 22.75 | 0.163342682 | 0.089691605 | Presence in Lymphoma |
| 12 | 130 | 33.5 | 0.190563594 | 0.148661263 | Presence in Lymphoma |
| 13 | 406 | 18 | 0.093426309 | 0.075561315 | Presence in Lymphoma |
| 14 | 270 | 23.25 | 0.114536951 | 0.056118783 | Presence in Lymphoma |
| 15 | 135 | 23.5 | 0.159941064 | 0.104567082 | Presence in Lymphoma |
| 16 | 452 | 7 | 0.034141832 | 0.02207464 | Presence in Lymphoma |
| 17 | 498 | 2 | 0.009682876 | 0.006340396 | Presence in Lymphoma |
| 18 | 225 | 97.75 | 0.608040664 | 0.516296715 | Presence in Healthy Control |
| 19 | 357 | 9.25 | 0.060876258 | 0.035136821 | Presence in Lymphoma |
| 20 | 451 | 12 | 0.055525573 | 0.036945939 | Presence in Lymphoma |
| 21 | 225 | 94.5 | 0.550385123 | 0.521495378 | Presence in Healthy Control |
| 22 | 257 | 32.5 | 0.167821507 | 0.104567082 | Presence in Lymphoma |

TABLE 10
Prostate cancer risk group categories.

| | | PSA | Gleason | |
|---|---|---|---|---|
| Category | Risk | (ng/ml) | score | T stage |
| 1 | Low risk | <10 | <6 | T1-T2a |
| 2 | Intermediate | 10-20 | 7 | T2b |
| | risk | | | |
| 3 | High risk | >20 | 8-10 | T2c^{*}, |
| | | | | T3 or T4 |

*According to NCCN guidelines 2018 update T2c is considered intermediate risk.
Abbreviations. PSA: prostate specific antigen.

TABLE 11
Five-marker signature used for the diagnosis of prostate cancer.

| | Gene | | P |
|---|---|---|---|
| Markers | symbol | Gene name | value |
| PCa.57.59 | ETS1 | ETS proto-oncogene 1, transcription factor | 0.11 |
| PCa.81.83 | MAP3K14 | Mitogen-activated protein kinase kinase kinase 14 | 0.11 |
| PCa.73.75 | SLC22A3 | Solute carrier family 22 member 3 | 0.107 |
| PCa.77.79 | SLC22A3 | Solute carrier family 22 member 3 | 0.005 |
| PCa.189.191 | CASP2 | Caspase 2 | 0.137 |

TABLE 12
Comparison of pathology and EpiSwitch ™ results.

| | Pathology results | |
|---|---|---|
| EpiSwitch ™ diagnosis | PCa | Healthy |
| PCa | 8 | 2 |
| Healthy | 2 | 8 |
| Results from classification of blinded samples (n = 20). | | |
| Statistic | Value | 95% CI |
| Sensitivity | 80.00% | 44.39% to 97.48% |
| Specificity | 80.00% | 44.39% to 97.48% |
| Positive Likelihood Ratio | 4.00 | 1.11 to 14.35 |
| Negative Likelihood Ratio | 0.25 | 0.07 to 0.90 |
| Disease prevalence (*) | 50.00% | 27.20% to 72.80% |
| Positive Predictive Value (*) | 80.00% | 52.71% to 93.49% |
| Negative Predictive Value (*) | 80.00% | 52.71% to 93.49% |

(*) These values are dependent on disease prevalence.
Abbreviations. 95% CI: 95% confidence interval.

TABLE 13
Markers for high-risk category 3 vs low-risk category 1
and for high-risk category 3 vs intermediate-risk category 2.
Biomarkers for high-risk category 3 vs low-risk category 1

| | Gene | |
|---|---|---|
| Loci | location | Markers |
| 6_7724582_7733496_7801590_7806316_FF | BMP6 | PCa.119.37.39 |
| 21_39895678_39899145_39984806_39991905_RF | ERG | PCa.119.65.67 |
| 8_16195878_16203315_16396849_16400398_FF | MSR1 | PCa.119.77.79 |
| 1_155146523_155149986_155191807_155193554_FR | MUC1 | PCa.119.121.123 |
| 11_107955219_107960166_108013361_108018367_FF | ACAT1 | PCa.119.57.59 |
| 9_90064560_90073617_90140806_90142738_FR | DAPK1 | PCa.119.165.167 |

Biomarkers for high-risk category 3 vs intermediate-risk category 2

| | Gene | |
|---|---|---|
| Loci | location | Markers |
| 1_119912462_119915175_119959754_119963670_RR | HSD3B2 | PCa.119.129.131 |
| 4_177629821_177639626_177740221_177743175_FR | VEGFC | PCa.119.205.207 |
| 12_99061113_99062942_99098781_99108240_FF | APAF1 | PCa.119.49.51 |
| 1_155146523_155149986_155191807_155193554_FR | MUC1 | PCa.119.121.123 |
| 11_107955219_107960166_108013361_108018367_FF | ACAT1 | PCa.119.57.59 |
| 9_90064560_90073617_90140806_90142738_FR | DAPK1 | PCa.119.165.167 |

Shaded last three markers are common.
Abbreviations. ACAT1: acetyl-CoA acetyltransferase 1; APAF1: apoptotic peptidase activating factor 1; BMP6: bone morphogenetic protein 6; DAPK1: death associated protein kinase 1; ERG: ETS transcription factor ERG; HSD3B2: hydroxy-delta-5-steroid dehydrogenase, 3 beta- and steroid delta-isomerase 2; MSR1: macrophage scavenger receptor 1; MUC1: mucin 1, cell surface associated; VEGFC: vascular endothelial growth factor C.

TABLE 14
Detection of similar epigenetic markers in blood and in matching primary prostate
tumours at a fixed range of assay sensitivity.

| | Category | | | | | | | Total number | | | | | | Total number |
|---|---|---|---|---|---|---|---|---|---|---|---|---|---|---|
| Markers | Patient | | Blood samples | | | | | of positive | Tissue samples | | | | | of positive |
| for | Gene | | 1 | | | | 3 | markers in | 1 | | | | 3 | markers in |
| category | location | Markers | A | B | C | D | E | blood samples | A | B | C | D | E | tissue samples |
| 3 vs 1 | BMP6 | PCa.119.37.39 | 1 | 1 | 0 | 1 | 1 | 4 | 1 | 1 | 1 | 0 | 1 | 4 |
| | ERG | PCa.119.65.67 | 1 | 1 | 1 | 1 | 1 | 5 | 1 | 0 | 1 | 0 | 1 | 3 |
| | MSR1 | PCa.119.77.79 | 1 | 0 | 0 | 0 | 1 | 2 | 0 | 0 | 1 | 0 | 1 | 2 |
| Common | MUC1 | PCa.119.121.123 | 1 | 1 | 1 | 1 | 0 | 4 | 1 | 1 | 1 | 1 | 1 | 5 |
| | DAPK1 | PCa.119.165.167 | 1 | 0 | 1 | 1 | 0 | 3 | 0 | 0 | 1 | 0 | 1 | 2 |
| | ACAT1 | PCa.119.57.59 | 1 | 1 | 1 | 1 | 1 | 5 | 1 | 1 | 1 | 1 | 1 | 5 |
| 3 vs 2 | HSD3B2 | PCa.119.129.131 | 1 | 1 | 0 | 1 | 1 | 4 | 1 | 1 | 1 | 0 | 1 | 4 |
| | VEGFC | PCa.119.205.207 | 1 | 1 | 1 | 1 | 1 | 5 | 1 | 1 | 1 | 1 | 1 | 5 |
| | APAF1 | PCa.119.49.51 | 1 | 1 | 1 | 1 | 1 | 5 | 0 | 1 | 1 | 1 | 1 | 4 |

When a PCR band of the correct size is detected, it is given a score of 1.
When no band is detected, it is given a score of 0.
Abbreviations.
ACAT1: acetyl-CoA acetyltransferase 1;
APAF1: apoptotic peptidase activating factor 1;
BMP6: bone morphogenetic protein 6;
DAPK1: death associated protein kinase 1;
ERG: ETS transcription factor ERG;
HSD3B2: hydroxy-delta-5-steroid dehydrogenase, 3 beta- and steroid delta-isomerase 2;
MSR1: macrophage scavenger receptor 1;
MUC1: mucin 1, cell surface associated;
VEGFC: vascular endothelial growth factor C.

TABLE 15
Comparison of pathology and EpiSwitch ™ results for category 3 vs 1 classifier.

| | Pathology results | |
|---|---|---|
| EpiSwitch ™ diagnosis | Category 1 | Category 3 |
| Category 1 | 39 | 5 |
| Category 3 | 3 | 20 |

Results from classification of blinded samples for category 3 vs 1 classifier (n = 67).

| Statistic | Value | 95% CI |
|---|---|---|
| Sensitivity | 80.00% | 59.30% to 93.17% |
| Specificity | 92.86% | 80.52% to 98.50% |
| Positive Likelihood Ratio | 11.20 | 3.70 to 33.91 |
| Negative Likelihood Ratio | 0.22 | 0.10 to 0.47 |
| Disease prevalence (*) | 37.31% | 25.80% to 49.99% |
| Positive Predictive Value (*) | 86.96% | 68.77% to 95.28% |
| Negative Predictive Value (*) | 88.64% | 78.00% to 94.49% |

(*) These values are dependent on disease prevalence.
Abbreviations. 95% CI: 95% confidence interval.

TABLE 16
Comparison of pathology and EpiSwitch ™ results for category 3 vs 1 classifier.

| | Pathology results | |
|---|---|---|
| EpiSwitch ™ diagnosis | Category 2 | Category 3 |
| Category 2 | 16 | 4 |
| Category 3 | 2 | 21 |

Results from classification of blinded samples for category 3 vs 2 classifier (n = 43).

| Statistic | Value | 95% CI |
|---|---|---|
| Sensitivity | 84.00% | 63.92% to 95.46% |
| Specificity | 88.89% | 65.29% to 98.62% |
| Positive Likelihood Ratio | 7.56 | 2.02 to 28.24 |
| Negative Likelihood Ratio | 0.18 | 0.07 to 0.45 |
| Disease prevalence (*) | 58.14% | 42.13% to 72.99% |
| Positive Predictive Value (*) | 91.30% | 73.76% to 97.51% |
| Negative Predictive Value (*) | 30.00% | 61.62% to 90.88% |

(*) These values are dependent on disease prevalence.
Abbreviations. 95% CI: 95% confidence interval.

TABLE 17
Clinical characteristics of the
patients participated in the study.

| | | Number of |
|---|---|---|
| Characteristic | Category | patients |
| Gleason score | ≤6 | 39 |
| | 7 | 54 |
| | 8-10 | 29 |
| | Unknown | 18 |
| | Median | 7 |
| Stage | 1 | 36 |
| | 2 | 49 |
| | 3 | 25 |
| | 4 | 14 |
| | Unknown | 16 |
| Age | 45-54 | 12 |
| | 55-64 | 21 |
| | 65-74 | 44 |
| | 75+ | 63 |
| | Unknown | 0 |
| PSA | <10 | 55 |
| | 10-20 | 23 |
| | >20 | 51 |
| | Unknown | 11 |
| | Median | 12.2 |
| Metastatic patients | | 21 |

Abbreviation. PSA: prostate specific antigen.

TABLE 18
List of 425 prostate cancer-related genomic
loci tested in the initial array.

| | Array |
|---|---|
| Gene | probe |
| name | count |
| ABHD3 | 20 |
| ABR | 20 |
| ACAT1 | 20 |
| ACPP | 20 |
| ACTA1 | 20 |
| ACTR2 | 20 |
| ACTR3 | 20 |
| ADAM9 | 20 |
| ADRB2 | 20 |
| AGAP2 | 15 |
| AIP | 9 |
| AKT1 | 20 |
| AKT2 | 20 |
| AKT3 | 122 |
| AMACR | 20 |
| AP2M1 | 20 |
| APAF1 | 26 |
| APC | 37 |
| AR | 115 |
| ARAF | 20 |
| ARNT | 20 |
| ARTN | 20 |
| ASAH1 | 20 |
| ATM | 54 |
| AXIN1 | 20 |
| AXL | 20 |
| BAD | 20 |
| BCAR1 | 28 |
| BCL2L1 | 20 |
| BCORL1 | 20 |
| BGLAP | 11 |
| BIRC5 | 4 |
| BMI1 | 20 |
| BMP6 | 71 |
| BMP7 | 20 |
| BRAF | 20 |
| BRCA1 | 20 |
| BRCA2 | 20 |
| CA1 | 20 |
| CALR | 15 |
| CAP1 | 20 |
| CARS | 20 |
| CASP2 | 20 |
| CASP3 | 20 |
| CASP9 | 20 |
| CAV1 | 20 |
| CBL | 20 |
| CCDC67 | 31 |
| CCND1 | 20 |
| CCND2 | 20 |
| CCNE1 | 20 |
| CCNJ | 6 |
| CD244 | 20 |
| CD4 | 20 |
| CD44 | 75 |
| CD82 | 20 |
| CD8A | 20 |
| CDC25A | 20 |
| CDC25B | 20 |
| CDC25C | 20 |
| CDC37 | 20 |
| CDC45 | 20 |
| CDH1 | 20 |
| CDK2 | 2 |
| CDK4 | 20 |
| CDK6 | 111 |
| CDKN1A | 20 |
| CDKN1B | 20 |
| CDKN2A | 20 |
| CDKN2B | 16 |
| CDKN2C | 10 |
| CDKN2D | 20 |
| CENPBD1 | 7 |
| CHAMP1 | 20 |
| CHEK2 | 20 |
| CHUK | 29 |
| CLU | 28 |
| COMMD3- | 20 |
| BMI1 | |
| CREBBP | 20 |
| CSE1L | 20 |
| CSF2 | 11 |
| CSF2RA | 3 |
| CSNK1A1 | 20 |
| CTBP1 | 20 |
| CTNNA1 | 43 |
| CTNNB1 | 45 |
| CTNND1 | 20 |
| CXCL16 | 19 |
| CXCR1 | 20 |
| CXCR2 | 19 |
| CXCR4 | 20 |
| CXCR6 | 19 |
| CYCS | 18 |
| CYP17A1 | 20 |
| CYP19A1 | 149 |
| CYP1B1 | 19 |
| DAND5 | 20 |
| DAPK1 | 56 |
| DDIT4 | 16 |
| DOK4 | 20 |
| DPP4 | 103 |
| E2F1 | 20 |
| E2F4 | 20 |
| EDN1 | 20 |
| EDNRA | 35 |
| EED | 20 |
| EGF | 26 |
| EGFR | 200 |
| EIF4E | 20 |
| EIF4EBP1 | 20 |
| EIF6 | 20 |
| ELAC2 | 20 |
| ENPP2 | 74 |
| EP300 | 20 |
| EPHA2 | 20 |
| EPHB4 | 20 |
| EPHB6 | 6 |
| EPS15 | 111 |
| ERBB2 | 20 |
| ERBB3 | 20 |
| ERBB4 | 200 |
| ERG | 67 |
| ERRFI1 | 20 |
| ESR1 | 200 |
| ESR2 | 20 |
| ETS1 | 105 |
| ETV1 | 47 |
| ETV4 | 20 |
| ETV5 | 20 |
| EZH2 | 31 |
| FASN | 20 |
| FGD4 | 52 |
| FGF19 | 20 |
| FGF2 | 39 |
| FGF6 | 20 |
| FGF8 | 10 |
| FGFR1 | 20 |
| FGFR4 | 12 |
| FHL2 | 20 |
| FLNA | 9 |
| FLT1 | 97 |
| FLT4 | 10 |
| FNI | 20 |
| FOLH1 | 35 |
| FOSB | 11 |
| FOXA1 | 18 |
| FOXO1 | 21 |
| FOXO3 | 139 |
| FOXP1 | 200 |
| FZD1 | 18 |
| GABI | 107 |
| GAS6 | 20 |
| GLIPR1 | 15 |
| GNRH1 | 20 |
| GNRHR | 3 |
| GRB2 | 20 |
| GSK3B | 92 |
| GSTP1 | 20 |
| HDAC1 | 20 |
| HDAC3 | 20 |
| HGF | 27 |
| HIF1A | 20 |
| HIPK2 | 20 |
| HRAS | 6 |
| HSD3B1 | 20 |
| HSD3B2 | 20 |
| HSP90AA1 | 20 |
| HSP90AB1 | 20 |
| HSPA1A | 20 |
| HSPB1 | 20 |
| IGF1 | 20 |
| IGFIR | 102 |
| IGFBP3 | 14 |
| IGFBP5 | 20 |
| IL16 | 82 |
| IL2 | 2 |
| IL6 | 20 |
| IL6R | 20 |
| IL8 | 17 |
| INPPL1 | 20 |
| INS | 17 |
| IRAK1 | 20 |
| IRS1 | 20 |
| ITK | 24 |
| JAK1 | 20 |
| JAK2 | 20 |
| JAK3 | 20 |
| JUN | 20 |
| KAT7 | 20 |
| KCNH2 | 20 |
| KDM6B | 20 |
| KIT | 107 |
| KLF4 | 20 |
| KLK2 | 20 |
| KLK3 | 8 |
| KLK4 | 17 |
| KRAS | 20 |
| LPAR1 | 30 |
| LPAR2 | 5 |
| LPAR3 | 31 |
| LPAR4 | 5 |
| LRP5 | 20 |
| LRP6 | 35 |
| MAGEA11 | 20 |
| MAP2K1 | 20 |
| MAP2K2 | 20 |
| MAP2K5 | 196 |
| MAP3K14 | 20 |
| MAP3K2 | 24 |
| MAPK1 | 20 |
| MAPK3 | 20 |
| MAPKAP1 | 31 |
| MCM7 | 20 |
| MDM2 | 20 |
| MDM4 | 23 |
| MED1 | 20 |
| MEN1 | 20 |
| MET | 105 |
| MIF | 12 |
| MIR125B1 | 4 |
| MIR149 | 3 |
| MIR151A | 31 |
| MIR152 | 15 |
| MIR16-1 | 20 |
| MIR183 | 20 |
| MIR197 | 7 |
| MIR204 | 20 |
| MIR222 | 6 |
| MIR224 | 20 |
| MIR23B | 20 |
| MIR24-2 | 20 |
| MIR26B | 3 |
| MIR27B | 20 |
| MIR335 | 20 |
| MIR34A | 20 |
| MIR361 | 20 |
| MIR365A | 5 |
| MIR376A1 | 9 |
| MIR454 | 11 |
| MIR500A | 20 |
| MIR582 | 3 |
| MIR619 | 20 |
| MIR636 | 20 |
| MIR648 | 20 |
| MIR671 | 20 |
| MIR766 | 15 |
| MIR877 | 13 |
| MIR887 | 18 |
| MIR93 | 20 |
| MIR98 | 17 |
| MIRLET7G | 3 |
| MLST8 | 2 |
| MMP14 | 20 |
| MMP9 | 20 |
| MSMB | 20 |
| MSR1 | 200 |
| MTA1 | 1 |
| MTCH1 | 20 |
| MTOR | 32 |
| MTRR | 20 |
| MUC1 | 7 |
| MYB | 20 |
| MYC | 20 |
| NCOA1 | 170 |
| NCOA2 | 200 |
| NCOA3 | 20 |
| NCOA4 | 18 |
| NCOR1 | 35 |
| NCOR2 | 26 |
| NEDD4 | 85 |
| NEDD4L | 128 |
| NET1 | 20 |
| NF1 | 190 |
| NFKB1 | 66 |
| NFKBIA | 20 |
| NGF | 21 |
| NKX3-1 | 20 |
| NOVA1 | 34 |
| NOX5 | 43 |
| NPDC1 | 1 |
| NR0B1 | 10 |
| NR3C1 | 25 |
| NR4A3 | 20 |
| NRAS | 20 |
| NRIP1 | 69 |
| NTF3 | 31 |
| NTRK1 | 20 |
| PA2G4 | 20 |
| PAQR7 | 20 |
| PAX8 | 20 |
| PCBP2 | 20 |
| PCYT1A | 20 |
| PDGFA | 20 |
| PDGFB | 20 |
| PDGFRA | 24 |
| PDGFRB | 20 |
| PDPK1 | 20 |
| PIAS1 | 89 |
| PIAS2 | 21 |
| PIAS3 | 20 |
| PIAS4 | 20 |
| PIK3C2A | 20 |
| PIK3C2B | 21 |
| PIK3C2G | 200 |
| PIK3CA | 20 |
| PIK3CB | 21 |
| PIK3CD | 20 |
| PIK3CG | 20 |
| PIK3R1 | 81 |
| PIK3R2 | 20 |
| PLD1 | 189 |
| PLD2 | 20 |
| PLD3 | 20 |
| PML | 20 |
| POU2F1 | 26 |
| POU2F2 | 20 |
| PPP1CA | 20 |
| PPP2CA | 20 |
| PRKCA | 148 |
| PRKCB | 176 |
| PRKCD | 20 |
| PRKCG | 20 |
| PRKCH | 200 |
| PRKCI | 43 |
| PRKCQ | 59 |
| PRKCZ | 20 |
| PRSS3 | 23 |
| PRSS8 | 20 |
| PSAP | 20 |
| PSCA | 20 |
| PSG1 | 19 |
| PTEN | 23 |
| PTGS2 | 20 |
| PTK2B | 54 |
| PTK7 | 20 |
| PTPN11 | 20 |
| PTPN12 | 20 |
| PTPN14 | 135 |
| PTPRF | 20 |
| PTPRR | 200 |
| PTPRT | 200 |
| PXN | 20 |
| RAB9B | 16 |
| RAD51 | 9 |
| RAF1 | 20 |
| RAN | 20 |
| RB1 | 82 |
| RCHY1 | 20 |
| REL | 25 |
| RGS6 | 200 |
| RHEB | 20 |
| RHOA | 20 |
| RICTOR | 38 |
| RNASEL | 13 |
| RNF14 | 20 |
| RNF20 | 20 |
| RNF40 | 20 |
| ROCK1 | 67 |
| ROR2 | 193 |
| RPS6KA1 | 20 |
| RPS6KB1 | 20 |
| RPTOR | 68 |
| RREB1 | 85 |
| RYBP | 20 |
| S100A4 | 20 |
| S100P | 20 |
| SAGE1 | 20 |
| SATB1 | 20 |
| SCGB1A1 | 20 |
| SFTPA1 | 20 |
| SFTPA2 | 8 |
| SHC1 | 11 |
| SIRT1 | 20 |
| SKP2 | 16 |
| SLC22A3 | 56 |
| SMAD3 | 20 |
| SMAD4 | 67 |
| SMARCE1 | 20 |
| SOAT1 | 20 |
| SOS1 | 103 |
| SOX9 | 20 |
| SP1 | 20 |
| SPDEF | 17 |
| SPINK1 | 20 |
| SPOPL | 20 |
| SRC | 20 |
| SRD5A1 | 13 |
| SRD5A2 | 79 |
| SRD5A3 | 20 |
| SREBF1 | 20 |
| SRY | 3 |
| STAT3 | 20 |
| SUZ12 | 20 |
| SVIL | 51 |
| TBC1D8 | 43 |
| TERT | 20 |
| TGFB1 | 20 |
| TGFB1I1 | 20 |
| TGFB2 | 75 |
| TGFB3 | 15 |
| TGFBR1 | 20 |
| TGFBR2 | 65 |
| TIMP1 | 20 |
| TMF1 | 20 |
| TMPRSS2 | 36 |
| TNK2 | 20 |
| TOP2A | 20 |
| TOP2B | 24 |
| TP53 | 15 |
| TRAF3 | 20 |
| TRAF6 | 20 |
| TSC1 | 20 |
| TSC2 | 2 |
| TUBB | 4 |
| VEGFA | 20 |
| VEGFC | 57 |
| VIM | 9 |
| WAS | 12 |
| WNT1 | 20 |
| WNT2 | 40 |
| WNT3 | 20 |
| WNT5A | 23 |
| ZAP70 | 20 |
| ZFAND1 | 20 |
| ZMYND10 | 20 |
| Total | 14241 |

TABLE 19
Markers for prognostic array stratifications category 1 vs 3 and category 2 vs 3. Top 181 markers produced from the prognostic array.

| Probes | GeneLocus | logFC | AveExpr | t | P.Value |
|---|---|---|---|---|---|
| ACAT1_11_107955219_107960166_108013361_ | ACAT1 | −0.436725529 | −0.436725529 | −11.90723067 | 1.37E−07 |
| 108018367_FF | | | | | |
| ACTA1_1_229547333_229551721_229600994_ | ACTA1 | 0.417850291 | 0.417850291 | 8.736657363 | 2.98E−06 |
| 229605798_FR | | | | | |
| AKT3_1_243680126_243690814_243946602_ | AKT3 | 0.652970743 | 0.652970743 | 16.8960264 | 3.63E−09 |
| 243948601_FR | | | | | |
| AKT3_1_243680126_243690814_243915703_ | AKT3 | 0.598435451 | 0.598435451 | 11.73324265 | 1.59E−07 |
| 243918596_FR | | | | | |
| AKT3_1_243680126_243690814_243727939_ | AKT3 | 0.520843747 | 0.520843747 | 19.9294303 | 6.33E−10 |
| 243733240_FF | | | | | |
| AKT3_1_243680126_243690814_243860421_ | AKT3 | 0.410316196 | 0.410316196 | 12.44251976 | 8.76E−08 |
| 243862288_FR | | | | | |
| APAF1_12_99061113_99062942_99098781_ | APAF1 | −0.441488336 | −0.441488336 | −13.23940926 | 4.63E−08 |
| 99108240_FF | | | | | |
| APC_5_112020873_112029146_112079758_ | APC | 0.399930381 | 0.399930381 | 6.922678201 | 2.63E−05 |
| 112082452_FF | | | | | |
| AR_X_66792540_66795953_66818342_ | AR | −0.33854166 | −0.33854166 | −6.221155823 | 6.77E−05 |
| 66825862_RF | | | | | |
| AR_X_66736338_66750729_66875649_ | AR | 0.760948793 | 0.760948793 | 21.04618466 | 3.54E−10 |
| 66881776_RR | | | | | |
| AR_X_66736338_66750729_66906874_ | AR | 0.563742659 | 0.563742659 | 20.89428568 | 3.82E−10 |
| 66911452_RR | | | | | |
| AR_X_66750729_66754087_66950367_ | AR | 0.528294859 | 0.528294859 | 9.248783162 | 1.71E−06 |
| 66956132_FR | | | | | |
| AR_X_66818342_66825862_66950367_ | AR | 0.388697407 | 0.388697407 | 10.14296783 | 6.90E−07 |
| 66956132_FF | | | | | |
| AR_X_66911452_66916150_66950367_ | AR | 0.377269056 | 0.377269056 | 7.449040178 | 1.34E−05 |
| 66956132_RR | | | | | |
| AR_X_66736338_66750729_66875649_ | AR | 0.370736884 | 0.370736884 | 5.172651841 | 0.000316014 |
| 66881776_FR | | | | | |
| ATM_11_108112750_108115594_108208085_ | ATM | −0.370811815 | −0.370811815 | −8.707610279 | 3.07E−06 |
| 108223747_FR | | | | | |
| ATM_11_108155279_108156687_108208085_ | ATM | 0.363186489 | 0.363186489 | 6.602286154 | 4.02E−05 |
| 108223747_RR | | | | | |
| BMP6_6_7724582_7733496_7801590_ | BMP6 | −0.468602239 | −0.468602239 | −8.973309325 | 2.30E−06 |
| 7806316_FF | | | | | |
| BMP6_6_7724582_7733496_7743581_ | BMP6 | −0.388036314 | −0.388036314 | −5.889142945 | 0.000108497 |
| 7746369_FR | | | | | |
| CD44_11_35172600_35178637_35204720_ | CD44 | −0.398048925 | −0.398048925 | −5.668665177 | 0.000149604 |
| 35210484_FR | | | | | |
| CDH1_16_68794947_68799115_68857468_ | CDH1 | 0.49540487 | 0.49540487 | 10.65592887 | 4.22E−07 |
| 68863222_FR | | | | | |
| CTNNB1_3_41228301_41234483_41281934_ | CTNNB1 | 0.427937487 | 0.427937487 | 10.8349435 | 3.57E−07 |
| 41304993_FR | | | | | |
| DPP4_2_162933505_162942299_162961246_ | DPP4 | −0.512949291 | −0.512949291 | −14.57522664 | 1.71E−08 |
| 162964936_FR | | | | | |
| DPP4_2_162946178_162949954_162972154_ | DPP4 | −0.445665864 | −0.445665864 | −3.573437421 | 0.004427816 |
| 162979139_RF | | | | | |
| EGFR_7_55080257_55086091_55224588_ | EGFR | −0.493404965 | −0.493404965 | −11.0577665 | 2.91E−07 |
| 55235839_RR | | | | | |
| EPS15_1_51804255_51813510_51945067_ | EPS15 | 0.386206462 | 0.386206462 | 8.698890643 | 3.10E−06 |
| 51946855_FF | | | | | |
| ERBB4_2_213060845_213063716_213336205_ | ERBB4 | −0.681305564 | −0.681305564 | −14.78217408 | 1.48E−08 |
| 213346911_FR | | | | | |
| ERBB4_2_212789287_212798405_212962659_ | ERBB4 | −0.435496836 | −0.435496836 | −5.826671395 | 0.000118755 |
| 212969505_FR | | | | | |
| ERBB4_2_213052672_213059531_213336205_ | ERBB4 | −0.425728775 | −0.425728775 | −12.76218491 | 6.75E−08 |
| 213346911_FR | | | | | |
| ERBB4_2_212789287_212798405_212846041_ | ERBB4 | −0.356744537 | −0.356744537 | −6.538422232 | 4.38E−05 |
| 212850086_RF | | | | | |
| ERBB4_2_212556994_212565232_212622803_ | ERBB4 | 0.688270832 | 0.688270832 | 10.79268571 | 3.71E−07 |
| 212628844_FR | | | | | |
| ERBB4_2_213182054_213190315_213317793_ | ERBB4 | 0.447473513 | 0.447473513 | 10.33352546 | 5.73E−07 |
| 213323368_RR | | | | | |
| ERBB4_2_212622803_212628844_212789287_ | ERBB4 | 0.416082272 | 0.416082272 | 5.631318317 | 0.000158077 |
| 212798405_RF | | | | | |
| ERBB4_2_213151813_213159540_213182054_ | ERBB4 | 0.378853461 | 0.378853461 | 3.217386837 | 0.008284402 |
| 213190315_FF | | | | | |
| ERBB4_2_212556994_212565232_212858137_ | ERBB4 | 0.359671724 | 0.359671724 | 6.923758849 | 2.62E−05 |
| 212868453_FF | | | | | |
| ERG_21_39895678_39899145_39984806_ | ERG | −0.425291613 | −0.425291613 | −11.67074071 | 1.68E−07 |
| 39991905_RF | | | | | |
| ESR1_6_152151654_152158599_152307023_ | ESR1 | −0.334294612 | −0.334294612 | −9.744626114 | 1.03E−06 |
| 152319013_RF | | | | | |
| ETS1_11_128387417_128389914_128489818_ | ETS1 | −0.529524556 | −0.529524556 | −8.680536392 | 3.17E−06 |
| 128498866_FF | | | | | |
| ETS1_11_128431527_128436474_128489818_ | ETS1 | −0.370215655 | −0.370215655 | −5.977392038 | 9.56E−05 |
| 128498866_RF | | | | | |
| ETS1_11_128342943_128345136_128399358_ | ETS1 | −0.344088814 | −0.344088814 | −4.169479623 | 0.001593588 |
| 128409879_FF | | | | | |
| ETS1_11_128342943_128345136_128489818_ | ETS1 | −0.342780154 | −0.342780154 | −4.97505672 | 0.000429841 |
| 128498866_FF | | | | | |
| ETV1_7_13928482_13938998_14075713_ | ETV1 | −0.438646448 | −0.438646448 | −12.61661413 | 7.60E−08 |
| 14080964_FR | | | | | |
| ETV1_7_13928482_13938998_14040827_ | ETV1 | −0.420116215 | −0.420116215 | −7.525930383 | 1.22E−05 |
| 14042620_FR | | | | | |
| FOLH1_11_49157869_49163274_49234427_ | FOLH1 | −0.327026922 | −0.327026922 | −4.300654557 | 0.001279787 |
| 49241370_FF | | | | | |
| FOLH1_11_49214976_49217503_49234427_ | FOLH1 | 0.418767287 | 0.418767287 | 6.637609483 | 3.83E−05 |
| 49241370_RF | | | | | |
| FOLH1_11_49157869_49163274_49193665_ | FOLH1 | 0.359712744 | 0.359712744 | 10.54139329 | 4.70E−07 |
| 49198286_RR | | | | | |
| GLIPR1_12_75847260_75849629_75907812_ | GLIPR1 | −0.345396052 | −0.345396052 | −5.155710032 | 0.000324389 |
| 75913956_FR | | | | | |
| GSK3B_3_119542459_119548768_119722182_ | GSK3B | 0.449273935 | 0.449273935 | 10.95895529 | 3.18E−07 |
| 119724690_FR | | | | | |
| HGF_7_81320024_81325883_81430055_ | HGF | −0.43281007 | −0.43281007 | −4.979466125 | 0.000426875 |
| 81434910_FF | | | | | |
| IGFBP5_2_217560127_217567417_217584428_ | IGFBP5 | 0.375003854 | 0.375003854 | 12.14650233 | 1.12E−07 |
| 217589578_FR | | | | | |
| IL16_15_81429756_81433873_81539851_ | IL16 | −0.481896248 | −0.481896248 | −9.127530593 | 1.95E−06 |
| 81547011_FR | | | | | |
| IL6_7_22721376_22727129_22765455_ | IL6 | 0.364631786 | 0.364631786 | 4.801938715 | 0.00056537 |
| 22766829_FR | | | | | |
| JUN_1_59244918_59246918_59258836_ | JUN | −0.318607525 | −0.318607525 | −5.099115371 | 0.000354116 |
| 59260597_RF | | | | | |
| KIT_4_55553401_55555465_55610649_ | KIT | 0.392634635 | 0.392634635 | 7.232977499 | 1.76E−05 |
| 55618756_FR | | | | | |
| KRAS_12_25363357_25368892_25413300_ | KRAS | 0.414148209 | 0.414148209 | 11.99343016 | 1.28E−07 |
| 25418096_FR | | | | | |
| LPAR3_1_85307233_85315383_85371057_ | LPAR3 | −0.38895037 | −0.38895037 | −13.49428502 | 3.80E−08 |
| 85373099_RR | | | | | |
| LPAR3_1_85265679_85268722_85307233_ | LPAR3 | 0.402766702 | 0.402766702 | 11.08898448 | 2.82E−07 |
| 85315383_RR | | | | | |
| MAP2K5_15_67818519_67824995_68067482_ | MAP2K5 | 0.404437029 | 0.404437029 | 9.01303797 | 2.20E−06 |
| 68072379_FR | | | | | |
| MAPKAP1_9_128370452_128376700_128393518_ | MAPKAP1 | −0.396294954 | −0.396294954 | −12.18592711 | 1.08E−07 |
| 128397379_R | | | | | |
| MIR454_17_57199107_57202160_57227315_ | MIR454 | 0.373313547 | 0.373313547 | 4.540644288 | 0.000861835 |
| 57228782_FF | | | | | |
| MIR98_X_53595032_53600487_53628991_ | MIR98 | 0.742460493 | 0.742460493 | 23.55441603 | 1.06E−10 |
| 53630033_RR | | | | | |
| MIR98_X_53608013_53611637_53628991_ | MIR98 | 0.67511652 | 0.67511652 | 13.76185645 | 3.10E−08 |
| 53630033_RR | | | | | |
| MSR1_8_16213140_16220021_16405541_ | MSR1 | −0.589823054 | −0.589823054 | −8.317534796 | 4.77E−06 |
| 16412741_RR | | | | | |
| MSR1_8_16195878_16203315_16396849_ | MSR1 | −0.419369028 | −0.419369028 | −3.516010141 | 0.004895359 |
| 16400398_FF | | | | | |
| MSR1_8_16045879_16049928_16079226_ | MSR1 | −0.385893508 | −0.385893508 | −10.14385541 | 6.89E−07 |
| 16088483_FF | | | | | |
| MSR1_8_16142611_16149459_16195878_ | MSR1 | −0.337812738 | −0.337812738 | −6.009884545 | 9.13E−05 |
| 16203315_FF | | | | | |
| MSR1_8_16142611_16149459_16396849_ | MSR1 | −0.328903769 | −0.328903769 | −5.188518336 | 0.000308377 |
| 16400398_RF | | | | | |
| MSR1_8_16251114_16260512_16462527_ | MSR1 | −0.318902812 | −0.318902812 | −7.84658429 | 8.28E−06 |
| 16467449_FF | | | | | |
| MSR1_8_16195878_16203315_16433596_ | MSR1 | 0.420145921 | 0.420145921 | 3.761840375 | 0.003192302 |
| 16442100_FR | | | | | |
| NCOA1_2_24829718_24833469_24853776_ | NCOA1 | 0.383599799 | 0.383599799 | 7.061388057 | 2.19E−05 |
| 24866328_RR | | | | | |
| NCOA1_2_24696090_24698819_24840193_ | NCOA1 | 0.376458226 | 0.376458226 | 8.007884338 | 6.84E−06 |
| 24848780_RF | | | | | |
| NCOA1_2_24672976_24676297_24840193_ | NCOA1 | 0.368303096 | 0.368303096 | 8.47574282 | 3.98E−06 |
| 24848780_RF | | | | | |
| NEDD4L_18_55713082_55720762_55811019_ | NEDD4L | −0.444311776 | −0.444311776 | −12.22570821 | 1.05E−07 |
| 55814883_RR | | | | | |
| NEDD4L_18_55713082_55720762_55848311_ | NEDD4L | −0.390337066 | −0.390337066 | −12.14497251 | 1.12E−07 |
| 55850861_RR | | | | | |
| NEDD4L_18_55713082_55720762_55882560_ | NEDD4L | −0.37571287 | −0.37571287 | −12.60684517 | 7.66E−08 |
| 55885168_RR | | | | | |
| NEDD4L_18_55713082_55720762_55869254_ | NEDD4L | −0.360925867 | −0.360925867 | −11.91420916 | 1.36E−07 |
| 55872326_RR | | | | | |
| NEDD4L_18_55713082_55720762_55774243_ | NEDD4L | −0.35657074 | −0.35657074 | −9.240361998 | 1.73E−06 |
| 55779941_RR | | | | | |
| NEDD4L_18_55713082_55720762_55961376_ | NEDD4L | −0.356380382 | −0.356380382 | −8.945405461 | 2.37E−06 |
| 55965188_RR | | | | | |
| NEDD4L_18_55713082_55720762_55784812_ | NEDD4L | −0.354821703 | −0.354821703 | −12.0183087 | 1.25E−07 |
| 55787427_RR | | | | | |
| NEDD4L_18_55713082_55720762_55986600_ | NEDD4L | −0.349236239 | −0.349236239 | −11.79725824 | 1.51E−07 |
| 55989306_RR | | | | | |
| NEDD4L_18_55713082_55720762_55927256_ | NEDD4L | −0.343996768 | −0.343996768 | −8.731068109 | 3.00E−06 |
| 55929658_RR | | | | | |
| NEDD4L_18_55713082_55720762_55950636_ | NEDD4L | −0.326323266 | −0.326323266 | −12.21462858 | 1.06E−07 |
| 55953432_RR | | | | | |
| NEDD4L_18_55713082_55720762_55876126_ | NEDD4L | −0.319472154 | −0.319472154 | −10.93796227 | 3.24E−07 |
| 55882560_RR | | | | | |
| NF1_17_29477103_29483764_29709143_ | NF1 | 0.640366513 | 0.640366513 | 17.71768057 | 2.20E−09 |
| 29714529_FR | | | | | |
| NF1_17_29659279_29666456_29709143_ | NF1 | 0.558103066 | 0.558103066 | 14.14945638 | 2.33E−08 |
| 29714529_FR | | | | | |
| NF1_17_29477103_29483764_29651799_ | NF1 | 0.451959985 | 0.451959985 | 13.17645951 | 4.86E−08 |
| 29657368_FF | | | | | |
| NF1_17_29629862_29634257_29659279_ | NF1 | 0.36665184 | 0.36665184 | 7.2650224 | 1.69E−05 |
| 29666456_RF | | | | | |
| NFKB1_4_103436488_103442700_103548256_ | NFKB1 | 0.79980903 | 0.79980903 | 13.11977478 | 5.08E−08 |
| 103555520_FR | | | | | |
| NFKB1_4_103425294_103430395_103548256_ | NFKB1 | 0.430874236 | 0.430874236 | 14.09753934 | 2.42E−08 |
| 103555520_RR | | | | | |
| NOVA1_14_26999345_27006013_27046501_ | NOVA1 | −0.461947202 | −0.461947202 | −11.22100125 | 2.51E−07 |
| 27053973_FR | | | | | |
| NOVA1_14_26986332_26987866_27070837_ | NOVA1 | −0.325885172 | −0.325885172 | −7.120645256 | 2.03E−05 |
| 27086602_FF | | | | | |
| NR4A3_9_102621891_102624499_102636939_ | NR4A3 | −0.326733183 | −0.326733183 | −6.549075716 | 4.32E−05 |
| 102640160_FR | | | | | |
| PIAS2_18_44419921_44425175_44533399_ | PIAS2 | −0.376922678 | −0.376922678 | −4.811482513 | 0.000556831 |
| 44538938_FF | | | | | |
| PIK3C2A_11_17158103_17163660_17253125_ | PIK3C2A | −0.412653759 | −0.412653759 | −9.581839254 | 1.21E−06 |
| 17255535_FR | | | | | |
| PIK3C2G_12_18503466_18517448_18605599_ | PIK3C2G | −0.397193613 | −0.397193613 | −6.065111351 | 8.44E−05 |
| 18615448_FF | | | | | |
| PIK3C2G_12_18682015_18689955_18755082_ | PIK3C2G | −0.349259284 | −0.349259284 | −7.872086105 | 8.03E−06 |
| 18765416_FF | | | | | |
| PIK3C2G_12_18503466_18517448_18653437_ | PIK3C2G | 0.813156179 | 0.813156179 | 32.76884362 | 3.04E−12 |
| 18654550_FF | | | | | |
| PIK3C2G_12_18503466_18517448_18800920_ | PIK3C2G | 0.52068763 | 0.52068763 | 19.49409228 | 8.00E−10 |
| 18805991_FR | | | | | |
| PIK3C2G_12_18503466_18517448_18586459_ | PIK3C2G | 0.500422551 | 0.500422551 | 22.08623513 | 2.12E−10 |
| 18591749_FR | | | | | |
| PIK3C2G_12_18503466_18517448_18623979_ | PIK3C2G | 0.454500507 | 0.454500507 | 14.21973094 | 2.21E−08 |
| 18629934_FR | | | | | |
| PIK3C2G_12_18466993_18474305_18503466_ | PIK3C2G | 0.418315764 | 0.418315764 | 7.846493305 | 8.28E−06 |
| 18517448_FR | | | | | |
| PIK3C2G_12_18407299_18408850_18503466_ | PIK3C2G | 0.417537091 | 0.417537091 | 12.19551672 | 1.08E−07 |
| 18517448_FR | | | | | |
| PIK3C2G_12_18503466_18517448_18748288_ | PIK3C2G | 0.412545687 | 0.412545687 | 8.404871486 | 4.32E−06 |
| 18755082_FR | | | | | |
| PIK3C2G_12_18409429_18411730_18662602_ | PIK3C2G | 0.404135265 | 0.404135265 | 7.905355237 | 7.72E−06 |
| 18673822_RF | | | | | |
| PIK3C2G_12_18466993_18474305_18765637_ | PIK3C2G | 0.401350605 | 0.401350605 | 10.51089877 | 4.83E−07 |
| 18775643_FR | | | | | |
| PRKCB_16_23929937_23938239_24143206_ | PRKCB | 0.359909066 | 0.359909066 | 12.83614979 | 6.36E−08 |
| 24145438_FR | | | | | |
| PRKCH_14_61911060_61914582_62023126_ | PRKCH | −0.411325245 | −0.411325245 | −5.079346062 | 0.000365169 |
| 62035192_FR | | | | | |
| PRKCH_14_61772357_61775932_61963825_ | PRKCH | −0.335493699 | −0.335493699 | −7.529186724 | 1.22E−05 |
| 61969638_RR | | | | | |
| PTGS2_1_186630471_186639286_186675090_ | PTGS2 | −0.481332802 | −0.481332802 | −6.768676224 | 3.22E−05 |
| 186678395_RR | | | | | |
| PTPN14_1_214555543_214567111_214696754_ | PTPN14 | −0.656412698 | −0.656412698 | −6.179949212 | 7.18E−05 |
| 214699528_RR | | | | | |
| PTPN14_1_214555543_214567111_214590581_ | PTPN14 | −0.341216493 | −0.341216493 | −6.202805991 | 6.95E−05 |
| 214592230_FF | | | | | |
| PTPN14_1_214512778_214523707_214646434_ | PTPN14 | −0.318133487 | −0.318133487 | −4.495773109 | 0.000927417 |
| 214652454_FR | | | | | |
| PTPN14_1_214555543_214567111_214643240_ | PTPN14 | 0.743894345 | 0.743894345 | 14.95502699 | 1.31E−08 |
| 214644608_FR | | | | | |
| PTPRR_12_71045661_71048060_71347632_ | PTPRR | −0.321158046 | −0.321158046 | −5.612244267 | 0.0001626 |
| 71356891_FR | | | | | |
| PTPRR_12_71085097_71096639_71123929_ | PTPRR | 0.550492009 | 0.550492009 | 13.87391165 | 2.85E−08 |
| 71126257_FR | | | | | |
| PTPRR_12_71085097_71096639_71150835_ | PTPRR | 0.37642789 | 0.37642789 | 7.745377455 | 9.35E−06 |
| 71153565_FR | | | | | |
| PTPRT_20_40761966_40770575_40995945_ | PTPRT | 0.390974701 | 0.390974701 | 5.898131356 | 0.000107101 |
| 41003669_FR | | | | | |
| PTPRT_20_40695490_40704819_40853486_ | PTPRT | 0.363092378 | 0.363092378 | 6.022994423 | 8.96E−05 |
| 40862226_RF | | | | | |
| RAN_12_131315466_131318726_131332056_ | RAN | 0.409645167 | 0.409645167 | 4.697687617 | 0.000668175 |
| 131334187_RR | | | | | |
| RB1_13_48835536_48838517_49000831_ | RB1 | −0.421233583 | −0.421233583 | −6.218242572 | 6.80E−05 |
| 49010576_FR | | | | | |
| REL_2_61090704_61099366_61123363_ | REL | −0.396733033 | −0.396733033 | −8.151247086 | 5.78E−06 |
| 61128146_FF | | | | | |
| REL_2_61090704_61099366_61149976_ | REL | −0.36693886 | −0.36693886 | −7.752686136 | 9.27E−06 |
| 61161058_FF | | | | | |
| REL_2_61090704_61099366_61144132_ | REL | 0.677200702 | 0.677200702 | 15.31760281 | 1.02E−08 |
| 61147262_FR | | | | | |
| RGS6_14_72418571_72425681_72679959_ | RGS6 | 0.640246502 | 0.640246502 | 17.53962222 | 2.45E−09 |
| 72689252_RR | | | | | |
| ROR2_9_94323433_94326108_94448327_ | ROR2 | −0.317797399 | −0.317797399 | −13.17593415 | 4.86E−08 |
| 94455574_FF | | | | | |
| SCGB1A1_11_62128712_62135211_62160970_ | SCGB1A1 | −0.657694922 | −0.657694922 | −10.36431148 | 5.56E−07 |
| 62163465_FR | | | | | |
| SOS1_2_39227963_39230003_39353282_ | SOS1 | 0.674466946 | 0.674466946 | 13.89064783 | 2.82E−08 |
| 39361637_RF | | | | | |
| SOS1_2_39209340_39220780_39276526_ | SOS1 | 0.461207721 | 0.461207721 | 10.99304586 | 3.08E−07 |
| 39280091_FR | | | | | |
| SRD5A2_2_31741633_31747723_31778586_ | SRD5A2 | −0.437278492 | −0.437278492 | −17.05212796 | 3.30E−09 |
| 31789876_FF | | | | | |
| SRD5A2_2_31729027_31741633_31760980_ | SRD5A2 | 0.394046044 | 0.394046044 | 9.411702288 | 1.44E−06 |
| 31767977_FR | | | | | |
| SRD5A2_2_31760980_31767977_31778586_ | SRD5A2 | 0.36636811 | 0.36636811 | 11.1293664 | 2.72E−07 |
| 31789876_RF | | | | | |
| TGFB2_1_218504155_218510817_218542394_ | TGFB2 | −0.401060991 | −0.401060991 | −18.11165268 | 1.74E−09 |
| 218548723_RF | | | | | |
| TGFB2_1_218491029_218498929_218553354_ | TGFB2 | −0.386196098 | −0.386196098 | −7.834000204 | 8.41E−06 |
| 218556593_FF | | | | | |
| TMPRSS2_21_42841804_42850832_42927381_ | TMPRSS2 | 0.46760546 | 0.46760546 | 8.609824216 | 3.43E−06 |
| 42930038_FR | | | | | |
| TOP2A_17_38547618_38549511_38613131_ | TOP2A | −0.334752665 | −0.334752665 | −8.436098305 | 4.17E−06 |
| 38616534_RR | | | | | |
| TOP2A_17_38564762_38568693_38613131_ | TOP2A | −0.33269637 | −0.33269637 | −9.020450371 | 2.18E−06 |
| 38616534_RR | | | | | |
| TOP2B_3_25644985_25663188_25716096_ | TOP2B | 0.677625777 | 0.677625777 | 11.54384037 | 1.88E−07 |
| 25717154_FF | | | | | |
| VEGFC_4_177629821_177639626_177693384_ | VEGFC | 0.624813933 | 0.624813933 | 8.924483137 | 2.42E−06 |
| 177697283_FR | | | | | |
| VEGFC_4_177629821_177639626_177740221_ | VEGFC | 0.532875204 | 0.532875204 | 11.11732726 | 2.75E−07 |
| 177743175_FR | | | | | |
| VEGFC_4_177629821_177639626_177693384_ | VEGFC | 0.418296493 | 0.418296493 | 13.22565823 | 4.68E−08 |
| 177697283_FF | | | | | |
| EZH2_7_148496931_148503515_148602692_ | EZH2 | 0.221213903 | 0.221213903 | 6.686469603 | 3.59E−05 |
| 148606606_FF | | | | | |
| EZH2_7_148496931_148503515_148610251_ | EZH2 | 0.220468898 | 0.220468898 | 8.311487407 | 4.80E−06 |
| 148614284_FR | | | | | |
| SP1_12_53752782_53754759_53771263_ | SP1 | 0.197127842 | 0.197127842 | 4.00029596 | 0.002121214 |
| 53775550_RF | | | | | |
| SP1_12_53771263_53775550_53824264_ | SP1 | 0.193365041 | 0.193365041 | 3.676018582 | 0.003703744 |
| 53827278_FR | | | | | |
| DAPK1_9_90064560_90073617_90176237_ | DAPK1 | −0.210075392 | −0.210075392 | −5.311660617 | 0.000255365 |
| 90180153_FF | | | | | |
| DAPK1_9_90064560_90073617_90339152_ | DAPK1 | −0.289887636 | −0.289887636 | −3.250481175 | 0.007812935 |
| 90340776_FF | | | | | |
| DAPK1_9_90064560_90073617_90140806_ | DAPK1 | 0.299375751 | 0.299375751 | 7.197207444 | 1.84E−05 |
| 90142738_FR | | | | | |
| DAPK1_9_90064560_90073617_90140806_ | DAPK1 | 0.22308584 | 0.22308584 | 3.330633912 | 0.006781108 |
| 90142738_FF | | | | | |
| FGD4_12_32760791_32767406_32781508_ | FGD4 | −0.274580142 | −0.274580142 | −5.050237381 | 0.000382113 |
| 32786048_FR | | | | | |
| FGD4_12_32760791_32767406_32781508_ | FGD4 | −0.282349703 | −0.282349703 | −5.378249402 | 0.000230809 |
| 32786048_RR | | | | | |
| FGD4_12_32714978_32722972_32735447_ | FGD4 | 0.277743465 | 0.277743465 | 4.897130535 | 0.000486024 |
| 32738552_RR | | | | | |
| FGD4_12_32714978_32722972_32768073_ | FGD4 | 0.224649977 | 0.224649977 | 3.77524936 | 0.003119249 |
| 32772358_RR | | | | | |
| GAB1_4_144235957_144242111_144369687_ | GAB1 | 0.322283078 | 0.322283078 | 7.881995854 | 7.94E−06 |
| 144374560_FF | | | | | |
| GAB1_4_144272552_144276220_144402622_ | GAB1 | 0.232807271 | 0.232807271 | 5.862864002 | 0.000112692 |
| 144411096_FR | | | | | |
| GAB1_4_144254752_144257034_144321110_ | GAB1 | −0.1933323 | −0.1933323 | −3.299161865 | 0.00716858 |
| 144332903_RF | | | | | |
| GAB1_4_144298156_144300750_144321110_ | GAB1 | −0.203419346 | −0.203419346 | −6.533724514 | 4.41E−05 |
| 144332903_FR | | | | | |
| HSD3B2_1_119937390_119948935_119959754_ | HSD3B2 | 0.207425802 | 0.207425802 | 6.208022809 | 6.90E−05 |
| 119963670_FR | | | | | |
| HSD3B2_1_119937390_119948935_119959754_ | HSD3B2 | 0.167484698 | 0.167484698 | 5.70659508 | 0.000141491 |
| 119963670_FF | | | | | |
| HSD3B2_1_119912462_119915175_119959754_ | HSD3B2 | −0.168081632 | −0.168081632 | −3.274998031 | 0.007481356 |
| 119963670_RR | | | | | |
| KLK2_19_51340459_51344004_51390533_ | KLK2 | −0.233986179 | −0.233986179 | −5.085188859 | 0.000361865 |
| 51395187_FR | | | | | |
| KLK2_19_51317027_51319938_51340459_ | KLK2 | 0.259249519 | 0.259249519 | 3.256953029 | 0.007723978 |
| 51344004_FF | | | | | |
| KLK2_19_51317027_51319938_51346270_ | KLK2 | 0.251147882 | 0.251147882 | 3.297865414 | 0.007185018 |
| 51350944_FF | | | | | |
| MAP3K14_17_43358197_43360790_43375304_ | MAP3K14 | 0.227459078 | 0.227459078 | 4.562645956 | 0.000831476 |
| 43380378_FF | | | | | |
| MAP3K14_17_43360790_43364282_43409961_ | MAP3K14 | 0.198862372 | 0.198862372 | 4.316518127 | 0.00124648 |
| 43415408_FF | | | | | |
| MAP3K14_17_43358197_43360790_43375304_ | MAP3K14 | −0.205626917 | −0.205626917 | −6.208573083 | 6.89E−05 |
| 43380378_RR | | | | | |
| MIF_22_24194490_24195811_24245843_ | MIF | 0.254727783 | 0.254727783 | 10.76065239 | 3.82E−07 |
| 24254074_RR | | | | | |
| MIF_22_24206371_24208274_24245843_ | MIF | 0.207093745 | 0.207093745 | 5.104189064 | 0.000351337 |
| 24254074_FR | | | | | |
| MUC1_1_155176403_155179713_155191807_ | MUC1 | −0.209333417 | −0.209333417 | −6.160281821 | 7.38E−05 |
| 155193554_FR | | | | | |
| MUC1_1_155146523_155149986_155191807_ | MUC1 | −0.218468452 | −0.218468452 | −5.993061073 | 9.35E−05 |
| 155193554_FR | | | | | |
| RAD51_15_40972719_40979675_41025213_ | RAD51 | 0.352545734 | 0.352545734 | 4.666716555 | 0.00070238 |
| 41027977_RF | | | | | |
| RAD51_15_40937212_40938851_41025213_ | RAD51 | −0.242505623 | −0.242505623 | −4.646371743 | 0.000725849 |
| 41027977_RF | | | | | |
| RAD51_15_41009919_41011826_41025213_ | RAD51 | −0.243901539 | −0.243901539 | −6.598967119 | 4.03E−05 |
| 41027977_RF | | | | | |
| RNASEL_1_182541376_182556600_182605098_ | RNASEL | 0.268497887 | 0.268497887 | 3.858975685 | 0.002700604 |
| 182607856_FR | | | | | |
| RNASEL_1_182541376_182556600_182577916_ | RNASEL | 0.25710383 | 0.25710383 | 3.235840558 | 0.008018044 |
| 182584530_FF | | | | | |
| SRC_20_35928873_35935451_35989678_ | SRC | 0.206516047 | 0.206516047 | 6.456694813 | 4.89E−05 |
| 35993330_FR | | | | | |
| SRC_20_35928873_35935451_35989678_ | SRC | 0.198581558 | 0.198581558 | 6.507686654 | 4.56E−05 |
| 35993330_FF | | | | | |
| SRD5A3_4_56188038_56191526_56242301_ | SRD5A3 | 0.266992266 | 0.266992266 | 4.835274287 | 0.000536131 |
| 56245314_RF | | | | | |
| SRD5A3_4_56209429_56213336_56242301_ | SRD5A3 | 0.239396914 | 0.239396914 | 4.842348143 | 0.000530134 |
| 56245314_RF | | | | | |
| WNT1_12_49327866_49332429_49386082_ | WNT1 | 0.171379721 | 0.171379721 | 4.015889993 | 0.002065728 |
| 49387249_RF | | | | | |
| WNT1_12_49361168_49364315_49377006_ | WNT1 | −0.188758659 | −0.188758659 | −6.377243699 | 5.46E−05 |
| 49380965_RF | | | | | |
| WNT1_12_49327866_49332429_49364343_ | WNT1 | −0.289012147 | −0.289012147 | −10.04098231 | 7.63E−07 |
| 49365445_FF | | | | | |
| Probes | adj.P.Val | B | FC | FC_1 | Binary |
| ACAT1_11_107955219_107960166_108013361_ | 3.10E−05 | 8.114204006 | 0.738809576 | −1.353528748 | −1 |
| 108018367_FF | | | | | |
| ACTA1_1_229547333_229551721_229600994_ | 0.000260244 | 5.025483662 | 1.335935441 | 1.335935441 | 1 |
| 229605798_FR | | | | | |
| AKT3_1_243680126_243690814_243946602_ | 3.08E−06 | 11.560433 | 1.572402697 | 1.572402697 | 1 |
| 243948601_FR | | | | | |
| AKT3_1_243680126_243690814_243915703_ | 3.45E−05 | 7.966660757 | 1.514073719 | 1.514073719 | 1 |
| 243918596_FR | | | | | |
| AKT3_1_243680126_243690814_243727939_ | 1.07E−06 | 13.10148999 | 1.434794129 | 1.434794129 | 1 |
| 243733240_FF | | | | | |
| AKT3_1_243680126_243690814_243860421_ | 2.55E−05 | 8.554504322 | 1.328977055 | 1.328977055 | 1 |
| 243862288_FR | | | | | |
| APAF1_12_99061113_99062942_99098781_ | 1.71E−05 | 9.174110234 | 0.736374546 | −1.358004571 | −1 |
| 99108240_FF | | | | | |
| APC_5_112020873_112029146_112079758_ | 0.000993557 | 2.789251424 | 1.319444238 | 1.319444238 | 1 |
| 112082452_FF | | | | | |
| AR_X_66792540_66795953_66818342_ | 0.001660581 | 1.810263084 | 0.790840324 | −1.264477758 | −1 |
| 66825862_RF | | | | | |
| AR_X_66736338_66750729_66875649_ | 7.78E−07 | 13.59167252 | 1.694604721 | 1.694604721 | 1 |
| 66881776_RR | | | | | |
| AR_X_66736338_66750729_66906874_ | 7.78E−07 | 13.52715233 | 1.478098751 | 1.478098751 | 1 |
| 66911452_RR | | | | | |
| AR_X_66750729_66754087_66950367_ | 0.000187011 | 5.588100983 | 1.442223604 | 1.442223604 | 1 |
| 66956132_FR | | | | | |
| AR_X_66818342_66825862_66950367_ | 9.11E−05 | 6.507167389 | 1.309210798 | 1.309210798 | 1 |
| 66956132_FF | | | | | |
| AR_X_66911452_66916150_66950367_ | 0.000639022 | 3.480123787 | 1.298880815 | 1.298880815 | 1 |
| 66956132_RR | | | | | |
| AR_X_66736338_66750729_66875649_ | 0.004216579 | 0.216960602 | 1.293013093 | 1.293013093 | 1 |
| 66881776_FR | | | | | |
| ATM_11_108112750_108115594_108208085_ | 0.000260244 | 4.992731905 | 0.773347206 | −1.293080251 | −1 |
| 108223747_FR | | | | | |
| ATM_11_108155279_108156687_108208085_ | 0.001238401 | 2.350592936 | 1.28626374 | 1.28626374 | 1 |
| 108223747_RR | | | | | |
| BMP6_6_7724582_7733496_7801590_ | 0.000229279 | 5.288915333 | 0.722664415 | −1.383768149 | −1 |
| 7806316_FF | | | | | |
| BMP6_6_7724582_7733496_7743581_ | 0.002198909 | 1.322801574 | 0.764169025 | −1.30861101 | −1 |
| 7746369_FR | | | | | |
| CD44_11_35172600_35178637_35204720_ | 0.002652749 | 0.990360448 | 0.758883891 | −1.317724638 | −1 |
| 35210484_FR | | | | | |
| CDH1_16_68794947_68799115_68857468_ | 6.84E−05 | 7.000965624 | 1.409716315 | 1.409716315 | 1 |
| 68863222_FR | | | | | |
| CTNNB1_3_41228301_41234483_41281934_ | 6.15E−05 | 7.167935447 | 1.345308914 | 1.345308914 | 1 |
| 41304993_FR | | | | | |
| DPP4_2_162933505_162942299_162961246_ | 1.09E−05 | 10.1259903 | 0.700788356 | −1.426964348 | −1 |
| 162964936_FR | | | | | |
| DPP4_2_162946178_162949954_162972154_ | 0.023189841 | −2.491256189 | 0.734245353 | −1.361942565 | −1 |
| 162979139_RF | | | | | |
| EGFR_7_55080257_55086091_55224588_ | 5.37E−05 | 7.372042198 | 0.710346599 | −1.407763479 | −1 |
| 55235839_RR | | | | | |
| EPS15_1_51804255_51813510_51945067_ | 0.000260244 | 4.982882133 | 1.306952276 | 1.306952276 | 1 |
| 51946855_FF | | | | | |
| ERBB4_2_213060845_213063716_213336205_ | 1.00E−05 | 10.26456915 | 0.623600693 | −1.603590265 | −1 |
| 213346911_FR | | | | | |
| ERBB4_2_212789287_212798405_212962659_ | 0.002319347 | 1.229315586 | 0.739439062 | −1.352376485 | −1 |
| 212969505_FR | | | | | |
| ERBB4_2_213052672_213059531_213336205_ | 2.15E−05 | 8.808033687 | 0.744462573 | −1.343250872 | −1 |
| 213346911_FR | | | | | |
| ERBB4_2_212789287_212798405_212846041_ | 0.001298756 | 2.261469357 | 0.780924761 | −1.280533093 | −1 |
| 212850086_RF | | | | | |
| ERBB4_2_212556994_212565232_212622803_ | 6.29E−05 | 7.128763987 | 1.611351047 | 1.611351047 | 1 |
| 212628844_FR | | | | | |
| ERBB4_2_213182054_213190315_213317793_ | 8.45E−05 | 6.693320715 | 1.363650103 | 1.363650103 | 1 |
| 213323368_RR | | | | | |
| ERBB4_2_212622803_212628844_212789287_ | 0.002758619 | 0.933355245 | 1.334299258 | 1.334299258 | 1 |
| 212798405_RF | | | | | |
| ERBB4_2_213151813_213159540_213182054_ | 0.035699071 | −3.122932982 | 1.300308063 | 1.300308063 | 1 |
| 213190315_FF | | | | | |
| ERBB4_2_212556994_212565232_212858137_ | 0.000993557 | 2.790707369 | 1.283133896 | 1.283133896 | 1 |
| 212868453_FF | | | | | |
| ERG_21_39895678_39899145_39984806_ | 3.57E−05 | 7.913111034 | 0.744688192 | −1.342843905 | −1 |
| 39991905_RF | | | | | |
| ESR1_6_152151654_152158599_152307023_ | 0.000124223 | 6.107253454 | 0.793171853 | −1.260760825 | −1 |
| 152319013_RF | | | | | |
| ETS1_11_128387417_128389914_128489818_ | 0.000260244 | 4.962121749 | 0.692783005 | −1.443453423 | −1 |
| 128498866_FF | | | | | |
| ETS1_11_128431527_128436474_128489818_ | 0.002017625 | 1.453907443 | 0.77366684 | −1.292546027 | −1 |
| 128498866_RF | | | | | |
| ETS1_11_128342943_128345136_128399358_ | 0.012099379 | −1.449395395 | 0.787805386 | −1.269349026 | −1 |
| 128409879_FF | | | | | |
| ETS1_11_128342943_128345136_128489818_ | 0.005055727 | −0.100832117 | 0.788520324 | −1.26819813 | −1 |
| 128498866_FF | | | | | |
| ETV1_7_13928482_13938998_14075713_ | 2.29E−05 | 8.693428053 | 0.737826521 | −1.355332144 | −1 |
| 14080964_FR | | | | | |
| ETV1_7_13928482_13938998_14040827_ | 0.000603601 | 3.57803973 | 0.747364419 | −1.338035334 | −1 |
| 14042620_FR | | | | | |
| FOLH1_11_49157869_49163274_49234427_ | 0.010350202 | −1.224472556 | 0.7971776 | −1.254425613 | −1 |
| 49241370_FF | | | | | |
| FOLH1_11_49214976_49217503_49234427_ | 0.00120659 | 2.399644945 | 1.336784849 | 1.336784849 | 1 |
| 49241370_RF | | | | | |
| FOLH1_11_49157869_49163274_49193665_ | 7.47E−05 | 6.892707121 | 1.28317038 | 1.28317038 | 1 |
| 49198286_RR | | | | | |
| GLIPR1_12_75847260_75849629_75907812_ | 0.004293149 | 0.189926553 | 0.787091873 | −1.270499715 | −1 |
| 75913956_FR | | | | | |
| GSK3B_3_119542459_119548768_119722182_ | 5.68E−05 | 7.282033839 | 1.365352943 | 1.365352943 | 1 |
| 119724690_FR | | | | | |
| HGF_7_81320024_81325883_81430055_ | 0.00504444 | −0.093681464 | 0.740817421 | −1.349860265 | −1 |
| 81434910_FF | | | | | |
| IGFBP5_2_217560127_217567417_217584428_ | 2.78E−05 | 8.313515985 | 1.296843019 | 1.296843019 | 1 |
| 217589578_FR | | | | | |
| IL16_15_81429756_81433873_81539851_ | 0.000208593 | 5.457383883 | 0.716035863 | −1.396578093 | −1 |
| 81547011_FR | | | | | |
| IL6_7_22721376_22727129_22765455_ | 0.005957969 | −0.383664127 | 1.28755297 | 1.28755297 | 1 |
| 22766829_FR | | | | | |
| JUN_1_59244918_59246918_59258836_ | 0.004543222 | 0.099326736 | 0.801843436 | −1.247126254 | −1 |
| 59260597_RF | | | | | |
| KIT_4_55553401_55555465_55610649_ | 0.000779657 | 3.200921573 | 1.312788617 | 1.312788617 | 1 |
| 55618756_FR | | | | | |
| KRAS_12_25363357_25368892_25413300_ | 3.02E−05 | 8.186481963 | 1.332511707 | 1.332511707 | 1 |
| 25418096_FR | | | | | |
| LPAR3_1_85307233_85315383_85371057_ | 1.61E−05 | 9.363780155 | 0.76368502 | −1.309440376 | −1 |
| 85373099_RR | | | | | |
| LPAR3_1_85265679_85268722_85307233_ | 5.32E−05 | 7.400314167 | 1.322040801 | 1.322040801 | 1 |
| 85315383_RR | | | | | |
| MAP2K5_15_67818519_67824995_68067482_ | 0.000221829 | 5.332553143 | 1.323572323 | 1.323572323 | 1 |
| 68072379_FR | | | | | |
| MAPKAP1_9_128370452_128376700_128393518_ | 2.78E−05 | 8.345964775 | 0.759807072 | −1.316123575 | −1 |
| 128397379_R | | | | | |
| MIR454_17_57199107_57202160_57227315_ | 0.007908614 | −0.818047279 | 1.295324487 | 1.295324487 | 1 |
| 57228782_FF | | | | | |
| MIR98_X_53595032_53600487_53628991_ | 5.41E−07 | 14.56868412 | 1.673026728 | 1.673026728 | 1 |
| 53630033_RR | | | | | |
| MIR98_X_53608013_53611637_53628991_ | 1.43E−05 | 9.558686586 | 1.596725728 | 1.596725728 | 1 |
| 53630033_RR | | | | | |
| MSR1_8_16213140_16220021_16405541_ | 0.000337002 | 4.54380866 | 0.664424394 | −1.50506214 | −1 |
| 16412741_RR | | | | | |
| MSR1_8_16195878_16203315_16396849_ | 0.024952597 | −2.59289798 | 0.747751587 | −1.337342532 | −1 |
| 16400398_FF | | | | | |
| MSR1_8_16045879_16049928_16079226_ | 9.11E−05 | 6.508042059 | 0.765304873 | −1.306668799 | −1 |
| 16088483_FF | | | | | |
| MSR1_8_16142611_16149459_16195878_ | 0.00198016 | 1.501898146 | 0.791239998 | −1.263839041 | −1 |
| 16203315_FF | | | | | |
| MSR1_8_16142611_16149459_16396849_ | 0.004162163 | 0.242242178 | 0.796141201 | −1.256058596 | −1 |
| 16400398_RF | | | | | |
| MSR1_8_16251114_16260512_16462527_ | 0.000473286 | 3.978390344 | 0.801679333 | −1.247381538 | −1 |
| 16467449_FF | | | | | |
| MSR1_8_16195878_16203315_16433596_ | 0.018914227 | −2.159017761 | 1.338062886 | 1.338062886 | 1 |
| 16442100_FR | | | | | |
| NCOA1_2_24829718_24833469_24853776_ | 0.000893094 | 2.974854782 | 1.304593006 | 1.304593006 | 1 |
| 24866328_RR | | | | | |
| NCOA1_2_24696090_24698819_24840193_ | 0.000434693 | 4.175013545 | 1.298151018 | 1.298151018 | 1 |
| 24848780_RF | | | | | |
| NCOA1_2_24672976_24676297_24840193_ | 0.000302485 | 4.72794807 | 1.290833654 | 1.290833654 | 1 |
| 24848780_RF | | | | | |
| NEDD4L_18_55713082_55720762_55811019_ | 2.78E−05 | 8.378595915 | 0.734934827 | −1.360664869 | −1 |
| 55814883_RR | | | | | |
| NEDD4L_18_55713082_55720762_55848311_ | 2.78E−05 | 8.312254649 | 0.76295133 | −1.310699595 | −1 |
| 55850861_RR | | | | | |
| NEDD4L_18_55713082_55720762_55882560_ | 2.29E−05 | 8.685686665 | 0.770724485 | −1.297480512 | −1 |
| 55885168_RR | | | | | |
| NEDD4L_18_55713082_55720762_55869254_ | 3.10E−05 | 8.120075504 | 0.778664702 | −1.284249816 | −1 |
| 55872326_RR | | | | | |
| NEDD4L_18_55713082_55720762_55774243_ | 0.000187011 | 5.579071341 | 0.781018843 | −1.28037884 | −1 |
| 55779941_RR | | | | | |
| NEDD4L_18_55713082_55720762_55961376_ | 0.00023 | 5.258165908 | 0.781121902 | −1.28020991 | −1 |
| 55965188_RR | | | | | |
| NEDD4L_18_55713082_55720762_55784812_ | 3.02E−05 | 8.207242683 | 0.781966277 | −1.278827526 | −1 |
| 55787427_RR | | | | | |
| NEDD4L_18_55713082_55720762_55986600_ | 3.34E−05 | 8.021205936 | 0.784999566 | −1.273886055 | −1 |
| 55989306_RR | | | | | |
| NEDD4L_18_55713082_55720762_55927256_ | 0.000260244 | 5.019188722 | 0.787855651 | −1.269268043 | −1 |
| 55929658_RR | | | | | |
| NEDD4L_18_55713082_55720762_55950636_ | 2.78E−05 | 8.36951882 | 0.797566509 | −1.253813932 | −1 |
| 55953432_RR | | | | | |
| NEDD4L_18_55713082_55720762_55876126_ | 5.69E−05 | 7.262808228 | 0.801363023 | −1.2478739 | −1 |
| 55882560_RR | | | | | |
| NF1_17_29477103_29483764_29709143_ | 2.49E−06 | 12.01137937 | 1.5587251 | 1.5587251 | 1 |
| 29714529_FR | | | | | |
| NF1_17_29659279_29666456_29709143_ | 1.29E−05 | 9.83363673 | 1.472332041 | 1.472332041 | 1 |
| 29714529_FR | | | | | |
| NF1_17_29477103_29483764_29651799_ | 1.71E−05 | 9.126648681 | 1.367897363 | 1.367897363 | 1 |
| 29657368_FF | | | | | |
| NF1_17_29629862_29634257_29659279_ | 0.000765315 | 3.242712391 | 1.289357057 | 1.289357057 | 1 |
| 29666456_RF | | | | | |
| NFKB1_4_103436488_103442700_103548256_ | 1.72E−05 | 9.083698686 | 1.740870671 | 1.740870671 | 1 |
| 103555520_FR | | | | | |
| NFKB1_4_103425294_103430395_103548256_ | 1.29E−05 | 9.797304101 | 1.348050213 | 1.348050213 | 1 |
| 103555520_RR | | | | | |
| NOVA1_14_26999345_27006013_27046501_ | 5.00E−05 | 7.519006961 | 0.726005709 | −1.377399637 | −1 |
| 27053973_FR | | | | | |
| NOVA1_14_26986332_26987866_27070837_ | 0.000844533 | 3.053363996 | 0.797808737 | −1.253433252 | −1 |
| 27086602_FF | | | | | |
| NR4A3_9_102621891_102624499_102636939_ | 0.001283905 | 2.276375868 | 0.797339926 | −1.254170233 | −1 |
| 102640160_FR | | | | | |
| PIAS2_18_44419921_44425175_44533399_ | 0.005918513 | −0.367966827 | 0.770078446 | −1.298569003 | −1 |
| 44538938_FF | | | | | |
| PIK3C2A_11_17158103_17163660_17253125_ | 0.000138409 | 5.939488583 | 0.751240236 | −1.331132109 | −1 |
| 17255535_FR | | | | | |
| PIK3C2G_12_18503466_18517448_18605599_ | 0.001886444 | 1.583120211 | 0.759333934 | −1.316943647 | −1 |
| 18615448_FF | | | | | |
| PIK3C2G_12_18682015_18689955_18755082_ | 0.000473286 | 4.009686303 | 0.784987026 | −1.273906404 | −1 |
| 18765416_FF | | | | | |
| PIK3C2G_12_18503466_18517448_18653437_ | 3.10E−08 | 17.09863144 | 1.757051136 | 1.757051136 | 1 |
| 18654550_FF | | | | | |
| PIK3C2G_12_18503466_18517448_18800920_ | 1.16E−06 | 12.9000512 | 1.434638875 | 1.434638875 | 1 |
| 18805991_FR | | | | | |
| PIK3C2G_12_18503466_18517448_18586459_ | 7.17E−07 | 14.016338 | 1.414627832 | 1.414627832 | 1 |
| 18591749_FR | | | | | |
| PIK3C2G_12_18503466_18517448_18623979_ | 1.29E−05 | 9.882576362 | 1.370308292 | 1.370308292 | 1 |
| 18629934_FR | | | | | |
| PIK3C2G_12_18466993_18474305_18503466_ | 0.000473286 | 3.978278546 | 1.336366539 | 1.336366539 | 1 |
| 18517448_RF | | | | | |
| PIK3C2G_12_18407299_18408850_18503466_ | 2.78E−05 | 8.35384099 | 1.335645449 | 1.335645449 | 1 |
| 18517448_RF | | | | | |
| PIK3C2G_12_18503466_18517448_18748288_ | 0.000313738 | 4.645813078 | 1.331032398 | 1.331032398 | 1 |
| 18755082_FR | | | | | |
| PIK3C2G_12_18409429_18411730_18662602_ | 0.000473286 | 4.050395593 | 1.323295505 | 1.323295505 | 1 |
| 18673822_RF | | | | | |
| PIK3C2G_12_18466993_18474305_18765637_ | 7.57E−05 | 6.863693215 | 1.32074377 | 1.32074377 | 1 |
| 18775643_FR | | | | | |
| PRKCB_16_23929937_23938239_24143206_ | 2.09E−05 | 8.865730305 | 1.283345005 | 1.283345005 | 1 |
| 24145438_FR | | | | | |
| PRKCH_14_61911060_61914582_62023126_ | 0.004598091 | 0.067573492 | 0.751932339 | −1.329906892 | −1 |
| 62035192_FR | | | | | |
| PRKCH_14_61772357_61775932_61963825_ | 0.000603601 | 3.582169975 | 0.792512887 | −1.261809134 | −1 |
| 61969638_RR | | | | | |
| PTGS2_1_186630471_186639286_186675090_ | 0.001116912 | 2.580151439 | 0.716315566 | −1.396032765 | −1 |
| 186678395_RR | | | | | |
| PTPN14_1_214555543_214567111_214696754_ | 0.001709697 | 1.750618263 | 0.634453925 | −1.576158584 | −1 |
| 214699528_RR | | | | | |
| PTPN14_1_214555543_214567111_214590581_ | 0.001671497 | 1.783732259 | 0.789375423 | −1.266824341 | −1 |
| 214592230_FF | | | | | |
| PTPN14_1_214512778_214523707_214646434_ | 0.008276791 | −0.89351305 | 0.802106947 | −1.246716543 | −1 |
| 214652454_FR | | | | | |
| PTPN14_1_214555543_214567111_214643240_ | 9.50E−06 | 10.37860359 | 1.674690326 | 1.674690326 | 1 |
| 214644608_FR | | | | | |
| PTPRR_12_71045661_71048060_71347632_ | 0.002808651 | 0.904163728 | 0.80042712 | −1.249332981 | −1 |
| 71356891_FR | | | | | |
| PTPRR_12_71085097_71096639_71123929_ | 1.38E−05 | 9.639060097 | 1.464585085 | 1.464585085 | 1 |
| 71126257_FR | | | | | |
| PTPRR_12_71085097_71096639_71150835_ | 0.000508734 | 3.85340498 | 1.298123721 | 1.298123721 | 1 |
| 71153565_FR | | | | | |
| PTPRT_20_40761966_40770575_40995945_ | 0.002179292 | 1.336206195 | 1.31127902 | 1.31127902 | 1 |
| 41003669_FR | | | | | |
| PTPRT_20_40695490_40704819_40853486_ | 0.001959674 | 1.521218337 | 1.286179837 | 1.286179837 | 1 |
| 40862226_RF | | | | | |
| RAN_12_131315466_131318726_131332056_ | 0.00665819 | −0.555916376 | 1.328359062 | 1.328359062 | 1 |
| 131334187_RR | | | | | |
| RB1_13_48835536_48838517_49000831_ | 0.001663343 | 1.806054195 | 0.746785809 | −1.339072045 | −1 |
| 49010576_FR | | | | | |
| REL_2_61090704_61099366_61123363_ | 0.000384323 | 4.347158457 | 0.759576389 | −1.316523281 | −1 |
| 61128146_FF | | | | | |
| REL_2_61090704_61099366_61149976_ | 0.000506975 | 3.862472978 | 0.775426067 | −1.289613597 | −1 |
| 61161058_FF | | | | | |
| REL_2_61090704_61099366_61144132_ | 7.96E−06 | 10.6128694 | 1.599034097 | 1.599034097 | 1 |
| 61147262_FR | | | | | |
| RGS6_14_72418571_72425681_72679959_ | 2.49E−06 | 11.91593523 | 1.558595442 | 1.558595442 | 1 |
| 72689252_RR | | | | | |
| ROR2_9_94323433_94326108_94448327_ | 1.71E−05 | 9.126251542 | 0.802293826 | −1.246426144 | −1 |
| 94455574_FF | | | | | |
| SCGB1A1_11_62128712_62135211_62160970_ | 8.40E−05 | 6.723090349 | 0.633890292 | −1.57756005 | −1 |
| 62163465_FR | | | | | |
| SOS1_2_39227963_39230003_39353282_ | 1.38E−05 | 9.651002 | 1.596006963 | 1.596006963 | 1 |
| 39361637_RF | | | | | |
| SOS1_2_39209340_39220780_39276526_ | 5.60E−05 | 7.313177343 | 1.376693805 | 1.376693805 | 1 |
| 39280091_FR | | | | | |
| SRD5A2_2_31741633_31747723_31778586_ | 3.05E−06 | 11.6482057 | 0.738526456 | −1.354047634 | −1 |
| 31789876_FF | | | | | |
| SRD5A2_2_31729027_31741633_31760980_ | 0.000162975 | 5.761374339 | 1.314073565 | 1.314073565 | 1 |
| 31767977_FR | | | | | |
| SRD5A2_2_31760980_31767977_31778586_ | 5.28E−05 | 7.436768688 | 1.289103509 | 1.289103509 | 1 |
| 31789876_RF | | | | | |
| TGFB2_1_218504155_218510817_218542394_ | 2.22E−06 | 12.21825228 | 0.757301142 | −1.320478664 | −1 |
| 218548723_RF | | | | | |
| TGFB2_1_218491029_218498929_218553354_ | 0.000476069 | 3.962917896 | 0.765144376 | −1.306942888 | −1 |
| 218556593_FF | | | | | |
| TMPRSS2_21_42841804_42850832_42927381_ | 0.000272613 | 4.88179253 | 1.382812413 | 1.382812413 | 1 |
| 42930038_FR | | | | | |
| TOP2A_17_38547618_38549511_38613131_ | 0.000304987 | 4.682072992 | 0.792920063 | −1.261161178 | −1 |
| 38616534_RR | | | | | |
| TOP2A_17_38564762_38568693_38613131_ | 0.000221829 | 5.340676459 | 0.79405103 | −1.259364905 | −1 |
| 38616534_RR | | | | | |
| TOP2B_3_25644985_25663188_25716096_ | 3.83E−05 | 7.803482889 | 1.599505304 | 1.599505304 | 1 |
| 25717154_FF | | | | | |
| VEGFC_4_177629821_177639626_177693384_ | 0.00023044 | 5.235055692 | 1.542011936 | 1.542011936 | 1 |
| 177697283_FR | | | | | |
| VEGFC_4_177629821_177639626_177740221_ | 5.28E−05 | 7.425914159 | 1.446809728 | 1.446809728 | 1 |
| 177743175_FR | | | | | |
| VEGFC_4_177629821_177639626_177693384_ | 1.71E−05 | 9.163763582 | 1.336348688 | 1.336348688 | 1 |
| 177697283_FF | | | | | |
| EZH2_7_148496931_148503515_148602692_ | 0.001181426 | 2.467211314 | 1.165714021 | 1.165714021 | 1 |
| 148606606_FF | | | | | |
| EZH2_7_148496931_148503515_148610251_ | 0.000337006 | 4.53671325 | 1.165112204 | 1.165112204 | 1 |
| 148614284_FR | | | | | |
| SP1_12_53752782_53754759_53771263_ | 0.014464632 | −1.742110313 | 1.146413768 | 1.146413768 | 1 |
| 53775550_RF | | | | | |
| SP1_12_53771263_53775550_53824264_ | 0.02071572 | −2.31009962 | 1.143427617 | 1.143427617 | 1 |
| 53827278_FR | | | | | |
| DAPK1_9_90064560_90073617_90176237_ | 0.00370395 | 0.437247364 | 0.864492054 | −1.156748631 | −1 |
| 90180153_FF | | | | | |
| DAPK1_9_90064560_90073617_90339152_ | 0.034291978 | −3.064141545 | 0.817965763 | −1.222545056 | −1 |
| 90340776_FF | | | | | |
| DAPK1_9_90064560_90073617_90140806_ | 0.000805368 | 3.154114326 | 1.230611817 | 1.230611817 | 1 |
| 90142738_FR | | | | | |
| DAPK1_9_90064560_90073617_90140806_ | 0.031049051 | −2.92175958 | 1.167227549 | 1.167227549 | 1 |
| 90142738_FF | | | | | |
| FGD4_12_32760791_32767406_32781508_ | 0.004709684 | 0.020720467 | 0.82669087 | −1.209642004 | −1 |
| 32786048_FR | | | | | |
| FGD4_12_32760791_32767406_32781508_ | 0.003536515 | 0.541798821 | 0.822250734 | −1.216174043 | −1 |
| 32786048_RR | | | | | |
| FGD4_12_32714978_32722972_32735447_ | 0.005488137 | −0.227642215 | 1.212297233 | 1.212297233 | 1 |
| 32738552_RR | | | | | |
| FGD4_12_32714978_32722972_32768073_ | 0.018634903 | −2.135456788 | 1.168493717 | 1.168493717 | 1 |
| 32772358_RR | | | | | |
| GAB1_4_144235957_144242111_144369687_ | 0.000473286 | 4.021826263 | 1.250307607 | 1.250307607 | 1 |
| 144374560_FF | | | | | |
| GAB1_4_144272552_144276220_144402622_ | 0.002243704 | 1.283544618 | 1.175119334 | 1.175119334 | 1 |
| 144411096_FR | | | | | |
| GAB1_4_144254752_144257034_144321110_ | 0.03218585 | −2.977661138 | 0.874583297 | −1.143401668 | −1 |
| 144332903_RF | | | | | |
| GAB1_4_144298156_144300750_144321110_ | 0.001301693 | 2.25489123 | 0.868489706 | −1.151424126 | −1 |
| 144332903_FR | | | | | |
| HSD3B2_1_119937390_119948935_119959754_ | 0.001671497 | 1.791279777 | 1.154626147 | 1.154626147 | 1 |
| 119963670_FR | | | | | |
| HSD3B2_1_119937390_119948935_119959754_ | 0.002556892 | 1.048050136 | 1.123098683 | 1.123098683 | 1 |
| 119963670_FF | | | | | |
| HSD3B2_1_119912462_119915175_119959754_ | 0.033194645 | −3.020586815 | 0.890025372 | −1.123563476 | −1 |
| 119963670_RR | | | | | |
| KLK2_19_51340459_51344004_51390533_ | 0.004584902 | 0.076963796 | 0.850282306 | −1.176079983 | −1 |
| 51395187_FR | | | | | |
| KLK2_19_51317027_51319938_51340459_ | 0.033974819 | −3.052644106 | 1.196855945 | 1.196855945 | 1 |
| 51344004_FF | | | | | |
| KLK2_19_51317027_51319938_51346270_ | 0.032204968 | −2.979964123 | 1.190153686 | 1.190153686 | 1 |
| 51350944_FF | | | | | |
| MAP3K14_17_43358197_43360790_43375304_ | 0.00777086 | −0.781135175 | 1.170771132 | 1.170771132 | 1 |
| 43380378_FF | | | | | |
| MAP3K14_17_43360790_43364282_43409961_ | 0.010161606 | −1.197399861 | 1.147792913 | 1.147792913 | 1 |
| 43415408_FF | | | | | |
| MAP3K14_17_43358197_43360790_43375304_ | 0.001671497 | 1.792075671 | 0.867161784 | −1.15318735 | −1 |
| 43380378_RR | | | | | |
| MIF_22_24194490_24195811_24245843_ | 6.37E−05 | 7.098970594 | 1.193110598 | 1.193110598 | 1 |
| 24254074_RR | | | | | |
| MIF_22_24206371_24208274_24245843_ | 0.004526272 | 0.107467258 | 1.154360424 | 1.154360424 | 1 |
| 24254074_FR | | | | | |
| MUC1_1_155176403_155179713_155191807_ | 0.001740993 | 1.722065484 | 0.864936775 | −1.156153871 | −1 |
| 155193554_FR | | | | | |
| MUC1_1_155146523_155149986_155191807_ | 0.001993646 | 1.477069135 | 0.859477364 | −1.163497775 | −1 |
| 155193554_FR | | | | | |
| RAD51_15_40972719_40979675_41025213_ | 0.006893753 | −0.607362881 | 1.276811662 | 1.276811662 | 1 |
| 41027977_RF | | | | | |
| RAD51_15_40937212_40938851_41025213_ | 0.007053284 | −0.641225269 | 0.845275991 | −1.183045551 | −1 |
| 41027977_RF | | | | | |
| RAD51_15_41009919_41011826_41025213_ | 0.001240188 | 2.34597507 | 0.844458518 | −1.184190791 | −1 |
| 41027977_RF | | | | | |
| RNASEL_1_182541376_182556600_182605098_ | 0.016960458 | −1.988638304 | 1.204553012 | 1.204553012 | 1 |
| 182607856_FR | | | | | |
| RNASEL_1_182541376_182556600_182577916_ | 0.03489118 | −3.090150814 | 1.195077211 | 1.195077211 | 1 |
| 182584530_FF | | | | | |
| SRC_20_35928873_35935451_35989678_ | 0.001394478 | 2.146589494 | 1.153898277 | 1.153898277 | 1 |
| 35993330_FR | | | | | |
| SRC_20_35928873_35935451_35989678_ | 0.001334586 | 2.218375228 | 1.147569522 | 1.147569522 | 1 |
| 35993330_FF | | | | | |
| SRD5A3_4_56188038_56191526_56242301_ | 0.005815136 | −0.328887879 | 1.203296575 | 1.203296575 | 1 |
| 56245314_RF | | | | | |
| SRD5A3_4_56209429_56213336_56242301_ | 0.005781472 | −0.317283393 | 1.180499078 | 1.180499078 | 1 |
| 56245314_RF | | | | | |
| WNT1_12_49327866_49332429_49386082_ | 0.014255673 | −1.715014756 | 1.126134949 | 1.126134949 | 1 |
| 49387249_RF | | | | | |
| WNT1_12_49361168_49364315_49377006_ | 0.001476292 | 2.0340141 | 0.877360306 | −1.139782588 | −1 |
| 49380965_RF | | | | | |
| WNT1_12_49327866_49332429_49364343_ | 9.95E−05 | 6.406185639 | 0.81846229 | −1.221803389 | −1 |
| 49365445_FF | | | | | |

Abbreviations.
logFC: logarithm of the fold change;
AveExpr: Average expression;
adj.P-Val: Adjusted p-value;
B: B-statistic (log-odds that that gene is differentially expressed);
FC: Fold change;
FC_1: Fold change centered around 1;
Binary: Binary call for loop presence/absence.

TABLE 20
DLBCL cell lines used in this study.
Cell lines were obtained from the American
Type Culture Collection (ATCC),
the German Collection of Microorganisms and
Cell Cultures (DSMZ), and the Japan Health
Sciences Foundation Resource Bank (JHSF).

| | | | DLBCL |
|---|---|---|---|
| Cell Line | Cat# | Source | Subtype |
| Toledo | CRL-2631 | ATCC | ABC |
| Pfeiffer | CRL-2632 | ATCC | ABC |
| RC-K8 | ACC 561 | DSMZ | ABC |
| Ri-1 | ACC 585 | DSMZ | ABC |
| A4/Fukada | JCRB 0097 | JHSF | ABC |
| A3/Kawakami | JCRB 0101 | JHSF | ABC |
| RL | CRL 2261 | ATCC | GBC |
| HT | CRL-2260 | ATCC | GBC |
| DB | CRL-2289 | ATCC | GBC |
| Karpas-422 | ACC 32 | DSMZ | GBC |
| SU-DHL-10 | ACC 576 | DSMZ | GBC |
| SU-DHL-16 | ACC 577 | DSMZ | GBC |

TABLE 21
The 97 genomic
loci used in the
initial biomarker
discovery screen.
Gene Symbol
ABCG2
ANXA11
ARRB2
ASPSCR1
BAX
BBS9
BCL2A1
BCL2L10
BCL6
BRCA1
BTK
C13orf34
C15orf55
c21orf45
CABLES1
CAMK2D
CASP10
CASP3
CCR9
CD22
CD40
CD80
CDKN2C
CREB3L2
CTNNB1
CXCL8
DBF4
ERC1
ETV6
FCGR2A
FOS
FOXO1
FOXP1
FRAP1
FZD10
GATA4
GDF6
GRAP2
HLF
IFNAR1
IL-10
IL10RA
IL-15
IL22RA1
IL-2RA
IL-2RB
IL-6
IL-7
ITGA5
ITPR3
JAK3
JDP2
LRP6
MAP3K7
MAPK10
MAPK13
MEF2B
MLLT3
MMP14
MMP2
MMP9
MTHFR
MyD88
NAE1
NCKIPSD
NFATc1
NFKB1
NFKB2
NFKBIA
NFKBIB
PAK1
PCDHGA6/B2/B4
PIK3CG
PIM1
PRDM1
PRKCZ
PTGS2
RBL1
RCA1
REL
SFPQ
SIAH1
SIRT1
SKP1
SOCS7
STAT3
TAL2
TET2
TLR4
TNF
TNFRSF12A
TNFRSF13B
TNFRSF13C
TOP1
WNT11
WNT9A
ZMYM3

TABLE 22
Composite prediction probabilities for the DLBCL-CCS in the Discovery cohort.

| | | EpiSwitch_ | | | EpiSwitch_ |
|---|---|---|---|---|---|
| | | ABC_GCB_ | | | ABC_GCB_ |
| Patient_ID | Class | Prob | Patient_ID | Class | Prob |
| G349424 | ABC | 0.3932724 | G680501 | GCB | 0.8140926 |
| G [symbol] 1 | ABC | 0.20099 | G832590 | GCB | 0.813454 |
| G779156 | ABC | 0.2130736 | G139629 | GCB | [symbol] |
| G426812 | ABC | 0.232716 | G2547772 | GCB | [symbol] |
| G486618 | ABC | 0.2139196 | G [symbol] | GCB | [symbol] |
| G268685 | ABC | [symbol] | G3383 | GCB | 0.8038896 |
| GA83940 | ABC | 0.2166548 | G694634 | GCB | 0.7945872 |
| G635399 | ABC | 0.216758 | G158912 | GCB | 0.7817138 |
| G61310 | ABC | 0.218381 | G833689 | GCB | 0.780263 |
| G936357 | ABC | 0.2184674 | G259245 | GCB | 0.7739536 |
| G232946 | ABC | 0.2190392 | G81760 | GCB | 0.7730372 |
| G783641 | ABC | 0.2227972 | G547813 | GCB | 0.7723438 |
| G421631 | ABC | 0.223797 | G913775 | GCB | [symbol] |
| G707842 | ABC | 0.225338 | G474390 | GCB | 0.7670176 |
| G78398 | ABC | 0.2266852 | G213298 | GCB | 0.763692 |
| G655052 | ABC | 0.2267892 | G153040 | GCB | 0.762577 |
| G986511 | ABC | 0.2373018 | G106325 | GCB | 0.762146 |
| G529836 | ABC | 0.2380336 | G243731 | GCB | 0.7621028 |
| G373235 | ABC | [symbol] | G819965 | GCB | 0.7593464 |
| G418054 | ABC | 0.2411416 | G701045 | GCB | 0.7575728 |
| G [symbol] | ABC | 0.2428652 | G544037 | GCB | 0.756705 |
| G416338 | ABC | 0.2478544 | G370848 | GCB | 0.7554284 |
| G175733 | ABC | 0.2580098 | G739109 | GCB | 0.754522 |
| G852875 | ABC | 0.2596216 | [symbol] | GCB | 0.7539074 |
| G292009 | ABC | 0.2633818 | G779214 | GCB | 0.7536828 |
| G52801 | ABC | 0.2763932 | G572374 | GCB | 0.7526758 |
| G544695 | ABC | 0.276862 | G901049 | GCB | 0.7491072 |
| G [symbol] | ABC | 0.2856876 | G937404 | GCB | 0.7285326 |
| G181400 | ABC | 0.297829 | G254120 | GCB | 0.7000562 |
| GA18564 | ABC | 0.2980288 | G557427 | GCB | 0.6935706 |

[symbol]

indicates data missing or illegible when filed

TABLE 23
DLBCL-CCS and Fluidigm subtype calls in the Discovery cohort. Subtype calls made by
the EpiSwitch DLBCL-CCS and the Fluidigm assays on samples of known DLBCL
subtypes. 60 out of 60 samples were identically called as ABC or GCB by both assays.

| ABC | | | GBC | | |
|---|---|---|---|---|---|
| Patient ID | Fluidigm | EpiSwitch | Patient ID | Fluidigm | EpiSwitch |
| RG332787 | ABC | ABC | RG949161 | GCB | GCB |
| RG857282 | ABC | ABC | RG552773 | GCB | GCB |
| RG639274 | ABC | ABC | RG385960 | GCB | GCB |
| RG227462 | ABC | ABC | RG713290 | GCB | GCB |
| RG898976 | ABC | ABC | RG874071 | GCB | GCB |
| RG469063 | ABC | ABC | RG475279 | GCB | GCB |
| RG235350 | ABC | ABC | RG885516 | GCB | GCB |
| RG341829 | ABC | ABC | RG681434 | GCB | GCB |
| RG769788 | ABC | ABC | RG231526 | GCB | GCB |
| RG401919 | ABC | ABC | RG855093 | GCB | GCB |
| RG849927 | ABC | ABC | RG458634 | GCB | GCB |
| RG714326 | ABC | ABC | RG279476 | GCB | GCB |
| RG109735 | ABC | ABC | RG373871 | GCB | GCB |
| RG563907 | ABC | ABC | RG853726 | GCB | GCB |
| RG847865 | ABC | ABC | RG101525 | GCB | GCB |
| RG698196 | ABC | ABC | RG525277 | GCB | GCB |
| RG208608 | ABC | ABC | RG954268 | GCB | GCB |
| RG126501 | ABC | ABC | RG521469 | GCB | GCB |
| RG988758 | ABC | ABC | RG673708 | GCB | GCB |
| RG436104 | ABC | ABC | RG386174 | GCB | GCB |
| RG611396 | ABC | ABC | RG380741 | GCB | GCB |
| RG565461 | ABC | ABC | RG132060 | GCB | GCB |
| RG513781 | ABC | ABC | RG118710 | GCB | GCB |
| RG549011 | ABC | ABC | RG578086 | GCB | GCB |
| RG233693 | ABC | ABC | RG542280 | GCB | GCB |
| RG192075 | ABC | ABC | RG313590 | GCB | GCB |
| RG374916 | ABC | ABC | RG387871 | GCB | GCB |
| RG410219 | ABC | ABC | RG108874 | GCB | GCB |
| RG216984 | ABC | ABC | RG883839 | GCB | GCB |
| RG538574 | ABC | ABC | RG489043 | GCB | GCB |

TABLE 24
Enrichment of biological functions in the top 10 DLBCL-CCS loci.

| Analysis | Pathway | Pathway | Gene | | Matching |
|---|---|---|---|---|---|
| Type | ID | Description | Count | FDR | Proteins |
| GO-Molecular | GO.0001228 | transcriptional | 4 | 0.0365 | M [symbol] 28, NFATC1, |
| Function | | activator activity, | | | NFKB1, STAT3 |
| | | RNA polymerase | | | |
| | | II transcription | | | |
| GO-Biological | GO.0045893 | regulatory region | 7 | 0.00187 | CD40, IFNAR1, |
| Process | | positive regulation | | | MAP3K7, M [symbol] 2B, |
| | | of transcription, | | | NFATC1, NFKB1, |
| | | DNA-templated | | | STAT3 |
| KEGG Pathway | 4620 | Toll-like | 4 | 2.21E−05 | CD40, |
| | | receptor | | | IFNAR1, |
| | | signaling | | | MAP3K7, |
| | | pathway | | | NFKB1 |

[symbol]

indicates data missing or illegible when filed

TABLE 25.a

| | Marker Details | | | |
|---|---|---|---|---|
| | | Genome | | |
| | | Mapped | | |
| No. | Type | to | Probe | GeneLocus |
| 1 | Diagnostic | GRCh37 | ETS1_11_128419843_128421939_128481262_128489818_RR | ETS1 |
| 2 | Diagnostic | GRCh37 | SLC22A3_6_160805748_160812960_160839018_160842982_RR | SLC22A3 |
| 3 | Diagnostic | GRCh37 | SLC22A3_6_160805748_160812960_160884099_160888471_RR | SLC22A3 |
| 4 | Diagnostic | GRCh37 | MAP3K14_17_43360790_43364282_43409961_43415408_FR | MAP3K14 |
| 5 | Diagnostic | GRCh37 | CASP2_7_142940014_142947169_142963973_142967512_FR | CASP2 |
| 6 | Prognostic 3v1 | GRCh37 | BMP6_6_7724582_7733496_7801590_7806316_FF | BMP6 |
| 7 | Prognostic 3v1 | GRCh37 | ACAT1_11_107955219_107960166_108013361_108018367_FF | ACAT1 |
| 8 | Prognostic 3v1 | GRCh37 | ERG_21_39895678_39899145_39984806_39991905_RF | ERG |
| 9 | Prognostic 3v1 | GRCh37 | MSR1_8_16195878_16203315_16396849_16400398_FF | MSR1 |
| 10 | Prognostic 3v1 | GRCh37 | MUC1_1_155146523_155149986_155191807_155193554_FR | MUC1 |
| 11 | Prognostic 3v1 | GRCh37 | DAPK1_9_90064560_90073617_90140806_90142738_FR | DAPK1 |
| 12 | Prognostic 3v2 | GRCh37 | ACAT1_11_107955219_107960166_108013361_108018367_FF | ACAT1 |
| 13 | Prognostic 3v2 | GRCh37 | MUC1_1_155146523_155149986_155191807_155193554_FR | MUC1 |
| 14 | Prognostic 3v2 | GRCh37 | DAPK1_9_90064560_90073617_90140806_90142738_FR | DAPK1 |
| 15 | Prognostic 3v2 | GRCh37 | APAF1_12_99061113_99062942_99098781_99108240_FF | APAF1 |
| 16 | Prognostic 3v2 | GRCh37 | HSD3B2_1_119912462_119915175_119959754_119963670_RR | HSD3B2 |
| 17 | Prognostic 3v2 | GRCh37 | VEGFC_4_177629821_177639626_177740221_177743175_FR | VEGFC |

TABLE 25.b

| | Hyper G array stats | | | | | | |
|---|---|---|---|---|---|---|---|
| | Probe_ | Probe_ | | | | | |
| | Count_ | Count_ | HyperG_ | FDR_ | Percent_ | Microarray output | |
| No. | Total | Sig | Stats | HyperG | Sig | logFC | AveExpr |
| 1 | 100 | 22 | 0.143767534 | 0.706164223 | 22 | 0.788832719 | 0.788832719 |
| 2 | 54 | 16 | 0.019214151 | 0.218625878 | 29.63 | 0.739725229 | 0.739725229 |
| 3 | 54 | 16 | 0.019214151 | 0.218625878 | 29.63 | 0.729027457 | 0.729027457 |
| 4 | 11 | 5 | 0.029574086 | 0.259389379 | 45.45 | 0.735407293 | 0.735407293 |
| 5 | 13 | 3 | 0.402919615 | 1 | 23.08 | −0.469997725 | −0.469997725 |
| 6 | 69 | 8 | 0.366815399 | 1 | 11.59 | −0.468602239 | −0.468602239 |
| 7 | 15 | 2 | 0.441893041 | 1 | 13.33 | −0.436725529 | −0.436725529 |
| 8 | 52 | 4 | 0.765503518 | 1 | 7.69 | −0.425291613 | −0.425291613 |
| 9 | 191 | 41 | 1.07E−06 | 0.000448644 | 21.47 | −0.419369028 | −0.419369028 |
| 10 | 5 | 3 | 0.008132099 | 0.285301135 | 60 | −0.218468452 | −0.218468452 |
| 11 | 46 | 9 | 0.032709022 | 0.548212211 | 19.57 | 0.299375751 | 0.299375751 |
| 12 | 15 | 2 | 0.441893041 | 1 | 13.33 | −0.436725529 | −0.436725529 |
| 13 | 5 | 3 | 0.008132099 | 0.285301135 | 60 | −0.218468452 | −0.218468452 |
| 14 | 46 | 9 | 0.032709022 | 0.548212211 | 19.57 | 0.299375751 | 0.299375751 |
| 15 | 10 | 1 | 0.644810187 | 1 | 10 | −0.441488336 | −0.441488336 |
| 16 | 20 | 5 | 0.040338404 | 0.548212211 | 25 | −0.168081632 | −0.168081632 |
| 17 | 57 | 16 | 8.02E−05 | 0.006755982 | 28.07 | 0.532875204 | 0.532875204 |

TABLE 25.c

| | Microarray output | | | | | |
|---|---|---|---|---|---|---|
| No. | T | P.Value | adj.P.Val | B | FC | FC_1 |
| 1 | 15.59116667 | 0.0000000108 | 0.00000135 | 10.64875918 | 1.727676038 | 1.727676038 |
| 2 | 18.80485468 | 0.00000000155 | 0.00000124 | 12.53177853 | 1.669857773 | 1.669857773 |
| 3 | 19.34951235 | 0.00000000115 | 0.00000112 | 12.81371179 | 1.657521354 | 1.657521354 |
| 4 | 15.29282549 | 0.0000000131 | 0.00000138 | 10.45220415 | 1.664867419 | 1.664867419 |
| 5 | −13.28933415 | 0.000000055 | 0.00000252 | 9.016293707 | 0.721965736 | −1.385107284 |
| 6 | −8.973309325 | 0.00000230 | 0.000229279 | 5.288915333 | 0.722664415 | −1.383768149 |
| 7 | −11.90723067 | 0.000000137 | 0.0000310 | 8.114204006 | 0.738809576 | −1.353528748 |
| 8 | −11.67074071 | 0.000000168 | 0.0000357 | 7.913111034 | 0.744688192 | −1.342843905 |
| 9 | −3.516010141 | 0.004895359 | 0.024952597 | −2.59289798 | 0.747751587 | −1.337342532 |
| 10 | −5.993061073 | 0.0000935 | 0.001993646 | 1.477069135 | 0.859477364 | −1.163497775 |
| 11 | 7.197207444 | 0.0000184 | 0.000805368 | 3.154114326 | 1.230611817 | 1.230611817 |
| 12 | −11.90723067 | 0.000000137 | 0.0000310 | 8.114204006 | 0.738809576 | −1.353528748 |
| 13 | −5.993061073 | 0.0000935 | 0.001993646 | 1.477069135 | 0.859477364 | −1.163497775 |
| 14 | 7.197207444 | 0.0000184 | 0.000805368 | 3.154114326 | 1.230611817 | 1.230611817 |
| 15 | −13.23940926 | 0.0000000463 | 0.0000171 | 9.174110234 | 0.736374546 | −1.358004571 |
| 16 | −3.274998031 | 0.007481356 | 0.033194645 | −3.020586815 | 0.890025372 | −1.123563476 |
| 17 | 11.11732726 | 0.000000275 | 0.0000528 | 7.425914159 | 1.446809728 | 1.446809728 |

TABLE 25.d

| | Microarray output | | | |
|---|---|---|---|---|
| | | Loop | Probe sequence | SEQ ID |
| No. | LS | Detected | 60 mer | NO: |
| 1 | 1 | Pea | CCATGGTGTG | 452 |
| | | | AGTGTGGATT | |
| | | | TAGGTGAATC | |
| | | | GAAAGATCTA | |
| | | | GTAGGTTCTG | |
| | | | TCCAGACTGT | |
| 2 | 1 | Pea | AATTCTGAGG | 453 |
| | | | GTGGAAGGAA | |
| | | | GGTGGGAGTC | |
| | | | GATGGCTCTT | |
| | | | ATGCAGCATT | |
| | | | ATTTATCAAT | |
| 3 | 1 | Pea | AATTCTGAGG | 454 |
| | | | GTGGAAGGAA | |
| | | | GGTGGGAGTC | |
| | | | GAGGGACTTT | |
| | | | CAGGTAGAGG | |
| | | | AGCCACCAAG | |
| 4 | 1 | Pea | AGGGGCTGAT | 455 |
| | | | CAGTTTGTGG | |
| | | | AGTTCTGATC | |
| | | | GAGGGAGAGG | |
| | | | AGTGGCAGTG | |
| | | | GGGGAGTGGA | |
| 5 | −1 | HC | TCCAGAAGCT | 456 |
| | | | GAGCTTGAGC | |
| | | | CAAGGTGTTC | |
| | | | GAACTCCTGG | |
| | | | GCTGAAGCAA | |
| | | | TCTCCTGCCT | |
| 6 | −1 | 2_3 | ACGTCGTTAC | 457 |
| | | | AGTTTTAATT | |
| | | | TTTCTACTTC | |
| | | | GATGTTAATC | |
| | | | TCCTAAAAAA | |
| | | | CATCCAACCA | |
| 7 | −1 | 1 | CAATTGGTGG | 458 |
| | | | ATATAGAAAG | |
| | | | GTCTAAATTC | |
| | | | GATAAGTATA | |
| | | | GACTCAGAAT | |
| | | | GCAAAAATGT | |
| 8 | −1 | 2_3 | TCTTGAATGT | 459 |
| | | | GCTTAGTATT | |
| | | | ATTCAGACTC | |
| | | | GAAAACATAA | |
| | | | TTTGAAAGGA | |
| | | | ATTCATTCTG | |
| 9 | −1 | 2_3 | CACCAGTTGG | 460 |
| | | | TAATTCTATG | |
| | | | TGTAAGTTTC | |
| | | | GAGCTTATAA | |
| | | | GATCAATCAG | |
| | | | GAATTATTCC | |
| 10 | −1 | 3 | GCAGGGTGGC | 461 |
| | | | TATAGCTCAG | |
| | | | GAGAGTGCTC | |
| | | | GACGGAGTCT | |
| | | | TGCTCTTTCA | |
| | | | CCCAGGCTGG | |
| 11 | 1 | 3 | ACTAATCCCC | 462 |
| | | | TGAAGAAGCA | |
| | | | AATTAACTTC | |
| | | | GAGTATCCCT | |
| | | | TTAAGTTTGT | |
| | | | TTTTAAAATA | |
| 12 | −1 | 1 | CAATTGGTGG | 463 |
| | | | ATATAGAAAG | |
| | | | GTCTAAATTC | |
| | | | GATAAGTATA | |
| | | | GACTCAGAAT | |
| | | | GCAAAAATGT | |
| 13 | −1 | 3 | GCAGGGTGGC | 464 |
| | | | TATAGCTCAG | |
| | | | GAGAGTGCTC | |
| | | | GACGGAGTCT | |
| | | | TGCTCTTTCA | |
| | | | CCCAGGCTGG | |
| 14 | 1 | 3 | ACTAATCCCC | 465 |
| | | | TGAAGAAGCA | |
| | | | AATTAACTTC | |
| | | | GAGTATCCCT | |
| | | | TTAAGTTTGT | |
| | | | TTTTAAAATA | |
| 15 | −1 | 3 | CCTAATTTAC | 466 |
| | | | TTAACCAAAC | |
| | | | TCTAGTTATC | |
| | | | GAACATCCAG | |
| | | | GATGTTATAA | |
| | | | GAATTCAATG | |
| 16 | −1 | 3 | TCAGTTTCTGC | 467 |
| | | | TCTCAAGAAG | |
| | | | CTTACAGTCG | |
| | | | AAGGTCCCAA | |
| | | | GTTAGATTAC | |
| | | | GGCAAAGCT | |
| 17 | 1 | 3 | TTTTATGAAA | 468 |
| | | | CATCCAACTT | |
| | | | AAATATAATC | |
| | | | GAATGCATTA | |
| | | | CATTTACAGA | |
| | | | ACTATTTCCA | |

TABLE 25.e

| | Probe Location | | | | | 4 kb Sequence Location | | | |
|---|---|---|---|---|---|---|---|---|---|
| No | Chr | Start1 | End1 | Start2 | End2 | Chr | Start1 | End1 | Start2 |
| 1 | 11 | 128419843 | 128419873 | 128481262 | 128481292 | 11 | 128419843 | 128423843 | 128481262 |
| 2 | 6 | 160805748 | 160805778 | 160839018 | 160839048 | 6 | 160805748 | 160809748 | 160839018 |
| 3 | 6 | 160805748 | 160805778 | 160884099 | 160884129 | 6 | 160805748 | 160809748 | 160884099 |
| 4 | 17 | 43364251 | 43364282 | 43409961 | 43409991 | 17 | 43360281 | 43364282 | 43409961 |
| 5 | 7 | 142947138 | 142947169 | 142963973 | 142964003 | 7 | 142943168 | 142947169 | 142963973 |
| 6 | 6 | 7733465 | 7733496 | 7806286 | 7806316 | 6 | 7729496 | 7733496 | 7802316 |
| 7 | 11 | 107960135 | 107960166 | 108018337 | 108018367 | 11 | 107956166 | 107960166 | 108014367 |
| 8 | 21 | 39895678 | 39895708 | 39991875 | 39991905 | 21 | 39895678 | 39899678 | 39987905 |
| 9 | 8 | 16203284 | 16203315 | 16400368 | 16400398 | 8 | 16199315 | 16203315 | 16396398 |
| 10 | 1 | 155149955 | 155149986 | 155191807 | 155191837 | 1 | 155145986 | 155149986 | 155191807 |
| 11 | 9 | 90073586 | 90073617 | 90140806 | 90140836 | 9 | 90069617 | 90073617 | 90140806 |
| 12 | 11 | 107960135 | 107960166 | 108018337 | 108018367 | 11 | 107956166 | 107960166 | 108014367 |
| 13 | 1 | 155149955 | 155149986 | 155191807 | 155191837 | 1 | 155145986 | 155149986 | 155191807 |
| 14 | 9 | 90073586 | 90073617 | 90140806 | 90140836 | 9 | 90069617 | 90073617 | 90140806 |
| 15 | 12 | 99062911 | 99062942 | 99108209 | 99108240 | 12 | 99058941 | 99062942 | 99104239 |
| 16 | 1 | 119912462 | 119912492 | 119959754 | 119959784 | 1 | 119912462 | 119916462 | 119959754 |
| 17 | 4 | 177639595 | 177639626 | 177740221 | 177740251 | 4 | 177635625 | 177639626 | 177740221 |

TABLE 25.f

| | 4 kb Sequence Location | | Inner_primers |
|---|---|---|---|
| No. | End2 | Probe | PCR-Primer1_ID |
| 1 | 128485262 | ETS1_11_128419843_128421939_128481262_128489818_RR | PCa-57 |
| 2 | 160843018 | SLC22A3_6_160805748_160812960_160839018_160842982_RR | PCa-73 |
| 3 | 160888099 | SLC22A3_6_160805748_160812960_160884099_160888471_RR | PCa-77 |
| 4 | 43413961 | MAP3K14_17_43360790_43364282_43409961_43415408_FR | PCa-81 |
| 5 | 142967973 | CASP2_7_142940014_142947169_142963973_142967512_FR | PCa-189 |
| 6 | 7806316 | BMP6_6_7724582_7733496_7801590_7806316_FF | PCa119-37 |
| 7 | 108018367 | ACAT1_11_107955219_107960166_108013361_108018367_FF | PCa119-57 |
| 8 | 39991905 | ERG_21_39895678_39899145_39984806_39991905_RF | PCa119-65 |
| 9 | 16400398 | MSR1_8_16195878_16203315_16396849_16400398_FF | PCa119-77 |
| 10 | 155195807 | MUC1_1_155146523_155149986_155191807_155193554_FR | PCa119-121 |
| 11 | 90144806 | DAPK1_9_90064560_90073617_90140806_90142738_FR | PCa119-165 |
| 12 | 108018367 | ACAT1_11_107955219_107960166_108013361_108018367_FF | PCa119-57 |
| 13 | 155195807 | MUC1_1_155146523_155149986_155191807_155193554_FR | PCa119-121 |
| 14 | 90144806 | DAPK1_9_90064560_90073617_90140806_90142738_FR | PCa119-165 |
| 15 | 99108240 | APAF1_12_99061113_99062942_99098781_99108240_FF | PCa119-49 |
| 16 | 119963754 | HSD3B2_1_119912462_119915175_119959754_119963670_RR | PCa119-129 |
| 17 | 177744221 | VEGFC_4_177629821_177639626_177740221_177743175_FR | PCa119-205 |

TABLE 25.g

| | | Inner_primers | | | |
|---|---|---|---|---|---|
| | | | | | SEQ |
| | SEQ | | PCR- | | ID |
| | ID | | Primer2_ | PCR_ | NO: |
| No. | NO: | PCR_Primer1 | ID | Primer2 | |
| 1 | 469 | CACTGCATGA | PCa-59 | CCTCTGTCTG | 486 |
| | | GGGTAGTATA | | CATCATACC | |
| | | G | | | |
| 2 | 470 | TGATGAGGCA | PCa-75 | ACACGCCCAG | 487 |
| | | CACAGATAAA | | AAACAATAC | |
| | | G | | | |
| 3 | 471 | GAGACATGAT | PCa-79 | GTGTGAGTTG | 488 |
| | | GAGGCACAC | | ATAGCTGACC | |
| 4 | 472 | TGGAATGGGA | PCa-83 | GAGACTCCAG | 489 |
| | | AGGGATGAG | | GCAAGAATTT | |
| | | | | G | |
| 5 | 473 | ATGAAGACAG | PCa-191 | CAGTGGAACT | 490 |
| | | AAAGCCTATG | | TCCTGAGAAC | |
| | | G | | | |
| 6 | 474 | CGGCCAGGAA | PCa119-39 | GTAAGCGAGG | 491 |
| | | TGACTATTG | | TCATCATAGA | |
| | | | | AG | |
| 7 | 475 | AGTAGTGTAT | PCa119-59 | TCTTGGTAAC | 492 |
| | | CAGGACTGGG | | CTTGAAAAGT | |
| | | T | | TTGAT | |
| 8 | 476 | CAGCCTACCT | PCa119-67 | ATGGGCCATC | 493 |
| | | TGCCTGACAC | | ACTGGGCTTT | |
| | | T | | | |
| 9 | 477 | AATCCTCTTG | PCa119-79 | TAGGCCCAAA | 494 |
| | | AGCACAGACC | | TGGCTCAC | |
| 10 | 478 | TGTTGCTAGC | PCa119-123 | AGATCAAGCC | 495 |
| | | TCAGGAAGCC | | ACTGTGCTCC | |
| 11 | 479 | ACTGGTCACA | PCa119-167 | AGGTGTGAAT | 496 |
| | | GGGAACGATG | | GTTACTGAAC | |
| | | G | | ACAAA | |
| 12 | 480 | AGTAGTGTAT | PCa119-59 | TCTTGGTAAC | 497 |
| | | CAGGACTGGG | | CTTGAAAAGT | |
| | | T | | TTGAT | |
| 13 | 481 | TGTTGCTAGC | PCa119-123 | AGATCAAGCC | 498 |
| | | TCAGGAAGCC | | ACTGTGCTCC | |
| 14 | 482 | ACTGGTCACA | PCa119-167 | AGGTGTGAAT | 499 |
| | | GGGAACGATG | | GTTACTGAAC | |
| | | G | | ACAAA | |
| 15 | 483 | GGTATTCCAA | PCa119-51 | TACTGTGCCA | 500 |
| | | TAAATACTTG | | GATGCTCTCA | |
| | | TGCCC | | | |
| 16 | 484 | TCACATCAGT | PCa119-131 | GGAGGGAGGC | 501 |
| | | TTCTGCTCTC | | TCAGAGAAGC | |
| | | AAG | | | |
| 17 | 485 | TCTCTGACTG | PCa119-207 | CTCCTTCTAC | 502 |
| | | CAGTGCAAAA | | ATTCACGTGC | |
| | | TAAT | | TTTCA | |

TABLE 25.h

| | PCR Stats | | |
|---|---|---|---|
| No. | Gene | Marker | GLMNET |
| 1 | ETS1 | Pca-57.59 | −0.00000007417665 |
| 2 | SLC22A3 | Pca-73.75 | 0.00000001852548 |
| 3 | SLC22A3 | Pca-77.79 | 0.00000002568381 |
| 4 | MAP3K14 | Pca-81.83 | 0.00000001902257 |
| 5 | CASP2 | Pca-189.191 | 0.0000001325828 |
| 6 | BMP6 | PCa-119-37.39 | 0.000009609007 |
| 7 | ACAT1 | PCa-119-57.59 | 0.000004371579 |
| 8 | ERG | PCa-119-65.67 | 0.000006321361 |
| 9 | MSR1 | PCa-119-77.79 | 0.000005500154 |
| 10 | MUC1 | PCa-119-121.123 | 0.00000006234414 |
| 11 | DAPK1 | PCa-119-165.167 | −0.00001571847 |
| 12 | ACAT1 | PCa-119-57.59 | 0.000004371579 |
| 13 | MUC1 | PCa-119-121.123 | 0.00000006234414 |
| 14 | DAPK1 | PCa-119-165.167 | −0.00001571847 |
| 15 | APAF1 | PCa-119-49.51 | 0.000003531754 |
| 16 | HSD3B2 | PCa-119-129.131 | 0.0000004472913 |
| 17 | VEGFC | PCa-119-205.207 | −0.0000006807692 |

1. A process for detecting a chromosome state which represents a subgroup in a population comprising determining whether a chromosome interaction relating to that chromosome state is present or absent within a defined region of the genome; and
wherein the subgroup relates to prognosis for prostate cancer and wherein the chromosome interaction corresponds to any one of the chromosome interactions represented by any probe shown in Table 6,
or
wherein the subgroup relates to prognosis for DLBCL and the chromosome interaction b) corresponds to any one of the chromosome interactions represented by any probe shown in Table 5;
or
wherein the subgroup relates to prognosis for lymphoma and the chromosome interaction corresponds to any one of the chromosome interactions shown in Table 8.

2. A process according to claim 1 wherein:
said prognosis for prostate cancer relates to whether or not the cancer is aggressive or indolent;
and/or
said prognosis for DLBCL relates to survival.

3. A process according to claim 1 wherein the subgroup relates to prostate cancer and a specific combination of chromosome interactions are typed:
(i) comprising all of the chromosome interactions represented by the probes in Table 6; and/or
(ii) comprising at least 1, 2, 3 or 4 of the chromosome interactions represented by the probes in Table 6.

4. A process according to claim 1 wherein the subgroup relates to DLBCL and a specific combination of chromosome interactions are typed:
(i) comprising all of the chromosome interactions represented by the probes in Table 5; and/or
(ii) comprising at least 10, 20, 30, 50 or 80 of the chromosome interactions represented by the probes in Table 5.

5. A process according to claim 1 wherein the subgroup relates to DLBCL and a specific combination of chromosome interactions are typed:
(i) comprising all of the chromosome interactions shown in Table 7; and/or
(ii) comprising at least 1, 2, 5 or 8 of the chromosome interactions shown in Table 7.

6. A process according to claim 1 wherein the subgroup relates to lymphoma and a specific combination of chromosome interactions are typed:
(i) comprising all of the chromosome interactions shown in Table 8; and/or
(ii) comprising at least 10, 20, 30 or 50 of the chromosome interactions shown in Table 8 or preferably a specific combination of chromosome interactions are typed:
(a) comprising all of the chromosome interactions shown in Table 9; and/or
(b) comprising at least 5, 10 or 15 of the chromosome interactions shown in Table 9.

7. A process according to claim 1 wherein at least 10, 20, 30, 40 or 50, chromosome interactions are typed, and preferably at least 10 chromosome interactions are typed.

8. A process according to claim 1 in which the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, wherein detection of the ligated nucleic acid is preferably by:
(i) in the case of prognosis of prostate cancer by a probe that has at least 70% identity to any of the specific probe sequences mentioned in Table 6, and/or (ii) by a primer pair which has at least 70% identity to any primer pair in Table 6; or
(ii) in the case of prognosis of DLBCL a probe that has at least 70% identity to any of the specific probe sequences mentioned in Table 5, and/or (b) by a primer pair which has at least 70% identity to any primer pair in Table 5.

9. A process according to claim 1 in which the chromosome interactions are typed by detecting the presence of a ligated nucleic acid which is generated during said typing and whose sequence comprises two regions each corresponding to the regions of the chromosome which come together in the chromosome interaction, wherein detection of the ligated nucleic acid in the case of prognosis of lymphoma is by:
a probe that has at least 70% identity to any of the specific probe sequences mentioned in Table 5, and/or
by a primer pair which has at least 70% identity to any primer pair in Table 5, and/or
by a primer pair which has at least 70% identify to any primer pair in Table 8.

10-11. (canceled)

12. A process according to claim 1, wherein the chromosome interaction is detected by a method comprising the steps of: —
(i) cross-linking of chromosome regions which have come together in a chromosome interaction;
(ii) subjecting said cross-linked regions to cleavage, optionally by restriction digestion cleavage with an enzyme; and
(iii) ligating said cross-linked cleaved DNA ends to form the first set of nucleic acids and
(iv) detecting the presence or absence of a ligated nucleic acid corresponding to the chromosome interaction.

13. (canceled)

14. A process according to claim 1 which is carried out to determine whether a prostate cancer is aggressive or indolent which comprises typing at least 5 chromosome interactions as defined in Table 6.

15. A process according to claim 1 which is carried out to determine prognosis of DLBLC which comprises typing at least 5 chromosome interactions as defined in Table 5.

16. A process according to claim 1 which is carried out to identify or design a therapeutic agent for prostate cancer;
wherein preferably said process is used to detect whether a candidate agent is able to cause a change to a chromosome state which is associated with a different level of prognosis;
wherein the chromosomal interaction is represented by any probe in Table 6; and/or
the chromosomal interaction is present in any region or gene listed in Table 6;
and wherein optionally:
the chromosomal interaction has been identified by the method of determining which chromosomal interactions are relevant to a chromosome state as defined in claim 1, and/or
the change in chromosomal interaction is monitored using (i) a probe that has at least 70% identity to any of the probe sequences mentioned in Table 6, and/or (ii) by a primer pair which has at least 70% identity to any primer pair in Table 6.

17. A process according to claim 1 which is carried out to identify or design a therapeutic agent for DLBCL;
wherein preferably said process is used to detect whether a candidate agent is able to cause a change to a chromosome state which is associated with a different level of prognosis;
wherein the chromosomal interaction is represented by any probe in Table 5; and/or
the chromosomal interaction is present in any region or gene listed in Table 5;
and wherein optionally:
the chromosomal interaction has been identified by the method of determining which chromosomal interactions are relevant to a chromosome state as defined in claim 1, and/or
the change in chromosomal interaction is monitored using (i) a probe that has at least 70% identity to any of the probe sequences mentioned in Table 5, and/or (ii) by a primer pair which has at least 70% identity to any primer pair in Table 5.

18. A process according to claim 1 to 15 which is carried out to identify or design a therapeutic agent for lymphoma;
wherein preferably said process is used to detect whether a candidate agent is able to cause a change to a chromosome state which is associated with a different level of prognosis;
wherein the chromosomal interaction is represented by any probe in Table 8 or 9; and/or
the chromosomal interaction is present in any region or gene listed in Table 8 or 9;
and wherein optionally:
the chromosomal interaction has been identified by the method of determining which chromosomal interactions are relevant to a chromosome state as defined in claim 1, and/or
the change in chromosomal interaction is monitored using (i) a probe that has at least 70% identity to any of the probe sequences mentioned in Table 5, and/or (ii) by a primer pair which has at least 70% identity to any primer pair in Table 5 or 8.

19. A process according to claim 1 which comprises selecting a target based on detection of the chromosome interactions, and preferably screening for a modulator of the target to identify a therapeutic agent for immunotherapy, wherein said target is optionally a protein.

20. A process according to claim 1 wherein said prognosis is in a human or canine.

21. A process according to claim 1, wherein the typing or detecting comprises specific detection of the ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:
an oligonucleotide which specifically binds to said ligated product, and/or
a fluorophore covalently attached to the 5′ end of the oligonucleotide, and/or
a quencher covalently attached to the 3′ end of the oligonucleotide, and optionally
said fluorophore is selected from HEX, Texas Red and FAM; and/or
said probe comprises a nucleic acid sequence of length 10 to 40 nucleotide bases, preferably a length of 20 to 30 nucleotide bases.

22. A process according to claim 1 wherein:
the result of the process is provided in a report, and/or
the result of the process is used to select a patient treatment schedule, and preferably to select a specific therapy for the individual.

23. A method of treating prostate cancer, DLBCL or lymphoma in an individual that has been identified as being in need of treatment by a process according to claim 1, comprising administering to the individual a therapeutic agent for prostate cancer, DLBCL or lymphoma.

24. A process according to claim 1 wherein:
the subgroup relates to prostate cancer and at least one chromosome interaction from Table 25 is typed; and/or
the subgroup relates to prostate cancer and at least one of the following combinations of interactions from Table 25 is typed:
(i) ETS1, MAP3K14, SLC22A3 and CASP2, or
(ii) BMP6, ERG, MSR1, MUC1, ACAT1 and DAPK1, or
(iii) HSD3B2, VEGFC, APAF1, MUC1, ACAT1 and DAPK1;
and/or
the subgroup relates to DLBCL and at least one of the first 10 markers shown in Table 5 is typed, preferably corresponding to one or more of the following genes: STAT3, TNFRSF13B, ANXA11, MAP3K7, MEF2B and IFNAR1; and/or
the subgroup relates to lymphoma and at least one of the first 11 markers shown in FIG. 6 is typed, preferably corresponding to one or more of the following genes: STAT3, TNFRSF13B, ANXA11, MAP3K7, MEF2B and IFNAR1.


SEQUENCE LISTING (502 sequences)

SEQ ID NO: 1 - DNA, 14 bp, Homo sapiens
  n (6)..(6): n is a, c, g, or t
  misc_feature (6)..(6): n is a, c, g, or t
  n (9)..(9): n is a, c, g, or t
  misc_feature (9)..(9): n is a, c, g, or t
ccgcgnggnggcag

SEQ ID NO: 2 - DNA, 60 bp, Homo sapiens
gggtttcaccatgttggccaggctggtctcgaactcccgacctcaggtgatccgcccgcc

SEQ ID NO: 3 - DNA, 60 bp, Homo sapiens
gaggggcctctggagggggcgggttctctcgatgcctggcctccacagcacatgtgagca

SEQ ID NO: 4 - DNA, 60 bp, Homo sapiens
gaggcttttatgcaggaaagtgtcccagtcgagggactggcagcagggggacagcaaggg

SEQ ID NO: 5 - DNA, 60 bp, Homo sapiens
acctctcttaattttctcagccattctttcgaccgcctctgccccgctctcgctctgcac

SEQ ID NO: 6 - DNA, 60 bp, Homo sapiens
tgtgaagggaggggaggagaaaagaaaatcgaaacaagcttagaagcagacacttgccca

SEQ ID NO: 7 - DNA, 60 bp, Homo sapiens
tgggggagctctggggtgggggtagcggtcgatgggtcctgatgcctctcagaaggcctt

SEQ ID NO: 8 - DNA, 60 bp, Homo sapiens
acatttcaaatcctctcttctagctacctcgaacttctgagctcaagcaatcttccacct

SEQ ID NO: 9 - DNA, 60 bp, Homo sapiens
ctagaggagagagggatgccaggctctatcgagtctgagttcgtccacgtggtggccatc

SEQ ID NO: 10 - DNA, 60 bp, Homo sapiens
tctttatggtgtctctttatatatttactcgaggctgcagtgagctataattgcaccact

SEQ ID NO: 11 - DNA, 60 bp, Homo sapiens
acgggcagacaggaccccagcccatgcctcgacccactcccggggggatcgggacaccgc

SEQ ID NO: 12 - DNA, 60 bp, Homo sapiens
tccctgcctctctggcgctctcggaccctcgaaccctccctttgatctattccattctca

SEQ ID NO: 13 - DNA, 60 bp, Homo sapiens
agatccgtgtctgcctgcagatacaaaatcgaggtggatcgcccaggggcgggcagtccc

SEQ ID NO: 14 - DNA, 60 bp, Homo sapiens
gggggggggagcgcgccggtccccgcgctcgaaggctgccctcctctctgaatttgggtt

SEQ ID NO: 15 - DNA, 60 bp, Homo sapiens
acccaaacacgcgcagacacccgcacactcgacccactcccggggggatcgggacaccgc

SEQ ID NO: 16 - DNA, 60 bp, Homo sapiens
cacacccgccctactggatccaagtcactcgagacaacactgaaaacacaaaggcattta

SEQ ID NO: 17 - DNA, 60 bp, Homo sapiens
tagactagcgccagctttgtgcacaaggtcgacacccctctccccaaccctctgtcagaa

SEQ ID NO: 18 - DNA, 60 bp, Homo sapiens
agggtttcaccatgttgccaggctggtctcgagaccatcctggctaatacggtgaaaccc

SEQ ID NO: 19 - DNA, 60 bp, Homo sapiens
caacttcattcccacggtcactgccatctcgacccaccaatagagcaactccctgagagg

SEQ ID NO: 20 - DNA, 60 bp, Homo sapiens
ccatcagcaaagatgaacctggcacctctcgacgccataagcatggtgagccagggtggg

SEQ ID NO: 21 - DNA, 60 bp, Homo sapiens
ttccagttcataaagatttaaacaacattcgagaagagaaagggggggaagctgctaggt

SEQ ID NO: 22 - DNA, 60 bp, Homo sapiens
ccccgtgcacagatcccaccacccagggtcgaagcccctccgggcccctcacgggagggg

SEQ ID NO: 23 - DNA, 60 bp, Homo sapiens
aattgctccattatggctcactgcagcctcgaaggtttagcttattcattaaaatcagta

SEQ ID NO: 24 - DNA, 60 bp, Homo sapiens
gctgaaagttattactttgtttttcccatcgaggtcccgcgcacacgcccccgcgcgcac

SEQ ID NO: 25 - DNA, 60 bp, Homo sapiens
agctgttcctcctttaagggtgactccctcgacccccacgtgctgagggctccagccaga

SEQ ID NO: 26 - DNA, 60 bp, Homo sapiens
gccatgacggggctggaggaccaggagttcgaccctgggtggtgggatctgtgcacgggg

SEQ ID NO: 27 - DNA, 60 bp, Homo sapiens
gggactgcccgcccctgggcgatccacctcgatgtccaaatggttcttgccttcacctct

SEQ ID NO: 28 - DNA, 60 bp, Homo sapiens
gggtttcaccgtgttagccaggatggtctcgagaccagcctggccaacatggcaaaaccc

SEQ ID NO: 29 - DNA, 60 bp, Homo sapiens
ctgtattagattttcacatgcatgagactcgaaccgagcccccgcaacacactttcaaga

SEQ ID NO: 30 - DNA, 60 bp, Homo sapiens
acagtcaccgccgcttacctgcgcctcctcgaccatgaatatactaccaaggaaatattt

SEQ ID NO: 31 - DNA, 60 bp, Homo sapiens
catgtgttatttccccaatctggaagactcgaccgcctctgccccgctctcgctctgcac

SEQ ID NO: 32 - DNA, 60 bp, Homo sapiens
tgcccctcaagccctcagactacaacaatcgacaacgcgatccaccgggcccgaaagaag

SEQ ID NO: 33 - DNA, 60 bp, Homo sapiens
accaggggccccaaagagggggtcaggctcgaatcaaagggtttctggatccctaggtgt

SEQ ID NO: 34 - DNA, 60 bp, Homo sapiens
tctagaggggtatcctcccaaatcccactcgacccagcctctggaccagtgctcctgcca

SEQ ID NO: 35 - DNA, 60 bp, Homo sapiens
cctgtggtgcccccatctcaccaggctctcgatgatgccacaagtgccgtgccacagcag

SEQ ID NO: 36 - DNA, 60 bp, Homo sapiens
gggttttgccatgtgggccaggctggtctcgagataggcaaagagagatagactaactcg

SEQ ID NO: 37 - DNA, 60 bp, Homo sapiens
cacagacacaacccaggcctccatctactcgatcacagtacttatctgtcttacgtacac

SEQ ID NO: 38 - DNA, 60 bp, Homo sapiens; identical to SEQ ID NO: 6

SEQ ID NO: 39 - DNA, 60 bp, Homo sapiens; identical to SEQ ID NO: 5

SEQ ID NO: 40 - DNA, 60 bp, Homo sapiens
ctagaggagagagggatgccaggctctatcgatgactttcctccggggcgcgcggcgctg

SEQ ID NO: 41 - DNA, 60 bp, Homo sapiens
tcaagaactcatggttcttaaagatcactcgaggctgcagtgagctatgataatgccaca

SEQ ID NO: 42 - DNA, 60 bp, Homo sapiens
ccaccatccacctggggctgaggggacctcgagtttgagcaccccctcctgggtcctcag

SEQ ID NO: 43 - DNA, 60 bp, Homo sapiens; identical to SEQ ID NO: 2

SEQ ID NO: 44 - DNA, 60 bp, Homo sapiens
ataccaaccccagaaataaagtcattcctcgaggctgcagtgagctataattgcaccact

SEQ ID NO: 45 - DNA, 60 bp, Homo sapiens
gttcctcaccctgatcacacctggtttatcgaactctctcaggttcacccagaccaaaga

SEQ ID NO: 46 - DNA, 60 bp, Homo sapiens
tatctggcttaggcagaaggtagggggctcgagtgattatagaaatccatatatatattg

SEQ ID NO: 47 - DNA, 60 bp, Homo sapiens; identical to SEQ ID NO: 9

SEQ ID NO: 48 - DNA, 60 bp, Homo sapiens
aacagcagcattagattctcataggaactcgagtgtcatgaacaatctttttctttaaca

SEQ ID NO: 49 - DNA, 60 bp, Homo sapiens
cattcctagtgccaggacccatctcaggtcgaccccctcccaagccagccgccgcagcag

SEQ ID NO: 50 - DNA, 60 bp, Homo sapiens
cactactacccaggaaagtgatgggaggtcgagattgcaggaaatggagagtacatgcct

SEQ ID NO: 51 - DNA, 60 bp, Homo sapiens
agtggcgcaatcttggctaactgcagcctcgagaccatcctacatggtgaaaccccgtct

SEQ ID NO: 52 - DNA, 60 bp, Homo sapiens
acgggcagacaggaccccagcccatgcctcgagctgaaggaacatgctggcaggtagctc

SEQ ID NO: 53 - DNA, 60 bp, Homo sapiens
atattaaattgcttacatagaatgaaggtcgaggataatgaagggaacctgcccttgcac

SEQ ID NO: 54 - DNA, 21 bp, Homo sapiens
ggaagaccctttgtgacctgg

SEQ ID NO: 55 - DNA, 18 bp, Homo sapiens
caagacctcacccaatgc

SEQ ID NO: 56 - DNA, 18 bp, Homo sapiens
gaggaagggtgtgctttg

SEQ ID NO: 57 - DNA, 20 bp, Homo sapiens
tggtcagacgagatgccaag

SEQ ID NO: 58 - DNA, 22 bp, Homo sapiens
gtttgggacatcagaaatacag

SEQ ID NO: 59 - DNA, 21 bp, Homo sapiens
ctaagtcttaaagggccagag

SEQ ID NO: 60 - DNA, 20 bp, Homo sapiens
cagagaggatagccttacac

SEQ ID NO: 61 - DNA, 21 bp, Homo sapiens
tgcttcatgaaactcagatgg

SEQ ID NO: 62 - DNA, 20 bp, Homo sapiens
acagcagtccaacaatagtc

SEQ ID NO: 63 - DNA, 19 bp, Homo sapiens
gttgaggcagacagaagag

SEQ ID NO: 64 - DNA, 23 bp, Homo sapiens
tcggaggttcctggctctctgat

SEQ ID NO: 65 - DNA, 23 bp, Homo sapiens
tttctcaataaagattctcagat

SEQ ID NO: 66 - DNA, 23 bp, Homo sapiens
taggattcactgagaaggtccct

SEQ ID NO: 67 - DNA, 23 bp, Homo sapiens
cctctctctgagtcttgagtttc

SEQ ID NO: 68 - DNA, 26 bp, Homo sapiens
gatggagaaaggagcaaggaaccagg

SEQ ID NO: 69 - DNA, 24 bp, Homo sapiens
ggctgatggtatgggaatgggtgg

SEQ ID NO: 70 - DNA, 23 bp, Homo sapiens
acccagttacttgttgtatttgc

SEQ ID NO: 71 - DNA, 23 bp, Homo sapiens
ggctttccccttctgttttgttc

SEQ ID NO: 72 - DNA, 23 bp, Homo sapiens
ctctgacaagcaactctgaatcc

SEQ ID NO: 73 - DNA, 26 bp, Homo sapiens
gcttcaaagagtgtgattatgtaaaa

SEQ ID NO: 74 - DNA, 23 bp, Homo sapiens
aataactgtggcatcggagaggt

SEQ ID NO: 75 - DNA, 23 bp, Homo sapiens
aagtctcaatgccacccaggctg

SEQ ID NO: 76 - DNA, 23 bp, Homo sapiens
tgtatccctcctgttatcatccc

SEQ ID NO: 77 - DNA, 23 bp, Homo sapiens
cagacacctcagggctaagagcg

SEQ ID NO: 78 - DNA, 23 bp, Homo sapiens
gggagaaccgaacccctggcggc

SEQ ID NO: 79 - DNA, 23 bp, Homo sapiens
taccccaccccgaccactccgta

SEQ ID NO: 80 - DNA, 23 bp, Homo sapiens
ggaatacaagtgtgtgccaccac

SEQ ID NO: 81 - DNA, 23 bp, Homo sapiens
ctttgggcttgaaggctttgttc

SEQ ID NO: 82 - DNA, 26 bp, Homo sapiens
agcctcagccgtttctggagtctcgg

SEQ ID NO: 83 - DNA, 23 bp, Homo sapiens
tctaaccccagttctgccagtaa

SEQ ID NO: 84 - DNA, 23 bp, Homo sapiens
cggttctcactttccttctttgc

SEQ ID NO: 85 - DNA, 23 bp, Homo sapiens
caaatgagagcctccaagacagc

SEQ ID NO: 86 - DNA, 23 bp, Homo sapiens
tggttcacggcaaagtagtcaca

SEQ ID NO: 87 - DNA, 23 bp, Homo sapiens
tctatcactttcctgggcatcag

SEQ ID NO: 88 - DNA, 23 bp, Homo sapiens
cctgcctcagcctcccaagtagc

SEQ ID NO: 89 - DNA, 26 bp, Homo sapiens
tggatggaacccctgagccacacagc

SEQ ID NO: 90 - DNA, 23 bp, Homo sapiens
ggttaggtcttctgccttcaaag

SEQ ID NO: 91 - DNA, 23 bp, Homo sapiens
cagacgagatgccaagtgcttta

SEQ ID NO: 92 - DNA, 23 bp, Homo sapiens
tgctggagtgaaaacgcctcttt

SEQ ID NO: 93 - DNA, 23 bp, Homo sapiens
tcataatgtcagtgtcctgttca

SEQ ID NO: 94 - DNA, 23 bp, Homo sapiens
gctttctgaatctttccctggtg

SEQ ID NO: 95 - DNA, 23 bp, Homo sapiens
cctgcctcagccgcccgagtagc

SEQ ID NO: 96 - DNA, 23 bp, Homo sapiens
cctcccacttttgatggcactgc

SEQ ID NO: 97 - DNA, 23 bp, Homo sapiens
cccacatttccttctttcctgtt

SEQ ID NO: 98 - DNA, 23 bp, Homo sapiens
cttctatgggtgatgacctgaca

SEQ ID NO: 99 - DNA, 23 bp, Homo sapiens
tgctggagtgaaaacgcctcttt

SEQ ID NO: 100 - DNA, 23 bp, Homo sapiens
ccatcgctcacatcattacctga

SEQ ID NO: 101 - DNA, 23 bp, Homo sapiens
acatacagtcagtaggagccttg

SEQ ID NO: 102 - DNA, 23 bp, Homo sapiens
gctccaacactcacatctaacac

SEQ ID NO: 103 - DNA, 24 bp, Homo sapiens
gtattttgtttgtttgtttgtttt

SEQ ID NO: 104 - DNA, 26 bp, Homo sapiens
ctccaagacaccactgccgttgaggc

SEQ ID NO: 105 - DNA, 23 bp, Homo sapiens
gcctcatttctgtcctcctttga

SEQ ID NO: 106 - DNA, 20 bp, Homo sapiens
tcaccattcgttcaacacac

SEQ ID NO: 107 - DNA, 20 bp, Homo sapiens
cagttgtggaggctcaatac

SEQ ID NO: 108 - DNA, 19 bp, Homo sapiens
ggaaggaaagccagtgaag

SEQ ID NO: 109 - DNA, 19 bp, Homo sapiens
accctagagtcttggacag

SEQ ID NO: 110 - DNA, 18 bp, Homo sapiens
atccctagggcactgaac

SEQ ID NO: 111 - DNA, 20 bp, Homo sapiens
catacaaggatggagtgacc

SEQ ID NO: 112 - DNA, 19 bp, Homo sapiens
agtgtcttgccctgtaatc

SEQ ID NO: 113 - DNA, 19 bp, Homo sapiens
agcctaagctgaggaactc

SEQ ID NO: 114 - DNA, 22 bp, Homo sapiens
aactcctaatgagaaagtctgc

SEQ ID NO: 115 - DNA, 19 bp, Homo sapiens
ggtcgggtagtagagagtg

SEQ ID NO: 116 - DNA, 23 bp, Homo sapiens
ggacaggtaactacgggtctccc

SEQ ID NO: 117 - DNA, 23 bp, Homo sapiens
taccccaccccgaccactccgta

SEQ ID NO: 118 - DNA, 26 bp, Homo sapiens
caccttgcgtagaggcagtagacccc

SEQ ID NO: 119 - DNA, 24 bp, Homo sapiens
aatgtcctccgagccgcctgctgg

SEQ ID NO: 120 - DNA, 26 bp, Homo sapiens
ggtgtgaggtaagaagtcatagccat

SEQ ID NO: 121 - DNA, 23 bp, Homo sapiens
cacagagcctgccatcctcacat

SEQ ID NO: 122 - DNA, 23 bp, Homo sapiens
actacaggtgcccgccacaaggc

SEQ ID NO: 123 - DNA, 26 bp, Homo sapiens
gggatggagcaggaaggagagagagg

SEQ ID NO: 124 - DNA, 26 bp, Homo sapiens
tatgtcttgccctgtgctgcggctcc

SEQ ID NO: 125 - DNA, 23 bp, Homo sapiens
atcaggtcccgacttccttgggc

SEQ ID NO: 126 - DNA, 26 bp, Homo sapiens
aacaccgagacacaccgagtccctcc

SEQ ID NO: 127 - DNA, 24 bp, Homo sapiens
gactgctcagggctatcctctcag

SEQ ID NO: 128 - DNA, 23 bp, Homo sapiens
agaggtgccagtgggtggaggcg

SEQ ID NO: 129 - DNA, 23 bp, Homo sapiens
gctcctcctcctgctgtcgccag

SEQ ID NO: 130 - DNA, 23 bp, Homo sapiens
gggcggctgtgaaactgaggtcc

SEQ ID NO: 131 - DNA, 23 bp, Homo sapiens
aggaaaggcttcactgagcatca

SEQ ID NO: 132 - DNA, 23 bp, Homo sapiens
tttgtattcttagtagagacggg

SEQ ID NO: 133 - DNA, 24 bp, Homo sapiens
gcccgccgccctgcctttctgaat

SEQ ID NO: 134 - DNA, 23 bp, Homo sapiens
ctcttgttggacagaaaccctac

SEQ ID NO: 135 - DNA, 26 bp, Homo sapiens
tgagcgaccagaccgttgctgtgtgc

SEQ ID NO: 136 - DNA, 26 bp, Homo sapiens
cgcccactgaactggaaagggtcgtg

SEQ ID NO: 137 - DNA, 23 bp, Homo sapiens
agaagtgccagtctacatacacc

SEQ ID NO: 138 - DNA, 26 bp, Homo sapiens
aggcagacacagagcagagcagaggc

SEQ ID NO: 139 - DNA, 26 bp, Homo sapiens
ggtctcccctcctaccacactggcat

SEQ ID NO: 140 - DNA, 23 bp, Homo sapiens
tgaagtttggtaaagaccgagtt

SEQ ID NO: 141 - DNA, 23 bp, Homo sapiens
tgttcttgctttcctccaggttg

SEQ ID NO: 142 - DNA, 26 bp, Homo sapiens
ctgtgggtggaagaggctcaggcatc

SEQ ID NO: 143 - DNA, 26 bp, Homo sapiens
tgagcgaccagaccgttgctgtgtgc

SEQ ID NO: 144 - DNA, 26 bp, Homo sapiens
tttctcctctcccgaagaccgcagcc

SEQ ID NO: 145 - DNA, 23 bp, Homo sapiens
ctctctctctgtcacccaggctg

SEQ ID NO: 146 - DNA, 26 bp, Homo sapiens
cgtaggcatccgtgggtgtgaccagt

SEQ ID NO: 147 - DNA, 23 bp, Homo sapiens
cgcctgtaatcccagaactttgg

SEQ ID NO: 148 - DNA, 23 bp, Homo sapiens
gtctcactctgttgcccaggctg

SEQ ID NO: 149 - DNA, 24 bp, Homo sapiens
ttcttgataaaatgaatcttctta

SEQ ID NO: 150 - DNA, 26 bp, Homo sapiens
tggagtttgctgtgggcactgaggcg

SEQ ID NO: 151 - DNA, 24 bp, Homo sapiens
ccaccaccatcagccagtgccacg

SEQ ID NO: 152 - DNA, 23 bp, Homo sapiens
caatgccaggtcttcatactcta

SEQ ID NO: 153 - DNA, 23 bp, Homo sapiens
acccagcgtcgccgtccaccgta

SEQ ID NO: 154 - DNA, 26 bp, Homo sapiens
ggtccacattctcacgaaccgcctcc

SEQ ID NO: 155 - DNA, 23 bp, Homo sapiens
cattctcctgcctcagcctcctg

SEQ ID NO: 156 - DNA, 23 bp, Homo sapiens
ctaaatgtgctgtgtcttggagc

SEQ ID NO: 157 - DNA, 26 bp, Homo sapiens
tgcttcaccaggaactccaccacccg

SEQ ID NO: 158 - DNA, 60 bp, Homo sapiens; identical to SEQ ID NO: 3

SEQ ID NO: 159 - DNA, 60 bp, Homo sapiens
aatgaggaactagcagcaggaggcagcatcgaaacctgggatgctagtaaccctaccctg

SEQ ID NO: 160 - DNA, 60 bp, Homo sapiens
tccaatcacctcccaccaggtccctccctcgatcctgtgcttttcctgctgcaggtttca

SEQ ID NO: 161 - DNA, 60 bp, Homo sapiens
agtggcgtgatcatggttcactgaagcctcgaaaagaggttggctagaaggccacggggt

SEQ ID NO: 162 - DNA, 60 bp, Homo sapiens
gaggacacggcggggggcccatcacccctcgaacaggagctgtccctcccaggagcaggc

SEQ ID NO: 163 - DNA, 60 bp, Homo sapiens
gagccaggttttgcaggacctgggatattcgagaccagtctgggcaacatagtgagaccc

SEQ ID NO: 164 - DNA, 60 bp, Homo sapiens
tgggggtcccggggaggtgggcgttgcctcgaatctggtcaaaccctacccaaactcatc

SEQ ID NO: 165 - DNA, 60 bp, Homo sapiens
agatccgtgtctgcctgcagatacaaaatcgagttgggctggggagaggaggagataggt

SEQ ID NO: 166 - DNA, 60 bp, Homo sapiens
cactcgggtggcagagatgcgtggagagtcgatgtgtcccaaattgatctcaccctccac

SEQ ID NO: 167 - DNA, 60 bp, Homo sapiens
tgtgaagggaggggaggagaaaagaaaatcgatcatctcaccggccgaagacgaggagga

SEQ ID NO: 168 - DNA, 60 bp, Homo sapiens; identical to SEQ ID NO: 8

SEQ ID NO: 169 - DNA, 60 bp, Homo sapiens
ttctgacagagggttggggagaggggtgtcgacctcctaaagtgctgggattacaggcgt

SEQ ID NO: 170 - DNA, 60 bp, Homo sapiens
ggaggatggggagggtatgtaaatattgtcgatagagcaaggaaaccagaaaggtgtaat

SEQ ID NO: 171 - DNA, 60 bp, Homo sapiens
gtatgagtgtgggtgtgtggatgtggcctcgagatcgcgccactgcactccagcctgggc

SEQ ID NO: 172 - DNA, 60 bp, Homo sapiens; identical to SEQ ID NO: 11

SEQ ID NO: 173 - DNA, 60 bp, Homo sapiens
agtggtgcgatctcagcttgttgcagcctcgaggaatttctaatgatagatccagacctc

SEQ ID NO: 174 - DNA, 60 bp, Homo sapiens
tcattctggggattatcttttcattttctcgaggctgcagtgagctataattgcaccact

SEQ ID NO: 175 - DNA, 60 bp, Homo sapiens; identical to SEQ ID NO: 7

SEQ ID NO: 176 - DNA, 60 bp, Homo sapiens; identical to SEQ ID NO: 4

SEQ ID NO: 177 - DNA, 60 bp, Homo sapiens
gagctggatgccaggcgggccaatgaggtcgattgcaatgcaggatcctatgctggattc

SEQ ID NO: 178 - DNA, 60 bp, Homo sapiens
aacaggcaggagcagctgttcctcagcatcgaacctatttatttacttatttttttgaga

SEQ ID NO: 179 - DNA, 60 bp, Homo sapiens; identical to SEQ ID NO: 10

SEQ ID NO: 180 - DNA, 60 bp, Homo sapiens
gagctggatgccaggcgggccaatgaggtcgaacacgatatgaacaggacatctgttaca

SEQ ID NO: 181 - DNA, 60 bp, Homo sapiens
gggtggagtcagggaggggtgggggacgtcgagtcttgcttgaccccagagcagctccct

SEQ ID NO: 182 - DNA, 60 bp, Homo sapiens
gggtttcaccgtgttactcaggctggtctcgaagtcctgggctcaagcaatccacccgct

SEQ ID NO: 183 - DNA, 60 bp, Homo sapiens
caaatactcatgtgtatgggcaaaaaactcgagtagttggaacttcaagtgtcaaaacat

SEQ ID NO: 184 - DNA, 60 bp, Homo sapiens
gacgggccgattgcctgagctcaggagttcgacccttctcacgtgggctaagggcctgac

SEQ ID NO: 185 - DNA, 60 bp, Homo sapiens
actagctgggtgaccctagacagtttgttcgaggctacagtgagctgtgatagtgccact

SEQ ID NO: 186 - DNA, 60 bp, Homo sapiens
tgttgtatccattattgaaagtggagtatcgaggctgcagtgagctgagatcattccact

SEQ ID NO: 187 - DNA, 60 bp, Homo sapiens
gacaggcagattgcctgagctcaggagttcgacatctctacactcattctttctactcag

SEQ ID NO: 188 - DNA, 60 bp, Homo sapiens
acatttcaaatcctctcttctagctacctcgaaacaccactacttgtcagtttacaatga

SEQ ID NO: 189 - DNA, 60 bp, Homo sapiens
cggtgtctggtgagttttaacatccttgtcgagctgcagacttggctttggaagaatcac

SEQ ID NO: 190 - DNA, 60 bp, Homo sapiens
gctcagcaaatgaatgttttcaaagcactcgattgcaatgcaggatcctatgctggattc

SEQ ID NO: 191 - DNA, 60 bp, Homo sapiens
gtgcttcaagcagagcttcctccctccgtcgaactcctgacctcgtgatccgcctgcctc

SEQ ID NO: 192 - DNA, 60 bp, Homo sapiens
atcctaactgctgaagtctgtgttttcatcgaactcctgacctcgtgatccgcctgcctc

SEQ ID NO: 193 - DNA, 60 bp, Homo sapiens
ggcttgcctaaaaaagtaaacaaaacagtcgaactcctgctcatgatccgcctgccttgg

SEQ ID NO: 194 - DNA, 60 bp, Homo sapiens
ccaaggcaggcggatcatgagcaggagttcgagaccagcctggccaagatagtgaaaccc

SEQ ID NO: 195 - DNA, 60 bp, Homo sapiens
gccgaggcgggtggatcaggtcaggagttcgagaccagcctggccaagatagtgaaaccc

SEQ ID NO: 196 - DNA, 60 bp, Homo sapiens
ggcgggtggatcacttgaggtcaggagttcgagaccagcctggccaagatagtgaaaccc

SEQ ID NO: 197 - DNA, 60 bp, Homo sapiens; identical to SEQ ID NO: 50

SEQ ID NO: 198 - DNA, 26 bp, Homo sapiens
ggctcgtaacaaacccctgaccccag

SEQ ID NO: 199 - DNA, 26 bp, Homo sapiens
tccccattaccccatcagtgctcccc

SEQ ID NO: 200 - DNA, 26 bp, Homo sapiens
ggagaggcagagcagagagtgaaggg

SEQ ID NO: 201 - DNA, 23 bp, Homo sapiens
gacagcagtttctaagcctggca

SEQ ID NO: 202 - DNA, 23 bp, Homo sapiens
tttggaggactgggacttgccgt

SEQ ID NO: 203 - DNA, 23 bp, Homo sapiens
aactgaaagaaagacccagaggc

SEQ ID NO: 204 - DNA, 23 bp, Homo sapiens
gacccaaagggcaataccagagc

SEQ ID NO: 205 - DNA, 23 bp, Homo sapiens
cacgctcgcccatcattgaaaac

SEQ ID NO: 206 - DNA, 23 bp, Homo sapiens
tcccttcatccacaggaatacct

SEQ ID NO: 207 - DNA, 23 bp, Homo sapiens
ggttaggtcttctgccttcaaag

SEQ ID NO: 208 - DNA, 23 bp, Homo sapiens
gtgtaacaatcaagtcagggaat

SEQ ID NO: 209 - DNA, 23 bp, Homo sapiens
cacagagcctgccatcctcacat

SEQ ID NO: 210 - DNA, 23 bp, Homo sapiens
aaataagtaaggacaaagagtgc

SEQ ID NO: 211 - DNA, 26 bp, Homo sapiens
tcgcctacggcttgtttacgcacagc

SEQ ID NO: 212 - DNA, 23 bp, Homo sapiens
gcttatttacaagacgaacccgc

SEQ ID NO: 213 - DNA, 23 bp, Homo sapiens
ttctgttgtccaggcttgagtgc

SEQ ID NO: 214 - DNA, 23 bp, Homo sapiens
cactattgagttctaagagttct

SEQ ID NO: 215 - DNA, 26 bp, Homo sapiens
ggaacccacgccctcccctaagtctt

SEQ ID NO: 216 - DNA, 23 bp, Homo sapiens
ggtgtgctttgccaggataagaa

SEQ ID NO: 217 - DNA, 23 bp, Homo sapiens
tctccctggcgacctcgtcccta

SEQ ID NO: 218 - DNA, 23 bp, Homo sapiens
tgtttgctttatggacacacaga

SEQ ID NO: 219 - DNA, 23 bp, Homo sapiens
catttactcactctcataccata

SEQ ID NO: 220 - DNA, 26 bp, Homo sapiens
actctgccgctcggtcaccaacctga

SEQ ID NO: 221 - DNA, 23 bp, Homo sapiens
gacaagggagggaggaggatggg

SEQ ID NO: 222 - DNA, 23 bp, Homo sapiens
cctgcctcagcctcccaagtagc

SEQ ID NO: 223 - DNA, 26 bp, Homo sapiens
gtgaactcagccaagcacagtggtgg

SEQ ID NO: 224 - DNA, 23 bp, Homo sapiens
ttctttacccctgtcactcacct

SEQ ID NO: 225 - DNA, 23 bp, Homo sapiens
tggttggaagtagccctgattca

SEQ ID NO: 226 - DNA, 23 bp, Homo sapiens
gttgccttgttatctgcctggtt

SEQ ID NO: 227 - DNA, 23 bp, Homo sapiens
gtaatcctaacactgtgggaggc

SEQ ID NO: 228 - DNA, 26 bp, Homo sapiens
gggagcattgtgggctaacaggagac

SEQ ID NO: 229 - DNA, 23 bp, Homo sapiens
tcgtaggcaacatcgtcaaggat

SEQ ID NO: 230 - DNA, 23 bp, Homo sapiens
ctgggcaacagagtgagagcctg

SEQ ID NO: 231 - DNA, 23 bp, Homo sapiens
tgctacctctgactacagggtgg

SEQ ID NO: 232 - DNA, 23 bp, Homo sapiens
gctgactgaagattctgcctttc

SEQ ID NO: 233 - DNA, 23 bp, Homo sapiens
taggatggcaagcagcattggct

SEQ ID NO: 234 - DNA, 25 bp, Homo sapiens
cacgcctgtaatcccagcactttgg

SEQ ID NO: 235 - DNA, 24 bp, Homo sapiens
cacgcctgtaatcccagcactctg

SEQ ID NO: 236 - DNA, 24 bp, Homo sapiens
atgcctgtaatcccagcactttgg

SEQ ID NO: 237 - DNA, 23 bp, Homo sapiens
ccaccattcgtgctccaacactc

SEQ ID NO: 238 - DNA, 23 bp, Homo sapiens
acagttgtggaggctcaatacct

SEQ ID NO: 239 - DNA, 23 bp, Homo sapiens
cggtaacagacacggagtgaaat

SEQ ID NO: 240 - DNA, 23 bp, Homo sapiens
gcagggactgagaaacataggat

SEQ ID NO: 241 - DNA, 23 bp, Homo sapiens
tggaccccagggcagggcttcat

SEQ ID NO: 242 - DNA, 23 bp, Homo sapiens
tcagaccctccttcccacctctc

SEQ ID NO: 243 - DNA, 26 bp, Homo sapiens
ccccttctcctgctgctaccatccag

SEQ ID NO: 244 - DNA, 23 bp, Homo sapiens
ctcagggagaccaaggcagtgac

SEQ ID NO: 245 - DNA, 23 bp, Homo sapiens
gggactggagggaaggaagtggg

SEQ ID NO: 246 - DNA, 26 bp, Homo sapiens
ggagcagtgtagggcagggtgtcaga

SEQ ID NO: 247 - DNA, 26 bp, Homo sapiens
atgtctacagcctctgccgcctcctc

SEQ ID NO: 248 - DNA, 23 bp, Homo sapiens
gccctgtaatcccagcactttgg

SEQ ID NO: 249 - DNA, 23 bp, Homo sapiens
ccccagggactgaggacttgtgt

SEQ ID NO: 250 - DNA, 23 bp, Homo sapiens
aacaatctattttaccaacctat

SEQ ID NO: 251 - DNA, 26 bp, Homo sapiens
caggtagtgtgttttccaactctgtt

SEQ ID NO: 252 - DNA, 26 bp, Homo sapiens
tagtagagagtgcggtgcccacaggc

SEQ ID NO: 253 - DNA, 23 bp, Homo sapiens
ggcaaggtctccagtggtgaggt

SEQ ID NO: 254 - DNA, 23 bp, Homo sapiens
gtctcactctgttgcccaggctg

SEQ ID NO: 255 - DNA, 23 bp, Homo sapiens
tggattttctgcggctctgtttg

SEQ ID NO: 256 - DNA, 26 bp, Homo sapiens
agtcccctctctgggtctcagccaag

SEQ ID NO: 257 - DNA, 23 bp, Homo sapiens
tatggcattttccccttccagta

SEQ ID NO: 258 - DNA, 23 bp, Homo sapiens
cactccagcctgagagacagagc

SEQ ID NO: 259 - DNA, 23 bp, Homo sapiens
gtctcactctgttgcccaggctg

SEQ ID NO: 260 - DNA, 23 bp, Homo sapiens
cagggttgttgtgagggttatgt

SEQ ID NO: 261 - DNA, 23 bp, Homo sapiens
gtccctgctctcttagccccaga

SEQ ID NO: 262 - DNA, 23 bp, Homo sapiens
agacctttggtttctacatctat

SEQ ID NO: 263 - DNA, 26 bp, Homo sapiens
ggtatcaaatgttccacaagtgttgc

SEQ ID NO: 264 - DNA, 25 bp, Homo sapiens
ccaggatgtcttaccgccccgtcag

SEQ ID NO: 265 - DNA, 23 bp, Homo sapiens
gggtctcactctgttgcccaagc

SEQ ID NO: 266 - DNA, 26 bp, Homo sapiens
cgtcttgctctgtctgttgcccaggc

SEQ ID NO: 267 - DNA, 23 bp, Homo sapiens
ggcaatagggatgattctgtgaa

SEQ ID NO: 268 - DNA, 23 bp, Homo sapiens
gcacaggagggttacttcacaag

SEQ ID NO: 269 - DNA, 26 bp, Homo sapiens
gcttcacgggaggagggtagactctc

SEQ ID NO: 270 - DNA, 23 bp, Homo sapiens
tatggcattttccccttccagta

SEQ ID NO: 271 - DNA, 23 bp, Homo sapiens
atgttagtcccttcccaccctat

SEQ ID NO: 272 - DNA, 23 bp, Homo sapiens
atgttagtcccttcccaccctat

SEQ ID NO: 273 - DNA, 23 bp, Homo sapiens
acgcctgtaatcccagcactttg

SEQ ID NO: 274 - DNA, 23 bp, Homo sapiens
gattctcctgcctcagcctcccg

SEQ ID NO: 275 - DNA, 24 bp, Homo sapiens
cgattctcctgcctcagcctcccg

SEQ ID NO: 276 - DNA, 24 bp, Homo sapiens
cgattctcctgcctcagcctcccg

SEQ ID NO: 277 - DNA, 23 bp, Homo sapiens
ctcacgaaccgcctcctttcctc

SEQ ID NO: 278 - DNA, 60 bp, Homo sapiens
agttgtatttttagaaagtagtgtttaatcgatagaaatataacatgaaacacatatata

SEQ ID NO: 279 - DNA, 60 bp, Homo sapiens
actaatcccctgaagaagcaaattaacttcgagtatccctttaagtttgtttttaaaata

SEQ ID NO: 280 - DNA, 60 bp, Homo sapiens
tcagtttctgctctcaagaagcttacagtcgaaggtcccaagttagattacggcaaagct

SEQ ID NO: 281 - DNA, 60 bp, Homo sapiens
tcttgaatgtgcttagtattattcagactcgaaaacataatttgaaaggaattcattctg

SEQ ID NO: 282 - DNA, 60 bp, Homo sapiens
aggaggtaacgattggtcagctgcttaatcgaggcagaagtctatttgaaacgtaagata

SEQ ID NO: 283 - DNA, 60 bp, Homo sapiens
ggcctttaaggcccctctgaaatccagcatcgaagagggaaactgcatcacagttgatgg

SEQ ID NO: 284 - DNA, 21 bp, Homo sapiens
aagaagggatgggacgggact

SEQ ID NO: 285 - DNA, 21 bp, Homo sapiens
actggtcacagggaacgatgg

SEQ ID NO: 286 - DNA, 21 bp, Homo sapiens
acttggattcccaaaacgcca

SEQ ID NO: 287 - DNA, 21 bp, Homo sapiens
cagcctaccttgcctgacact

SEQ ID NO: 288 - DNA, 25 bp, Homo sapiens
tccattttcctttccctttgctctg

SEQ ID NO: 289 - DNA, 22 bp, Homo sapiens
ggggagtggatgggataaggtg

SEQ ID NO: 290 - DNA, 25 bp, Homo sapiens
ggtacacgaattaactattccctgt

SEQ ID NO: 291 - DNA, 25 bp, Homo sapiens
aggtgtgaatgttactgaacacaaa

SEQ ID NO: 292 - DNA, 21 bp, Homo sapiens
ctcttccccggtgagtttcca

SEQ ID NO: 293 - DNA, 20 bp, Homo sapiens
aaagcccagtgatggcccat

SEQ ID NO: 294 - DNA, 21 bp, Homo sapiens
ccacacagggccctaatgacc

SEQ ID NO: 295 - DNA, 20 bp, Homo sapiens
tgggcctggttgaaaagcat

SEQ ID NO: 296 - DNA, 35 bp, Homo sapiens
agtgtttaatcgatagaaatataacatgaaacaca

SEQ ID NO: 297 - DNA, 28 bp, Homo sapiens
agggatactcgaagttaatttgcttctt

SEQ ID NO: 298 - DNA, 26 bp, Homo sapiens
aagaagcttacagtcgaaggtcccaa

SEQ ID NO: 299 - DNA, 36 bp, Homo sapiens
attcctttcaaattatgttttcgagtctgaataata

SEQ ID NO: 300 - DNA, 26 bp, Homo sapiens
aaatagacttctgcctcgattaagca

SEQ ID NO: 301 - DNA, 29 bp, Homo sapiens
atccagcatcgaagagggaaactgcatca

SEQ ID NO: 302 - DNA, 24 bp, Canis lupus
gccagagaacagatgtgtgtgtct

SEQ ID NO: 303 - DNA, 26 bp, Canis lupus
gcctctctggtgccacatcttatctt

SEQ ID NO: 304 - DNA, 25 bp, Canis lupus
ctgcctgtgtgtagtcacgagaagc

SEQ ID NO: 305 - DNA, 25 bp, Canis lupus
ctgacagcagaagcacgaaaaggtc

SEQ ID NO: 306 - DNA, 26 bp, Canis lupus
ccatccaccccacagttcctatgaaa

SEQ ID NO: 307 - DNA, 23 bp, Canis lupus
cccaacgaggtcaggaagggaga

SEQ ID NO: 308 - DNA, 26 bp, Canis lupus
tgtctcagtatctatttcccaagtgc

SEQ ID NO: 309 - DNA, 23 bp, Canis lupus
caggacccagacttgcccaaacc

SEQ ID NO: 310 - DNA, 23 bp, Canis lupus
agacccaatgcctgccacacgga

SEQ ID NO: 311 - DNA, 25 bp, Canis lupus
ctgcctgtgtgtagtcacgagaagc

SEQ ID NO: 312 - DNA, 26 bp, Canis lupus
gcataactcagagaaagccactgtga

SEQ ID NO: 313 - DNA, 25 bp, Canis lupus
ctgacagcagaagcacgaaaaggtc

SEQ ID NO: 314 - DNA, 25 bp, Canis lupus
ctgcctgtgtgtagtcacgagaagc

SEQ ID NO: 315 - DNA, 23 bp, Canis lupus
cccaacgaggtcaggaagggaga

SEQ ID NO: 316 - DNA, 23 bp, Canis lupus
cacccatccaccccacagttcct

SEQ ID NO: 317 - DNA, 23 bp, Canis lupus
cccaacgaggtcaggaagggaga

SEQ ID NO: 318 - DNA, 26 bp, Canis lupus
cctctctggtgccacatcttatctta

SEQ ID NO: 319 - DNA, 23 bp, Canis lupus
ttgacctgggctcacatcgctga

SEQ ID NO: 320 - DNA, 26 bp, Canis lupus
gtcttcaagccacagagcaggattcc

SEQ ID NO: 321 - DNA, 26 bp, Canis lupus
ggtctgaaaatgtgaatgtcttgtgt

SEQ ID NO: 322 - DNA, 23 bp, Canis lupus
gtgcccttgagtccagccgtcat

SEQ ID NO: 323 - DNA, 23 bp, Canis lupus
tgtctctctcctaaggtgtcccc

SEQ ID NO: 324 - DNA, 25 bp, Canis lupus
ctgcctgtgtgtagtcacgagaagc

SEQ ID NO: 325 - DNA, 23 bp, Canis lupus
gcactttctctccaggtcaccct

SEQ ID NO: 326 - DNA, 23 bp, Canis lupus
ctgcttgggctggtctttggttg

SEQ ID NO: 327 - DNA, 23 bp, Canis lupus
ggcactttctctccaggtcaccc

SEQ ID NO: 328 - DNA, 23 bp, Canis lupus
tgagcggtcactgctgttgtagg

SEQ ID NO: 329 - DNA, 23 bp, Canis lupus
ttccatcctgctgtccgtcctgc

SEQ ID NO: 330 - DNA, 23 bp, Canis lupus
cggagagaaggcggagaaaccgt

SEQ ID NO: 331 - DNA, 23 bp, Canis lupus
gagcggtcactgctgttgtaggc

SEQ ID NO: 332 - DNA, 23 bp, Canis lupus
cattcctggtatcgtgttgccgc

SEQ ID NO: 333 - DNA, 23 bp, Canis lupus
ggacttcctcctcgcctaatgcg

SEQ ID NO: 334 - DNA, 23 bp, Canis lupus
tcctcccatcctcactggaccac

SEQ ID NO: 335 - DNA, 23 bp, Canis lupus
agggctctgcgtttactccaggc

SEQ ID NO: 336 - DNA, 26 bp, Canis lupus
ctggagcctgagtaatgaataggagc

SEQ ID NO: 337 - DNA, 23 bp, Canis lupus
gccccaatcccatccagaatcca

SEQ ID NO: 338 - DNA, 24 bp, Canis lupus
ctttctctcttccctcgtccctgg

SEQ ID NO: 339 - DNA, 26 bp, Canis lupus
tttgataatgagggctggctgggcat

SEQ ID NO: 340 - DNA, 25 bp, Canis lupus
ggatgccttagttcctattgacact

SEQ ID NO: 341 - DNA, 26 bp, Canis lupus
ctgctggaggagtgacacaaagtttc

SEQ ID NO: 342 - DNA, 26 bp, Canis lupus
gcctgctggaggagtgacacaaagtt

SEQ ID NO: 343 - DNA, 26 bp, Canis lupus
cctttcctcttccatctactcattcc

SEQ ID NO: 344 - DNA, 26 bp, Canis lupus
ttctatccctccacaagatgctcata

SEQ ID NO: 345 - DNA, 23 bp, Canis lupus
gggagacggaggaaaagcctatc

SEQ ID NO: 346 - DNA, 26 bp, Canis lupus
aacctcctcaaagagagagccttccc

SEQ ID NO: 347 - DNA, 26 bp, Canis lupus
aggtcttcaaccaaacaccaccagtg

SEQ ID NO: 348 - DNA, 26 bp, Canis lupus
cctcctgtatttctacttccactcag

SEQ ID NO: 349 - DNA, 26 bp, Canis lupus
gcaggtcttcaaccaaacaccaccag

SEQ ID NO: 350 - DNA, 26 bp, Canis lupus
aacctcctcaaagagagagccttccc

SEQ ID NO: 351 - DNA, 26 bp, Canis lupus
cagtgtgaaagcaccttcgctcttgc

SEQ ID NO: 352 - DNA, 26 bp, Canis lupus
gggcaatgtgaggctgttatgcttgt

SEQ ID NO: 353 - DNA, 26 bp, Canis lupus
ccagggcaatgtgaggctgttatgct

SEQ ID NO: 354 - DNA, 23 bp, Canis lupus
tttgagggcagagcaggaagggt

SEQ ID NO: 355 - DNA, 23 bp, Canis lupus
gtccctgctccactgccaatgag

SEQ ID NO: 356 - DNA, 23 bp, Canis lupus
gtgccctggatggagaacttgct

SEQ ID NO: 357 - DNA, 23 bp, Canis lupus
tacagaaagccctcgctgggagc

SEQ ID NO: 358 - DNA, 23 bp, Canis lupus
aagtgtagcacggaccagagagc

SEQ ID NO: 359 - DNA, 23 bp, Canis lupus
ctgcctccagaaggtgtctcaga

SEQ ID NO: 360 - DNA, 23 bp, Canis lupus
gtgccctggatggagaacttgct

SEQ ID NO: 361 - DNA, 23 bp, Canis lupus
ggacaagcatcctggttgagcca

SEQ ID NO: 362 - DNA, 23 bp, Canis lupus
ggacaagcatcctggttgagcca

SEQ ID NO: 363 - DNA, 26 bp, Canis lupus
gacccagaaatgaacccaaaagatga

SEQ ID NO: 364 - DNA, 26 bp, Canis lupus
gcactccctacacacaaatccttaga

SEQ ID NO: 365 - DNA, 26 bp, Canis lupus
gcaacagttcataaccgagtgccaac

SEQ ID NO: 366 - DNA, 26 bp, Canis lupus
gcaacagttcataaccgagtgccaac

SEQ ID NO: 367 - DNA, 26 bp, Canis lupus
cagttcataaccgagtgccaacagaa

SEQ ID NO: 368 - DNA, 26 bp, Canis lupus
ggtgactgatgagactccaggaaagt

SEQ ID NO: 369 - DNA, 26 bp, Canis lupus
gacccagaaatgaacccaaaagatga

SEQ ID NO: 370 - DNA, 26 bp, Canis lupus
gacccagaaatgaacccaaaagatga

SEQ ID NO: 371 - DNA, 26 bp, Canis lupus
cccacctccctgctccaacaagattt

SEQ ID NO: 372 - DNA, 26 bp, Canis lupus
gacccagaaatgaacccaaaagatga

SEQ ID NO: 373 - DNA, 23 bp, Canis lupus
gcagcctttggcagcactctctg

SEQ ID NO: 374 - DNA, 26 bp, Canis lupus
cccttctggaactggatgagccctta

SEQ ID NO: 375 - DNA, 23 bp, Canis lupus
tgagcccttagtcaatgggaccg

SEQ ID NO: 376 - DNA, 23 bp, Canis lupus
ccagttcaccaaggttgagtgcc

SEQ ID NO: 377 - DNA, 24 bp, Canis lupus
aaaactcccacctgtctgtgtcac

SEQ ID NO: 378 - DNA, 26 bp, Canis lupus
gcataactcagagaaagccactgtga

SEQ ID NO: 379 - DNA, 26 bp, Canis lupus
gacagcagaagcacgaaaaggtcatt

SEQ ID NO: 380 - DNA, 23 bp, Canis lupus
tgtccctccagcctctgttaccc

SEQ ID NO: 381 - DNA, 26 bp, Canis lupus
ggtctgaaagcacctgtaactctgga

SEQ ID NO: 382 - DNA, 23 bp, Canis lupus
cccttgagtccagccgtcattac

SEQ ID NO: 383 - DNA, 26 bp, Canis lupus
acacgatgagacagagcaccagagtc

SEQ ID NO: 384 - DNA, 23 bp, Canis lupus
ggtgagttctgacctgggctttc

SEQ ID NO: 385 - DNA, 23 bp, Canis lupus
tctgaggtcctgatggagcacag

SEQ ID NO: 386 - DNA, 26 bp, Canis lupus
cctctctggtgccacatcttatctta

SEQ ID NO: 387 - DNA, 26 bp, Canis lupus
gtcttcaagccacagagcaggattcc

SEQ ID NO: 388 - DNA, 26 bp, Canis lupus
ccatcttctgtaaccctgaacggagt

SEQ ID NO: 389 - DNA, 26 bp, Canis lupus
cgttatctatggtcccactactgtgt

SEQ ID NO: 390 - DNA, 23 bp, Canis lupus
gcaggttattagaggaccgaggc

SEQ ID NO: 391 - DNA, 23 bp, Canis lupus
cgccaccaagaatgtcatctccg

SEQ ID NO: 392 - DNA, 23 bp, Canis lupus
cgatgagacagagcaccagagtc

SEQ ID NO: 393 - DNA, 26 bp, Canis lupus
gtcttcaagccacagagcaggattcc

SEQ ID NO: 394 - DNA, 23 bp, Canis lupus
gtggctacctgtggtcctctcct

SEQ ID NO: 395 - DNA, 26 bp, Canis lupus
gccaccaagaatgtcatctccgattt

SEQ ID NO: 396 - DNA, 26 bp, Canis lupus
ggcttcgttatctatggtcccactac

SEQ ID NO: 397 - DNA, 23 bp, Canis lupus
cgccaccaagaatgtcatctccg

SEQ ID NO: 398 - DNA, 23 bp, Canis lupus
caggacccagacttgcccaaacc

SEQ ID NO: 399 - DNA, 26 bp, Canis lupus
ctgtaaccctgaacggagtagaatag

SEQ ID NO: 400 - DNA, 23 bp, Canis lupus
ggcggagaaaccgttcgtgtgtg

SEQ ID NO: 401 - DNA, 24 bp, Canis lupus
ggcaagggaccactcttagtctgc

SEQ ID NO: 402 - DNA, 23 bp, Canis lupus
tccccttatcaaccaactcgggc

SEQ ID NO: 403 - DNA, 23 bp, Canis lupus
ttggtggtcaggactggagtgcc

SEQ ID NO: 404 - DNA, 23 bp, Canis lupus
ctgcttgggctggtctttggttg

SEQ ID NO: 405 - DNA, 23 bp, Canis lupus
tccccttatcaaccaactcgggc

SEQ ID NO: 406 - DNA, 23 bp, Canis lupus
gaggtcaagggaagagacaggga

SEQ ID NO: 407 - DNA, 23 bp, Canis lupus
tgtggaatgagcctccgtccctg

SEQ ID NO: 408 - DNA, 23 bp, Canis lupus
cattcctggtatcgtgttgccgc

SEQ ID NO: 409 - DNA, 23 bp, Canis lupus
ccagaacatctcttcgtggtggg

SEQ ID NO: 410 - DNA, 23 bp, Canis lupus
gatgctgtccctgtgctatgagc

SEQ ID NO: 411 - DNA, 26 bp, Canis lupus
gtcatcaacactctttccctgctcct

SEQ ID NO: 412 - DNA, 23 bp, Canis lupus
ccattgcctgaatcctccctggc

SEQ ID NO: 413 - DNA, 23 bp, Canis lupus
tgagggctggctgggcattcata

SEQ ID NO: 414 - DNA, 26 bp, Canis lupus
gtcatcaacactctttccctgctcct

SEQ ID NO: 415 - DNA, 25 bp, Canis lupus
cagccccaatcccatccagaatcca

SEQ ID NO: 416 - DNA, 26 bp, Canis lupus
ctgtgattcccttgttatggttttga

SEQ ID NO: 417 - DNA, 26 bp, Canis lupus
gcctctgtcctgtgtgttatgaaact

SEQ ID NO: 418 - DNA, 26 bp, Canis lupus
ctacaagggaactgcctgcttcgcta

SEQ ID NO: 419 - DNA, 26 bp, Canis lupus
aacaggcttacctcttcggactgctc

SEQ ID NO: 420 - DNA, 24 bp, Canis lupus
ctcctcaaagagagagccttcccg

SEQ ID NO: 421 - DNA, 26 bp, Canis lupus
gcgtgtgagagaggagataaatggat

SEQ ID NO: 422 - DNA, 26 bp, Canis lupus
ctggctggctcttgactttgctattg

SEQ ID NO: 423 - DNA, 26 bp, Canis lupus
aacctcctcaaagagagagccttccc

SEQ ID NO: 424 - DNA, 26 bp, Canis lupus
cctcctgtatttctacttccactcag

SEQ ID NO: 425 - DNA, 26 bp, Canis lupus
gactgattgtaggaggactcacagat

SEQ ID NO: 426 - DNA, 26 bp, Canis lupus
cctcctgtatttctacttccactcag

SEQ ID NO: 427 - DNA, 26 bp, Canis lupus
atcattggtttggagtgacaactact

SEQ ID NO: 428 - DNA, 26 bp, Canis lupus
ggtagtgtctgttttctggactttac

SEQ ID NO: 429 - DNA, 23 bp, Canis lupus
ggtgtgggtgtgtaagagggacc

SEQ ID NO: 430 - DNA, 23 bp, Canis lupus
ctgcctccagaaggtgtctcaga

SEQ ID NO: 431 - DNA, 23 bp, Canis lupus
tacagaaagccctcgctgggagc

SEQ ID NO: 432 - DNA, 23 bp, Canis lupus
agggtgtgggtgtgtaagaggga

SEQ ID NO: 433 - DNA, 23 bp, Canis lupus
ccactgtgccctggatggagaac

SEQ ID NO: 434 - DNA, 23 bp, Canis lupus
ggtgtgggtgtgtaagagggacc

SEQ ID NO: 435 - DNA, 23 bp, Canis lupus
ttgagggcagagcaggaagggtg

SEQ ID NO: 436 - DNA, 24 bp, Canis lupus
gggatacccagagagaagggcaag

SEQ ID NO: 437 - DNA, 23 bp, Canis lupus
agacctgaggaaggagggtggac

SEQ ID NO: 438 - DNA, 26 bp, Canis lupus
gtgagaggcagagacagcacagacta

SEQ ID NO: 439 - DNA, 26 bp, Canis lupus
gtgagaggcagagacagcacagacta

SEQ ID NO: 440 - DNA, 26 bp, Canis lupus
ggtgactgatgagactccaggaaagt

SEQ ID NO: 441 - DNA, 26 bp, Canis lupus
gcctaaactttctctctcagtcagcg

SEQ ID NO: 442 - DNA, 26 bp, Canis lupus
gcctctgtcattcgtgcttccagtgt

SEQ ID NO: 443 - DNA, 26 bp, Canis lupus
gcctaaactttctctctcagtcagcg

SEQ ID NO: 444 - DNA, 24 bp, Canis lupus
tgttcacgcacaacctcggctctg

SEQ ID NO: 445 - DNA, 23 bp, Canis lupus
gcagcctttggcagcactctctg

SEQ ID NO: 446 - DNA, 26 bp, Canis lupus
cccagaaactttgctaactcctattg

SEQ ID NO: 447 - DNA, 26 bp, Canis lupus
gcctctgtcattcgtgcttccagtgt

SEQ ID NO: 448 - DNA, 23 bp, Canis lupus
gcctctgtcattcgtgcttccag

SEQ ID NO: 449 - DNA, 26 bp, Canis lupus
aagtgcctgttttatggagaactggc

SEQ ID NO: 450 - DNA, 23 bp, Canis lupus
cccttctggaactggatgagccc

SEQ ID NO: 451 - DNA, 23 bp, Canis lupus
cacagccgaagagccactgaagc

SEQ ID NO: 452 - DNA, 60 bp, Homo sapiens
ccatggtgtgagtgtggatttaggtgaatcgaaagatctagtaggttctgtccagactgt

SEQ ID NO: 453 - DNA, 60 bp, Homo sapiens
aattctgagggtggaaggaaggtgggagtcgatggctcttatgcagcattatttatcaat

SEQ ID NO: 454 - DNA, 60 bp, Homo sapiens
aattctgagggtggaaggaaggtgggagtcgagggactttcaggtagaggagccaccaag

SEQ ID NO: 455 - DNA, 60 bp, Homo sapiens
aggggctgatcagtttgtggagttctgatcgagggagaggagtggcagtgggggagtgga

SEQ ID NO: 456 - DNA, 60 bp, Homo sapiens
tccagaagctgagcttgagccaaggtgttcgaactcctgggctgaagcaatctcctgcct

SEQ ID NO: 457 - DNA, 60 bp, Homo sapiens
acgtcgttacagttttaatttttctacttcgatgttaatctcctaaaaaacatccaacca

SEQ ID NO: 458 - DNA, 60 bp, Homo sapiens
caattggtggatatagaaaggtctaaattcgataagtatagactcagaatgcaaaaatgt

SEQ ID NO: 459 - DNA, 60 bp, Homo sapiens; identical to SEQ ID NO: 281

SEQ ID NO: 460 - DNA, 60 bp, Homo sapiens
caccagttggtaattctatgtgtaagtttcgagcttataagatcaatcaggaattattcc

SEQ ID NO: 461 - DNA, 60 bp, Homo sapiens
gcagggtggctatagctcaggagagtgctcgacggagtcttgctctttcacccaggctgg

SEQ ID NO: 462 - DNA, 60 bp, Homo sapiens; identical to SEQ ID NO: 279

SEQ ID NO: 463 - DNA, 60 bp, Homo sapiens; identical to SEQ ID NO: 458

SEQ ID NO: 464 - DNA, 60 bp, Homo sapiens; identical to SEQ ID NO: 461

SEQ ID NO: 465 - DNA, 60 bp, Homo sapiens; identical to SEQ ID NO: 279

SEQ ID NO: 466 - DNA, 60 bp, Homo sapiens
cctaatttacttaaccaaactctagttatcgaacatccaggatgttataagaattcaatg

SEQ ID NO: 467 - DNA, 60 bp, Homo sapiens; identical to SEQ ID NO: 280

SEQ ID NO: 468 - DNA, 60 bp, Homo sapiens
ttttatgaaacatccaacttaaatataatcgaatgcattacatttacagaactatttcca

SEQ ID NO: 469 - DNA, 21 bp, Homo sapiens
cactgcatgagggtagtatag

SEQ ID NO: 470 - DNA, 21 bp, Homo sapiens
tgatgaggcacacagataaag

SEQ ID NO: 471 - DNA, 19 bp, Homo sapiens
gagacatgatgaggcacac

SEQ ID NO: 472 - DNA, 19 bp, Homo sapiens
tggaatgggaagggatgag

SEQ ID NO: 473 - DNA, 21 bp, Homo sapiens
atgaagacagaaagcctatgg

SEQ ID NO: 474 - DNA, 19 bp, Homo sapiens
cggccaggaatgactattg

SEQ ID NO: 475 - DNA, 21 bp, Homo sapiens
agtagtgtatcaggactgggt

SEQ ID NO: 476 - DNA, 21 bp, Homo sapiens
cagcctaccttgcctgacact

SEQ ID NO: 477 - DNA, 20 bp, Homo sapiens
aatcctcttgagcacagacc

SEQ ID NO: 478 - DNA, 20 bp, Homo sapiens
tgttgctagctcaggaagcc

SEQ ID NO: 479 - DNA, 21 bp, Homo sapiens
actggtcacagggaacgatgg

SEQ ID NO: 480 - DNA, 21 bp, Homo sapiens
agtagtgtatcaggactgggt

SEQ ID NO: 481 - DNA, 20 bp, Homo sapiens
tgttgctagctcaggaagcc

SEQ ID NO: 482 - DNA, 21 bp, Homo sapiens
actggtcacagggaacgatgg

SEQ ID NO: 483 - DNA, 25 bp, Homo sapiens
ggtattccaataaatacttgtgccc

SEQ ID NO: 484 - DNA, 23 bp, Homo sapiens
tcacatcagtttctgctctcaag

SEQ ID NO: 485 - DNA, 24 bp, Homo sapiens
tctctgactgcagtgcaaaataat

SEQ ID NO: 486 - DNA, 19 bp, Homo sapiens
cctctgtctgcatcatacc

SEQ ID NO: 487 - DNA, 19 bp, Homo sapiens
acacgcccagaaacaatac

SEQ ID NO: 488 - DNA, 20 bp, Homo sapiens
gtgtgagttgatagctgacc

SEQ ID NO: 489 - DNA, 21 bp, Homo sapiens
gagactccaggcaagaatttg

SEQ ID NO: 490 - DNA, 20 bp, Homo sapiens
cagtggaacttcctgagaac

SEQ ID NO: 491 - DNA, 22 bp, Homo sapiens
gtaagcgaggtcatcatagaag

SEQ ID NO: 492 - DNA, 25 bp, Homo sapiens
tcttggtaaccttgaaaagtttgat

SEQ ID NO: 493 - DNA, 20 bp, Homo sapiens
atgggccatcactgggcttt

SEQ ID NO: 494 - DNA, 18 bp, Homo sapiens
taggcccaaatggctcac

SEQ ID NO: 495 - DNA, 20 bp, Homo sapiens
agatcaagccactgtgctcc

SEQ ID NO: 496 - DNA, 25 bp, Homo sapiens
aggtgtgaatgttactgaacacaaa

SEQ ID NO: 497 - DNA, 25 bp, Homo sapiens
tcttggtaaccttgaaaagtttgat

SEQ ID NO: 498 - DNA, 20 bp, Homo sapiens
agatcaagccactgtgctcc

SEQ ID NO: 499 - DNA, 25 bp, Homo sapiens
aggtgtgaatgttactgaacacaaa

SEQ ID NO: 500 - DNA, 20 bp, Homo sapiens
tactgtgccagatgctctca

SEQ ID NO: 501 - DNA, 20 bp, Homo sapiens
ggagggaggctcagagaagc

SEQ ID NO: 502 - DNA, 25 bp, Homo sapiens
ctccttctacattcacgtgctttca